(19) United States
(12) Patent Application Publication
(10) Pub. No.: US 2025/0305026 A1
(43) Pub. Date: Oct. 2, 2025

(54) SPATIALLY RESOLVED PLANAR AND VOLUMETRIC GENE NEIGHBORHOOD NETWORKS FOR FUNCTIONAL MAPPING OF CELL IDENTITY, COMMUNICATION, AND POTENCY

(71) Applicant: Georgia Tech Research Corporation, Atlanta, GA (US)

(72) Inventors: Ahmet F. Coskun, Atlanta, GA (US); Zhou Fang, Atlanta, GA (US); Nicholas Zhang, Atlanta, GA (US)

(21) Appl. No.: 19/096,894

(22) Filed: Apr. 1, 2025

Related U.S. Application Data
(60) Provisional application No. 63/572,730, filed on Apr. 1, 2024.

(51) Int. Cl. C12Q 1/68; A61K 35/15; A61K 35/28; C12N 5/00; C12N 5/0775; C12N 5/078; G16B 15/00; G16B 35/10; G16B 40/20

(52) U.S. Cl. CPC C12Q 1/68; A61K 35/15; A61K 35/28; C12N 5/0062; C12N 5/0634; C12N 5/0662; G16B 15/00; G16B 35/10; G16B 40/20

(57) ABSTRACT

The present disclosure provides for a method of characterizing a nucleic acid network comprising: providing a biological sample on a substrate, wherein the biological sample comprises the nucleic acid network; applying spatially-aware graph neural networks (spaGNN) to the biological sample to obtain characterization data; and analyzing the characterization data. Also provided herein is a method of providing stem cell therapy to a subject in need thereof comprising: performing the method disclosed herein to a stem cell; and administering the stem cell to the subject. Further disclosed herein is a kit comprising a stem cell targeting reagent and an organelle map, wherein the organelle map comprises data indicating identification and proximity of RNA inside of an organelle and outside of an organelle.

### CROSS REFERENCE TO RELATED APPLICATIONS

This application claims the benefit of U.S. Provisional Application No. 63/572,730, filed Apr. 1, 2024, which is incorporated herein by reference in its entirety.

### STATEMENT OF GOVERNMENT SUPPORT

This invention was made with government support under Grant No. GM142616, awarded by the National Institutes of Health. The government has certain rights in the invention.

### INCORPORATION BY REFERENCE

The contents of the XML file named “10034-347US1-ST26” which was created on Feb. 5, 2025, and is 12 KB in size, are hereby incorporated by reference in their entirety.

### BACKGROUND

Image-based spatial omics methods such as fluorescence in situ hybridization (FISH) generate molecular profiles of single cells at single-molecule resolution. Current spatial transcriptomics methods focus on the distribution of single genes, but fail to analyze the spatial transcriptomics distribution of single genes.

The methods and kit disclosed herein address these and other needs.

### SUMMARY

In accordance with the purposes of the disclosed materials and methods, as embodied and broadly described herein, the disclosed subject matter, in one aspect, relates to stem cells and methods related thereto.

In one example, a method of characterizing a nucleic acid network is provided, including providing a biological sample on a substrate, wherein the biological sample comprises the nucleic acid network; applying spatially-aware graph neural networks (spaGNN) to the biological sample to obtain characterization data; and analyzing the characterization data.

In a further example, a method of providing stem cell therapy to a subject in need thereof is provided, including performing the method disclosed herein to a stem cell; and administering the stem cell to the subject.

Additionally, a kit is provided, including a stem cell targeting reagent and an organelle map, wherein the organelle map comprises data indicating identification and proximity of RNA inside of an organelle and outside of an organelle.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIG. 1 shows spatially resolved planar and volumetric gene neighborhood networks for functional mapping of cell identity, communication, and potency.

FIG. 2 shows anti-RBP-priming of stem cells for controlling cell depositomics in regenerative medicine. Cells are activated with an RNA-binding protein (RBP) to increase the ECM depositions. A multiplex RNA-protein proximity assay will be developed. Live ECM deposition will be acquired from stem cells in 3D hydrogels. A tendon repair model will be used to evaluate the spatially resolved RBPs in rat tissues.

FIG. 3 shows collagen-I regulation by RBPs. ER, LARP6, CRTH2, Vimentin, and Myosin control collagen-I depositomics at the transcriptional level.

FIGS. 4A-4B shows spatial genomics.

FIG. 4A shows Sequential FISH-HCR imaging of 16 gene targets in single cells.

FIG. 4B shows a Small portion of HCH was shown. (Green/Magenta).

FIGS. 5A-5D shows spatial gene-protein neighborhoods indicate ConA (ER) and collagen 1 interactions.

FIG. 5A shows RNA images of collagen and ACTB genes in HUC.

FIG. 5B shows RNA images of collagen and ConA in HCH.

FIG. 5C-D show Collagen is enriched on ER-based ConA stains.

FIGS. 6A-6E shows spatial gene-protein neighborhoods indicate ConA (ER) and collagen 1 interactions.

FIG. 6A shows SpaGNN model of a single HCH cell.

FIG. 6B shows Multiplexing data in an HCH cell.

FIG. 6C-6E show Collagen interacts with ER/ConA.

FIG. 7 shows multiplexed RNA-Protein Interaction (mRPI) method. Multiple cycles are performed to profile proteins that are binding to RNA targets. Cycle 1 detects RNA1 and Protein 1 interactions. Cycle 2 detects RNA 1 and Protein 2 interactions. Cycle 3 detects RNA 2 and Protein 3 interactions. This modular design works by constructing RPI assemblies using proximity ligation assay (PLA) and a DNase I assay to digest the PLA assembly. The following cycle then constructs and digests again. After constructing the RPI assembly, the images are acquired. After digesting, only the background cell is imaged. This design will enable interactions of collagen 1 and LARP6 and vimentin, for instance, for depositomics.

FIG. 8 shows probe design of proximity ligation for protein-protein interactions. Sequence information and step-by-step fluorescent amplification are outlined.

FIG. 9 shows spatial genomics in 3D PEG-based Hydrogels. Ten genes were multiplexed to target β-actin, IL8, IL6, CCL11, SOX9, EEF2, SPP1, COL1A1, RUNX1, and PDL1 in BM-MSCs.

FIG. 10 shows lattice light sheet imaging. Microscale deposition profiling in 3D hydrogel.

FIG. 11 shows in vivo tendon model using rotary cuff repair to screen depositomics. MSCs or Anti-RBP primed MSCs will be directly injected to the tendons during the reattachment surgery, two weeks after original tendon injury. N=390 mice.

FIG. 12 shows spatial transcriptional analysis of c γMSC potency in the 3D hydrogel. T-cell suppression assays via γMSC on 3D PEG-4MAL hydrogels. Aim 1: γMSC potency by spatial transcriptomics and proteomics. Aim 2: γMSC Potency comparisons to the outcome of GVHD response to γMSC and T cell infusions.

FIG. 13 shows sequential FISH-HCR in MSCs. Glass coverslips coated with collagen gel enables cell adherence. Cell types of interest, namely HUC (n=121) and HBM (n=237), were seeded, fixed, and processed via multiplexed RNA-FISH and followed by cell painting for protein analysis. Hybridization chain reaction (HCR) was used to enable single-molecule RNA detection in MSCs. Representative images were shown for 12 genes in HBM cells. The quantification of RNA count per cell yielded higher gene expression in HUC than HBM.

FIG. 14 shows spatial protein profiling of mammalian cells in cancer tissues. Multiplexed CODEX proteomic data in thin tissues sections from chronic lymphocytic leukemia (CLL) biopsies. A subset of 20-markers was shown in pseudo-colored images in each panel.

FIG. 15 shows spatial multi-omics. EZH2 AND H3K27me3 proteins were multiplexed with COL1A1 and B-Actin RNA expression in the same tissue.

FIG. 16 shows RNA-seqFISH-HCR in 3D Hydrogels. Twenty-eight genes were multiplexed in BM- and UC-MSCs. The hydrogel containing PEG-MAL gel. Images are from BM-MSCs. Single cell quantification of gene expression per marker.

FIGS. 17A-17C show multiplexed gene expression comparisons of MSCs and γMSC. Five genes were measured in MSCs and γMSC. COL1A1 is a collagen depositome gene, IL8 and IL6 are the cytokines, PDL1 is the immunomodulatory gene, and ACTB is the housekeeping gene. γMSC exhibited higher COL1A1, IL6, and IL8 gene expression, while there was no significant difference in ACTB and PDL1 gene expression. Microfluidic IFN-γ treated BM MSC n=15 and Microfluidic untreated BM MSC n=15 across 3 donors performed in 3D hydrogel-based potency chip. Star significance for P<0.005; ns is non-significant, p>0.05. Z sections were optically acquired with Nikon microscope using 60× magnification.

FIG. 18 shows spatial RNA imaging of MSC-immune co-culture in the 3D gel. ActB, IL6, and IL8 RNA markers were acquired by 60× magnification. Four (4) Field-of-views were shown.

FIG. 19 shows 3i lattice light-sheet imaging of RNA gene expression in MSCs. Single MSCs mounted on 5 mm round coverslips were imaged by 3i Lattice light-sheet, providing single molecule sensitive at potentially >500 μm depth.

FIG. 20 shows validation of the SpatialVizScore immunoscore tool rationale on breast cancer H&E images with ICI response data. Annotating tumor regions on patients' H&E images (left), classifying cell phenotypes based on their morphologies, generating their network, and quantifying their neighborhood score. Responder patients scored higher immunoscores than non-responder patients (right).

FIG. 21 shows data analysis. RNA & Protein data integration using Seurat-v4.0. Multivariate model optimizes spatial data, predicting auto/para signaling.

FIGS. 22A-22I show a spatially resolved gene neighborhood network analysis pipeline reveals gene proximity relationship in a single cell.

FIG. 22A shows Visualization of MERFISH data in fibroblast. Each dot in the scatterplot is a detected RNA transcript.

FIG. 22B shows Patch separation of transcripts using the Leiden clustering algorithm.

FIG. 22C shows Patch correlation map of the presented fibroblast. The heatmap includes 130 genes labeled in the published dataset.

FIG. 22D shows A portion of the patch correlation heatmap.

FIG. 22E shows Visualization of gene pairs with positive and negative correlations. FBN2-FLNA, PRPF8-SRRM2, and SRRM2-MKI67 all have positive correlations, while MALAT1-FLNA, PRPF8-NUMA1, and THBS1-MALAT1 all have negative correlations. Gene pairs with positive correlations localize in similar subcellular positions, while gene pairs with negative correlations localize in distinct subcellular positions.

FIG. 22F shows Local neighborhoods in a patch near the nucleus. The scatterplot indicates the RNA location in the patch. A local neighborhood is highlighted by the connection between the center RNA and its neighbors.

FIG. 22G shows Gene neighborhood networks of the patch shown in FIG. 22F. The edge color indicates the proximity score between the gene pair, and the size of the node indicates the enrichment of the gene in the patch.

FIG. 22H shows A subpart of the gene neighborhood networks.

FIG. 221 shows Visualization of gene pairs with positive and negative proximity scores. TEAD1-LRP1, IGF2R-FBN2, and FLNA-FBN2 all have positive proximity scores, while TNC-FLNC, PRPF8-COL5A1, and THBS1-MALAT1 all have negative correlations.

FIGS. 23A-23E show a patch-level analysis reveals subcellular heterogeneity in spatial transcriptomic profiles.

FIG. 23A shows Transcript location and patch correlation of genes in fibroblast. The scatterplot shows the location of transcripts of the labeled genes. The heatmap shows the patch correlation between the labeled genes.

FIG. 23B shows Transcript location and patch correlation of genes in U2-OS. The scatterplot shows the location of transcripts of the labeled genes. The heatmap shows the patch correlation between the labeled genes. Several differences in patch correlation between the two cells are shown, such as PRPF8-IGF2R, TPR-LRP1, and PRPF8-PRKCA.

FIG. 23C shows RRM2 and PRPF8 visualization in fibroblast and U2-OS. The gene pair presents similar patch correlations in both cells.

FIG. 23D shows RPF8 and IGF2R visualization in fibroblast and U2-OS. The gene pair presents opposite patch correlations.

FIG. 23E shows Boxplot of patch correlations between SRRM2-PRPF8 and PRPF8-IGF2R in fibroblast and U2-OS. p values: **: 0.001<p<0.01, ****: p<=0.0001.

FIGS. 24A-24G show SpaGNN reveals subcellular gene neighborhood network variability.

FIG. 24A shows Two patches in a fibroblast. The nearest neighborhood networks of the two patches are shown in FIG. 24C.

FIG. 24B shows Two patches in a U2-OS cell.

FIG. 24C shows Gene neighborhood networks of the two patches shown in FIG. 24A between THBS1, FBN2, and SRRM2. THBS1-FBN2 has a positive proximity score in the left patch and a negative proximity score in the right patch.

FIG. 24D shows Gene neighborhood networks of the two patches shown in FIG. 24B between THBS1, FBN2, and SRRM2. THBS1-FBN2 has a negative proximity score in the left patch and a positive proximity score in the right patch. Furthermore, THBS1-SRRM2 has a lower proximity score in the right patch than in the left patch.

FIG. 24E shows Visualization of THBS1, FBN2, and SRRM2 in the two patches shown in FIG. 24A. The scatterplot shows the position of transcripts, and the edge indicates detected neighbors.

FIG. 24F shows Visualization of THBS1, FBN2, and SRRM2 in the two patches shown in (B).

FIG. 24G shows Distribution of proximity scores of THBS1-FBN2, THBS1-SRRM2, and FBN2-SRRM2 among all patches of fibroblast and U2-OS. The proximity scores in the fibroblast shown are higher than in the U2-OS cell shown in this figure for all three pairs of genes. p values: ns: 0.05<p<=1.00, *: 0.01<p<=0.05, ****: p<=0.0001.

FIGS. 25A-25F show patch correlation and gene neighborhood network variability achieve better separation of cell types than single-cell RNA count.

FIG. 25A Cell type and clustering based on single-cell gene count on a t-SNE plot. The counts of each RNA type per cell were used as features for t-SNE and clustering of fibroblast (n=53) and U2-OS (n=53).

FIG. 25B shows Cell type and clustering based on patch correlation on a t-SNE plot. Marker pairwise correlations between patches of the same cell were used as features for UMAP and clustering of fibroblast and U2-OS.

FIG. 25C shows Cell type and clustering based on network variability visualized on a t-SNE. The mean and standard deviation of proximity score between marker pairs were used as features for t-SNE and clustering of fibroblast and U2-OS.

FIG. 25D-25F show Comparison of classification of fibroblasts and U2-OS using RNA count, patch correlation, and neighborhood network variability. All cells in the same cluster are predicted to be the same cell type as the majority of cell types in the cluster. The predicted cell type was then compared with the true cell type using confusion matrices. RNA count, patch correlation, and neighborhood network variability all classified the two cell types with high accuracy.

FIGS. 26A-26E show SpaGNN analysis reveals subcellular gene proximity relationships in HBM, HUC, and HCHs.

FIG. 26A shows Multiplexed RNA profiles of HUC, HBM, and HCH. The data were collected using HCR-based SeqFISH.

FIG. 26B shows Leiden clustering of positions of RNA molecules in an HUC cell. The presented cell contains 57 patches.

FIG. 26C shows Mean correlation map of HUCs (n=121). COL1A1 shows negative correlations with GAPDH and ACTb. EEF2 shows positive correlations with GADH and ACTb.

FIG. 26D shows Local neighborhoods in a patch near the nucleus and corresponding gene neighborhood network. The scatterplot indicates the transcripts' location in the patch. A local neighborhood is highlighted by the connection between the center transcript and its neighbors.

FIG. 26E shows Local neighborhoods in a patch away from the nucleus and the corresponding gene neighborhood network. The patch shows a low concentration of COL1A1.

FIGS. 27A-27C show distinct cell types exhibit heterogeneous gene neighborhood networks. Gene neighborhood networks of an HBM (FIG. 27A), an HUC (FIG. 27B), and an HCH (FIG. 27C). Cells present heterogeneous marker enrichment and connectivity between patches. The same marker pairs exhibit different connections in different patches.

FIGS. 28A-28F show patch correlation and gene neighborhood network achieve higher separability than single-cell RNA count to distinguish HBM, HUC, and HCH cell populations.

FIG. 28A shows Cell clustering and cell types based on single-cell RNA count visualized on a t-SNE plot. The count of each RNA type per cell was used as a feature for t-SNE and clustering of the HUCs (n=121), HBMs (n=237), and HCHs (n=247).

FIG. 28B shows Cell clustering and cell types based on patch correlations visualized on a t-SNE. Marker pairwise correlations between patches of the same cell were used as features for t-SNE and clustering of HUC, HBM, and HCH.

FIG. 28C shows Cell clustering and cell types based on network variability visualized on a t-SNE. The mean and standard deviation of connection between marker pairs were used as features for t-SNE and clustering of HUC, HBM, and HCH.

FIGS. 28D-28F show Comparison of classification of HBM, HUC, and HCH using RNA count (FIG. 28D), patch correlation (FIG. 28E), and neighborhood network variability (FIG. 28F). All cells in the same cluster are predicted to be the same cell type as the majority of cell types in the cluster. The predicted cell type was then compared with the true cell type using confusion matrices. Classification based on patch correlation and network variability is less likely to predict HUCs and HCHs as HBMs, and improved classification sensitivity and specificity of HUCs.

FIG. 29 shows a graphical abstract of subcellular spatially resolved gene neighborhood networks in single cells.

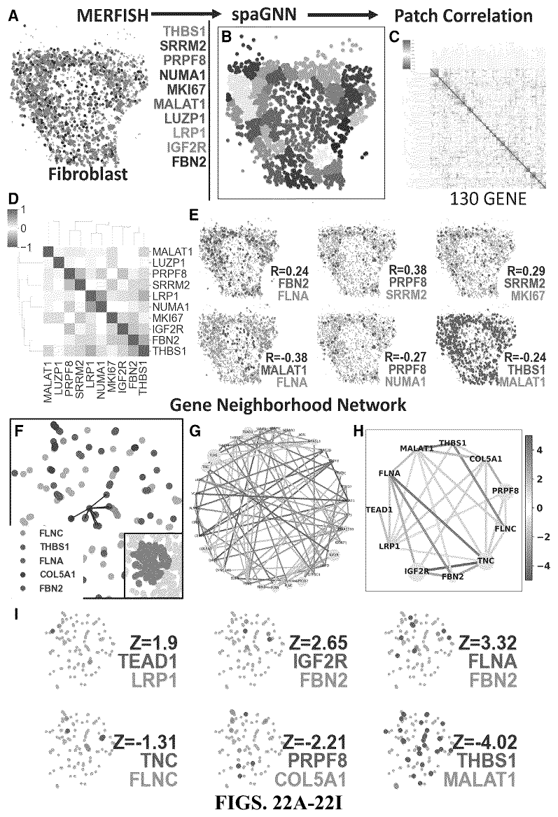

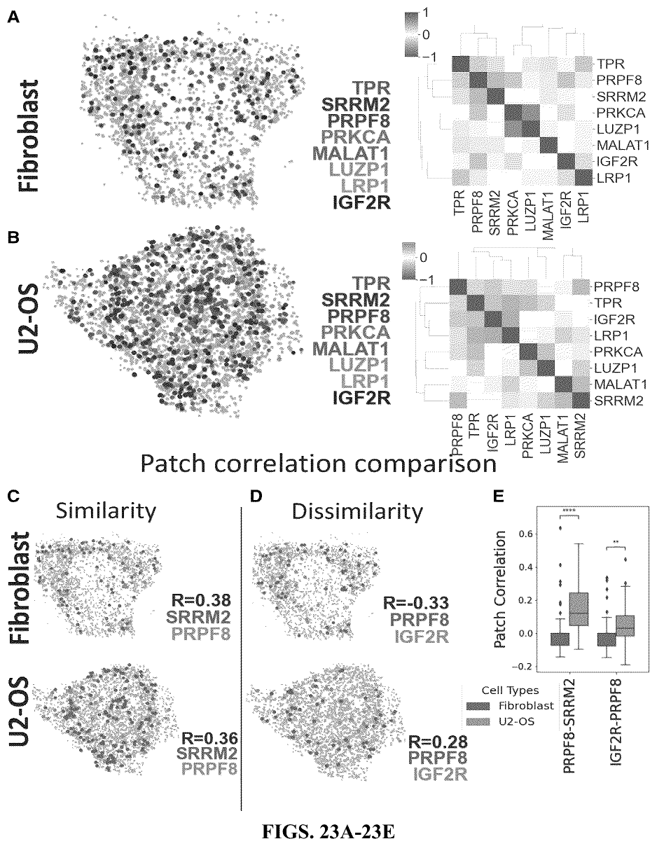

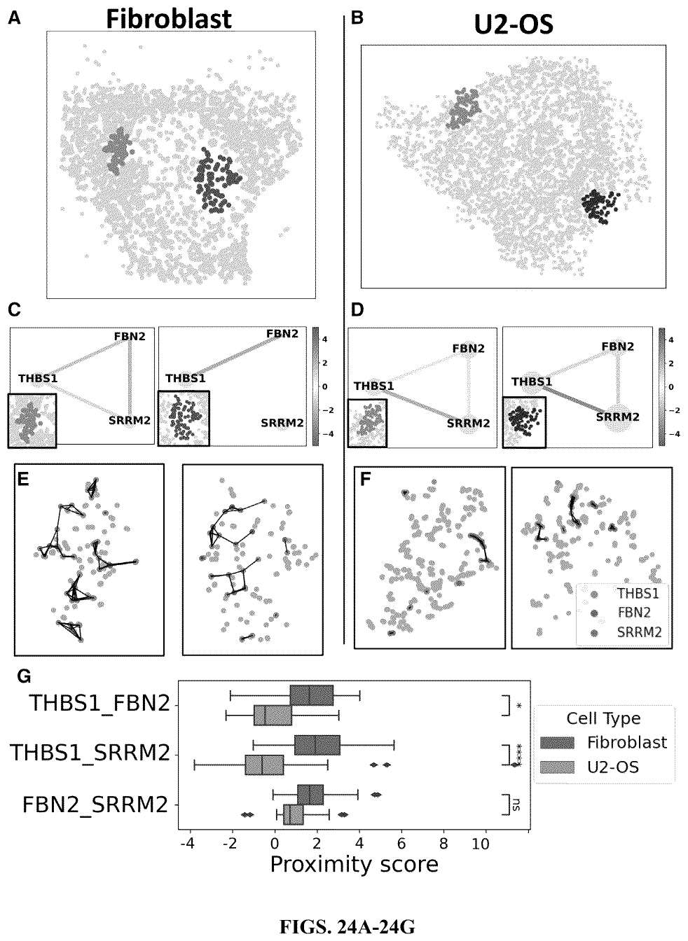

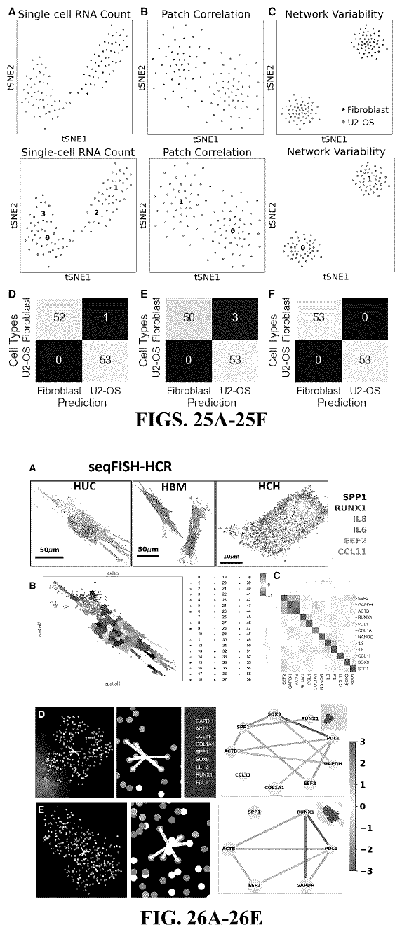

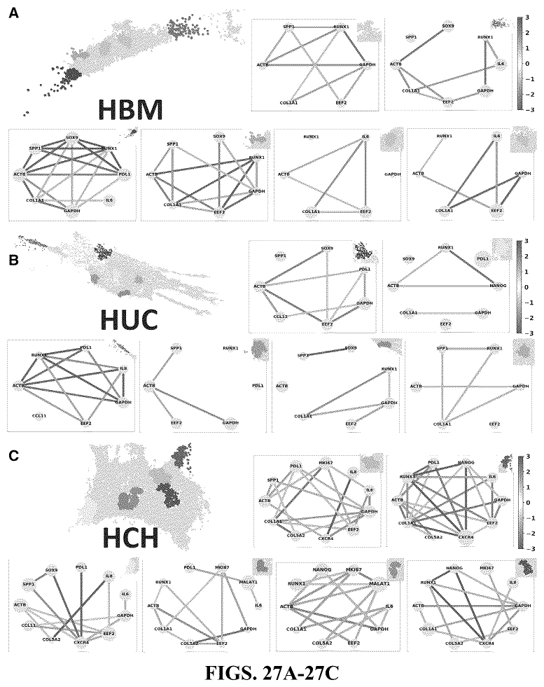

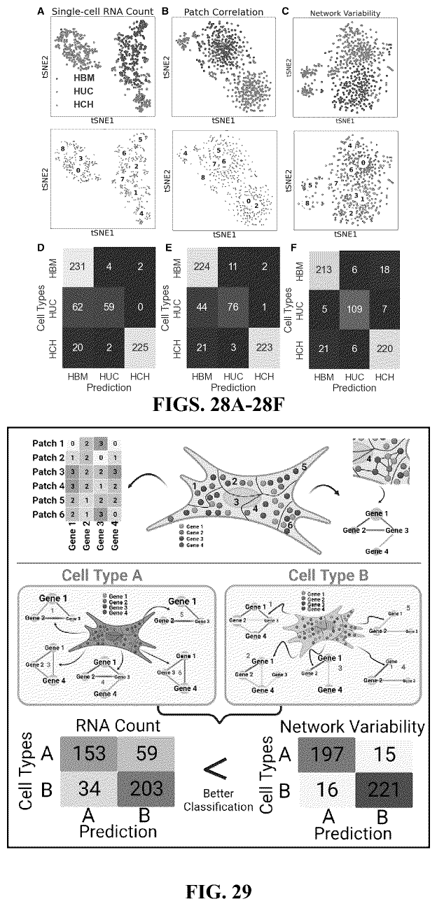

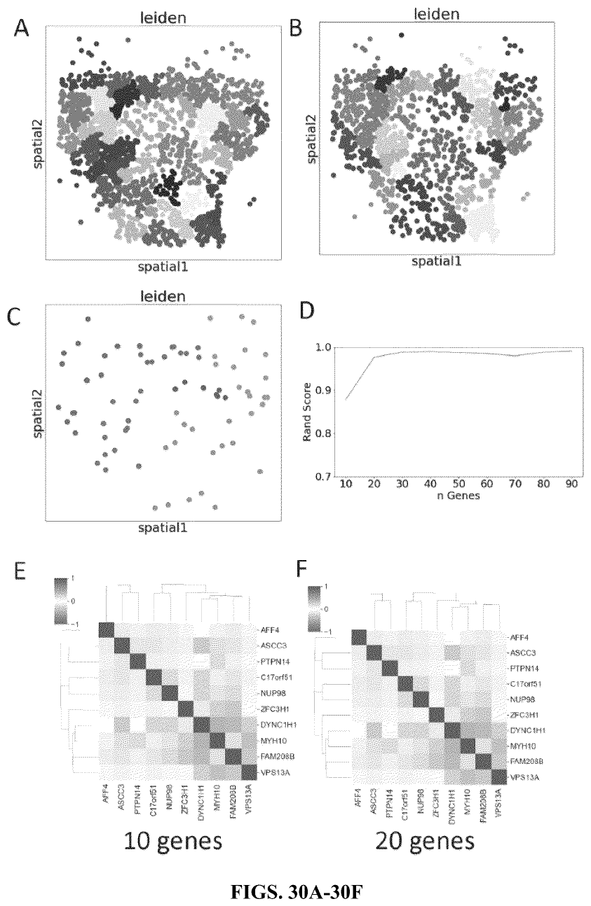

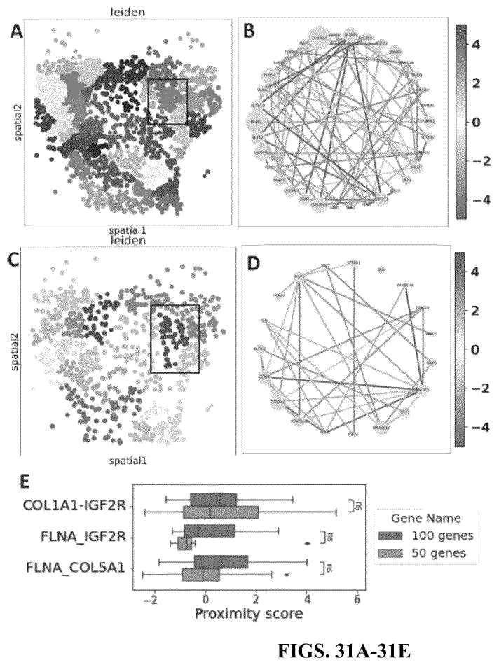

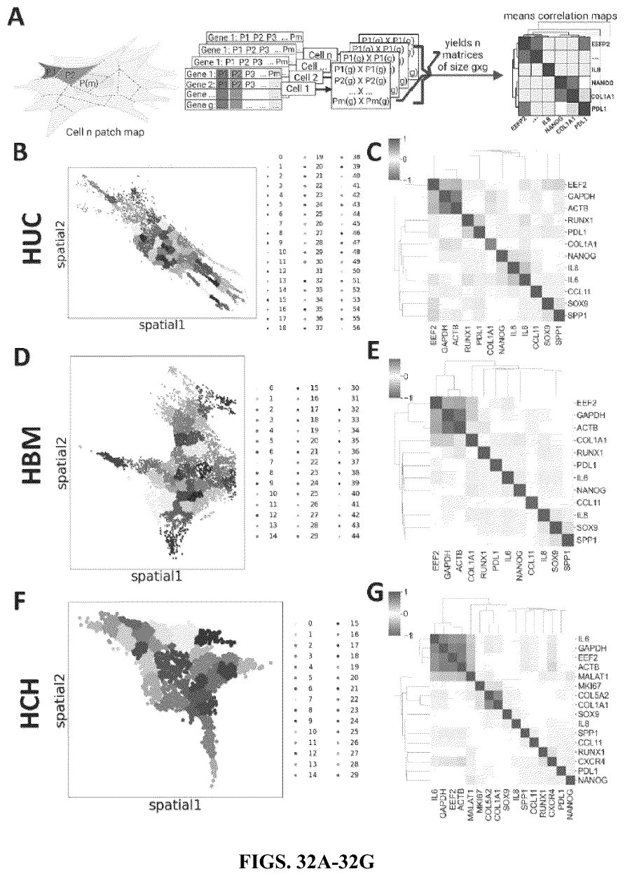

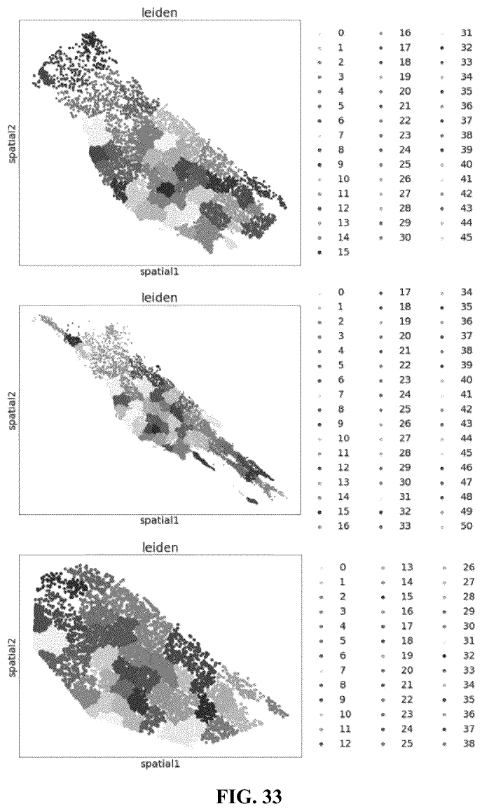

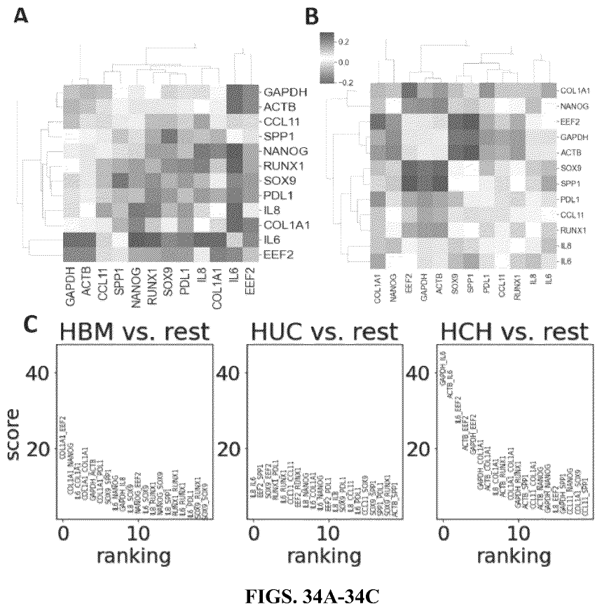

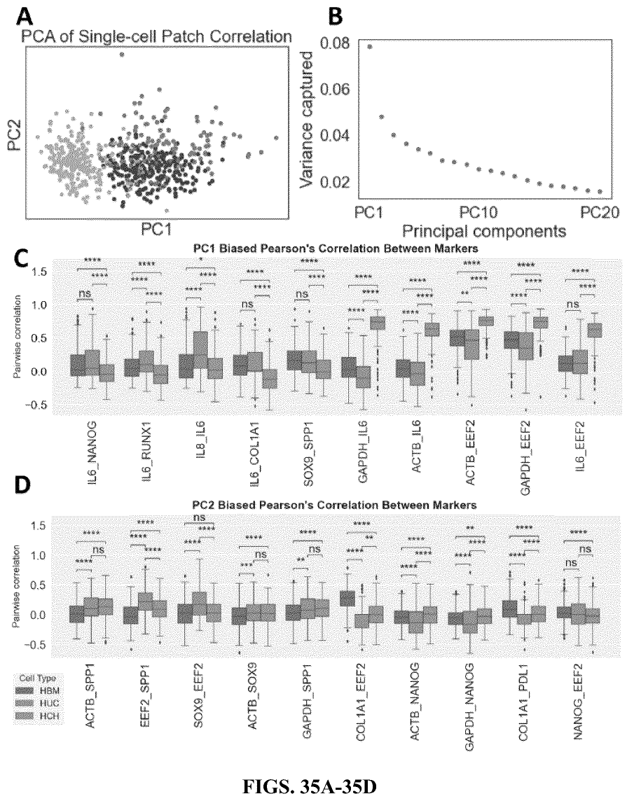

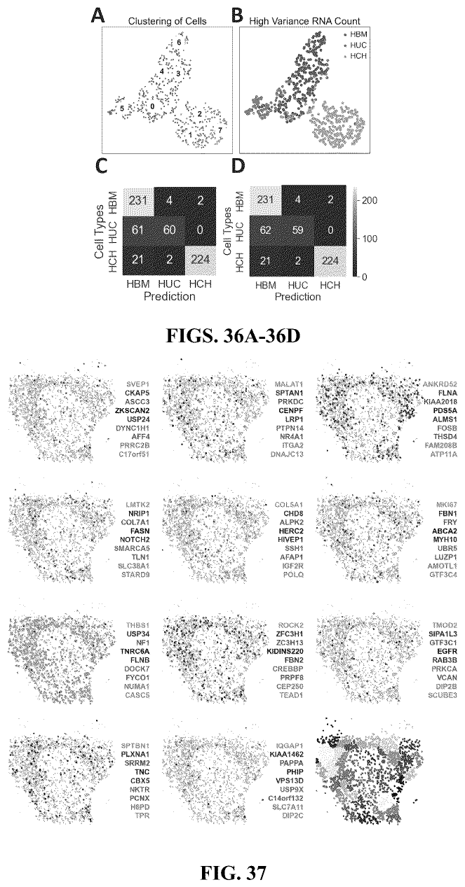

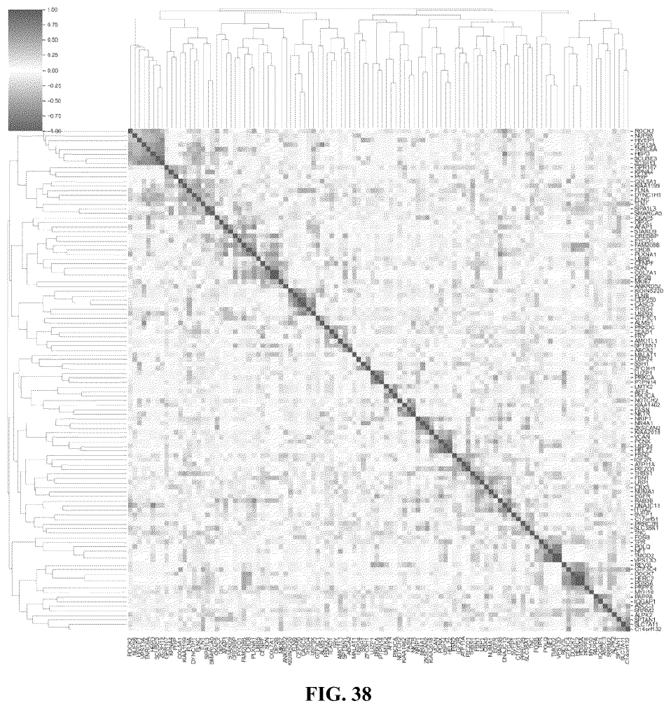

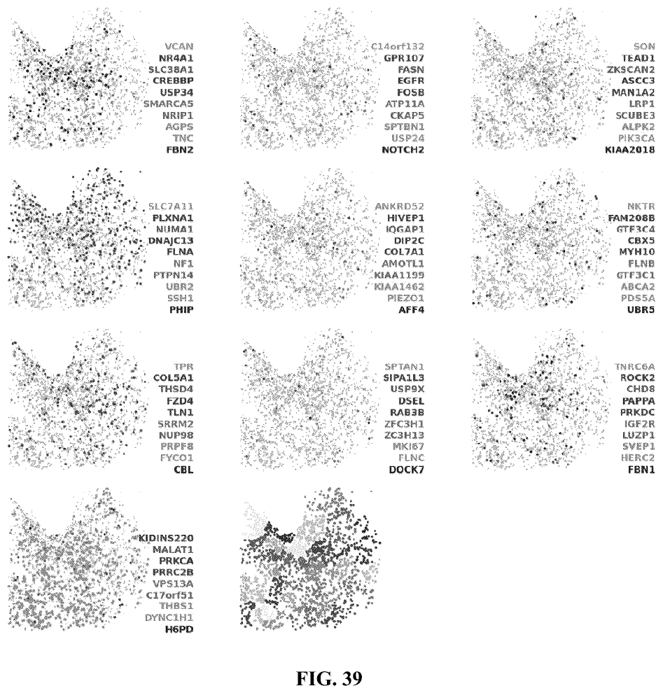

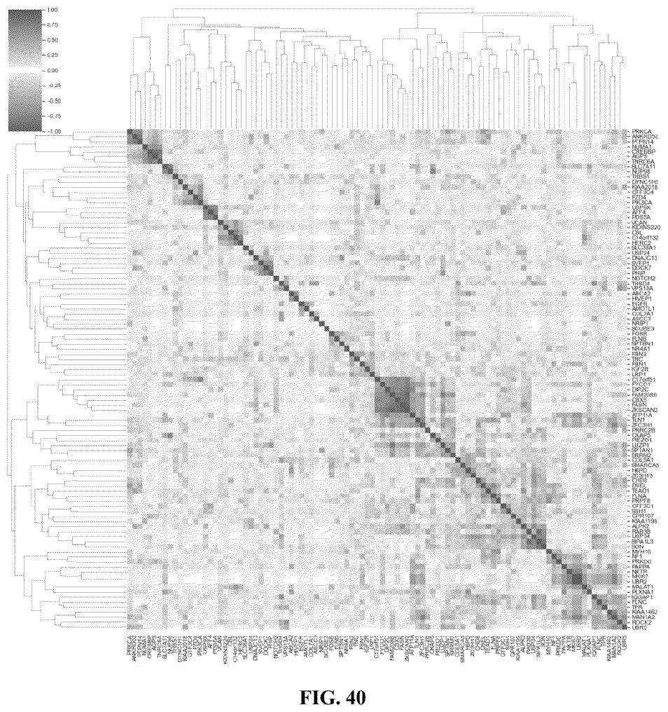

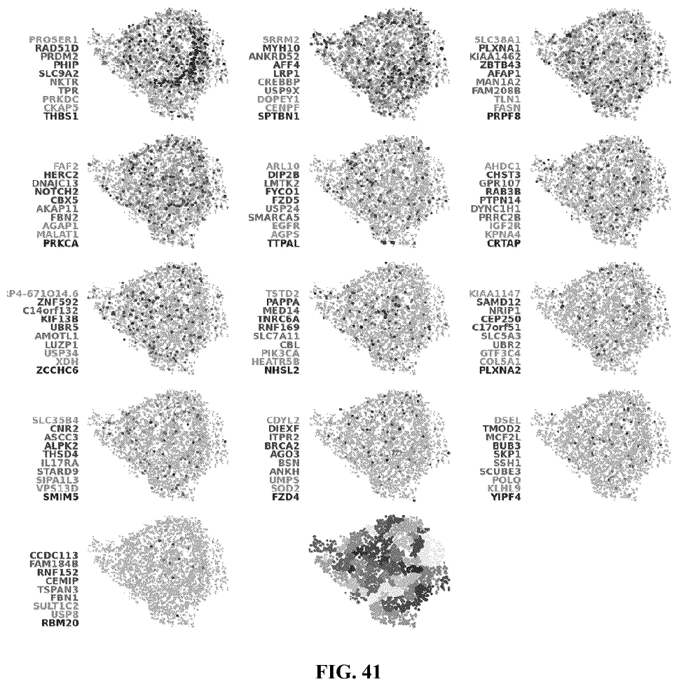

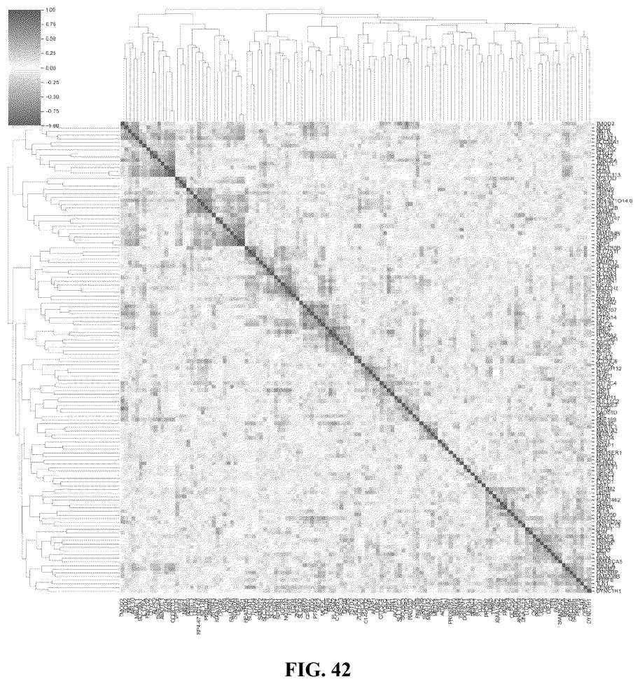

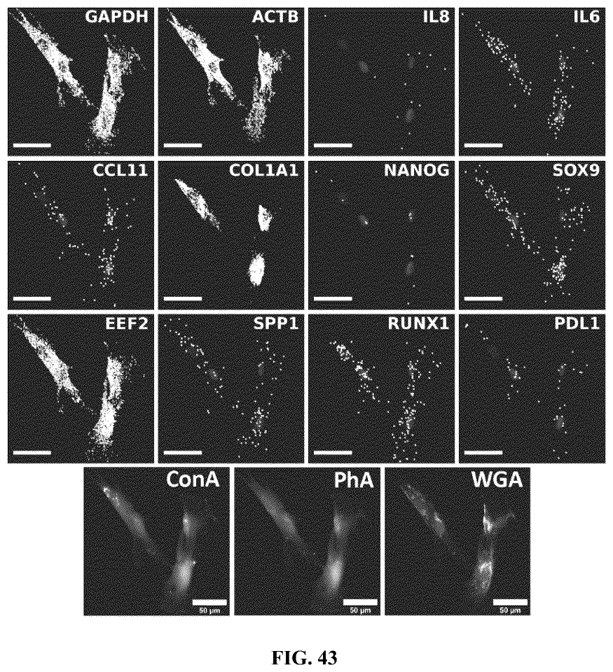

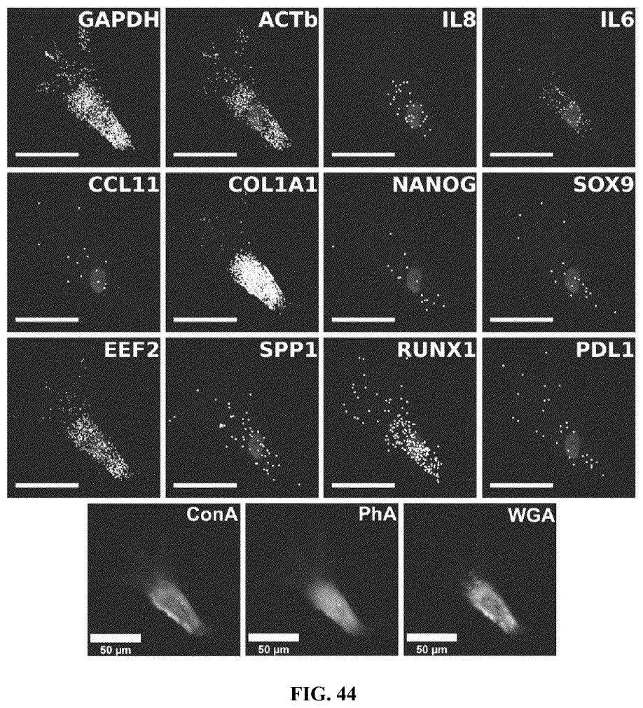

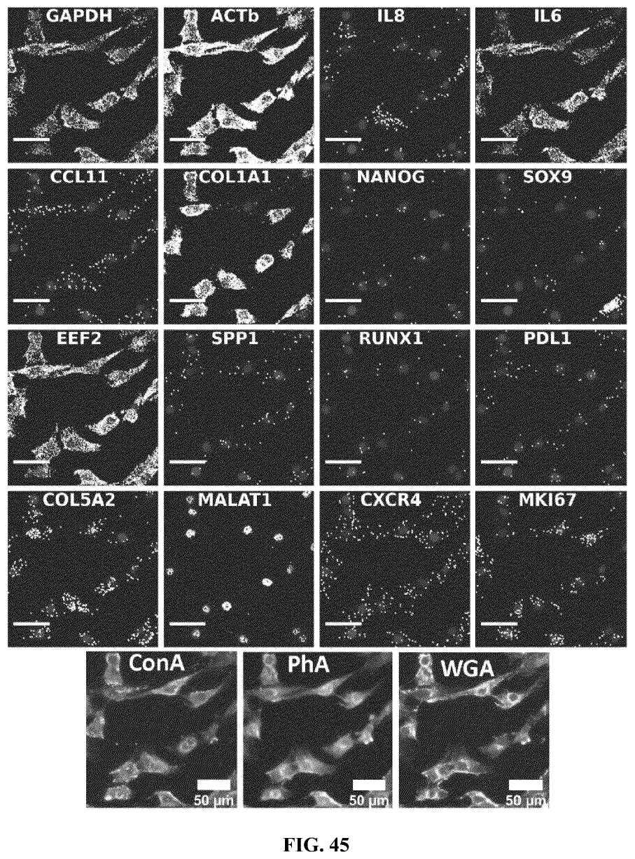

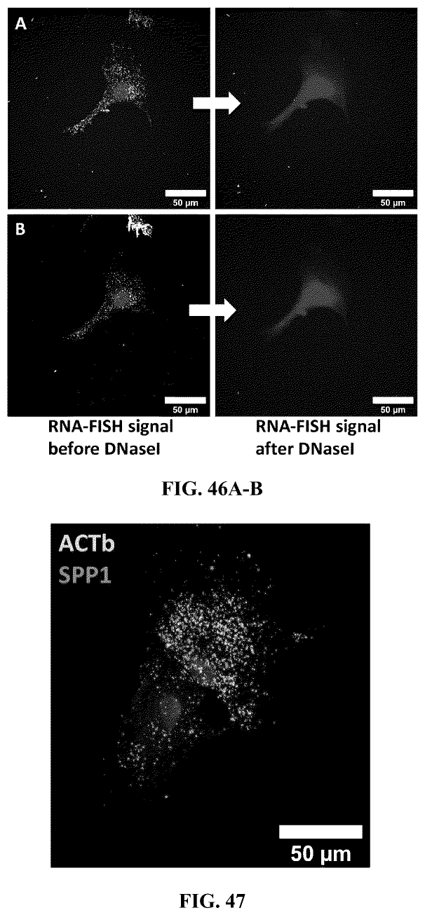

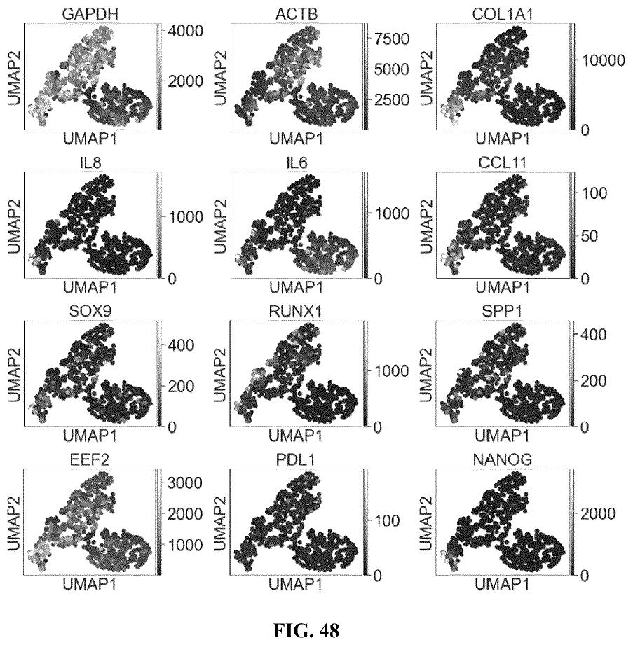

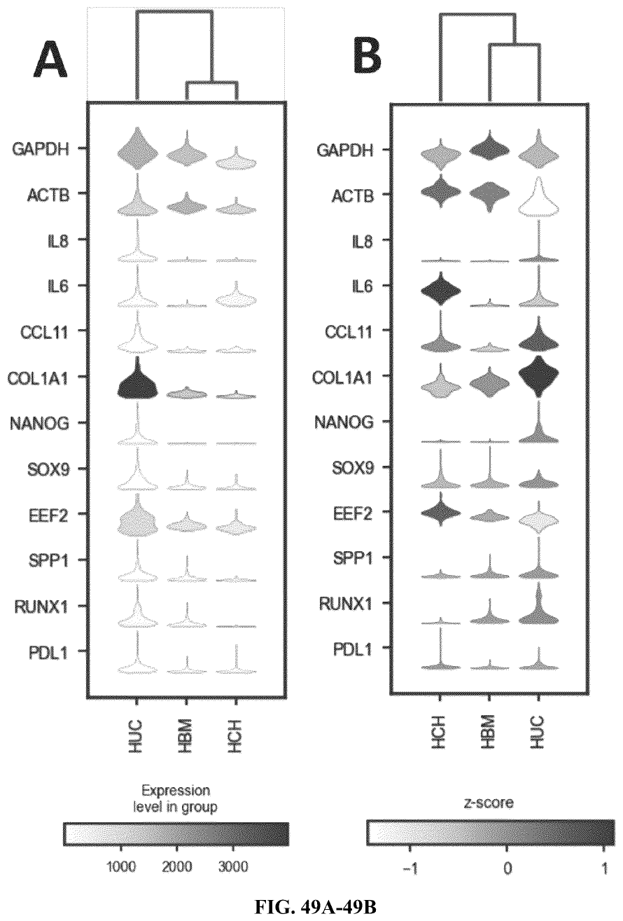

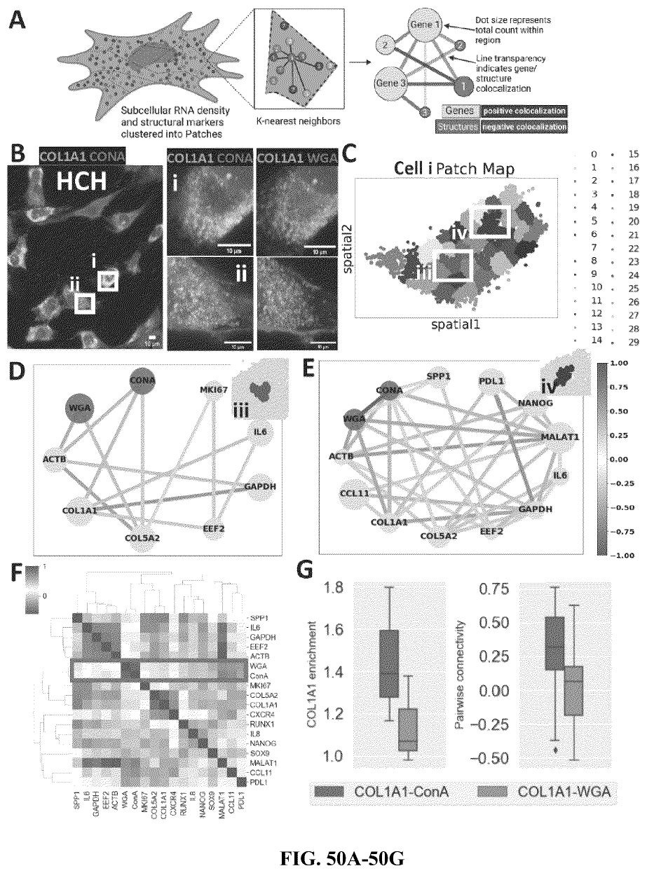

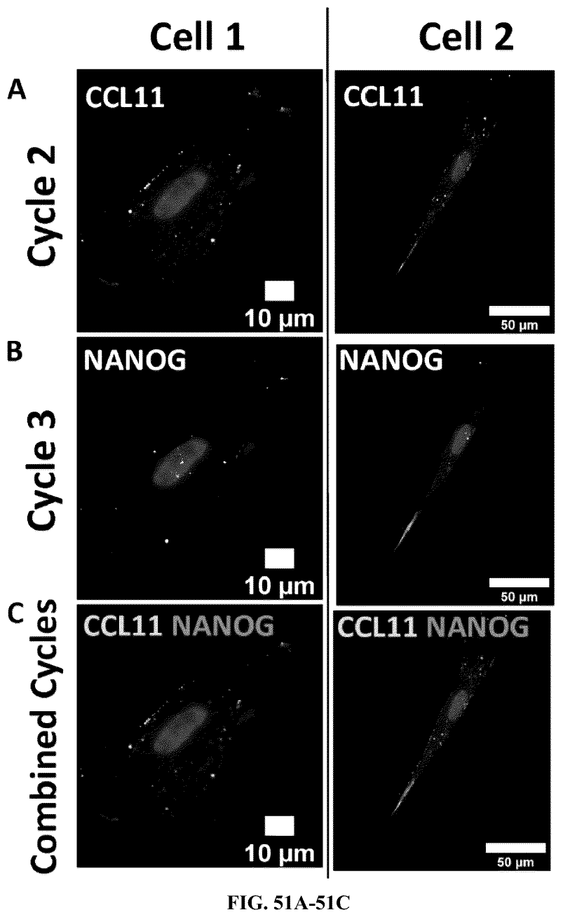

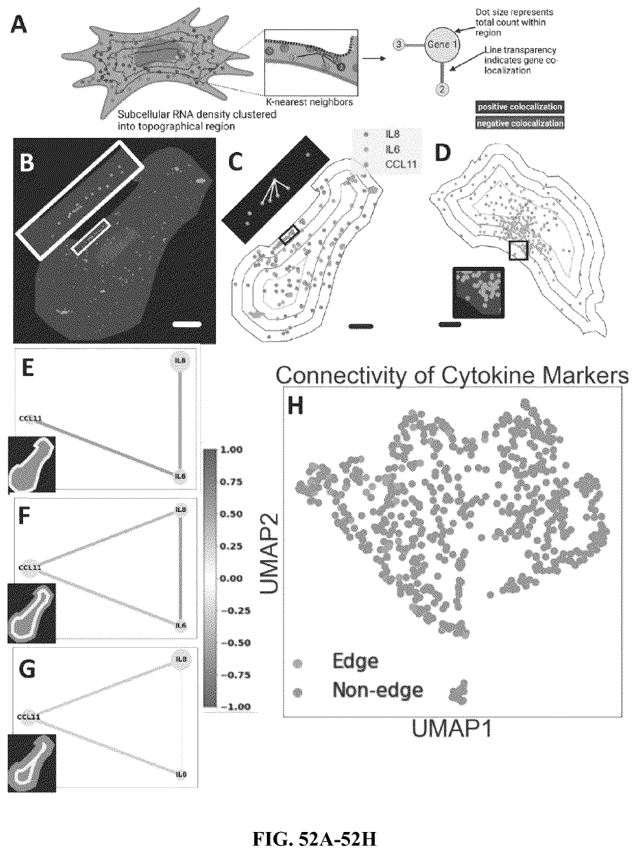

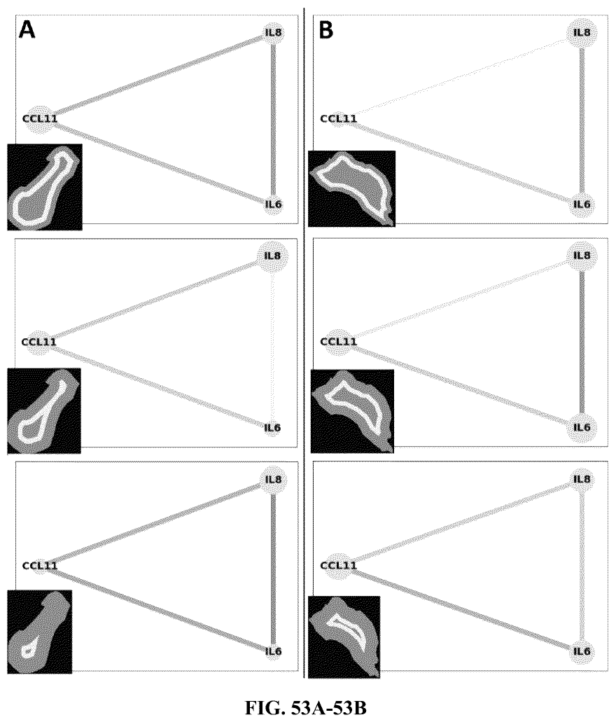

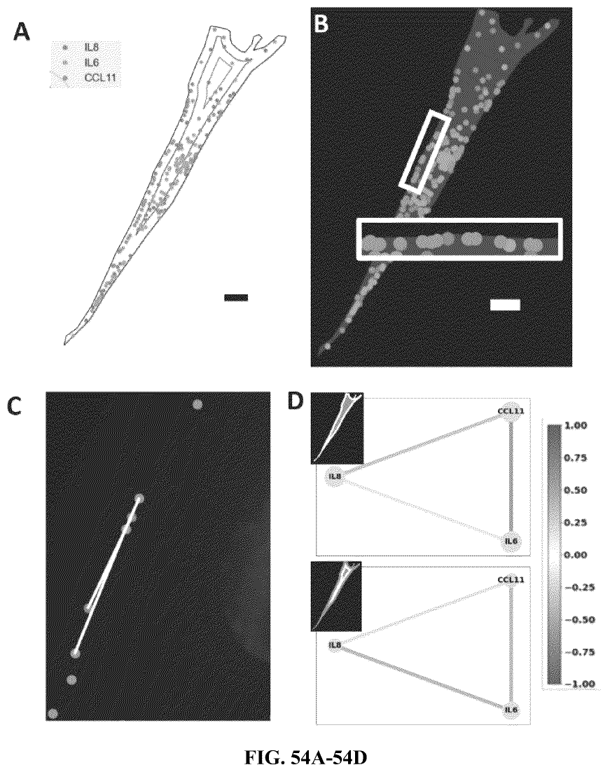

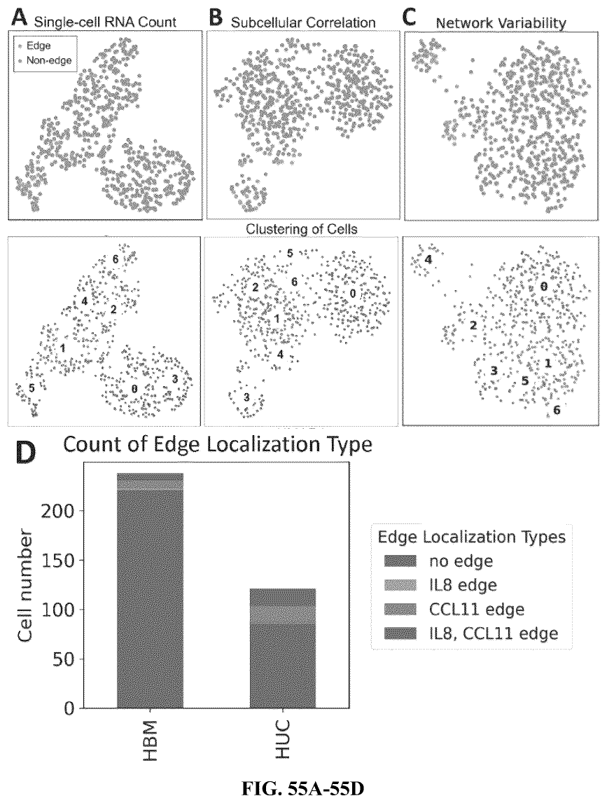

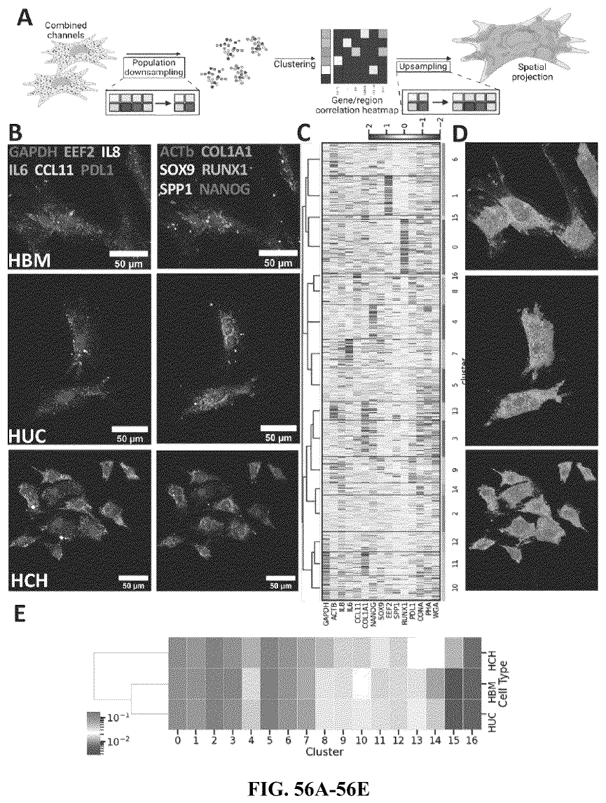

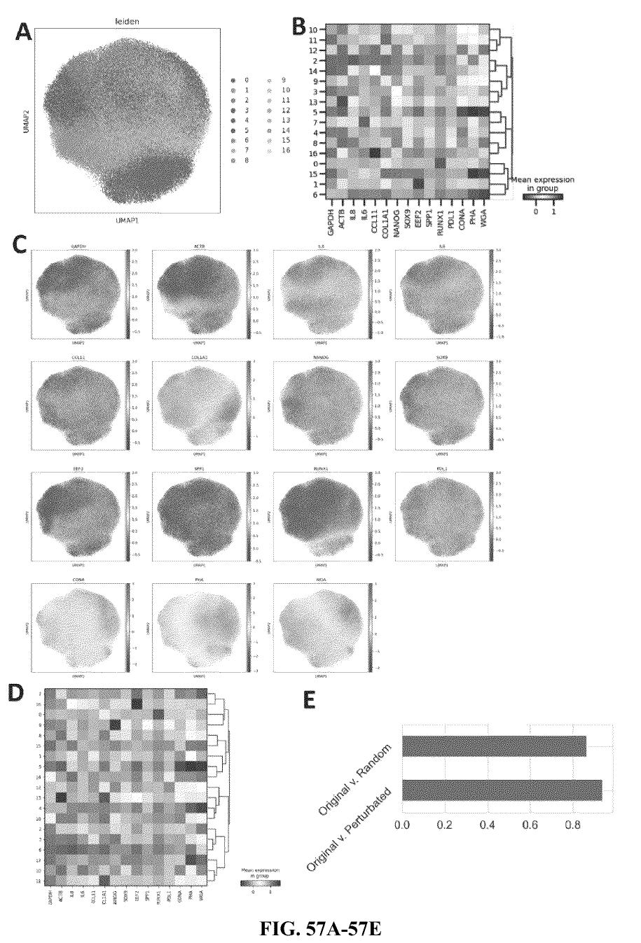

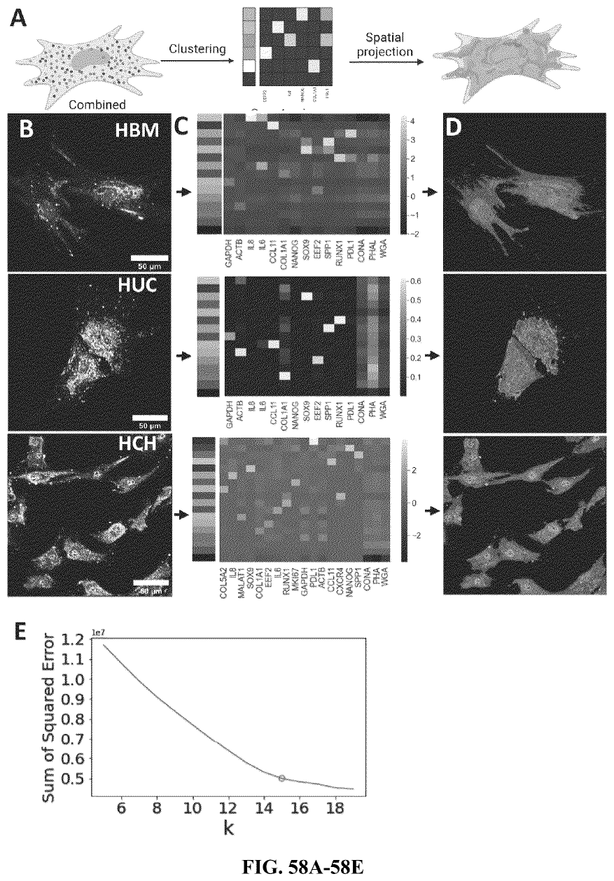

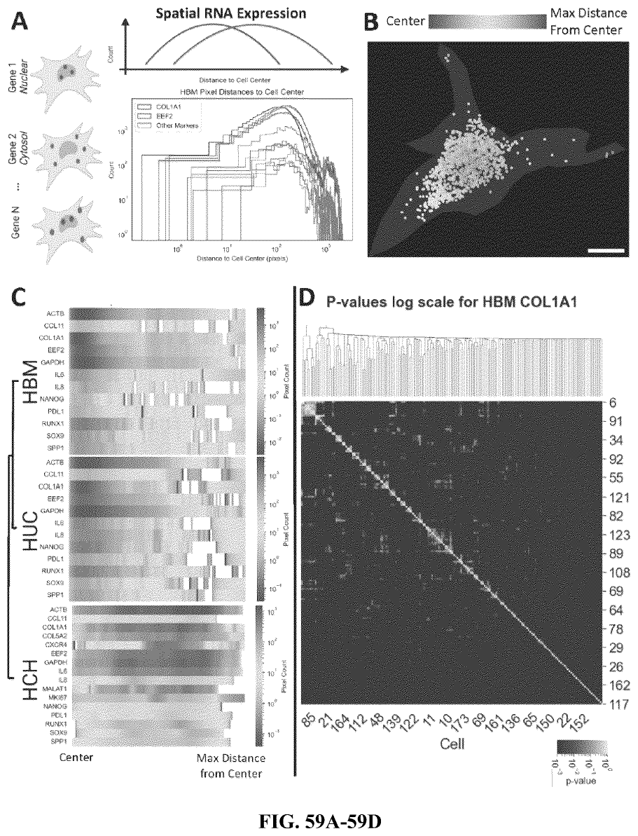

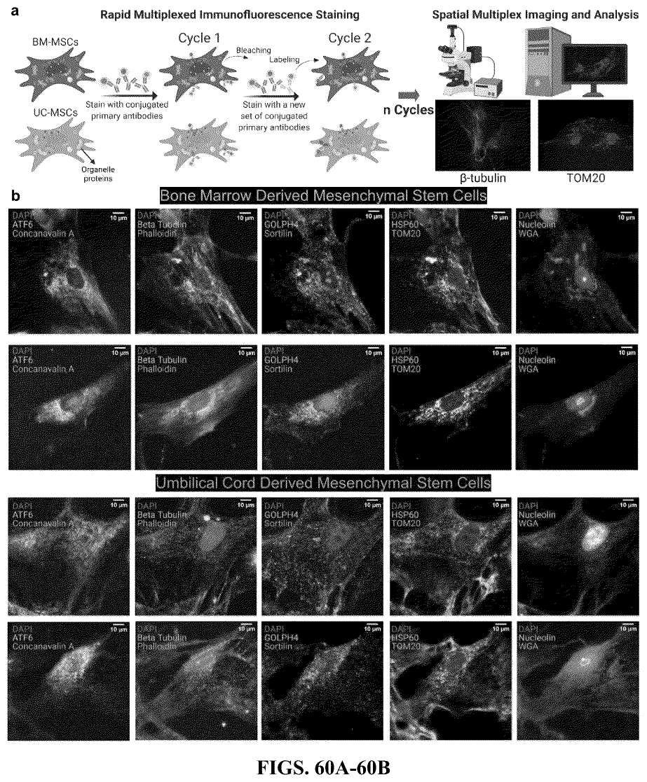

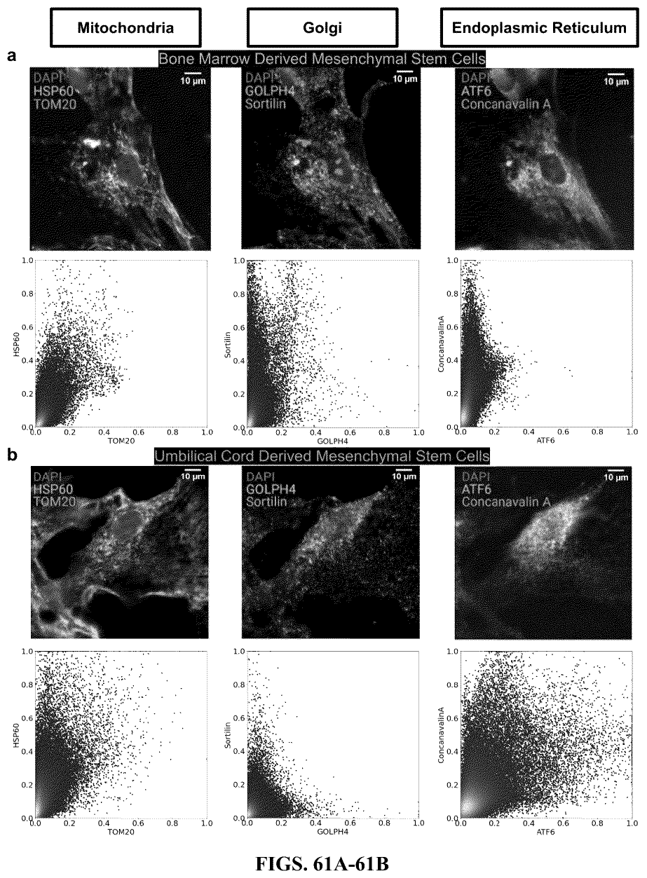

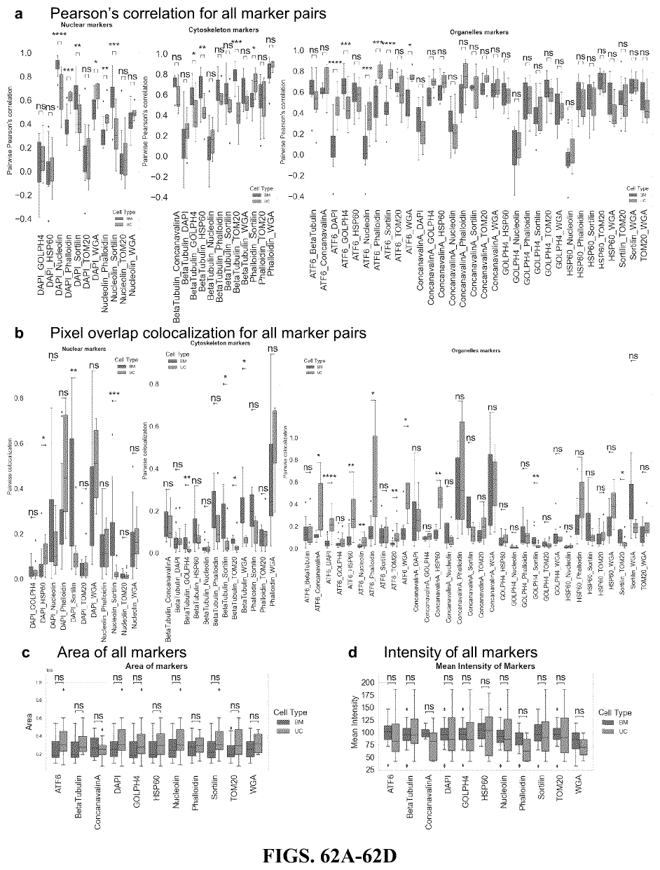

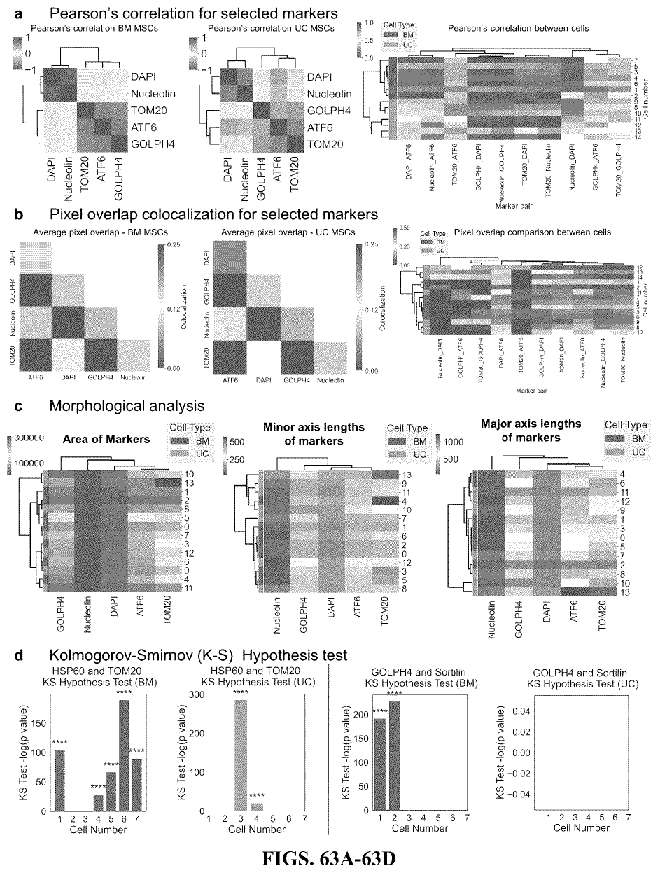

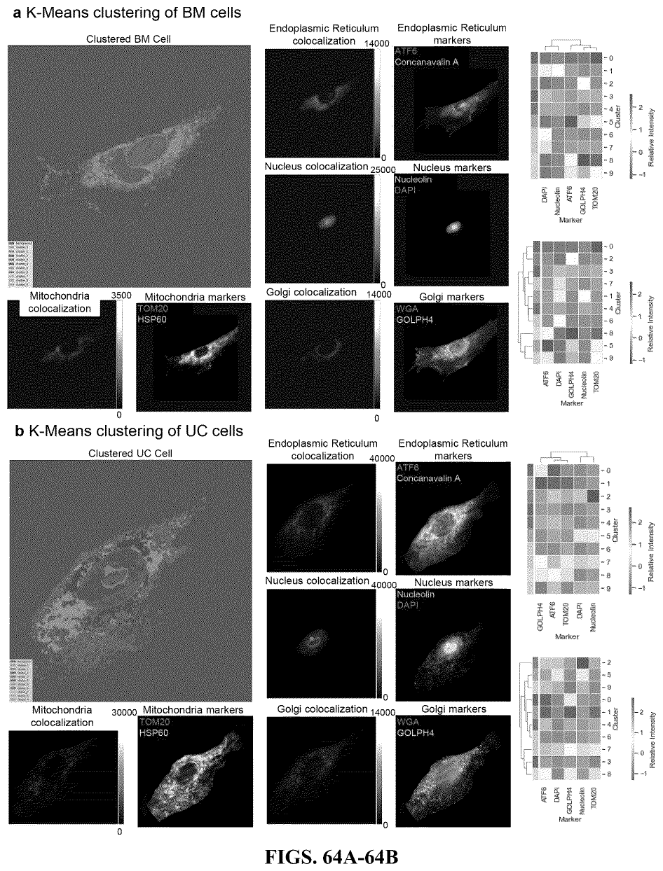

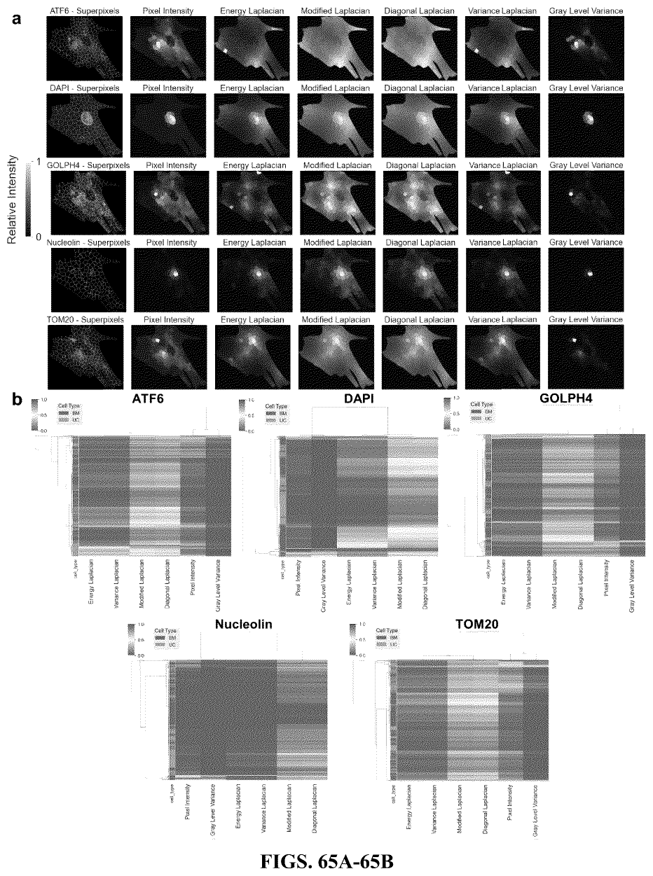

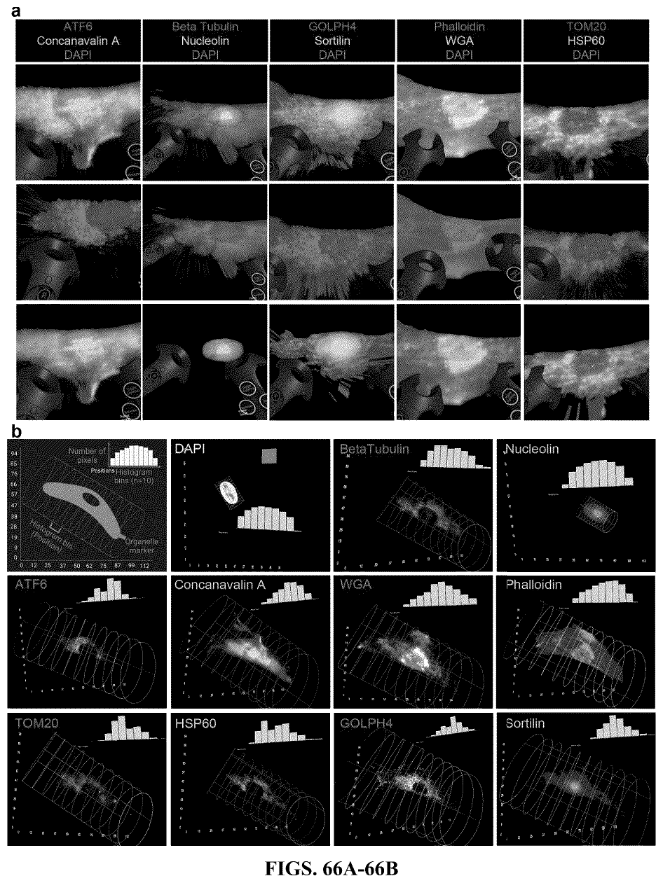

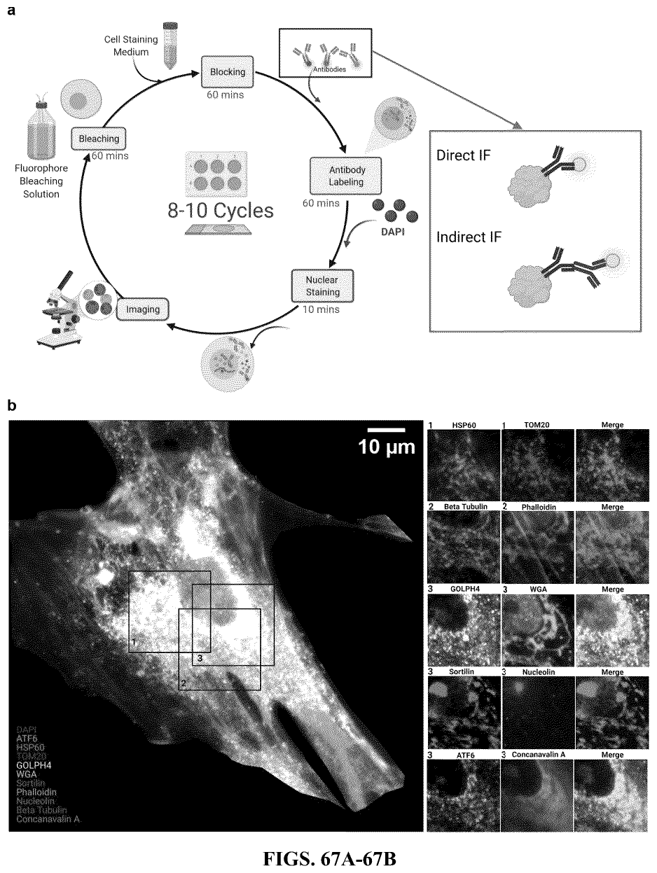

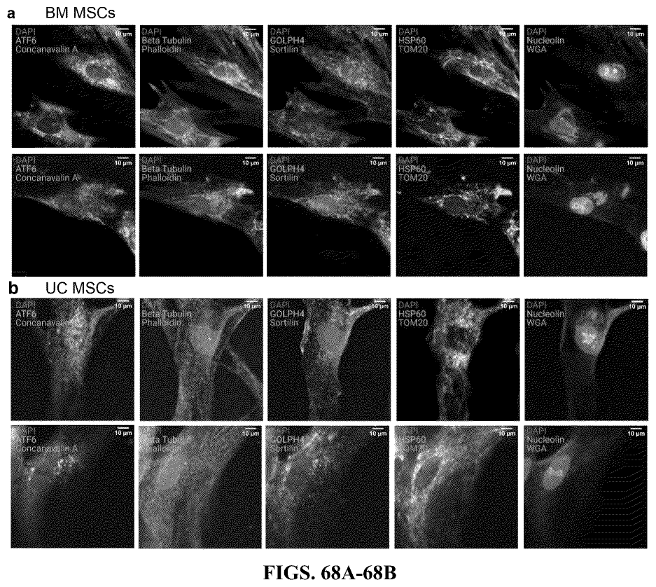

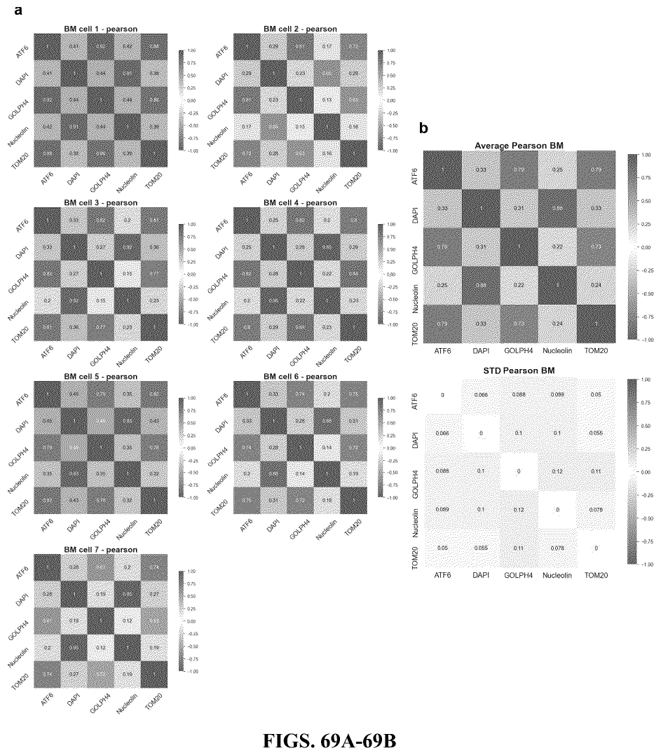

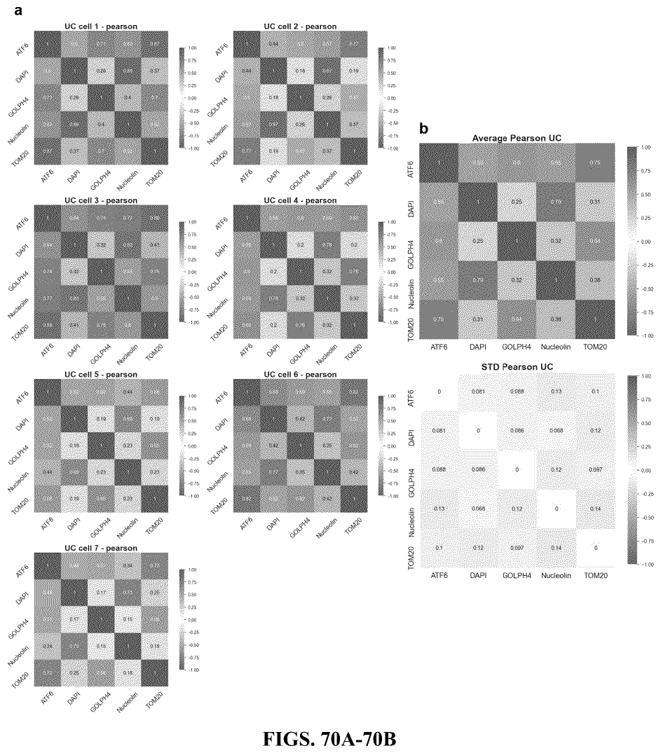

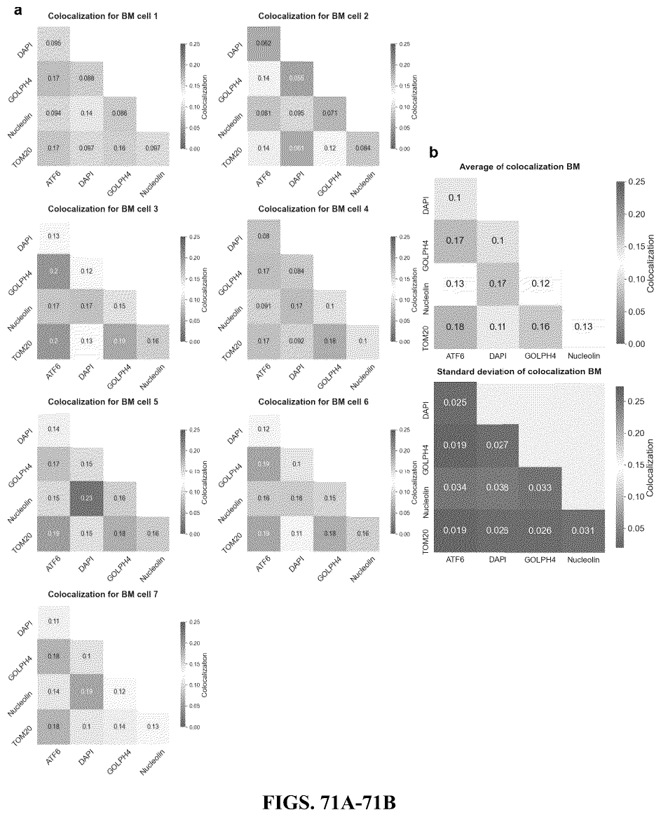

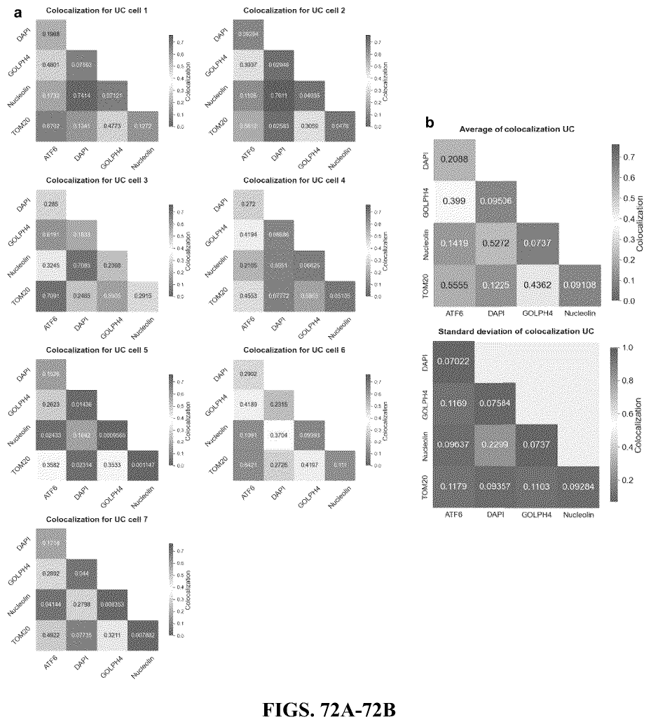

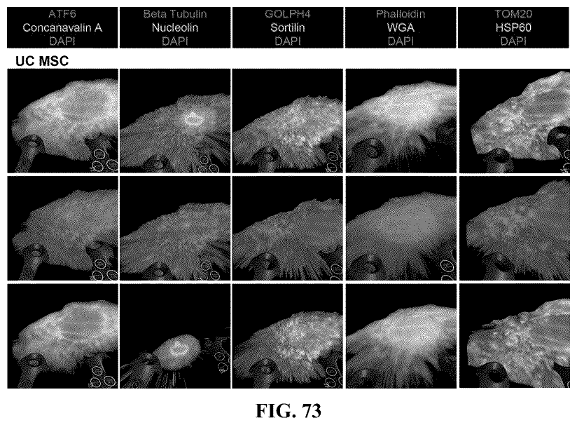

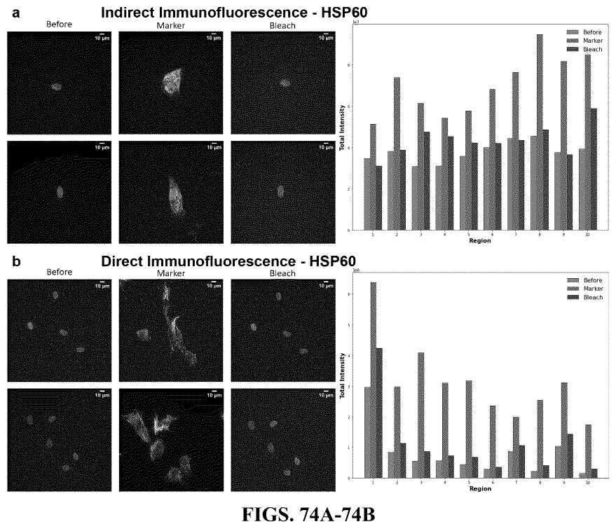

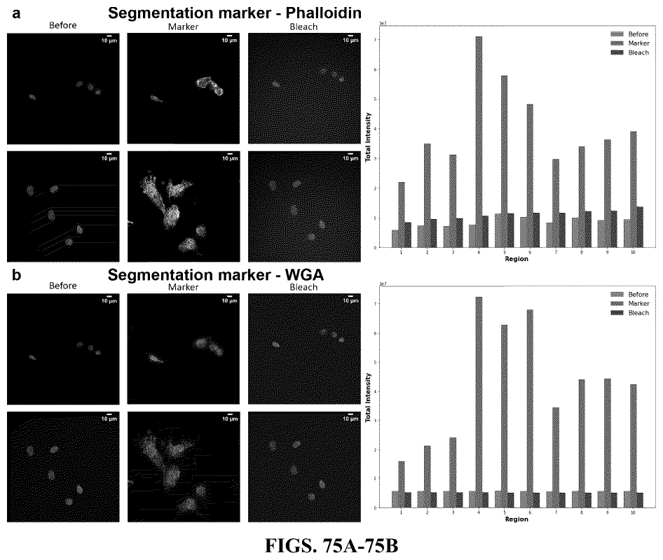

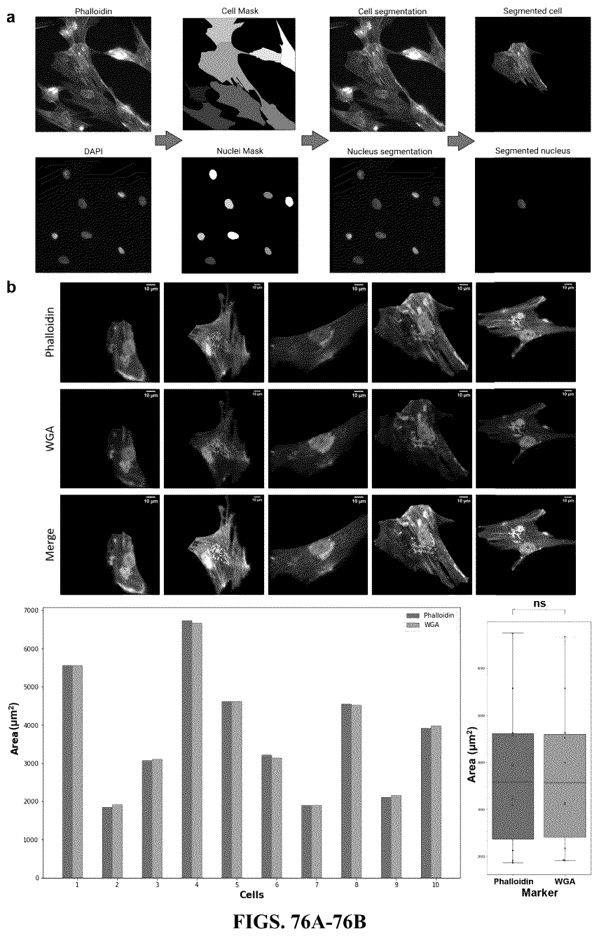

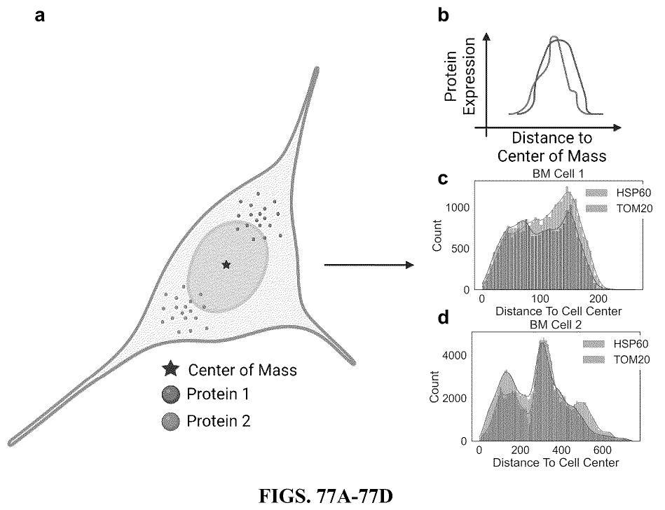

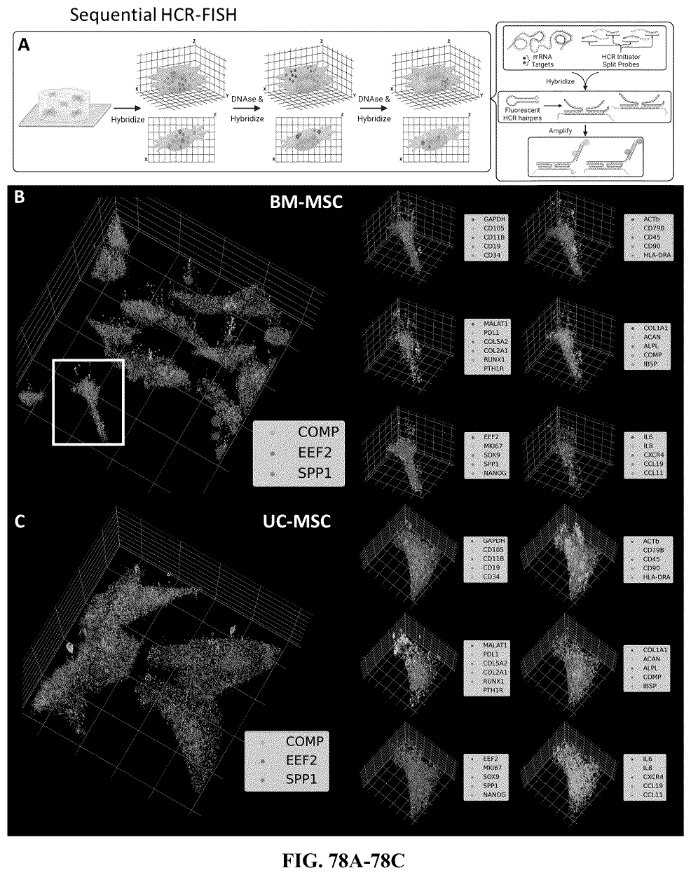

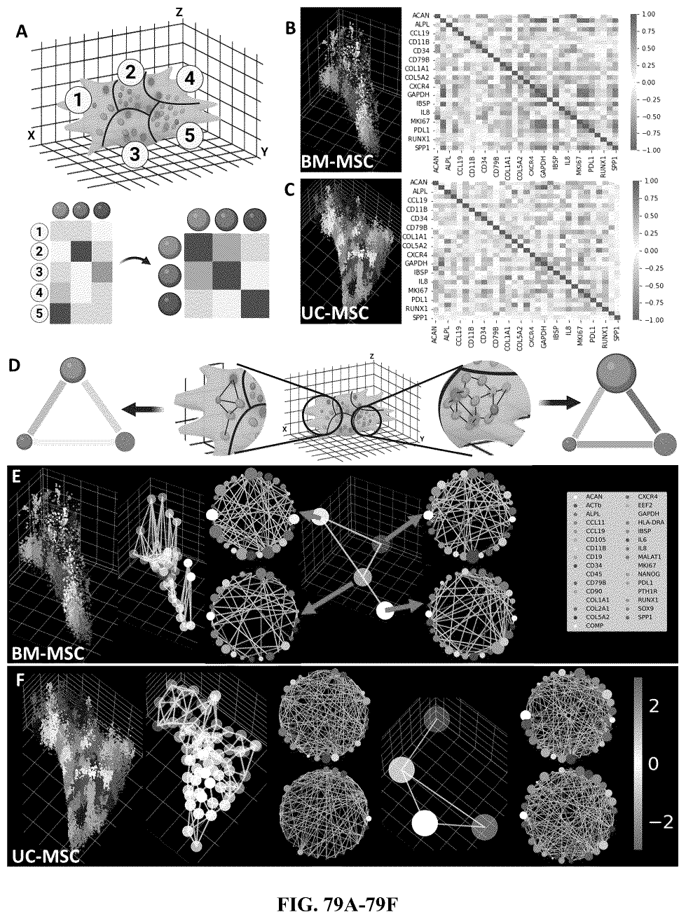

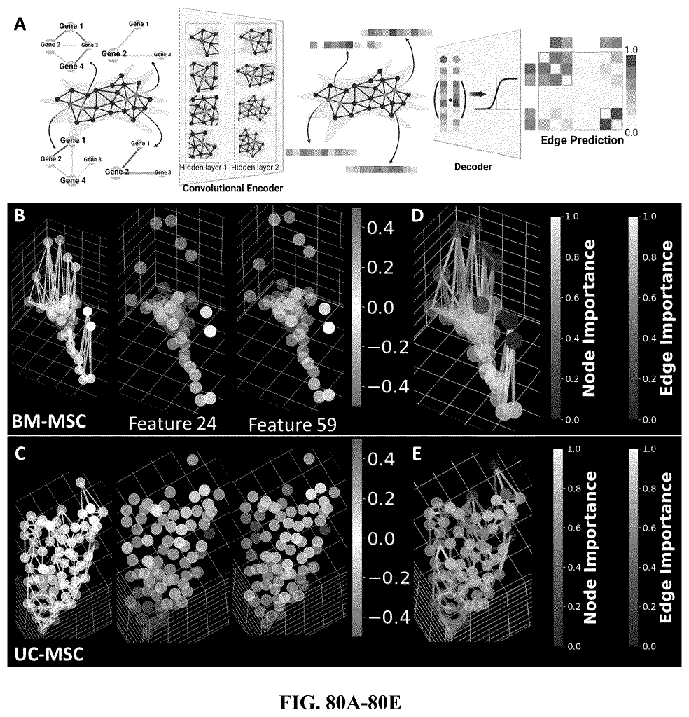

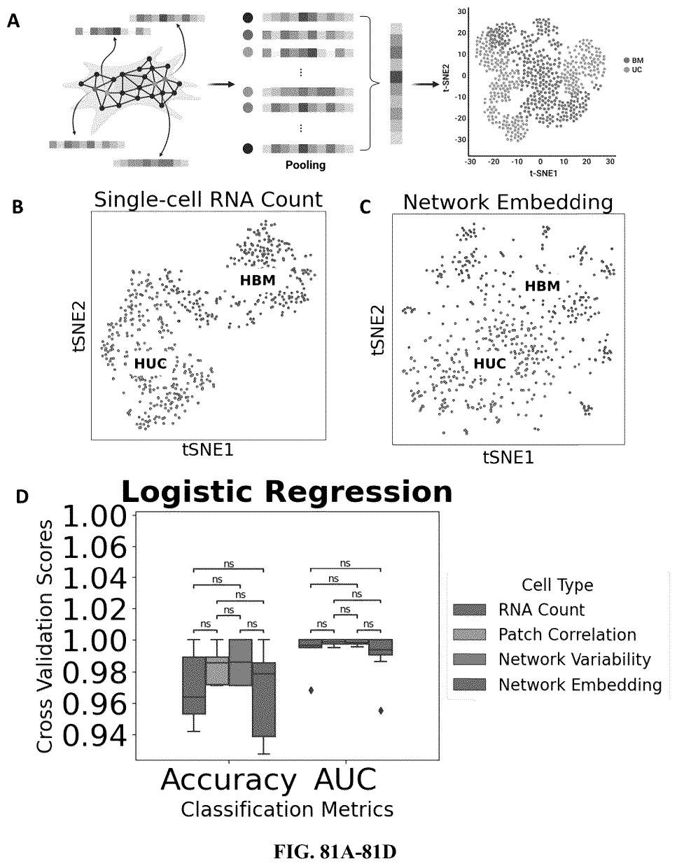

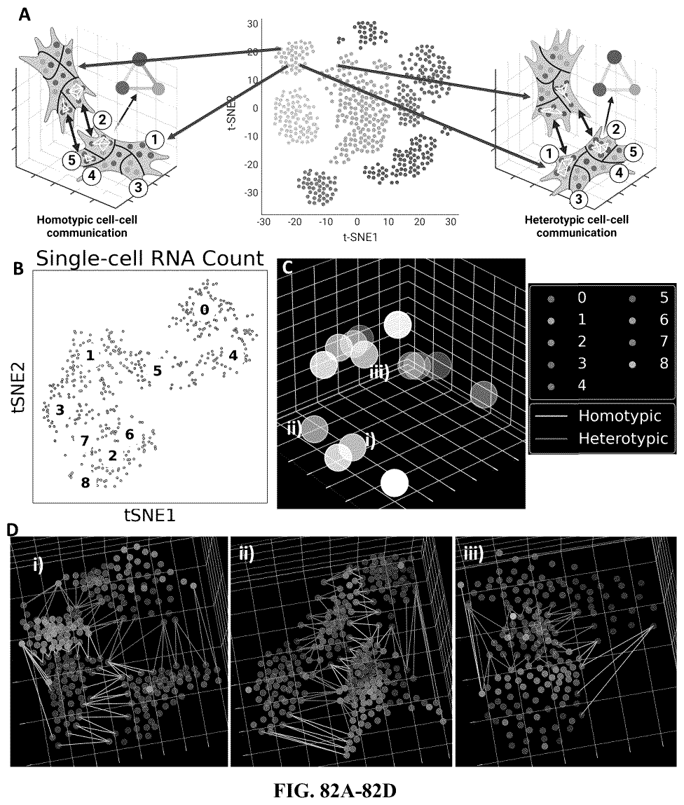

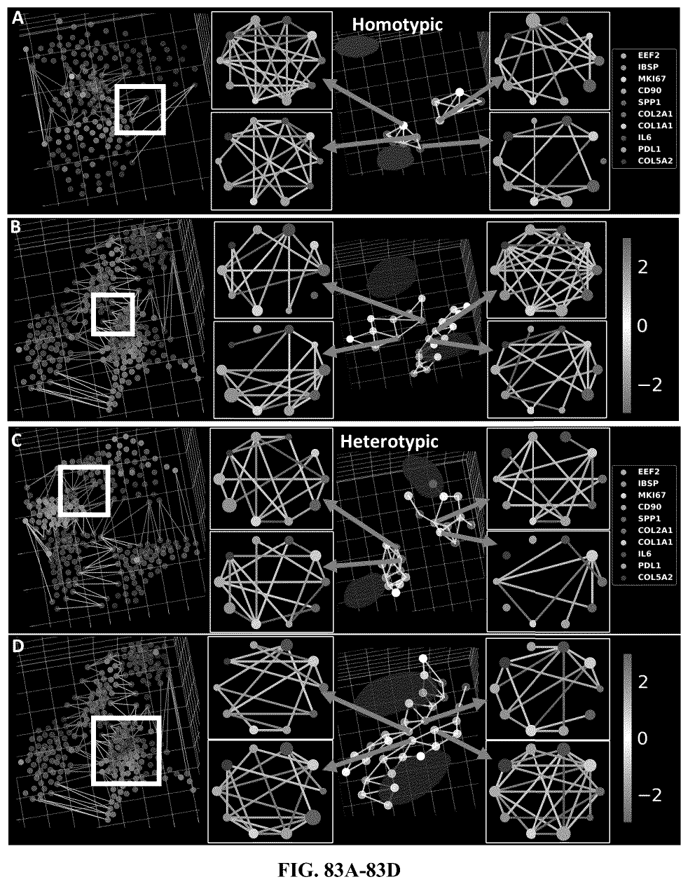

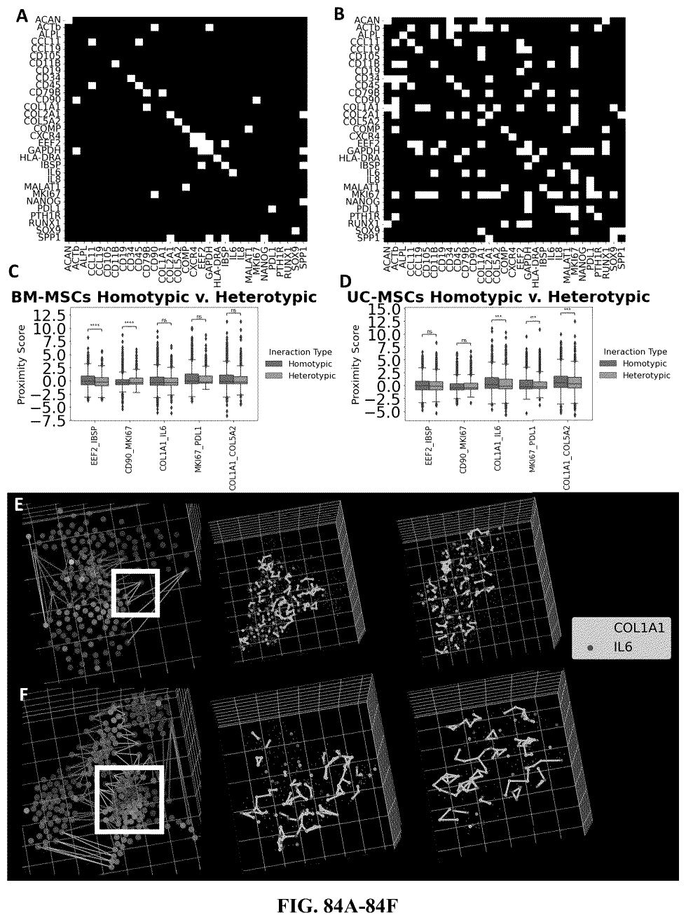

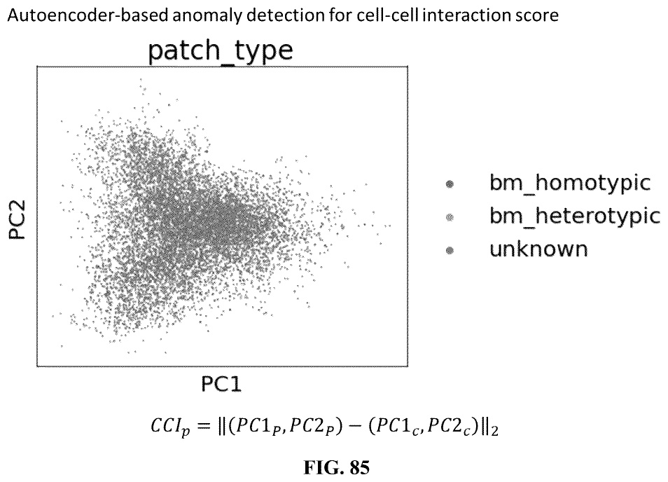

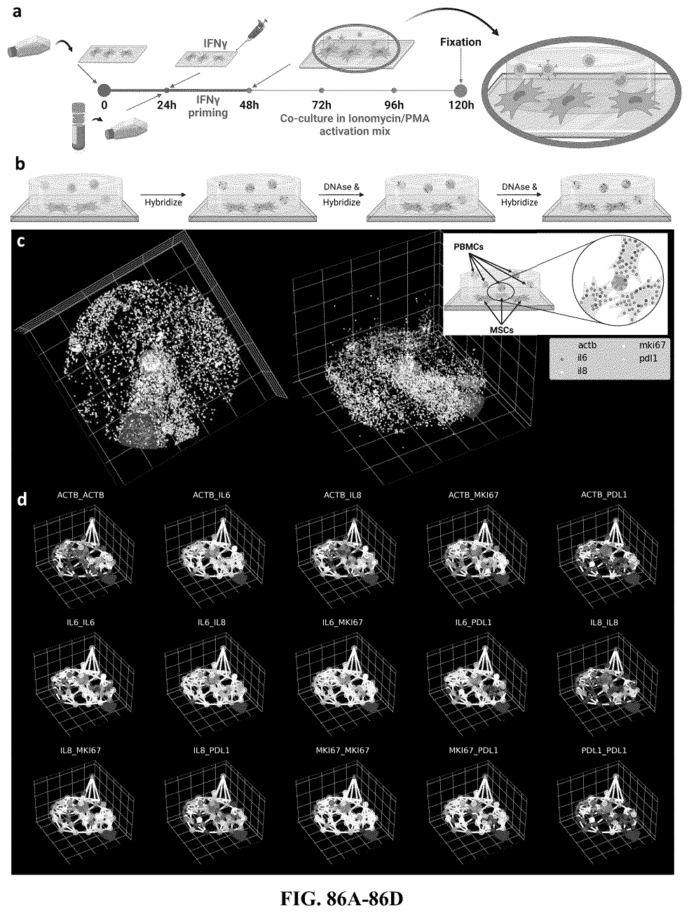

FIGS. 30A-30F show changing numbers of studied genes minimally affect patch division, related to FIGS. 22A-22E.

FIG. 30A shows Patch detected by Leiden clustering using 100 genes from the MERFISH fibroblast dataset. The cell is separated into 37 patches.

FIG. 30B shows Patch detected by Leiden clustering using 50 genes from the MERFISH fibroblast dataset. The cell is separated into 33 patches.

FIG. 30C shows Patch detected by Leiden clustering using 10 genes from the MERFISH fibroblast dataset. The cell is separated into 6 patches.

FIG. 30D shows Rand score of patch separation as the number of genes changes.

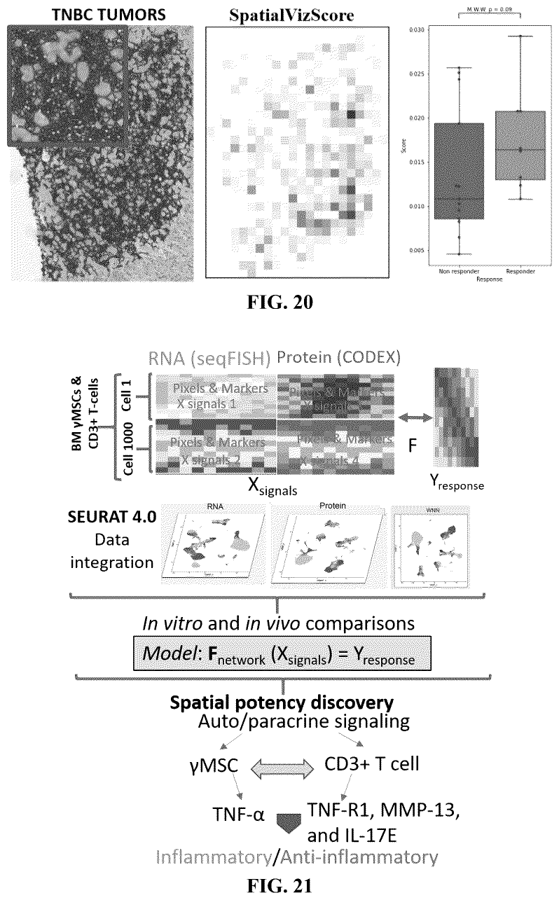

FIGS. 30E-30F show Patch correlation between the same pairs of genes when patches are separated using 10 genes (FIG. 30E) and 20 genes (FIG. 30F). The patch correlations are similar when different numbers of genes are used.

FIGS. 31A-31E show changing the number of studied genes alters resulting gene neighborhood networks, but does not significantly affect pair-wise proximity score mean and standard deviation, related to FIGS. 26A-26E, 27A-27C, 28A-28F.

FIG. 31A shows Patches were detected using 100 genes in a fibroblast.

FIG. 31B shows Gene neighborhood network of the highlighted patch in A.

FIG. 31C shows Patches were detected using 50 genes in fibroblast.

FIG. 31D shows Gene neighborhood network the highlighted patch in C.

FIG. 31E shows Comparison of mean and standard deviation of proximity score of the same pairs of genes using 50 genes and 100 genes. The difference between the proximity scores using different numbers of genes is not significant.

FIGS. 32A-32G show patch-level analysis reveals subcellular heterogeneity in spatial transcriptomic profiles, related to FIG. 30A-30E.

FIG. 32A shows Local high RNA density regions were detected using the Leiden clustering algorithm. Each species of RNA (g) was counted, and correlations of RNA count between all (m) patches of the same cell (n) were calculated. The process was repeated for all n, generating n means correlation maps of size g×g.

FIG. 32B shows Leiden clustering of positions of RNA molecules in a HUC cell. The presented cell contains 57 patches.

FIG. 32C shows Mean correlation map of HUCs (n=121). COL1A1 shows negative correlations with GAPDH and ACTb. EEF2 shows positive correlations with GADH and ACTb.

FIG. 32D shows Leiden clustering of positions of RNA molecules in an HBM cell. The presented cell contains 30 patches.

FIG. 32E shows Mean correlation map of HBMs (n=237). In addition to COL1A1, RUNX1 also shows negative correlations with GAPDH and ACTb.

FIG. 32F shows Leiden clustering of positions of RNA molecules in an HCH cell. The presented cell contains 56 patches.

FIG. 32G shows Mean correlation map of HCHs (n=247). The studied HCH population shows distinct correlation features. IL6 is highly correlated with GAPDH, EEF2, and ACTb. The addition of the nucleus-concentrated MALAT1 marker also shows negative correlations with cytosol-associated markers such as GAPDH, EEF2, ACTb, and IL6.

FIG. 33 shows morphology changes cause minimal changes in patch detection, related to FIGS. 26A-26E. The same HUC with patches was detected using different segmentation of the same cell. The Rand scores derived from the three altered morphologies are 0.986, 0.991, and 0.988. (Perfectly matched clustering has a Rand score of 1.) Therefore, the patch detection process is robust to morphology change.

FIGS. 34A-34C show PCA of patch correlation of HBM, HUC, and HCH, related to FIGS. 30A-30E.

FIG. 34A shows Loading of each pairwise correlation in PC1 visualized in a blue-red color-coded heatmap. The blue color indicates the correlation is negatively biased in PC1. The red color indicates the correlation is positively biased in PC1.

FIG. 34B shows Loading of each pairwise correlation in PC2 visualized in a blue-red color-coded heatmap.

FIG. 34C shows Defining patch correlation of HBM v. rest (left panel), HUC v. rest (middle panel), and HCH v. rest (right panel). Pairwise patch correlations are ranked based on the statistical comparison result of one v. rest comparison.

FIGS. 35A-35D show principal component analysis detects statistically significant features between HBM, HUC, and HCH cells, related to FIGS. 30A-30F.

FIG. 35A shows PC1-PC2 plot of patch-level correlation. The plot shows overlaps of HBM and HUC, with HCH cells displaying little to no overlap. Separation of HCH and MSCs occurs primarily along PC1. Separation of HBM and HUC is less apparent but appears along PC2.

FIG. 35B shows Variance captured plot of PCA analysis. PC1 and PC2 covered a large amount of variance.

FIG. 35C shows Statistical comparison of PC1 biased correlations between HBM, HUC, and HCH. MSCs and HCH are distinguished along PC1. Mann-Whitney-Wilcoxon tests were conducted to examine the differences in pair-wise correlation between cells.

FIG. 35D shows Statistical comparison of PC2 biased correlations between HBM, HUC, and HCH. MSCs and HCH are distinguished along PC2. Mann-Whitney-Wilcoxon tests were conducted to examine the differences in pair-wise correlation between cells.

FIGS. 36A-36D show clustering of cells based on high variance genes only, related to FIGS. 28A-28F.

FIG. 36A shows Cell clustering based on single-cell high variance RNA count visualized on a UMAP plot.

FIG. 36B shows cell types based on single-cell high variance RNA count visualized on a UMAP plot.

FIG. 36C-36D show Confusion of classification of HBM, HUC, and HCH using the count of high variance genes and the full set of genes. The classification accuracy is similar to using the full gene set.

FIG. 37 shows multiplexed spatial transcriptomics from MERFISH fibroblast dataset. Visualization of 130 genes labeled in the MERFISH fibroblast dataset. The patch separation based on the location of transcripts is shown in the last panel.

FIG. 38 shows patch correlation of genes in cells of FIG. 37.

FIG. 39 shows multiplexed spatial transcriptomics from MERFISH fibroblast dataset. Visualization of 130 genes labeled in the MERFISH fibroblast dataset. The patch separation based on the location of transcripts is shown in the last panel.

FIG. 40 shows patch correlation of genes in cells of FIG. 39.

FIG. 41 shows multiplexed spatial transcriptomics from MERFISH U2-OS dataset. Visualization of 130 genes labeled in the MERFISH U2-OS dataset. The patch separation based on the location of transcripts is shown in the last panel.

FIG. 42 shows patch correlation of genes in cells of FIG. 41.

FIG. 43 shows multiplexed HCR generates spatial transcriptomics profile of HBM. Scatter plot showing localization of RNA and adjusted images of protein markers in HBM. The positions of RNA molecules were plotted on the DAPI image. GAPDH, ACTB, EEF2, and COL1A1 are highly expressed, with COL1A1 showing significant enrichment around the nucleus. Brightness and contrast-adjusted ConA, PHA, and WGA images were overlaid on the DAPI image. Unlabeled scale bars have a length of 50 μm.

FIG. 44 shows multiplexed HCR generates spatial transcriptomics profile of HUC. Scatter plot showing localization of RNA and adjusted images of protein markers in HUC. The positions of RNA molecules were plotted on the DAPI image. GAPDH, ACTB, EEF2, and COL1A1 are highly expressed, with COL1A1 showing significant enrichment around the nucleus. Brightness and contrast-adjusted ConA, PHA, and WGA images were overlaid on the DAPI image. Unlabeled scale bars have a length of 50 μm.

FIG. 45 shows multiplexed HCR generates spatial transcriptomics profile of HCH. Scatter plot showing localization of RNA and adjusted images of protein markers in HCH. Positions of RNA molecules were plotted on the DAPI image. GAPDH, ACTb, IL6, EEF2, COL1A1, and MALAT1 are highly expressed, with COL1A1 showing significant enrichment around the nucleus, and MALAT1 showing exclusive localization in the nucleus. Brightness and contrast-adjusted ConA, PHA, and WGA images were overlaid on the DAPI image. Unlabeled scale bars have a length of 50 μm.

FIG. 46A-46B shows comparison of RNA-FISH signal before and after DNase I treatment indicates the removal of the fluorescent DNA construct. Removal of HCR constructs fluorescent DNA assembly on RNA. DNase I treatment followed by formamide washes removes fluorescent signals and preserves intact RNA molecules in different channels.

FIG. 47 shows symmetry of copy number of ACTb and SPP1 in HUC seqFISH image.

FIG. 48 shows single-cell gene enrichment is associated with different regions on UMAP. Single-cell RNA counts visualized in UMAP. The dots' color indicates the expression level of each gene of the cell.

FIGS. 49A-49B show single-cell gene enrichment is associated with different cell types. Single-cell RNA counts visualized in stacked violin plots by cell types. The color of each violin plot indicates the average expression level (raw or z-score normalized) of the gene, and the shape of the violin plot indicates the distribution of the expression of the cell.

FIGS. 50A-50G shows RNA-cellular structure neighborhood network detects COL1A1 colocalization with ER-targeting ConA staining.

FIG. 50A shows Application of spaGNN to detect RNA-cellular structure colocalization. Subcellular patches were detected using the Leiden clustering algorithm. The mean intensity of the RNA image around the transcripts indicates the local enrichment of the marker. The correlation between RNA and protein were studied at both patch and local neighborhood level. Correlation at the local neighborhood level was visualized as neighborhood networks. Created with BioRender.

FIG. 50B shows COL1A1 overlaid on ConA and WGA images in 2 regions of HCH highlighted as i and ii. The image shows COL1A1 localization on ConA positive regions and WGA negative regions.

FIG. 50C shows 30 patches of cells highlighted in Bi. Patch correlation and neighborhood network were analyzed according to the subcellular patches shown.

FIG. 50D shows RNA-protein neighborhood network of a patch near the nucleus. COL1A1 shows a positive correlation with ConA, and ACTb indicates a positive correlation with WGA.

FIG. 50E shows RNA-protein neighborhood network of a patch in the cytosolic region. COL1A1 is suppressed compared to the patch in D. The patch shows a positive pair-wise correlation between COL1A1, ConA, and WGA.

FIG. 50F shows Patch correlation of cell highlighted in Bi. The correlation between protein markers and RNA is highlighted.

FIG. 50G shows Comparison of COL1A1-ConA and COL1A1-WGA colocalization. The differential colocalization was first analyzed by comparing COL1A1 enrichment on ConA and WGA positive regions (Left). COL1A1 shows higher enrichment in ConA-positive regions than in WGA-positive regions. A paired t-test of ConA and WGA region COL1A1 enrichment results in a p-value of 0.046. (α=0.05) The difference in colocalization was confirmed by comparing the average connectivity in neighborhood graphs of COL1A1-ConA and COL1A1-WGA (Right).

FIGS. 51A-51C show image of sequential cycles indicate the removal of edge-localized cytokine RNA signal.

FIG. 51A shows Cy5 channel image of CCL11.

FIG. 51B shows Cy5 channel image of the following cycle. The gene labeled in Cy5 is NANOG.

FIG. 51C shows Comparison of Cy5 image of two sequential cycles. The change of fluorescent signal indicates the detected RNA signal is not residual staining.

FIG. 52A-52H shows the contour patch model resolves edge-localized gene neighborhood networks.

FIG. 52A shows Modified spaGNN pipeline to analyze cytokine RNA localization. Contour patches were defined by the distance from the edge of the cell. The same neighborhood network analysis was applied to each patch. Created with BioRender.

FIG. 52B shows Edge localization of CCL11 transcripts along cell edge. The scatter plot overlaid on the cell mask indicates the subcellular localization of detected RNA transcripts.

FIG. 52C shows Contour patch representation of single cells. RNA transcripts were assigned to contour patches based on their distance to the edge of the cell. Callout contains the Nearest neighbors of an RNA transcript in the patch closest to the cell edge, highlighted by the black box. Only the center RNA and the 4 nearest neighbors were included in the local neighborhood. Further, only transcripts within 5 μm can be considered neighbors of the central RNA.

FIG. 52D shows Contour patch model of a cell without edge-localization of cytokine transcripts.

FIG. 52E shows The neighborhood network of the outermost contour patch. The cell shows a CCL11 edge-localization pattern. IL6 shows negative correlations with both IL8 and CCL11.

FIG. 52F shows The neighborhood network of contour patch 1 region from cell edge. IL8 shows a moderate positive correlation with CCL11. IL6 shows negative correlations with IL8 and CCL11.

FIG. 52G shows The neighborhood network of patch 2 regions from the cell edge. IL8 shows a moderate positive correlation with IL6. CCL11 shows a negative correlation with IL8 and IL6.

FIG. 52H shows UMAP of marker pair-wise connectivity of contour patches used as features. Cells with and without edge localizations of cytokine genes are highlighted in red and blue, respectively. Scale bars have a length of 10 μm.

FIGS. 53A-53B show neighborhood networks of HUC show variability between different contour patches.

FIG. 53A shows Neighborhood networks of HUC with CCL11 edge localization.

FIG. 53B shows Neighborhood networks of HUC without any edge localization

FIG. 54A-54D shows HUC with both IL8 and CCL11 edge-localization indicates different neighborhood networks.

FIG. 54A shows Contour patch map of HUC. Detected RNA is overlaid on the contour plot. The gap between each equal-distance line is a patch.

FIG. 54B shows Edge localization of IL8 and CCL11 RNA.

FIG. 54C shows A local neighborhood of the outermost patch. A center transcript and its 4 nearest neighbors form a local neighborhood. Connections follow the distribution of RNA along the edge of the cell.

FIG. 54D shows Neighborhood networks of contour patches. In this cell, IL8 and CCL11 show a stronger negative correlation in the outermost patch. IL6 and IL8 show a stronger negative correlation in the inner patch. A negative correlation between IL6 and CCL11 is preserved between the patches Unlabeled scale bars have a length of 10 μm FIGS. 55A-55D shows Visualization and statistics showing the proportion of MSCs with edge-localization of cytokine RNA.

FIG. 55A shows Cells with cytokine RNA edge-localization pattern highlighted in single-cell RNA count-based UMAP.

FIG. 55B shows Cells with cytokine RNA edge-localization pattern highlighted in patch-level correlation-based UMAP.

FIG. 55C shows Cells with cytokine RNA edge-localization pattern highlighted in neighborhood network variability-based UMAP.

FIG. 55D shows The proportion of HBMs and HUCs with edge-localized cytokine RNA.

FIGS. 56A-56E shows subcellular clustering reveals spatial gradient patterns in single-cell transcriptomic profiles.

FIG. 56A shows Illustration of subcellular pixel-level clustering analysis. Pixels were first down-sampled to reduce data size. High-dimensional data for each pixel was clustered using the Leiden algorithm. All pixels were then assigned to a cluster based on the clustering result on the down-sampled data. Cells were then pseudo-colored based on cluster labels of each pixel to reveal the spatial distribution of each cluster. Created with BioRender.

FIG. 56B shows The registered images of RNA in cells. A phase cross-correlation registration method was used to correct for both translational and rotational shifts in images between cycles. The cross-registered images show alignment between images of all markers.

FIG. 56C shows Heat map of clustered pixels from HUCs (n=121), HBMs (n=237), and HCHs (n=247). All pixels involved in clustering are shown in the heatmap. Blue-red colors indicate the enhancement of markers for the corresponding pixels. The tendency of enrichment of pixels within the same cluster indicates the co-existence of markers in clusters.

FIG. 56D shows Pseudo-colored cell images according to the cluster assignment of each pixel. All three cell types exhibit a gradient of cluster changes from the center of cells toward the cell membrane.

FIG. 56E shows Heatmap of differential enrichment of each cluster in each cell type. The proportion of pixels belonging to each cluster was computed for each cell type. Enrichment of clusters 0-3, 5-7, 12, and 16 is common to all cell types. HBM shows suppression of clusters 4 and 10. HCH shows suppression of clusters 13, 14, and 15, and enrichment of clusters 8, 9, and 10. HUC demonstrates moderate enrichment of non-common clusters and suppression of clusters 14 and 15 similar to HBM.

FIGS. 57A-57E shows UMAP of down-sampled pixels in pixel-wise clustering.

FIG. 57A shows UMAP of clustering of pixels.

FIG. 57B shows The weight of genes in each cluster is indicated by a blue-red color-coded heatmap using the original pixel intensities.

FIG. 57C shows UMAP shows the enrichment of genes.

FIG. 57D shows The weight of genes in each cluster is indicated by a blue-red color-coded heatmap using perturbated pixel intensities. The clustering was performed under α-perturbation with α=2.

FIG. 57E shows Rand scores of clustering original v. perturbated data and original v. randomly assigned clusters. The comparison between original and perturbated clustering has higher similarities than original data and randomly assigned clusters.

FIGS. 58A-58E show subcellular clustering by a single image reveals a gradient pattern of gene enrichment region.

FIG. 58A shows Subcellular pixel-level clustering of aligned cell images. The image of each cell was analyzed using K-means clustering. The heatmap indicates the enrichment of each marker in each cluster. Cells were then pseudo-colored based on the cluster assignment of each pixel. Created with BioRender.

FIG. 58B shows Aligned images of HBM, HUC, and HCH cells.

FIG. 58C shows Heatmaps indicate the enrichment of markers in each cluster. Pixels from the same image inside cellular regions were collected and clustered using K-means clustering. The cluster centers were defined by computing the mean enrichment of each marker of all pixels assigned to the cluster. The enrichment of markers of each cluster center is shown in the heatmap.

FIG. 58D shows Pseudo-colored cell image according to clustering result. Cells were colored based on the cluster assignment of each pixel. Cells present similar gradient patterns of cluster distribution. The same pattern is observed in the same clustering analysis using pixels from multiple images.

FIG. 58E shows The sum of square error v. k value plot helps find the optimal k value. The elbow point in the sum of intra-cluster square error v. k value plot was used to determine the optimal k-value in k-means clustering of pixels. The elbow point is identified at k=15.

FIGS. 59A-59D show statistical comparisons of cell source populations confirm distinct spatial patterning of RNAs.

FIG. 59A shows Spatial RNA expression is measured with respect to each cell's center of mass. RNA molecules are nuclear or cytosol associated as illustrated in the histogram of each transcript's distance to the cell center. Examples highlighted in blue and red are COL1A1 and EEF2 genes respectively in a single HBM. Created with BioRender.

FIG. 59B shows Visual validation of spatial RNA pattern (blue/red) on cell mask (gray). The example shown is COL1A1 in an HBM. Blue dots indicate a shorter distance from the center of the cell and red dots indicate a longer distance from the center of the cell. The scale bar indicates a length of 10 μm.

FIG. 59C shows Spatial RNA histograms as heatmaps for each gene for HUCs (n=121), HBMs (n=237), and HCHs (n=247). HCH genes exhibit more cytosolic patterns than corresponding genes for HBM and HUC, while HUC and HBM present higher similarities in the spatial distribution of RNA.

FIG. 59D shows The graph shows the p-values of the Kolmogorov-Smirnov Hypothesis Test of COL1A1 spatial relationships for all pairwise cells in the HBM population

FIGS. 60A-60B show ten-plex organelle mapping in mesenchymal stem cells using rapid multiplexed immunofluorescence.

FIG. 60A shows Schematic of rapid multiplexed immunofluorescence for organelle analysis in MSCs. Each cycle contains 3 conjugated antibodies plus DAPI followed by bleaching of the signal before the next cycle consisting of 3 new antibodies. Imaging of the 3 antibodies before and after bleaching confirms the presence of signal and then signal removal. Multiplex imaging consists of multiple cycles (n) of antibody labeling and bleaching. BM MSCs (brown) and UC MSCs (cyan) are labeled with the same multiplex antibodies that target the same set of organelles. All images are acquired on Nikon widefield and registered across cycles to produce a final set of multiplex-labeled images. Example images show Beta Tubulin (magenta, left) and TOM20 (magenta, right) overlaid with the nucleus in DAPI (blue). Created with BioRender.

FIG. 60B shows Visualization of organelle markers in single cells from BM MSCs and UC MSCs. Each row corresponds to a distinct single cell. The top 2 rows show BM MSCs and the bottom 2 rows show UC MSCs. Multiplexed markers for the same cell are displayed across 4 columns (Column 1: ATF6 & Concavalin, Column 2: Beta Tubulin & Phalloidin, Column 3: GOLPH4 & Sortilin, Column 4: HSP60 & TOM20, Column 5: Nucleolin & WGA). Each image displays DAPI with a pair of organelle markers in magenta and green. DAPI is used to register the signals across cycles. Signal removal is confirmed with a widefield microscope after bleaching each cycle. All scale bars 10 μm.

FIGS. 61A-61B show comparison of protein markers targeting the same organelles in BM MSCs and UC MSCs.

FIG. 61A shows Single BM MSC example with various organelles across the columns. Each organelle is shown by a marker pair: HSP60 & TOM20 (mitochondria, left), GOLPH4 & Sortilin (Golgi, middle), ATF6 & Concanvalin A (ER, right). All scale bars 10 μm. The bottom row shows intensity scatter plots with 50000 pixels for each marker pair colocalized within the same organelle. The x and y-axis indicate the min-max scaled intensity values of the markers. More colocalized pixels appear closer to the y=x diagonal while less colocalized pixels appear closer to either axis, belonging more to that particular marker. In BM MSCs, mitochondria antibodies are more colocalized than either Golgi or ER.

FIG. 61B shows Single UC MSC example with various organelles across the columns. Each organelle is shown by a marker pair: HSP60 & TOM20 (mitochondria, left), GOLPH4 & Sortilin (Golgi, middle), ATF6 & Concanvalin A (ER, right). All scale bars 10 μm. The bottom row shows intensity scatter plots with 50000 pixels for each marker pair colocalized within the same organelle. The x and y-axis indicate the min-max scaled intensity values of the markers. More colocalized pixels appear closer to the y=x diagonal while less colocalized pixels appear closer to either axis, belonging more to that particular marker. In UC MSCs, ER antibodies are more colocalized than either mitochondria or Golgi.

FIGS. 62A-62D show spatial analysis of organelle colocalization in BM MSCs and UC MSCs.

FIG. 62A shows Boxplots of Pearson's correlation of marker pairs per cell between all BM MSCs (blue) and UC MSCs (orange) categorized by nuclear, cytoskeletal, and organelle markers. Box plots show the median, first and third quartile, minimum, and maximum (excluding outliers). The outliers are marked as individual points. Stars denote the statistical significance for pairwise comparison. β-value was calculated using the Mann-Whitney test (ns: p>=0.05, ****: p<=0.0001). BM MSCs and UC MSCs exhibit the greatest differences in colocalized expression among pairs that include mitochondria (TOM20, HSP60), cytoskeleton (Beta Tubulin, Phalloidin), and endoplasmic reticulum (Concanavalin A).

FIG. 62B shows Boxplots of pixel overlap colocalization values of marker pairs per cell between all BM MSCs (blue) and UC MSCs (orange) categorized by nuclear, cytoskeletal, and organelle markers. Box plots show the median, first and third quartile, minimum, and maximum (excluding outliers). The outliers are marked as individual points. Stars denote the statistical significance for pairwise comparison. β-value was calculated using the Mann-Whitney test (ns: p>=0.05, ****: p<=0.0001). UC MSC markers express a higher fraction of colocalized pixels in ER (ATF6, Concanavalin A) and mitochondria (HSP60) than those of BM MSCs, suggesting more active crosstalk of organelles in UC MSCs.

FIG. 62C shows Comparison of the total area of 11 markers per cell between all BM MSCs (blue) and UC MSCs (orange) using boxplots. Box plots show the median, first and third quartile, minimum, and maximum (excluding outliers). The outliers are marked as individual points. Stars denote the statistical significance for pairwise comparison. β-value was calculated using the Mann-Whitney test (ns: p>=0.05, ****: p<=0.0001). UC MSC markers generally express larger and more variable areas than those of BM MSCs, which suggests that these organelles serve a higher energetic role in UC MSCs.

FIG. 62D shows Comparison of the total intensity of 11 markers per cell between all BM MSCs (blue) and UC MSCs (orange) using boxplots. Box plots show the median, first and third quartile, minimum, and maximum (excluding outliers). The outliers are marked as individual points. Stars denote the statistical significance for pairwise comparison. β-value was calculated using the Mann-Whitney test (ns: p>=0.05, ****: p<=0.0001. Markers in UC MSCs express a larger range of intensities than in BM MSCs and thus these UC MSC organelles are in a more active state.

FIGS. 63A-63D show multiplexed protein analysis of organelle markers in BM MSCs and UC MSCs.

FIG. 63A shows Average Pearson's correlation of the total intensity of 5 markers in BM MSCs and UC MSCs per marker per cell plotted as a heatmap. BM MSCs are shown on the left, UC MSCs are shown in the middle, and combined Pearson's for each cell is shown on the right. The comparison of the correlation of 5 markers between BM MSCs (n=7) and UC MSCs (n=7) was provided as a heatmap using the average linkage method based on the correlation distance. Larger correlation values are shown in red and smaller correlation values are shown in blue. BM MSCs possess more separation between nuclear and cytosolic organelles while UC MSCs are less separated, which illustrates that UC MSC organelles are more spread across the cell. The right side shows the single-cell heatmap for all marker pairs. UC MSCs exhibit higher correlations in DAPI_ATF6, TOM20_ATF6, and Nucleolin_ATF6, suggesting more crosstalk between nuclear and cytosolic organelles. Single BM MSCs exhibit stronger correlations within nuclear or cytosol pairs e.g. Nucleolin_DAPI, GOLPH4_ATF6, TOM20_GOLPH4, implying that nucleus and cytosol organelles are more segregated in BM MSCs, which agrees with the left heatmap.

FIG. 63B shows Average pixel overlap colocalization between 5 markers in BM MSCs and UC MSCs per marker per cell plotted as a heatmap. The comparison of pixel overlap between BM MSCs (left) and UC MSCs (middle) was provided as a heatmap using the average linkage method based on the contact frequency distance. The combined pixel overlap on a single cell level is shown on the right. Large pixel overlap values are shown in red and small pixel overlap values are shown in blue. UC MSCs possess slightly higher pixel overlap values among organelles in different compartments (nuclear vs cytoskeleton) but this difference is less pronounced than in FIG. 63A. A few UC MSCs (10, 12, 13, 14) show distinct patterns from other UC MSCs in terms of weaker pixel overlap in Nucleolin_DAPI, GOLPH4_ATF6, TOM20_GOLPH4, Nucleolin_ATF6, TOM20_Nucleolin. UC MSCs exhibit higher variability in pixel overlap.

FIG. 63C shows Heatmaps were generated to compare the morphological features and to determine any close relationships or associations between markers and between BM MSCs (red) and UC MSCs (teal). Morphology is defined as area (left), minor axis (middle), and major axis (right). Both cell types exhibit more differences in minor and major axes and fewer differences in terms of area, implying that organelles differ more in shape and less in expression area. The most notable difference is TOM20, suggesting a difference in mitochondrial energetic activity. Most differences, such as TOM20 and ATF6, are attributable to UC MSCs, suggesting more single-cell variability among UC MSCs.

FIG. 63D shows Kolmogorov-Smirnov (K-S) hypothesis test was conducted between organelle marker pairs targeting the mitochondria (TOM20 and HSP60; left) and Golgi (GOLPH4 and Sortilin; right) to study if similar proteins express different spatial distributions within each cell. Single BM MSCs are shown in red while single UC MSCs are shown in teal. In terms of spatial mitochondrial expression, BM MSCs have more single-cell variability while UC MSCs express more uniformly. BM MSCs possess more single-cell variability than UC MSCs concerning spatial ER expression.

FIGS. 64A-64B show pixel level clustering of organelle markers in BM MSCs and UC MSCs.

FIG. 64A shows K Means clustering of intensities of markers for a single BM MSC. 10 clusters were chosen and colored back on the original cell (large left). Each cluster represents one distinct expression profile of the protein markers in the cells. A pair of images are shown for each organelle (mitochondria, Golgi, Nucleus, ER): 1) an overlay of the two markers that target the organelle (right) and 2) the colocalization of the two markers obtained by multiplying the two marker images pixel-wise from the right to highlight areas of overlap (left). Example: ATF6 and Concanavalin A forming the ER localization. Yellow values indicate areas of higher overlap while red values indicate areas of lower overlap. The right side shows the heatmap of marker intensity with a dendrogram based on marker intensities (right, top) and clustering results (right, bottom). From the dendrogram based on marker intensities, GOLPH4 and TOM20 are directly linked, along with ATF6. This suggests moderate crosstalk between ER and mitochondria, especially in cluster 8. Nuclear organelles DAPI and Nucleolin are associated in a separate cluster.

FIG. 64B shows K Means clustering of intensities of markers for a single UC MSC. 10 clusters were chosen and colored back on the original cell (large left). Each cluster represents one distinct expression profile of the protein markers in the cells. A pair of images are shown for each organelle (mitochondria, Golgi, Nucleus, ER): 1) an overlay of the two markers that target the organelle (right) and 2) the colocalization of the two markers obtained by multiplying the two marker images pixel-wise from the right to highlight areas of overlap (left). Example: ATF6 and Concanavalin A forming the ER localization. Yellow values indicate areas of higher overlap while red values indicate areas of lower overlap. The right side shows the heatmap of marker intensity with a dendrogram based on marker intensities (right, top) and clustering results (right, bottom). From the dendrogram based on marker intensities, ATF6 and TOM20 exhibit moderate crosstalk (clusters 3, 7, 8) but not as intensely as the BM MSC in (a).

FIGS. 65A-65B show superpixel segmentation and texture analysis of organelle markers in BM MSCs and UC MSCs.

FIG. 65A shows Superpixel segmentation and texture features using various cost functions (Pixel Intensity, Energy Laplacian, Modified Laplacian, Diagonal Laplacian, Variance Laplacian, and Gray Level Variance) calculated on superpixels for each marker of one BM MSC. Each column represents a different superpixel method. Segmentation identifies subcellular, regional hotspots of organelles. Modified Laplacian and diagonal Laplacian create a more spread signal for all markers. Pixel intensity and variance Laplacian reveal discontinuous hotspots of GOLPH4, ATF6, and TOM20.

FIG. 65B shows Heatmap of texture feature values for each superpixel in 7 BM MSCs and 7 UC MSCs for each marker. Each row denotes a single superpixel, and the column contains texture feature values for the corresponding superpixel. The feature values range from 0 (blue) to 1 (red) in each heatmap. Modified Laplacian and diagonal Laplacian yield the largest feature values. Each heatmap represents a different marker. UC and BM MSCs show similar overall patterns across various superpixels, implying that earlier, quantified differences are more minute and superpixel methods disguise these small differences when downsampling.

FIGS. 66A-66B show virtual reality-based visualization and analysis of organelle imaging data in MSCs.

FIG. 66A shows Visualization of organelle protein images (10 markers) for one BM MSC in ConfocalVR⁶³. For each cell, the top shows the RGB image of the combined markers, while the bottom two show a single marker image in either red or green with DAPI. Different organelle markers are illustrated across the columns. Merged images are shown in the first row. Column 1: ATF6 (middle row) & Concanavalin A (bottom row), Column 2: Beta Tubulin (middle row) & Nucleolin (bottom row), Column 3: GOLPH4 (middle row) & Sortilin (bottom row), Column 4: Phalloidin (middle row) & WGA (bottom row), Column 5: TOM20 (middle row) & HSP60 (bottom row). Handset toggle switches used to interact with the image are shown on the left and right sides.

FIG. 66B shows Visualization of organelle protein images (10 markers) for one BM MSC in Genuage⁶. The histogram of pixel count in 10 cylindrical bins (shown in white) of width 12.5 pixels (108 nm/pixel, bin width 1.354 μm) was plotted in the software. The top left image is an illustration explaining how cells are binned across the length to gauge spatial variability. In each box, red and blue colors illustrate the marker according to the upper left label. Beta Tubulin, ATF6, TOM20, and GOLPH4 show discontinuous regions of signal, supported by the uneven histograms, while the other signals are more continuous with more Gaussian histograms.

FIGS. 67A-67B show rapid multiplexed immunofluorescence for high-dimensional spatial profiling in single cells.

FIG. 67A shows Timeline for rapid multiplexed immunofluorescence staining. Each cycle consists of up to 3 antibodies plus DAPI. In between subsequent cycles, the antibodies were bleached and the same sample was re-blocked and stained with a new set of antibodies. Inset shows the difference between direct and indirect IF. While direct staining is a single-step process, indirect staining is a two-step requiring a primary antibody and a secondary antibody and is, therefore, more time-consuming. The duration of each step is labeled in green. Created with BioRender.

FIG. 67B shows 10-plex visualization of organelle markers in BM MSCs. On the right, zoomed-in regions are shown to illustrate regions of organelle colocalization and overlap of organelle marker presence. Row 1: HSP60 & TOM20, Row 2: Beta Tubulin & Phalloidin, Row 3: GOLPH4 & WGA, Row 4: Sortilin & Nucleolin, Row 5: ATF6 & Concanavalin A. The third column indicates the merge of the two markers in the first two columns.

FIGS. 68A-68B show 10-plex organelle mapping in mesenchymal stem cells.

FIG. 68A shows Visualization of organelle markers in single cells from BM MSCs. Each row corresponds to a unique cell and each image displays two organelle markers. Column 1: ATF6 (green) & Concanavalin A (magenta), Column 2: Beta Tubulin (green) & Phalloidin (magenta), Column 3: GOLPH4 (green) & Sortilin (magenta), Column 4: HSP60 (green) & TOM20 (magenta), Column 5: Nucleolin (green) & WGA (magenta). All scale bars 10 μm.

FIG. 68B shows Visualization of organelle markers in single cells from UC MSCs. Each row corresponds to a unique cell and each image displays two organelle markers. Column 1: ATF6 (green) & Concanavalin A (magenta), Column 2: Beta Tubulin (green) & Phalloidin (magenta), Column 3: GOLPH4 (green) & Sortilin (magenta), Column 4: HSP60 (green) & TOM20 (magenta), Column 5: Nucleolin (green) & WGA (magenta). All scale bars 10 μm.

FIGS. 69A-69B show Pearson's correlation of markers in BM MSCs (n=7).

FIG. 69A shows Correlation values of the total intensity of 5 markers (ATF6, DAPI, GOLPH4, Nucleolin, TOM20) in BM MSCs were calculated per marker for each cell using Pearson's Correlation and plotted as heatmaps. Pearson's correlation values range from −1 (blue) to +1 (red).

FIG. 69B shows Average (top) and standard deviation (bottom) of Pearson's correlation of intensity per marker across all BM MSCs for 5 markers (ATF6, DAPI, GOLPH4, Nucleolin, TOM20). The color bar ranges from −1 (blue) to +1 (red). Standard deviation values are close to 0 while average values agree well with every single cell in (a), indicating a more uniform organelle pattern among BM MSCs.

FIGS. 70A-70B show Pearson's correlation of markers in UC MSCs (n=7).

FIG. 70A shows Correlation values of the total intensity of 5 markers (ATF6, DAPI, GOLPH4, Nucleolin, TOM20) in UC MSCs were calculated per marker for each cell using Pearson's Correlation and plotted as heatmaps. Pearson's correlation values range from −1 (blue) to +1 (red).

FIG. 70B shows Average (top) and standard deviation (bottom) of Pearson's correlation of intensity per marker across all UC MSCs for 5 markers (ATF6, DAPI, GOLPH4, Nucleolin, TOM20). The color bar ranges from −1 (blue) to +1 (red). Standard deviation values are close to 0 while average values agree well with every single cell in FIG. 70A except for UC cells 5 and 7, indicating a less uniform organelle pattern among UC MSCs.

FIGS. 71A-71B show pixel overlap colocalization of markers in BM MSCs (n=7).

FIG. 71A shows Pixel overlap colocalization of 5 markers (ATF6, DAPI, GOLPH4, Nucleolin, TOM20) in BM MSCs per marker for each cell (n=7) plotted as heatmaps. The color bar ranges from 0 (blue) to 0.25 (red) and indicates the fraction of the cell occupied by the marker pair. BM cells 1, 2, and 4 exhibit slightly different organelle patterns than BM cells 3, 5, 6, and 7. DAPI & Nucleolin, ATF6 & TOM20, ATF6 & GOLPH4, and TOM20 & GOLPH4 exhibit the highest pixel overlap colocalizations.

FIG. 71B shows Average (top) and standard deviation (bottom) of pixel overlap colocalization per marker across all BM MSCs for 5 markers (ATF6, DAPI, GOLPH4, Nucleolin, TOM20). The color bar ranges from 0 (blue) to 0.25 (red). DAPI & Nucleolin, ATF6 & TOM20, ATF6 & GOLPH4, and TOM20 & GOLPH4 exhibit the highest pixel overlap colocalizations. Standard deviations are mostly low, indicating less single-cell variation.

FIGS. 72A-72B show pixel overlap colocalization of markers in UC MSCs (n=7).

FIG. 72A shows Pixel overlap colocalization of 5 markers (ATF6, DAPI, GOLPH4, Nucleolin, TOM20) in UC MSCs per marker for each cell (n=7) plotted as heatmaps. The color bar ranges from 0 (blue) to 0.76 (red). TOM20 and ATF6 show high colocalization in all cells except UC cells 4 and 5, suggesting crosstalk between mitochondria and ER in most UC MSCs. Nucleolin and DAPI also exhibit high colocalization, indicating some segregation of nuclear and cytosolic organelles.

FIG. 72B shows Average (top) and standard deviation (bottom) of pixel overlap colocalization per marker across all BM MSCs for 5 markers (ATF6, DAPI, GOLPH4, Nucleolin, TOM20). The color bar ranges from 0 (blue) to 0.76 (red) for average and 0 (blue) to 1 (red) for standard deviation. The highest average colocalization exists between DAPI & Nucleolin, and ATF6 & TOM20. Thus, many UC MSCs exhibit crosstalk between mitochondria and ER while nuclear organelles are more separated from cytosolic ones. The low standard deviation shows most cells behave similarly.

FIG. 73 shows VR-based visualization of organelle imaging data in MSCs Visualization of organelle protein images (10 markers) for one UC MSC in ConfocalVR. For each cell, the top shows the RGB image of the combined markers, while the bottom two show a single marker image in either red or green with DAPI. Different organelle markers are illustrated across the columns. Merged images are shown in the first row. Column 1: ATF6 (middle row) & Concanavalin A (bottom row), Column 2: Beta Tubulin (middle row) & Nucleolin (bottom row), Column 3: GOLPH4 (middle row) & Sortilin (bottom row), Column 4: Phalloidin (middle row) & WGA (bottom row), Column 5: TOM20 (middle row) & HSP60 (bottom row). Handset toggle switches used to interact with the image are shown on the left and right sides.

FIGS. 74A-74B show validation of bleaching protocol for indirect and direct IF with HSP60.

FIG. 74A shows images of before staining (left column), staining (middle column), and after bleaching (right column) of HSP60 (green) for indirect IF. Before and after bleaching images confirm signal addition and removal before the next staining cycle. All scale bars 10 μm. On the right, the intensity sum of ten regions before staining, staining, and after bleaching is quantified to validate the effect of the fluorophore bleaching process. Before (red) and after bleach (blue) show comparable intensities while marker (green) illustrates approximately 1.5-2-fold higher intensity.

FIG. 74B shows images of before staining (left column), staining (middle column), and after bleaching (right column) of HSP60 (green) for direct IF. Before and after bleaching images confirm signal addition and removal before the next staining cycle. Some green background remains after bleaching but this is uniform outside the cell and is easily removed. All scale bars 10 μm. On the right, the intensity sum of ten regions before staining, staining, and after bleaching is quantified to validate the effect of the fluorophore bleaching process. Before (red) and after bleach (blue) show comparable intensities while marker (green) illustrates approximately 2-fold higher intensity.

FIGS. 75A-75B show validation of bleaching protocol for segmentation markers Phalloidin and WGA.

FIG. 75A shows images of before staining (left column), staining (middle column), and after bleaching (right column) of Phalloidin (yellow). Before and after bleaching images confirm signal addition and removal before the next staining cycle. All scale bars 10 μm. On the right, the intensity sum of ten regions before staining, staining, and after bleaching is quantified to validate the effect of the fluorophore bleaching process. Before (red) and after bleach (blue) show comparable intensities while marker (green) illustrates approximately 2-3-fold higher intensity.

FIG. 75B shows images of before staining (left column), staining (middle column), and after bleaching (right column) of WGA (red). Before and after bleaching images confirm signal addition and removal before the next staining cycle. All scale bars 10 μm. On the right, the intensity sum of ten regions before staining, staining, and after bleaching is quantified to validate the effect of the fluorophore bleaching process. Before (red) and after bleach (blue) show comparable intensities while marker (green) illustrates approximately 2-7-fold higher intensity.

FIGS. 76A-76B show segmentation of single cells.

FIG. 76A shows segmentation of single cells using Phalloidin as the cell mask (top row) and DAPI as the nuclear mask (bottom row). Column 1: raw signal from Phalloidin or DAPI, Column 2: cell or nuclear mask label image from the raw signal, Column 3: cell or nuclear segmentation boundary example from the masks, Column 4: resulting segmented cell and its nucleus.

FIG. 76B shows comparing the area obtained by segmenting single cells (n=10) using two markers—Phalloidin (green, row 1) and WGA (magenta, row 2). The bottom bar graph shows the sum intensities of 10 image regions. The Mann-Whitney test shows no statistical significance between the areas of the two markers (p=0.85), indicating that the area distributions are the same for the two markers and either of them can be used for cell segmentation. All scale bars 10 μm.

FIGS. 77A-77D show conversion of 2D spatial coordinates to 1D spatial distributions for KS Hypothesis Testing.

FIG. 77A shows example illustration of 2D cell focusing on two arbitrary protein markers: green and magenta. The black star denotes the cell's center of mass computed from the cell segmentation mask. Created with BioRender.

FIG. 77B shows calculated distances from each protein (green, magenta) pixel to the center of mass resulting in 1D histograms of spatial distances. KS Hypothesis Testing determines if the two signals (green and magenta) come from significantly different underlying distributions. Created with BioRender.

FIG. 77C shows an example BM cell 1 with significantly (p-value ≤0.0001) different underlying distributions for the target mitochondria organelle labeled by HSP60 (magenta) and TOM20 (green) protein markers. See FIG. 63D for complete cells.

FIG. 77D shows an example BM cell 2 with no significant statistical difference in mitochondria spatial expression between HSP60 (magenta) and TOM20 (green) protein markers. See FIG. 63D for complete cells.

FIGS. 78A-78C show sequential HCR-FISH in MSCs in hydrogel.

FIG. 78A shows sequential HCR-FISH of MSCs in 3D hydrogel. MSCs were embedded in hydrogel. Amplification of fluorescence from HCR-FISH enhances the signal. The hybridization-DNase-hybridization cycle allows multiplexed detection of single transcripts.

FIG. 78B shows a 3D subcellular spatial transcriptomics profile of BM-MSCs.

FIG. 78C shows a 3D subcellular spatial transcriptomics profile of UC-MSCs.

FIGS. 79A-79F show SpaGNN analysis of MSCs in 3D.

FIG. 79A shows a patch correlation of MSCs in 3D. Cells were separated into spatially resolved patches by clustering the 3D positions of all detected transcripts. The copy number of each gene in each patch was calculated, and the correlation between genes among patches were computed.

FIG. 79B shows an example of BM-MSC patch correlation.

FIG. 79C shows an example of UC-MSC patch correlation.

FIG. 79D shows a pairwise gene proximity relationship within a patch. By finding the nearest neighbor graph and permutation analysis, we compute the pairwise gene proximity score that quantify the tendency of transcripts being immediate neighbors of each other.

FIG. 79E shows an example of heterogeneous gene proximity of a BM-MSC.

FIG. 79F shows an example of heterogeneous gene proximity of a UC-MSC.

FIGS. 80A-80E show graph autoencoder.

FIG. 80A shows graph autoencoder to embed the patch graphs constructed from single cells. Patch graphs were constructed among patches in each cell by defining each patch as nodes, and connecting each patch and its three nearest neighbors as edges. The pairwise gene proximity scores of each patch are defined as the node features. The convolution layers encode the node features into a new set of features of lower dimension. The inner product decoder uses the encoded features to predict the connection between each pair of nodes. The predicted edges were then compared to the actual edges and the parameters in the encoder were adjusted.

FIG. 80B shows embedded features of patches of a BM-MSC. The features were selected based on the significant differences between BM-MSCs and UC-MSCs. The feature values correspond to the color of the node.

FIG. 80C shows embedded features of patches of a UC-MSC. The feature values correspond to the color of the node.

FIG. 80D shows the node importance of a BM-MSC. A graph neural network explainer model was used to find important nodes and edges that influence the embedded features. The node and edge importance correspond to the color bar. Nodes and edges with importance values close to one have a high influence on the embedded features.

FIG. 80E shows the node and edge importance of a UC-MSC.

FIGS. 81A-81D show classification of cell types using single-cell RNA count and graph autoencoder embedded features.

FIG. 81A shows average pooling of embedded features. Features of all nodes in a cell were pooled by average to determine a set of features that describe a cell. The pooled features of each cell can be used to cluster and classify cell types.

FIG. 81B shows tSNE plot of single-cell RNA count.

FIG. 81C shows tSNE plot of single-cell pooled embedded features.

FIG. 81D shows classification performance of logistic regression when classifying BM-MSC or UC-MSC based on single-cell RNA count, patch correlation, network variability, and pooled network embedding features. All four datasets performs similarly when measured by accuracy and area under receive-operating curve (AUC).

FIGS. 82A-82D show homotypic and heterotypic cell interaction between MSCs in 3D hydrogel.

FIG. 82A shows homotypic and heterotypic interaction between MSCs in 3D hydrogel. The cells were clustered based on single-cell RNA count. Immediate spatial proximity between cells was considered a cell-cell interaction. The interaction is considered homotypic if the two cells are of the same cluster, and heterotypic if the cells were of different cluster.

FIG. 82B shows clustering of cell based on single-cell RNA count.

FIG. 82C shows location of captured cell-interactions in 3D hydrogel.

FIG. 82D shows examples of homotypic and heterotypic interactions. The scatter points is the center positions of detected patches. The scatter point colors correspond to the cluster of the cell. Edges between scatter points connects patches that are involved in interactions. These patches were considered border patches. Yellow connections indicate identified homotypic interactions and red edges indicate identified heterotypic interactions.

FIGS. 83A-83D show examples of local gene-proximity in border patches.

FIGS. 83A-83B show gene proximity of border patches in homotypic interactions.

FIGS. 83C-83D show gene proximity of border patches in heterotypic interactions.

FIGS. 84A-84F show comparison of gene proximity in border patches.

FIG. 84A shows statistical significance between pairwise gene proximity scores of homotypic and heterotypic border patches between BM-MSCs. The highlighted gene-pairs show statistically significant differences between homotypic and heterotypic border patches.

FIG. 84B shows statistical significance between pairwise gene proximity scores of homotypic and heterotypic border patches between UC-MSCs.

FIG. 84C shows a boxplot comparing proximity scores of border patches between BM-MSCs. Homotypic border patches show higher EEF2-IBSP and lower COMP-MKI67 proximity.

FIG. 84D shows a boxplot comparing proximity scores of border patches between UC-MSCs. Homotypic border patches show higher COL1A1-IL6, MKI67-PDL1, and COL1A1-COL5A2 proximity.

FIG. 84E shows COL1A1-IL6 neighborhoods of homotypic border patches between UC-MSCs.

FIG. 84F shows COL1A1-IL6 neighborhoods of heterotypic border patches between UC-MSCs.

FIG. 85 shows autoencoder-based anomaly detection for cell-cell interaction score.

FIGS. 86A-86D show single cell prediction in co-culture using graphs (Vertical multiplexing).

FIG. 86A shows a culturing protocol.

FIG. 86B shows DNase and hybridization diagram.

FIGS. 86C-86D show single cell prediction using graphs.

FIGS. 87A-87B show Single-cell prediction in co-culture using graphs (Vertical multiplexing).

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise. As used herein, the term “substantially free,” when used in the context of a composition or component of a composition that is substantially absent, is intended to refer to an amount that is then about 1% by weight or less, e.g., less than about 0.5% by weight, less than about 0.1% by weight, less than about 0.05% by weight, or less than about 0.01% by weight of the stated material, based on the total weight of the composition.

The term “subject” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

### Method

### Method of Characterizing a Nucleic Acid Network

The present disclosure, in one aspect, provides for a method of characterizing a nucleic acid network comprising: providing a biological sample on a substrate, wherein the biological sample comprises the nucleic acid network; applying spatially-aware graph neural networks (spaGNN) to the biological sample to obtain characterization data; and analyzing the characterization data.

In some examples, the substrate comprises a coverslip, a co-culture, or any combination thereof. In further examples, the substrate comprises glass. In certain examples, the substrate is from 100 to 150 μm thick. In specific examples, the substrate is coated with a layer of a hydrogel, Matrigel, collagen, cultrex, or any combination thereof.

In some examples, the biological sample comprises a tissue sample. In further examples, the tissue sample comprises an umbilical cord, adipose tissue, or bone marrow.

In certain examples, the biological sample comprises a stem cell. In specific examples, the stem cell comprises a mesenchymal stem cell (MSC). MSCs are stromal cells with the ability to self-renew and exhibit multilineage differentiation, such as into osteoblasts, chondrocytes, myocytes, and/or adipocytes. In some examples, MSCs are isolated from a variety of tissues including but not limited to umbilical cord, endometrial polyps, menses blood, bone marrow, molar cells, amniotic fluid, and adipose tissue.

In some examples, the MSC comprises an organelle. In further examples, the organelle is a nucleus, mitochondria, a Golgi, an endoplasmic reticulum, or any combination thereof. An organelle refers to a subcellular structure, usually from inside a cell, such as the nucleus, mitochondria, Golgi, and endoplasmic reticulum, wherein the nuclei stores genetic information, and the mitochondria produces chemical energy. In some examples, an organelle is obtained from an MSC.

In certain examples, the nucleic acid network comprises RNA, DNA, or any combination thereof.

Cells perform different functions like providing structure and support, facilitating growth, producing energy, etc. to support and sustain life. These activities are handled by organelles such as the nucleus, mitochondria, endoplasmic reticulum, and Golgi apparatus^{1e}. Organelles cooperate to form a network of interactions that enable different cellular activities^{2e}. The methods disclosed herein allow for examination of organelle interactions to more comprehensively understand how cells function.

In further examples, the method disclosed herein allows for collection of characterization data to account for cell-to-cell variability, such as between cells of the same type as well as cells of different types, which results in molecularly and functionally distinct cells^{3e}. Such differences may contribute to the health and function of the entire organism. Multiple factors, such as microenvironment variability, differences in the cellular stages, genetics or epigenetics, or fluctuations in gene expression levels, can cause this heterogeneity.

In specific examples, the characterization data comprises information relating to the nucleic acid network potency, identity, proximity, communication, therapeutic efficacy, interactions, associated energy, or a combination thereof. The data is obtained via analyzing nucleic acid networks within the same cell, in some examples in MSCS, and allows for an understanding of cell functions. In further examples, the characterization data is obtained from spatial information on the organelles within the cell.

Cells can display varying molecular profiles, differentiation potential, and therapeutic efficacy due to factors such as microenvironment variability, differences in cellular stages, genetics or epigenetics, or fluctuations in gene expression levels, which renders these various cells suitable for various application within a subject.

In some examples, analyzing the characterization data comprises obtaining a scatter plot of intensity, calculating a Pearson's correlation coefficient, calculating a pixel overlap colocalization, conducting the Kolmogorov-Smirnov hypothesis test, plotting average intensity values per cell as a boxplot, clustering pixel phenotypes, calculating pixel intensity, energy Laplacian, modified Laplacian, diagonal Laplacian, variance Laplacian, or gray level variance, or obtaining morphological features for each cell.

In further examples, providing the biological sample on the substrate comprises culturing the cells on the substrate. In some examples, culturing the cells comprises isolating MSCs. In certain examples, MSCs are isolated via the following protocol:
1. Obtain fresh tissue aspirates that are collected under aseptic conditions.
2. Filter the cell suspension through a 70-mm filter mesh (CLS431751) to remove any cell clumps.
3. Pellet the cells at 500 g for 5 min in a benchtop centrifuge.
4. Resuspend the cells and determine the yield and viability of cells by Trypan blue exclusion.
5. Culture cells in T75 culture dishes in 10 mL of complete MSC medium at a density of 25×10⁶ cells/mL. Incubate the plates at 37° C. with 5% CO₂ in a humidified chamber without disturbing them.
6. After 3 h, remove the non-adherent cells that accumulate on the surface of the dish by changing the medium and replacing with 10 mL fresh complete medium.
7. After an additional 8 h of culture, replace the medium with 10 mL of fresh complete medium. Thereafter, repeat this step every 8 h for up to 72 h of initial culture.
8. Cells can be frozen in MSC growth media plus 10% DMSO (D2650) at a density of 2×10⁶ cells/vial.

In further examples, culturing the cell comprises thawing MSCs, expanding MSCs, or a combination thereof.

In specific examples, providing the biological sample on the substrate comprises performing immunofluorescence on the biological sample, thereby staining the stem cell with a marker. Immunofluorescence is a light microscopy-based technique that enables visualization of components in a given tissue or cell type through the tagging of target molecules, such as antibodies, with fluorophores. In some examples, immunofluorescence is performed prior to applying spaGNN.

In certain examples, artificial intelligence and/or machine learning is used to analyze the characterization data. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally, one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight (e.g., Wl). ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to back-propagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

### Method of Providing Stem Cell Therapy

Also provided herein is a method of providing stem cell therapy to a subject in need thereof comprising: performing the method disclosed herein to a stem cell; and administering the stem cell to the subject.

In some examples, performing the method comprises identifying RNA expression on the stem cell.

In an alternative aspect, provided herein is a method of predicting a subject's response to a stem cell therapy, comprising (i) providing a biological sample on a substrate, wherein the biological sample comprises a nucleic acid network; (ii) applying spatially-aware graph neural networks (spaGNN) to the biological sample to obtain characterization data; (iii) analyzing the characterization data; and (iv) based upon the characterization data, administering the stem cell therapy to the subject. In some embodiments, the biological sample comprises a tissue sample. In some embodiments, the tissue sample comprises an umbilical cord, adipose tissue, or bone marrow. In some embodiments, the biological sample comprises a stem cell. In some embodiments, the stem cell is characterized by low or reduced inflammation. In some embodiments, the stem cell is characterized by low or reduced immunogenicity. In some embodiments, the stem cell is immune inhibitory. In some embodiments, the stem cell comprises an MSC. In some embodiments, the biological sample further comprises an immune cell. In some embodiments, the method further comprises administering to the subject the stem cell therapy.

In some embodiments, providing the biological sample on the substrate comprises co-culturing MSCs with one or more immune cells. In some embodiments, the biological sample comprises one or more MSCs and one or more immune cells, wherein the MSCs and immune cells are co-cultured on a 3D substrate. In some embodiments, the one or more immune cells are selected from T cells, Myeloid cells, macrophages, M1-like macrophages, and M2-like macrophages. In some embodiments, the one or more immune cells comprise T cells. In some embodiments, the T cells are CD3+.

In some embodiments, the characterization data comprises obtaining a scatter plot of intensity, calculating a Pearson's correlation coefficient, calculating a pixel overlap colocalization, conducting the Kolmogorov-Smirnov hypothesis test, plotting average intensity values per cell as a boxplot, clustering pixel phenotypes, calculating pixel intensity, energy Laplacian, modified Laplacian, diagonal Laplacian, variance Laplacian, or gray level variance, or obtaining morphological features for each cell. In some embodiments, the characterization data comprises an image-based proliferation (Ki67) or a survival (TUNEL) analysis. In some embodiments, the characterization data indicates activation or suppression of one or more immune cells. In some embodiments, if the characterization data indicates that the one or more immune cells are suppressed, then the stem cell therapy is administered to the subject.

In another aspect, provided herein is a method of providing an MSC therapy to a subject in need thereof, comprising (i) providing one or more MSCs and one or more immune cells on a 3D substrate, and wherein the biological sample comprises a nucleic acid network; (ii) applying spatially-aware graph neural networks (spaGNN) to the biological sample to obtain characterization data; (iii) analyzing the characterization data for markers of immune cell activation or suppression; and (iv) if the characterization data indicate that the one or more immune cells are suppressed, then administering the MSC therapy to the subject. In some embodiments, the MSCs reduce the proliferation, quantity, and/or activity of the one or more immune cells, optionally selected from T cells and NK cells. In some embodiments, the one or more immune cells comprise T cells. In some embodiments, the T cells are CD3+. In some embodiments, the T cell characterization data comprises analyzing TNF/Cytokine pathways. In some embodiments, the stem cell is formulated for infusion administration to the subject.

In alternative embodiments, the MSCs are derived from a subject who is not intended to receive the therapy. In some embodiments, the MSCs are allogeneic to the subject intended to receive the therapy.

In some embodiments, the subject has an inflammatory disorder. In some embodiments, the subject has a blood cancer. In some embodiments, the MSC therapy is used to enhance hematopoietic cell transplantation in a subject. In some embodiments, the method of providing the MSC therapy is for the prevention or treatment of graft-versus-host disease (GVHD).

In further examples, the subject has anemia, a blood disorder, an inherited red cell abnormality, a bone marrow cancer, leukemia, lymphoma, an inherited immune disorder, an inherited metabolic disorder, an inherited platelet abnormality, a phagocyte disorder, a solid tumor, or an immune and other system disorder.

In certain examples, anemia is Aplastic Anemia, Congenital Dyserythropoietic Anemia, Paroxysmal Nocturnal Hemoglobinuria and Fanconi Anemia.

In specific examples, the blood disorder or inherited red cell abnormality is Blackfan-Diamond Anemia, Beta Thalassemia Major, or Pure Red Cell Aplasia and Sickle Cell Disease.

In some examples, the bone marrow cancer is Plasma Cell Leukemia, Multiple Myeloma or Waldenstrom's Macroglobulinemia.

In further examples, leukemia is Acute Lymphoblastic Leukemia, Acute Biphenotypic Leukemia, Acute Myelogenous Leukemia, Acute Lymphoblastic Leukemia, Acute Undifferentiated Leukemia, Acute Myelogenous Leukemia (AML), Chronic Lymphocytic Leukemia, Refractory Anemia with Excess Blasts, Juvenile Chronic Myelogenous Leukemia Refractory Anemia, Juvenile Myelomonocytic Leukemia or Chronic Myelomonocytic Leukemia.

In certain examples, lymphoma is Hodgkin's lymphoma.

In specific examples, the inherited immune disorder is Omenn Syndrome, Kostmann Syndrome, Bare Lymphocyte Syndrome, Ataxia-Telangiectasia, DiGeorge Syndrome, Common Variable Immunodeficiency, Lymphoproliferative Disorders, Leukocyte Adhesion Deficiency, Wiskott-Aldrich Syndrome, Acute Myelofibrosis, Myeloproliferative disorders, Polycythemia Vera, Agnogenic Myeloid Metaplasia, Essential Thrombocythemia, SCID (X-linked), SCID (ADA-SCID) or SCID with absence of normal B cells and T cells.

In some examples, the inherited metabolic disorder is Hurler's Syndrome, Mucopolysaccharidoses, Scheie Syndrome, Sanfilippo Syndrome, Hunter's Syndrome, Morquio Syndrome, Maroteaux-Lamy Syndrome, Sly Syndrome, Mucolipidosis II, Adrenoleukodystrophy, Metachromatic Leukodystrophy, Krabbe Disease, Metachromatic Leukodystrophy, Gaucher Disease, Pelizaeus-Merzbacher Disease, Sandhoff Disease, Niemann-Pick Disease, Tay-Sachs Disease, Lesch-Nyhan Syndrome or Wolman Disease.

In further examples, the inherited platelet abnormality is Congenital Thrombocytopenia or Glanzmann Thrombasthenia.

In certain examples, the phagocyte disorder is Neutrophil Actin Deficiency, Chronic Granulomatous Disease, Chediak-Higashi Syndrome or Reticular Dysgenesis.

In specific examples, the solid tumor is Medulloblastoma, Retinoblastoma or Neuroblastoma.

In some examples, the immune and other system disorder is Cartilage-Hair Hypoplasia, Pearson's Syndrome, Gunther's Disease, Hermansky-Pudlak Syndrome, Systemic Mastocytosis or Shwachman-Diamond Syndrome.

In some examples, the stem cell is administered to the subject via intravenous (IV) infusion. In further examples, the stem cell is administered to the subject via intramuscular injection.

### Product

### Kit

Provided herein is a kit comprising a stem cell targeting reagent and an organelle map, wherein the organelle map comprises data indicating identification and proximity of RNA inside of an organelle and outside of an organelle.

In some examples, the map is software-based that employs logical operations, (1) as a sequence of computer-implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit and system memory. Depending on the exact configuration and type of computing device, system memory may be volatile (such as random-access memory (RAM)), non-volatile (such as read-only memory (ROM), flash memory, etc.), or some combination of the two.

The processing unit may be a programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture in order to store and execute the software components presented herein. It also should be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process.

### Example 1: Spatially Resolved Planar and Volumetric Gene Neighborhood Networks for Functional Mapping of Cell Identity, Communication, and Potency

Single-cell spatial analysis methods revealed cell states, phenotypes, and cell-cell interactions. Subcellular spatially resolved transcriptomics and proteomics were leveraged to study spatial gene networks in Mesenchymal stem cells (MSCs), particularly cultured MSCs on coverslips, MSCs in 3D gel (hydrogel, cultrex, or matrigel), and MSC-t-cell co-culture. Disclosed herein is a high-throughput screening of therapeutic MSCs potency via subcellular spatial analysis.

The spaGNN and broadly subcellular gene neighborhood and organelle networks revealed spatially resolved intracellular biology and this rapid, multiplex strategy was applicable to a broad range of cells used in pre-clinical and clinical research.

The spaGNN workflow employed spatially resolved transcriptomics techniques to study subcellular gene spatial networks in MSCs. The spaGNN pipeline studied cultured cells and tissue at subcellular resolution. This technology revealed MSC potency indications masked by traditional analysis methods. MSC-t-cell co-culture established models to study MSC and immune cell interactions in vitro to predict what would happen to the stem cells when encountered in stem cell transplants.

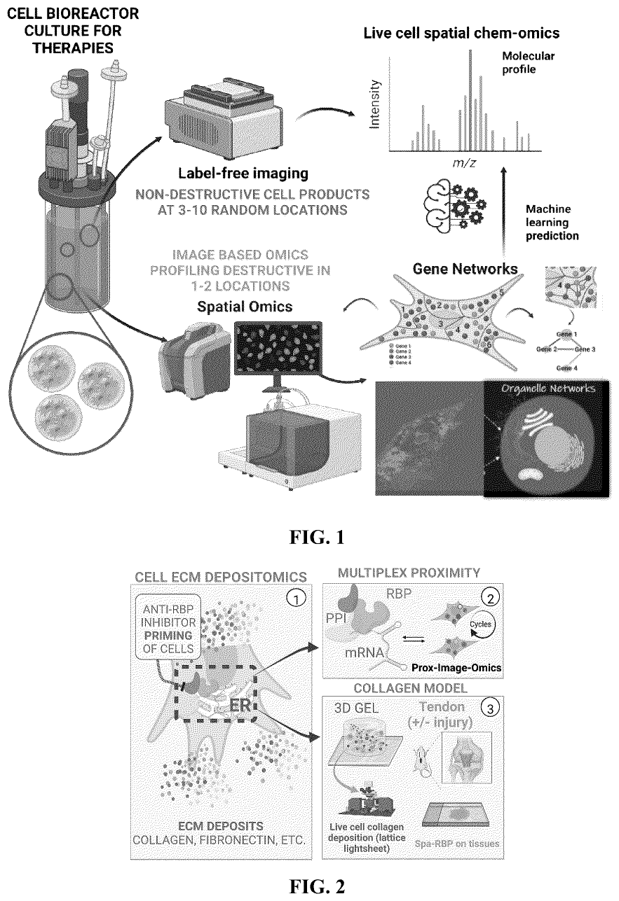

Destructive analysis of RNA, protein, and organelle networks of single stem cell products was used with spatial omics approaches, resulting in spatial omics maps. “Transfer learning” was used to identify good cell products in live cells from spatial omics maps. This allowed for integration of both non-destructive and destructive imaging on the same cell therapy characterization platform technology (FIG. 1).

### Example 2: Multiplexed RNA and Protein Image Proximity for Guiding Cellular ECM Depositomics

### Introduction

Extracellular matrix (ECM) is an assembly of non-cellular proteins that regulate cell function and response in all tissues and organs.^{1a} ECM exhibits “biochemical” characteristics to maintain tissue architecture, presents “bioadhesive” properties to enable cell migration, and generates “biochemical” cues to control cellular signaling and differentiation.^{2a} Recent molecular profiling techniques have identified 1027 genes defined as matrisome, encoding the functional supramolecular complexes of ECM proteins (e.g., collagens, laminins, fibronectin, vitronectin, and elastin) and ECM-interacting proteins in the human genome.^{3a} ECM is dynamically re-modeled in injury, aging, cancers, and cardiovascular diseases.^{4a} Cells deposit and degrade diverse ECM compositions in tissue microenvironments, controlling response to environmental stimuli and maintaining homeostasis. ECM remodeling rates are dependent on development, wound repair, infections, and many diseases. Thus, this example describes “Depositomics” to decipher the gene regulatory mechanisms of how cells deposit many ECM molecules and what the subtypes of ECM proteins are dominant in health and disease.

Regenerative medicine is an emerging field to restore damaged tissues using cell injections and immune modulation.^{5a} To this end, ECM diversity provides a “context” and “support” to therapeutic cells at the tissue site in need of repair in regenerative medicine.^{6a} Mesenchymal stem cell (MSC)-based therapies have widely been used for the treatment of many inflammatory diseases due to their immunomodulatory function based on the secretome and depositome. MSCs' secretion profiles are largely regulated by auto and paracrine signaling effects on other cells in target tissue sites and they produce inflammatory cytokines (e.g. CCL2, CXCL8, and many others) to modulate immune and stromal cells.^{7a} However, there is an intricate balance of MSCs' ECM deposition profiles and their bioactive secretome molecules in tissue repair sites. In some cases, MSCs interact with fibroblast-like cells (such as tenocytes) to perform repair activities.^{8a} In other cases, MSCs directly produce ECM (e.g., collagen-I), instead of encouraging other cells to deposit, eventually leading to fibrosis in graft-versus-host disease (GVHD) treatments.^{9a}

In parallel, activation of MSCs, known as priming MSCs, is used to enhance the healing activities and reparative potential in cell-based therapies. Further, a new mechanism of modulating RNA-binding proteins (RBPs) has been used to alter the stem cell fate of MSCs using RBP-PUM2 blockade in zebrafish larvae models.^{10a} Along these lines, we have recently identified MSCs' RNA localization for collagen-I genes on the endoplasmic reticulum (ER), suggesting the novel roles of ER-associated proteins in the regulation of collagen-I depositions by the MSCs.. Developed herein is (a) multiplexed RNA-protein interaction method using a proximity ligation assay, (b) Live-cell imaging probes of ECM-depositions of stem cells in 3D hydrogels imaged by lattice light sheet microscopy, and (c) a tendon healing preclinical model to test reparative activities of depositions by RBP-primed MSCs (FIG. 2). The hypothesis is that spatial RBP mapping and targeting modulate depositomics for treating ECM-disorders.

### SUMMARY

First, develop single-cell spatial RNA-protein interaction assay (Spa-RBP) profiling technology and apply this emerging approach to mesenchymal stem cell biology with and without RBP inhibitors.

Spa-RBP results for modulating MSC depositome are used to identify targets in vitro cultures on thin optically-clear cyclic olefin bottom, collagen-coated PhenoPlate 96-well microplates using high-resolution (60×) inspection of RNA-protein interactions.

Spa-RBP comparisons for modulating human (in vitro) and rat (in vivo) derived Bone Marrow-Derived MSCs (BM-MSCs) and Adipose Tissue-Derived MSCs are established to distinguish the “depositome differences and uniqueness” of each cell origins from distinct donors.

As a model system, we focus on the collagen 1 of an initial proof-of-concept to alter the MSC depositome as a result of RBP-LARP6 perturbations by small interfering RNAs (siRNAs), CRISPR-CAS9 knockouts, or overexpression assays. After LARP6 reduction, we perform the Spa-RBP assay, the collagen 1 RNA localization, and ELISA-based detection of secreted collagen 1 in the supernatant in comparison to controls. The expected outcome is to confirm the role of modulating collagen 1 using the RBP targeting approach. This finding allows for a novel priming of MSCs to “activate” or “inhibit” depositome pathways, making them amenable to treatments of ECM-dysregulated disorders, specifically the tendon repair models.

Second, the MSCs is studied in 3D hydrogel-fiber composites to evaluate depositome in response to physiologically relevant biomechanical and adhesive cues for improving MSC survival, proliferation, and function. To evaluate the real-time deposition of the depositome, we perform live imaging of MSCs' deposited ECM molecules using lattice light sheet imaging over the entire 3D MSC-laden hydrogel.

Human-derived BM-MSCs and AT-MSCs will be grown separately in composites consisting of highly crosslinked poly(ethylene glycol) hydrogel fibers^{12a} surrounded by PEG-gels (i.e., mimicking tendon) with matrix metalloproteinase (MMP) sensitivity to break down ECM proteins and bioadhesive peptides to introduce additional ECM composition. After 5 to 28 days with and without RBP-inhibitors, the samples will be fixed for Spa-RBP assays and the collagen 1 RNA localization that will be performed in each sample. In parallel, ELISA-based detection of secreted collagen 1 analysis will be measured in supernatant from each MSC-laden hydrogel in comparison to controls. The expected outcome is to quantify the basic role of ECM components in the hydrogel on Anti-RBP-primed MSCs. This data will serve as a predictive MSC depositome assay before starting pre-clinical applications.

To evaluate the depositome dynamics, collagen 1 is visualized in real-time using the incorporation of azide-L-proline into the neo-collagen produced by cells can then be detected by neo-collagens conjugated to a 10 mM of a Click-IT Alexa Fluor 488 DIBO Alkyne^{13a} that will be imaged using a rapid lattice light sheet microscopy platform over 2 mm×2 mm×0.2 mm 3D hydrogel volume. The expected outcome of this assay is to establish a time derivative of collagen-I deposition, revealing the 3D distribution and heterogeneity of “depositome” across the entire bioconstruct.

Finally, we will validate depositome in vivo using a preclinical tendon repair model after rotator cuff injury. MSCs injected into rats enhance tendon healing through a few mechanisms. MSC differentiation to tenocytes plays a key role to promote “depositome” for tendon regeneration. Tenocytes deposit collagen 1 and III in addition to MSCs to enable tissue healing. Anti-RBP-primed MSCs may thus enhance to “depositome” in tendons through mechanisms involving MSC-differentiation to tenocytes. To evaluate in vivo collagen 1 deposition, we will analyze the collagens and spatially resolved RBPs to monitor collagen concentration accumulating as a result of tendon healing after rotary cuff injury.

To demonstrate the clinical potential, Anti-RBP primed rat-derived BM and AT-MSCs and separately unprimed controls will be directly injected into the surgical^{14a} tendon repair model 2-weeks after rotator cuff injury in a rat. The post-14-day time window was selected to visualize collagen deposition in tendon repair right after inflammatory response to the injury. After the 14-30 days of regeneration, the tendons will be explanted for follow-up histology validation of Collagen-I staining and spatially resolved RBP maps of MSCs in tendon tissues. The expected outcome of this approach is to evaluate the in vivo performance of anti-RBP primed cells and relate them to the tendon regeneration metrics.

In sum, to test the hypothesis, RBP-primed MSCs will be analyzed in single cells, in 3D fiber-reinforced hydrogels, and in a collagen 1-driven rat tendon repair model, which can then be tailored to clinical use.

### Background

### RNA-Binding Protein Interaction Profiling Methods.

RNAs and proteins interact with other to regulate cellular function, dysfunction, and homeostasis.^{15a} Specifically, proteins bind to untranslated or coding regions of mRNAs to control gene expression.^{16a} Mostly, mass spectrometry and sequencing methods have been used to profile RBP-associated molecular maps in yeast, mammalian, and nematode models.^{19a} Live cell imaging assays have also been investigated to quantify real-time and spatial interactions of a limited number of RNA-protein pairs.^{20a-22a} Despite ample method development in this RBP field, there is a crucial need to map out the spatially resolved and multiplexed RBPs in “fixed” cells and tissues using image-based assessment of the native localization of “multiple” RNA and “multi” protein interaction events, otherwise unattainable by single-cell RNA-sequencing due to limitations of capturing only pairwise interactions. Adding spatial data to the RBP assays will allow mechanistic investigation of cells used for clinics, even in the archival patient tissues. The key features of our methods and approach are summarized in Table 1.

TABLE 1
Emerging RNA-binding protein interaction (RBP) assays and technologies.

| | | | | Spatially | | |
|---|---|---|---|---|---|---|
| | | | | resolved | Biological | |
| Method | Capability | Technology | Multiplex | ? | Sample | Reference |
| Spa-RBP | RBP mapping | Single- | Yes | Yes | Stem | Proposed |
| | with | molecule | | | cells; | Single Cell |
| | multiplexed | Imaging | | | ECM- | |
| | proximity | | | | depositing | |
| | ligation | | | | cells | |
| iCLIP, | RBP profiling | Sequencing | Yes | No | Mammalian | Bulk ^{23a-26a} |
| easyCLIP, | with | | | | | |
| eCLIP | immunoprecipitation | | | | | |
| Matrix- | RBP screening | Sequencing | Yes | No | Mammalian | Bulk ^{27a} |
| based | using rec-Y3H | | | | | |
| | matrix | | | | | |
| | screening | | | | | |
| RBDmap | Only detects | Mass spec. | Yes | No | HeLa | Bulk^{28a} |
| | RNA-binding | | | | | |
| | domains | | | | | |
| incPRINT | In-cell RNA- | Luminescence | Yes | No | Mammalian | Bulk^{28a} |
| | protein | | | | | |
| | interactions | | | | | |
| STAMP | Discover RNA | Sequencing | Yes | No | Mammalian | Single |
| | targets of RBPs | | | | | Cell^{29a} |
| Live | Spatio-temporal | Mass spec | Yes | Subcellular | Mammalian | Bulk/Single |
| cell/high- | phase | and | | but | | Cell^{20a, 30a} |
| throughput: | separation | Imaging | | not in | | |
| APEX-PS; | assay | | | situ | | |
| APEX-RIP | | | | | | |
| Live cell: | Real-time | In vitro/In | No | Yes | Neurons | Single |
| FRET, | tracking of | vivo | | | | Cell^{21a, 22a, 31a, 32a} |
| Trifluorescence | RNA-protein | Imaging | | | | |
| | interactions | | | | | |

Spa-RBP works based on sequential imaging and labeling strategy of RBPs in fresh or fixed cells and tissue at the single-molecule level. Matrix-based, STAMP and iCLIP derivatives use sequencing readout to map one protein to one RNA interactions for multiple pairs. RBDmap and APEX use mass spectrometry to localize coarse subcellular compartments for RBP mapping. Live cell FRET imaging only maps a few RBP pairs.

Thus, performed herein is multiplexing proximity ligation assay (PLA) to systematically target RNAs and proteins interacting within a 20-40 nm neighborhood. We refer to this method as spatial-RBP (Spa-RBP) which can provide multiplexing, spatial data, single cell sensitivity, subcellular localization, and novel methods to prime stem cells for revolutionizing regenerative medicine.

Impact: Why Spatial Single Cell RBP Analysis?

1) RNA seq-based RBP analysis methods capture pairwise interactions at the bulk level or detect only RNAs or protein targets of RBPs (STAMP is single cell but not spatial). APEX localizes subcellular parts but no single cell. Spatial RBP analysis can map multiple RNAs to multiple proteins by “localizing” them in the image of single cells.

2) RNA-seq fails to work in fixed tissues. For clinical use, Spa-RBP tracks the RBP state of single cells in archival biopsies, mapping tissue context of RBPs.

### RNA-Protein Interactions as Depositome Regulator

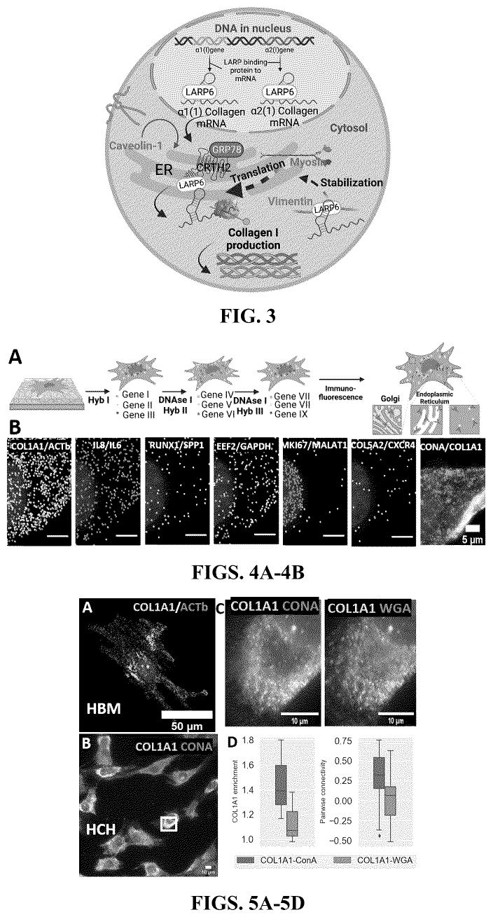

Gene and protein regulation mechanism of cell depositome is central to this proposal. Among 1,027 ECM-related genes, the focus will be to decipher the regulatory control of the collagen 1 gene, the most abundant protein in the human body. While the collagen 1 protein turns over slowly in health, the production of the Type I collagen is upregulated in profibrotic insults, for instance, in the wound healing process. Despite significant effort, no specific transcriptional regulator (e.g., transcription factor) could be found for collagen 1, suggesting an intricate regulation beyond a simple transcriptional activation in reparative activities. Initially, hepatic stellate cells (HSCs) were chosen as a model to examine the collagen 1 deposition at the gene regulation level.^{33a} Because major collagen 1 depositing cells include fibroblasts, myofibroblasts, osteoblasts, smooth muscle cells, and HSCs, the selection of the initial HSC cell model was justified to decipher the rapid production of collagen deposition in reparative processes. Early efforts identified a dilemma in upregulation of transcription only by 3-fold while producing up to 100-fold more mRNA molecules, yielding an elongated 24 h half-life of collagen 1 mRNA in activated HSCs. Consequently, the major mechanism has been associated with the stabilization of collagen 1 mRNA as a post-transcriptional control, including RNA and protein interactions acting to regulate the translation of depositome.^{34a} Thus, emerging roles of RNA binding proteins (RBPs) identified that 1) αCP binds to the 3′ UTR of Collagen al(I) mRNA, 2) LARP6 binds to the 5′ stem-loop of Collagen mRNAs, 3) other non-muscle myosins II (folding partner), vimentin (stabilizer to extend mRNA lifetime), and RNA helicase A (translation co-factor) proteins, 4) Caveolin-1 dependent CRTH2 protein^{35a} controlling LARP6 (FIG. 3). These targets suggested that anti-RBP small molecular inhibitors (e.g., anti-LARP6)^{36a} can be used to modulate depositome (e.g., collagen 1), providing the rationale to prime the stem cells before their use for cell therapies. Akin to secretome-based cytokine priming, depositome-based priming will be highly innovative in regenerative medicine. The high-risk part is to identify RBPs for other ECM molecules identified in the matrix profiles and repeat the same discovery process of the LARP6-RBP and collagen 1 association model to other RBP-associated ECM pairs or multiprotein complexes.

### Stem Cells and Depositome in Tendon Regeneration

More than 100 million people worldwide experience musculoskeletal injuries each year and a half of these are tendon injuries.^{37a} Interestingly, tendons are composed of tenocytes and tenoblasts, and their dry mass comprises 80% collagen 1 as an ECM. In injuries, ECM degrades due to sudden or gradual loss of cellular depositome. MSCs have mostly used stem cells for tendon therapies. To enable tendon healing, MSCs differentiate into mesenchymal tissues such as cartilage or tendon cells (e.g. tenocytes) to enable the production of the matrix. While preclinical work suggests MSC-induced tendon repairs after 4-weeks of injection,^{37a,38a} currently there is no FDA-approved therapeutics and there is a lack of understanding and tools to control collagen 1/III depositome in tendon healing. Thus, this proposal develops cellular engineering tools to target RBP-controlled depositome and seeks emerging ideas to treat tendon injuries using MSC-based collagen 1 depositome as a model system. This technology applies to other ECM disorders.

### Spatial Genomic Analysis of Collagen I and ER-Associated ConA MSCs and Chondrocytes

Spatial genomic analysis of collagen 1 and ER-associated ConA in MSCs and chondrocytes: To measure image-based gene expression, MSCs necessitate the use of multiplexed RNA labeling using the split-probe design of the Hybridization chain reaction (HCR) assay,^{39a} providing bright, single-molecule RNA images with high detection specificity. This multiplexing strategy simultaneously labels and records single-molecule images (60×microscope, 1.4 NA) up to three RNA targets per cycle. Each round of HCR and imaging is followed by enzymatic digestion of HCR components using a DNase I enzyme.^{40a} Subsequent rounds of HCR labeling then target different sets of RNA species, providing a linearly scalable method of cycle-number dependent multiplexing (FIGS. 4A-4B). For example, six sequential hybridizations with three RNA targets per hybridization would profile eighteen unique RNA markers in a single MSC. Next, multiplexed RNA maps in single MSCs are utilized in image-based morphological analysis. The workflow implements a cell painting strategy to co-detect proteins in the same MSC. After completing the multiplexed RNA imaging, the sample is stained with Concanavalin A (ConA, staining Endoplasmic reticulum), Phalloidin (PHA, staining Actin), and Wheat Germ Agglutinin (WGA, staining Golgi apparatus and plasma membrane) to visualize subcellular features of each MSC for segmentation and structural annotation. In this data, 12 markers were measured in the human bone marrow (HBM)-MSCs and 4 additional markers were measured in human chondrocytes (HCHs) (FIG. 5A-5B). By thresholding the IF images, we found more COL1A1 transcripts (˜1.3-1.5 fold) present in ConA positive regions. Thus, COL1A1 transcripts were observed to be enriched on ER structures as detected by ConA staining and were excluded from the Golgi region defined by the WGA stains (FIG. 5C-5D).

### Spatial Gene Neighborhood Network Model

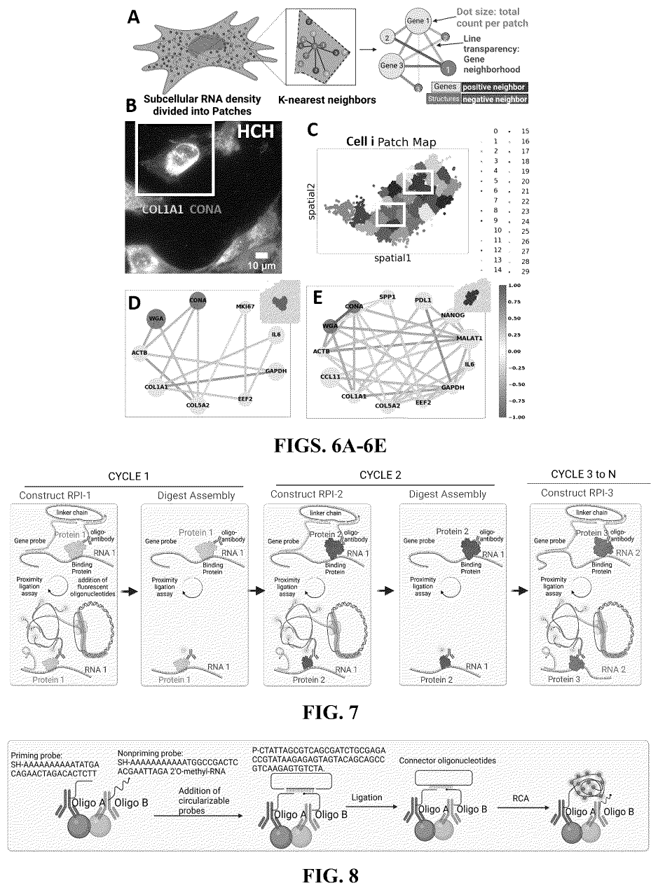

We developed a new analytical method, spatially resolved gene neighborhood network (spaGNN),^{41a} that constructs subcellular molecular density maps to distinguish cell state differences of individual MSCs and HCHs (FIG. 6A). We evaluated the RNA and protein densities in distinct subcellular regions using spaGNN. To quantify local subcellular regions, we created “patches” defined by local RNA density or subcellular spatial regions (FIG. 6B-6C). Average protein density around all transcripts in the same patch is defined as the enrichment of the protein within the patch. Average enrichment of protein around all transcripts indicates protein concentration in the local neighborhood. In a COL1A1 enriched patch, COL1A1 was highly colocalized with ConA, while R-actin was highly colocalized with WGA. We applied the subcellular RNA density patch and spatial neighborhood network analysis to confirm the spatial relationships between RNA and protein (FIG. 6D-6E). We found that COL1A1 tends to colocalize (>0.8 normalized interactions) with ConA (ER) and inversely (<negative 0.5) with WGA (Golgi). Thus, subcellular distributions of RNA may be used to increase secretome^{42a,43a}. These RNA-protein co-localizations will be validated by multiplexed proximity ligation assays developed in this proposal.

### Technology Development

### Multiplexed and Spatially Resolved RNA and Protein Ligation Methods

To determine the RNA-binding proteins (RBP) to mRNAs of MSC depositome generating genes, this proposal will establish a series of multiplexed proximity ligation assays (PLA). Particularly, two proximity assays will be key to studying RBP regulation: 1) image-based multiplexed RNA-protein interaction assay (mRPI) and 2) multiplexed protein-protein interaction assay (mPPI), collectively forming Spa-RBP.

### Multiplexed RNA-Protein Proximity Ligation Assay

This emerging mRPI assay will be built on a “gene probe” to detect RNA and an antibody conjugated to a “DNA oligo” for localizing proteins. When RNA and protein are bound, then the gene probe (magenta) is ligated to oligo (green), starting the rolling circle amplification (RCA) as shown in FIGS. 7 and 8. The resultant signal allows the detection of mRPI pairs. For instance, a 3-cycle experiment identifies RNA1 and Protein 1 in the single-molecule 60× microscopic image (first), followed by DNase I digestion of the PLA assembly (FIG. 7). In the second cycle, RNA 1 and Protein 2 interactions are imaged, followed by 4-hour DNase I enzymatic digestion. Finally, in the third cycle, the interactions of RNA 2 and protein 3 are acquired in the image. This sequence is designed based on RNA FISH probes; (B) 20-35 adenylates (AAA) that serve as a linker; and (C) a 25-nt sequence (TATGACAGAACTAGACACTCTT—SEQ ID NO: 1) that bridges the DNA circle and serves as a primer for rolling-circle amplification during PLA detection.^{45a} Regions B and C are identical for all probes. And the antibody-oligo follows the PLA-based sequences (e.g., SH-AAAAAAAAAAGACGCTAATAGTTA AGACGCTT—SEQ ID NO: 2). The multiplexing is akin to sequential FISH^{40a}, wherein the cycles are performed using DNase I to digest the PLA assembly in between the cycles. This high-risk proposal aims to quantify interactions of 20 ECM-related mRNAs with 20 proteins for each mRNA of interest to identify RBPs regulating depositomics. The mRPI assay should, in principle, be scalable to cover the entire matrisome consisting of 1,000 proteins in tissues.

A proof-of-concept experiment for mRPI will focus on collagen 1 mRNA. The RNA 1 in this scheme will target collagen 1 mRNA and Protein 1 will be RBP-LARP6. The second assembly will target RNA 1 for collagen 1 and Protein 2 will be vimentin. The third assembly will target RNA 1 for collagen 1 and Protein 3 for non-muscle myosin II. Then, to discover new RNAs, we will screen e.g., Collagen 5A2 mRNA (see FIG. 4). After identifying mRNA binding proteins for other collagens using the techniques listed (Table 1), the mRPI assay will expand the RBPs controlling many other collagen and ECM-genes for identifying targets to modulate depositomics

### Multiplexed Protein-Protein Proximity Ligation Assay

Proximity ligation assays detect proteins within 20-40 nm proximity to another protein. The proof-of-principle of mPPI was performed by using the two sets of protein targets using one set of distinct antibodies (Cyclin E & CDK2, PPI1) and another total and phosphorylated site of Akt (PPI2), providing unique PPI within the same cell during phosphorylation and Cyclin E and CDK2 interactions within proximity. Therefore, the interaction between p-AKT (Ser473) and AKT indicates the full activation of AKT. To probe the PPI1 of Cyclin E and CDK2, two sets of PLA assemblies (i.e., the design presented in FIG. 8) were performed in the fixed fresh tissues, yielding ubiquitous PPI (single-molecule images at 60×) in every single cell denoted by red dots (i.e., the same color for two distinct PPI validated multiplexing). After DNase I treatment, the PLA was removed. A second cycle was then performed by a different protein pair of Akt and p-Akt (PPI2) to detect previously unidentified PPIs (FIG. 6). The counts of PPIs (10-100 copies) in single cells will be quantified with and without small molecule treatments

### Spa-RBP Analysis and Real-Time Depositomics of MSCs in 3D Hydrogels

Tendons are composed of aligned collagen 1 fibers and native ECM compositions. To study the basic biology of Anti-RBP primed or control MSC depositions in a biomaterial that recapitulates the structure of tendon, pursued herein is an innovative approach to both include aligned fibers and distributed ECM/bioadhesives in a bioengineered platform (i.e., mimicking nature's collagen organization in tendons) coupled with a cutting-edge 3D imaging method. Specifically, depositome dynamics will be studied in a well-controlled model system, consisting of highly crosslinked poly(ethylene glycol) 100-μm thick hydrogel fibers^{12a} embedded in poly (ethylene glycol) diacrylate-based hydrogel materials that are susceptible to cleavage by MMPs, and including a variety of adhesive peptides and ECM molecules.^{46a} RBP-primed and control MSC deposition will be analyzed under static culture conditions.^{47a}As an alternative strategy, MSCs could also be cultured in cyclic tensile strain (10%, 1 Hz, 3 h of strain followed by 3 h without) or at 0% strain to enhance to collagen depositome. After 7, 14, 21, and 28 days, the MSCs (Density: 1×10⁶ cells/mL, n=20 human donors of AT and BM MSCs, 10 F/10M sex; Obtained from RoosterBio and The Marcus Center), growing and adhering to fiber scaffolds (100 μm diameter) inside the PEG-based hydrogel, will be fixed and analyzed by spatial genomics and the newly emerging Spa-RBP assay. Another innovation is to visualize collagen deposition in real-time using neo-antigen labeling and imaging of MSCs by lattice light sheet imaging at high resolution and single molecule sensitivity across 2 mm×2 mm×0.2 mm fiber-hydrogel composites. Currently, available chemistry to visualize collagen using 10 mM of a Click-IT Alexa Fluor 488 DIBO Alkyne¹³ will be evaluated and optimized in this fiber-hydrogel hybrid platform for real-time imaging of neo-collagens with and without anti-RBP primed MSCs over 7, 14, 21, and 28 days. Complementary to live assays, the presented spatially resolved proximity assays will be used to profile MSCs in hydrogels and collagen in the supernatant using ELISA. Multiplexing protocols were already validated (see Section 6.2.1) in PEG-gels and the sequential labeling and digestion chemistry are robust without affecting the mechanical robustness of the synthetic platform.

### Spatial 3D RNA and Protein Profiling of MSCs in PEG-Based Hydrogels

To measure image-based gene expression maps, we have developed a method to profile gene expression in single cells via in-situ “sequencing” by FISH. As proof of concept, we have compared single BM-MSCs cultured in physiological microenvironment conditions (FIG. 9). We conducted multiplexed HCR reactions for ten genes on BM-MSCs embedded in 3D hydrogels. The genes include β-actin, IL8, IL6, CCL11, SOX9, EEF2, SPP1, COL1A1, RUNX1, and PDL1. The hydrogel containing 6% four arms PEG-MAL was synthesized using PEG-MAL macromer, adhesives peptides, and crosslinkers (i.e., similar chemistry in Coskun, Advanced Materials^{52a}). We discovered that in MSCs in the hydrogel in a 3D environment, gene expression was spatially heterogeneous (5-10% Coefficient of Variation). Thus, multiplexed RNA screening will be feasible for MSC assays. The proposed PEG-based hydrogel will contain different biological moieties tailored for cell adhesion and spreading, but the multiplexed labeling of RNAs or proteins should, in principle, work in MSC depositomics proposal.

### Lattice Light Sheet Imaging of Real-Time Depositomics in 3D

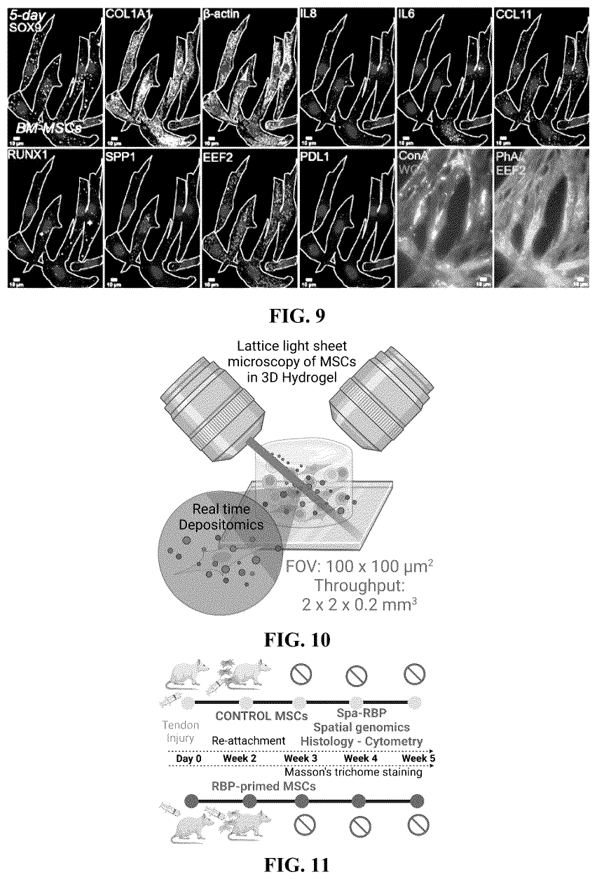

Visualization of depositomics in native and engineered MSCs will then be performed by microscopy using live-cell tags associated with neo-collagen deposition. To date, collagen 1 tags have been characterized at the second time resolution in 2D cultures.^{53a} The cellular depositome was dependent on signaling and cell types in the live collagen images. The GFP-tagged collagens accumulated in the range of nanogram per milliliter concentration range. Despite increasing interest, the spatial and temporal control of collagen has not been established in RBP-modulated depositomics studies. Thus, we will evaluate 1) live imaging assays of neo-collagen formations, and 2) cutting-edge lattice light sheet microscopy to super-resolve molecular concentrations of collagen 1 in thick hydrogels. The first live cell assay will evaluate and optimize the 10 mM of a Click-IT Alexa Fluor 488 DIBO Alkyne.¹³ The lattice light sheet imaging will be akin to visualizing cell and collagen 1 interactions in the original demonstration^{54a} in 3D embryos. This proposal will perform high spatiotemporal collagen 1 deposition rate (ms, see, to hours scales) in Fiber-Hydrogel 3D biomaterial at the single-molecule level (FIG. 10). The feasibility of this work is established using 3i lattice light sheet microscope. The MSCs will be visualized in fiber-hydrogel constructs (mounted on 5 mm round coverslips) on days 7, 14, 21, and 28. A 100 μm×100 μm area will be imaged per field-of-view, followed by rapid scanning to cover an area of 2 mm×2 mm×0.2 mm. Diffraction-limited images will be obtained from neo-antigen tags in RBP-primed and control MSCs. The expected outcome of this subtask 6.2.2. is to correlate Spa-RBP maps and live-cell collagen copy number, speed, and location in the hydrogel. Also, beyond density, the “spatial organization” of collagen 1 deposition will be a another key observation from this proposal. This sub-aim will shed light on the “quality” of collagen 1 that comprises density, alignment, and tensile strength in tendons.

### In Vivo Tendon Regeneration Model for Anti-RBP Primed MSC-Based Regeneration

### MSC-Treated Tendons

Rotator cuff injury is a widespread musculoskeletal problem, affecting 20% of the worldwide population. Severe tendon tears are typically treated with surgical reattachment of the tendons to the humeral head.^{14a} Surgical repair of severe rotator cuff injury tear unavoidably causes retear due to muscle degeneration. Novel strategies are needed to reverse muscle degeneration after rotator cuff tear to improve the clinical outcomes. Thus, MSCs or Anti-RBP primed rat MSCs will be directly injected into the tendons during the reattachment surgery, two weeks after the original tendon injury. Acute inflammation occurs typically within a week of surgery in the repair of tendon sites. Thus, MSCs (control) or Anti-RBP primed MSCs injections will be performed 2-weeks after the tendon injury, matching the need for MSC depositome to contribute to the collagen 1 incorporation into the newly formed tendon structures. One week to three weeks after MSC injections^{38a} (Time: Day-14 and Dose: 1×10⁶ cells/animal, the number of rat MSCs is feasible in this dosage^{55a}), molecular profiling methods will be performed to evaluate the structural and regenerative changes in the tendon healing process occurring up to 5-weeks (FIG. 11). These assays will include: (1) Spatial genomics analysis to quantify gene expression in control and RBP-primed MSCs, immune cells (Myeloid cells, macrophages, M1-like macrophages, and M2-like macrophages), and tenocyte/tenoblasts. Twenty genes^{41a} will be included to identify cell types and their inflammatory markers; (2) Spa-RBP assays will profile mRPI and mPPI networks (see Section 6.1) in MSCs to evaluate the level of “priming history” in the cells; and (3) Masson's trichrome staining will determine the degree of fibrous collagen in tendons. Complementary benchmarking will be assessed using flow cytometry to identify cell types and histology to study depositome and tissue compositions, 1-3 weeks after control MSC or RBP-primed MSC delivery to n=427 total Lewis rats^{56a} for multiple conditions and time points (Anti-RBP primed MSC vs control MSC, Weeks 3-5, and BM-AT MSCs), providing sufficient power^{57a} for spatially resolved molecular omics analysis of about 10,000 cells with spatial subcellular and tissue features. The expected outcome of in vivo tendon repair model is to evaluate RBP-primed MSCs in comparison to control MSCs and how they enhance the healing to modulate collagen 1 deposition (Rigor table; Sex, Age, Race, right). This approach may improve the matrix density and organization. (Table 2)

TABLE 2
Rigor table with sex, age, race.

| Model | Parameters | Sex, Age, Race | Condition |
|---|---|---|---|
| 1. MSC in | Human | Human, | Alpha-MEM; 37° C. |
| vitro | BM and | Female/Female = | Normal |
| cultures | AT MSCs | 10 each; | RBP inhibitors or stabilizers |
| 2. Anti-RBP | N = 20 | Caucasian/African | |
| primed | each | American = 10 | |
| MSC in | | each | |
| vitro | | Age of donors = | |
| cultures | | 2-80 y-old | |
| 3. MSC in | Human | Human, | Highly crosslinked poly(ethylene |
| vitro 3D | BM and | Female/Female = | glycol) hydrogel fibers scaffold |
| Hydrogel | AT MSCs; | 10 each; | embedded in PEG-DA hydrogel with |
| 4. Anti-RBP | N = 20 | Caucasian/African | adhesives (GRGDS/HA); 37° C. |
| primed | Fiber | American = 10 | |
| MSC in 3D | scaffold | each | |
| Hydrogel | (100 μm) | Age of donors = | |
| | PEG-DA | 2-80 y-old | |
| | gel with | | |
| | GRGDS | | |
| 5. MSC in | Dosage: | Rat, Male and | Sterile in a surgical suite |
| vivo Tendon | Rat 1 × | Female for RBP- | Body temperature |
| Rotary Cuff | 10⁶ MSCs | primed MSCs and | 4-weeks tendon repair |
| Injury | Time | MSC controls at | Molecular and mechanical testing |
| Model | points: | weeks 3-5 with | |
| 6. Anti-RBP | 14-day | AT/BM MSCs = | |
| MSC in | injection | 144 rats, | |
| vivo Tendon | | Rat, Male and | |
| Rotary Cuff | | Female, both | |
| Injury | | conditions, for | |
| | | mechanical | |
| | | testing at Week 5 = | |
| | | 240 rats | |
| | | Total 427 Lewis | |
| | | 50-55 days rats, | |
| | | including 43 rats | |
| | | for MSCs | |

### Overexpression or Suppression of MSC Depositome?

An open-ended question is to evaluate the effect of “enhanced” or “reduced” collagen 1 deposition on tendon healing after rotator cuff injury. 1) One model suggests that inhibition of collagen 1 synthesis would lead to a reduction in fibrosis and scar formation, allowing less collagen production so that collagen molecules can better get incorporated and organized for enhancing the load-bearing properties of the tissue. Thus, mechanical testing (n=10 rats per condition) of the tendon will be performed at the endpoint (Week 5) of tendon regeneration using a 100 N load cell at a constant 24 μm/s rate on an MTS Mini Bionix II system and characterized by a Manta G504B ASG digital imaging camera.^{58a} Recent identification of compound (C9)^{59a} and Bumetanide^{35a} suggests these would be ideal small molecules (nM-μM range) to inhibit MSC collagen 1 depositome and fibrosis. 2) Another model proposes that RBP-primed MSCs will retain overexpression of collagen 1 and directly deposit abundant collagen 1 to make up for missing ECM after injury. In the case that MSCs may directly differentiate into tenocytes, given the RBP-priming is preserved throughout the differentiation, and deposit collagen 1 to enable faster tendon repairs. To increase collagen 1 depositome, vimentin targeting drugs, artemisinin,^{60a} can be used (μM range) to enhance the stability of collagen mRNAs, resulting in robust production of collagen 1 depositions to the ECM and matrices.

### Example 3: Spatial Multi-Omics Features of BM γMSC Potency

### Introduction

Mesenchymal stromal cell (MSC) based therapies have great potential to support regeneration in the injured tissues. Another key application of MSC therapy in hematopoietic cell transplantation (HCT) is to prevent or treat graft-versus-host disease (GVHD), eventually augmenting HCT's success in blood disorders. Preclinical and clinical studies have proven MSC infusions to be safe and interferon-γ primed MSCs (γMSCs) to be highly effective prophylactic cell therapy; however, γMSCs have not been infused into patients. An ongoing clinical study is the first-in-human trial of γMSCs; such clinical protocol is focused on γMSC prophylaxis for GVHD after HCT for hematologic malignancies. However, it is unclear why different patients exhibit variable outcomes in their GVHD prophylaxis and how MSC therapy of GVHD works better in pediatric patients compared with adult patients. A distinct mechanism of an explicit activity remains to be elucidated for the development of clinically relevant potency assays to guide the in vivo administration of MSCs and γMSCs in human clinical trials. The traditional method for assessing MSCs' immunoregulatory potency is limited to the 2D co-cultures of MSCs with immune cells with/without interferon-γ stimulation, which is not clinically appropriate. The recently developed 3D biomaterial-based MSC potency assay matched the physiological MSC response in preclinical models, albeit without clinical demonstration. This proposal will uniquely evaluate the γMSC potency in vitro using previously established spatial transcriptomics technologies (Coskun) implemented within a hydrogel-based biomaterial chip (Garcfa), for outcome (GVHD prophylaxis or not) assessment of 21 adults and 12 pediatric leukemia patients undergoing HCT receiving a γMSC infusion. The overall goal of this project is to quantitatively define the 3D potency of clinically used γMSC by the spatially resolved transcriptional and proteomic profiles of allogeneic bone marrow (BM)-derived γMSCs that enable T-cell suppression in the 3D hydrogel environment. We will then correlate in vitro γMSC profiling to the clinical outcomes to identify patient/donor T cell pairs who are more likely to respond to γMSCs prophylaxis of GVHD. We hypothesize that inflammatory and anti-inflammatory secreted factors play spatially regulated opposing roles in the interactions of γMSCs and T cells located at distinct 3D positions of the hydrogel substrate. Moreover, multivariate transcriptional modeling of γMSC-T cell interactions will predict donor T cell-third party γMSC combinations that will effectively respond to γMSC prophylaxis of GVHD. Aim 1 will assess the spatial transcriptional and proteomic features and secretome in the 3D γMSC potency assay and analyze allogeneic BM-γMSC and donor-derived T cell neighborhoods in the 3D positions of the gel. Aim 2 will correlate BM-γMSC potency to the responder and non-responder patients from adult and pediatric cases treated with the BM-γMSCs. Our long-term goal is to generate new predictive insights into the in vitro γMSC influence on T cell responses for quantitative assessment of the clinical outcome of γMSC-based prevention of GVHD before the γMSCs are administered to the HCT patient.

The proposed research is relevant to public health because it will build a bioengineering platform for assessing cell states using a series of molecular imaging technologies to simultaneously visualize many markers in chemically activated and clinically tested mesenchymal stromal cells (MSCs) regulating nearby immune cells at their physical positions in three-dimensional (3D) biomaterial devices. To mimic the MSC infusion in patients, bone marrow-derived MSCs are mixed with a patient's immune cell in physiologically relevant biological materials, leading to differences in MSC's potential control of immune cells that can then be used to explain the efficacy of cell-based therapies when activated MSCs encounter other supporting cells in the patient's local tissues for suppressing immune rejection. The presented single-cell spatial molecular analysis methods will shed light on the alterations in the chemical releasing capacity of the potentially competent or impotent MSCs using a predictive model comparing the molecular profiles of bioengineered platforms to that of native tissue environments.

### Background

### Spatial Transcriptional Profiling in Single Cells

Developed and optimized herein is a single cell RNA imaging to measure image-based gene expression maps via in-situ “sequencing” by fluorescence in situ hybridization (FISH). Our sequential FISH (SeqFISH)^{3b} method is highly quantitative and does not require physically isolating single cells or using homogenates. To distinguish different mRNA species, we barcode mRNAs with the FISH probes using sequential rounds of hybridization. The sample is imaged and the FISH probes are removed by enzymatic digestion. Then the mRNA is hybridized in a subsequent round with the same FISH probes but now labeled with a different dye. As the transcripts are fixed in cells, the fluorescent spots corresponding to single mRNAs remain in place during multiple rounds of hybridization and can be aligned to read out a color sequence. Each mRNA species is therefore assigned a unique barcode. Thus, the number of each transcript in a given cell can be determined by counting the number of the corresponding barcode. The number of barcodes available with this approach scales as F^{N}, where F is the number of distinct fluorophores and N is the number of hybridization rounds. With 5 distinct dyes and 3 rounds of hybridization, one can detect 125 unique genes. As a recent demonstration in our recent preprint (Fang et al, BiorXiv 2022^{21b}), we barcoded 12 genes in single MSCs with 3 dyes and 4 rounds of hybridization, providing linear barcoding (3×4=12). This pipeline acquired multiplexed, spatially resolved subcellular RNA maps of single cells through sequential and multiplexed Hybridization Chain Reaction (HCR),^{51b} a robust amplification strategy utilized in the detection of single RNA molecules present in human bone marrow-derived MSCs (HBM) and Umbilical cord-derived MSCs (HUC). The sequential hybridization of HCR assembly, followed by DNase I digestion, was used for the multiplexing strategy. This is an advancement of seqFISH combined with HCR, providing the feasibility of seqFISH-HCR technology (FIG. 13). This proof-of-concept study of seqFISH will then be performed in single γMSC located on 3D engineered hydrogels after fixing and multiplexed labeling and analysis.

### Spatial Proteomic Profiling in Single Cells

As an innovative approach, we have developed a high-resolution and multiplexed Co-detection by Indexing (CODEX)^{6b,52b} spatial proteomics method. Multi-frame (100 images per slice) fluorescence images will be acquired from antibody-oligonucleotide labeled cells using confocal imaging at 60× magnification, followed by computational analysis of high-resolution protein features of individual cells. As a preliminary analysis, thin tissue sections from bone marrow biopsies of cancer patients were profiled by existing CODEX chemistry, providing 20-plex multiplexed proteomic features of single cells at a sub-300-nm spatial resolution over lateral (x-y) dimensions. To visualize cell-to-cell interactions at the molecular level across 3D, we developed a computational framework for reconstructing 3D proteomics distributions using high-resolution CODEX (FIG. 14). These spatially-resolved features in human tissues have the potential to detect ultrafine molecular aberrations of the receptor, cytosolic, and nuclear features in single γMSCs for accurate phenotyping based on established markers.

### Spatial Multi-Omics Profiling

Spatially resolved multimodal analysis in thick 3D PEG-4MAL hydrogel samples requires key protocol optimizations to boost the molecular signal and integrate the RNA and protein data. This challenge is akin to the molecular analysis complexity of nucleic acids and protein alterations in formalin-fixed paraffin-embedded (FFPE) tissues typically used in histological analysis for cancer diagnostics and prognosis. Thus, demonstrated herein is that prostate tumors express collagen 1 using multiplexed single-cell imaging in patient biopsies. To perform gene expression mapping in 5-μm thin FFPE tissues, the RNA detection protocol was integrated with an amplification method comprising a split-probe design of HCR. FISH-HCR measurements correspond to gene expression maps by visualizing RNA dots across the tissues, providing similar levels of COL1A1 RNA expression in tumor areas in distinct pathological cancer-rich regions of human tissues. After the RNA visualization in prostate cancer biopsies, FISH labels were digested by a DNase-I-based enzymatic treatment to remove the FISH signal on the tissues. To validate tumor-related protein expression on the same tissue sets, an immunofluorescence experiment was performed for visualization of EZH2 and H3K27me3 in CRPC-NEPC using antibodies (FIG. 4). This sequential multi-omics integration allowed RNA and protein mapping in the same cancer tissues, providing the optimized protocol for spatial multi-omics integration for γMSC analysis in 3D hydrogels.

### Summary

Mesenchymal stromal cells (MSCs) have been shown to exhibit potent immunomodulatory activity, as well as to stimulate tissue repair and regeneration in damaged organs or diseases. These advantageous characteristics provide opportunities to enhance hematopoietic cell transplantation (HCT) to treat blood cancers. MSC therapy is used for HCT patients to prevent or treat graft-versus-host disease (GVHD). While interferon-γ primed MSCs (γMSCs) have shown to be safe and highly effective prophylactic cell therapy, there is no clinical trial on γMSC therapies.^{1b} The first-in-human trial of γMSCs prophylaxis for GVHD after HCT for hematologic malignancies is carried out. The basis for diverse outcomes (GVHD treatment efficacies) in different patients and the scientific mechanism underlying the observation that MSC therapy of GVHD appears to be more effective in pediatric patients compared with adult patients is being explored. This knowledge can inform the development of clinically relevant potency assays, which do not currently exist but are crucial to accompany the in vivo administration of MSCs and γMSCs in human subjects.

MSCs secrete molecules to regulate immune responses as a therapeutic mechanism of action. The standard potency assay for accessing MSC immunomodulatory function is to co-culture MSCs with immune cells and/or interferon-γ (IFN-γ) stimulation on 2D planar substrates. However, these standard assays fail to recapitulate clinically relevant MSC functional and secretome profiles. Developed herein is a 3D biomaterial-based potency assay^{2b} in a microfluidic chip that can match the physiologically relevant level of MSC secretome. While imaging flow cytometry and secretome profiling of MSCs from microfluidic potency chips demonstrated similar secretory performance compared to an in vivo preclinical model, it remains to be elucidated how the inflammatory and anti-inflammatory secreted factors, particularly when co-culturing γMSCs with T-cells, enhance the predictive power of 3D potency of γMSCs mixed with donor's T cells infused to blood cancer patients in clinical trials for prophylaxis of GVHD.

Single-cell spatial analysis methods are emerging to reveal cell states and cellular communication in cultures and tissues. This proposal will uniquely leverage our published spatially resolved genomics and proteomics methods to the study of 3D γMSC potency in vitro. Our long-term goal is to evaluate the in vitro γMSC influence on T cell responses for quantitative assessment of the clinical outcome of γMSC-based prevention of GVHD before the γMSCs are administered to the HCT patient. The goal of this project is 1) to quantitatively define the 3D potency of clinically used allogeneic bone marrow (BM)-derived γMSCs that enable T-cell suppression in the 3D hydrogel and 2) to correlate spatial multi-omics features of BM γMSC to the clinical outcomes for identification of patient/donor T cell pairs who are more likely to respond to γMSCs prophylaxis of GVHD. We hypothesize that 1) the inflammatory and anti-inflammatory secreted factors play spatially regulated opposing roles in the interactions of γMSCs and T cells located at distinct 3D positions of the hydrogel substrate and 2) multivariate transcriptional and proteomic modeling of γMSC-T cell interactions will predict donor T cell-third party γMSC combinations that will effectively respond to γMSC prophylaxis of GVHD.

Leveraged herein are spatial multi-omics^{3b-6b} to quantify the 3D γMSC potency in vitro and correlate with the clinical outcome of patients receiving γMSC prophylaxis of GVHD. The following tasks were performed:
1) Evaluated the correlation of spatial multi-omics features of BM γMSC potency with in vitro T-cell suppression using a 3D biomaterial (PEG-4MAL)-enabled chip.
   a) Performed single cell transcriptional and proteomic analysis of BM γMSC on a 3D potency chip using a T-cell suppression assay with 5 distinct BM γMSC: CD3+ T-cell ratios (1:2, 1:10, and 1:50).
   b) Correlated spatial single-cell multi-omics features of TNF/Cytokine pathways and 3D cellular BM γMSC neighbors to the individual CD3+ T-cells suppressed at local regions (x-y-z) of the 3D chip.
2) Correlated spatial multi-omics features of BM γMSC potency suppressing T cells in vitro with the outcome of patients receiving γMSC prophylaxis of GVHD
   a) Profiled spatial multi-omics maps of 5 distinct BM γMSCs and neighboring 33 donor's T cells (21 adult and 12 pediatric subjects) at 1:2, 1:10, and 1:50 γMSCs: T cell ratios in a 3D potency chip using rapid light sheet imaging to generate the data for evaluation of the clinical outcome of GVHD prophylaxis.
   b) Performed multivariate modeling and machine learning classification of spatial multi-omics features of third-party γMSC and T cells from 33 leukemic patients undergoing HCT with γMSC prophylaxis.

This project will also significantly advance the development of an in vitro potency assay predictive of the clinical outcome of GVHD prophylaxis based on spatial multi-omics maps. This project brings together experts in single cell technology, MSC immune biology, GVHD, clinical HCT, biomaterials, and Bioinformatics to create scientific knowledge of γMSC potency that can be used to accompany clinical trials of γMSC-based GVHD therapies before γMSCs are infused to the HCT patient.

### Significance

### Development of Safe and Efficient γMSC Therapies for GVHD Prophylaxis

MSCs and γMSCs secrete molecules to regulate immune responses as a therapeutic mechanism of action in graft-versus-host disease (GVHD) treatment or prevention.^{7b,8b} The rationale behind γMSC prophylaxis and treatment of GVHD is based on the 3-stage schema^{7b} of acute GVHD pathogenesis, where Stage 1 includes a conditioning regimen to enable injuries in nonhematopoietic tissues for secretion of inflammatory factors, Stage 2 covers infusion of the hematopoietic graft to active adoptively transferred donor T cells by host antigen-presenting cells, and Stage 3 involves targeting host tissue by activated donor T cells to initiate alloreactive cytotoxic mechanisms. Similarly, IV infused γMSCs may migrate to inflammation sites (Stage 1), then may migrate to alloactivation sites for suppressing the proliferating T cells (Stage 2), and finally migrate to the sites of donor T cell host T tissue interaction sites to inhibit immune responses, providing effective treatment of GVHD (Stage 3). In addition to GVHD, γMSC may facilitate hematopoietic stem cell (HSC) engraftment by secreting hematopoietic cytokines and/or suppressing the residual host immunity, which can reject the transplanted graft.^{7b} Graft failure after autologous hematopoietic cell transplantation (HCT) is an especially serious complication; MSC therapies have demonstrated the potential to rescue hematopoiesis after autologous HCT graft failure. Indeed, immune-suppressive activity without significant toxicity is a major advantage over pharmaceuticals, such as JAK inhibitors, and could justify more widespread use in patients. A clinical trial is conducted of γMSCs prophylaxis for GVHD after HCT for hematologic malignancies using Interferon-gamma (IFNγ)-primed human bone marrow-derived mesenchymal stromal cells (BM γMSC). γMSCs were chosen because the recent evidence suggested only suppression activity of γMSCs against T cells through cell cycle arrest and polarization of T cells toward a regulatory phenotype that can impede inflammation. While other MSCs may enhance suppression activity, a randomized clinical trial is lacking to establish the efficacy of BM MSCs compared to others. Thus, we have chosen to evaluate BM γMSC in our clinical trials when they are isolated from third-party and activated before infusions.

### Molecular Determinants of γMSC Immunomodulatory Function in GVHD Prophylaxis

γMSCs exhibit many potentially immune-modulating activities; however, it is unlikely that the cells use all possible pathways in all settings. Hence, the distinct mechanism of an explicit activity, e.g., treatment of GVHD or stimulating tissue repair, must be understood. Such scientific knowledge will inform the development of a greatly needed clinically relevant potency assay, potentially increasing the success of γMSC-based GVHD therapies. The standard potency assay for accessing MSC immunomodulatory function is to co-culture γMSCs with immune cells on 2D planar substrates.^{10b} However, the 2D substrates do not seem to predict potency in the clinical (3D) setting. Thus, we do not yet understand the mechanism by which the auto and paracrine signaling networks suppress T cells. Our 3D potency models^{2b} should have far greater predictive power compared to planar substrates. We will use single-cell spatial analysis to reveal cell states, cellular communication, and cell neighborhoods in co-cultures to reveal γMSCs' previously unknown molecular heterogeneity.

Overcoming Limitations of Molecular Assays for Studying γMSC Interactions with Immune Cells

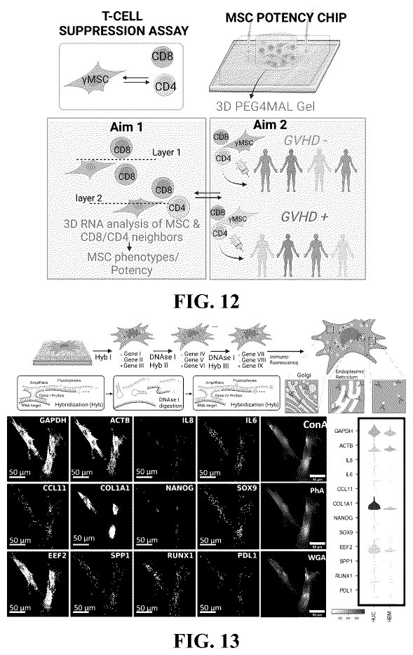

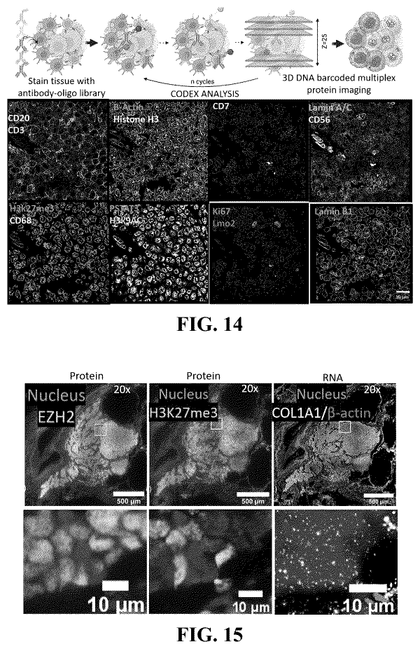

MSC heterogeneity has been profiled by mass spectrometry and RNA sequencing (RNA-seq) to study the unique functions of MSCs and γMSCs in differentiation potential, regulation of extracellular matrix, and inflammatory microenvironment.^{1b,11b-15b} However, these molecular profiles are limited to ensemble-level data of γMSCs, providing limited heterogeneity and masking the differences of individual cells. In the meantime, single-cell RNA-seq has been used to further shed light on the granularity of MSCs and γMSCs.^{16b-20b} Emerging single cell analysis methods determine molecular profiles and cell-cell interactions that regulate γMSC potency. Single cell spatial transcriptional and proteomic analysis methods^{3b-5b,21b} will be used to profile image-based 3D γMSC potency in physiologically relevant hydrogel biomaterials (FIG. 12).

### Innovation

### Study of Spatial Multi-Omics Analysis of Stem Cells

Powerful genome-wide, proteome-wide, and metabolomics technologies (e.g., ChIP-seq, RNA-seq, mass spectrometry) have broadened the field of molecular omics profiling for finding the gene, protein, and metabolic regulatory factors.^{22b} However, these approaches can only provide population average data that combine information from millions of cells. This population heterogeneity can be resolved by analyzing interactions of regulatory elements in single cells. Together with the single-cell RNA-seq and mass spectrometry analysis, current spatially resolved cellular profiling methods analyze unimodal molecular distributions either for RNA or protein or metabolite of interest in individual cells. Spatial RNA profiles were measured by sequential fluorescence in-situ hybridization (FISH)^{3b,23b}, multiplexed error-robust FISH (MERFISH)^{24b}, and other multiplex FISH technologies. Spatial Protein data was profiled by automated relabeling using bleach-based iterative analysis^{25b} and DNA barcoded antibodies^{26b}, or, isotope-conjugated antibodies that are labeled on tissues and imaged by imaging mass cytometry (IMC)^{27b} and multiplexed ion beam imaging (MIBI)^{28b}. Spatial metabolic maps were extracted by imaging mass spectrometry analysis in fixed tissues and cells.^{29b} While these single-cell analysis methods generally provide only one type of molecular information, there is an important need for the integration of technologies that can provide spatially resolved, multi-omics analysis of cells when they are interacting in health and disease. To provide a solution to this important need, here we will deploy multiple spatial multi-omics methods to study γMSC heterogeneity from distinct BM sources in biomaterial based 3D hydrogel platforms.

Study of identifying γMSC phenotypes in vitro using spatial transcriptional maps MSCs are complex biological identities, leading to unproven efficacies and potential adverse effects in therapies^{30b,31b}. MSC currently is defined by the expression of CD105, CD73, and CD90 markers, lack of expression of hematopoietic markers and HLA-DR, and the potential to differentiate into osteoblasts, adipocytes, and chondrocytes in vitro^{8b,32b} MSCs demonstrate cascaded complexity of heterogeneity that requires modeling of spatial molecular analysis beyond commonly used surface stains. Image-based gene expression analysis methods open doors to study γMSCs at the sub-300-nm resolution, potentially yielding γMSC identity and potency beyond protein markers^{4b,23b,24b,33b}.

### Identifying Single T-Cells Modulated by γMSCs in Hydrogels Using Spatial Transcriptional and Proteomic Maps

Spatially resolved single-cell data has been used to define cell types and their locations in mouse and human tissues^{34b,35b} In this platform, the cellular gene expression analysis platform profiles the spatial heterogeneity in 3D biomaterial-based γMSC co-cultures with T-cells within the context of the GVHD treatments. Our initial approach will be to assess RNA heterogeneity to identify unique spatial regulation of individual human γMSCs in self-assembled T-cell suppression assays (a recommended technique by the International Society for Cellular Therapy^{36b}) in 3D within in vitro conditions. In this assay, a lower suppression index value will correspond to greater T cell suppression and a more potent cell product.^{2b}We reasoned that the spatial expression maps would yield the MSC secretory potential and neighboring T-cell states.

Correlation of Spatial Multi-Omics Characteristics of IFN-γ Primed BM MSCs with In Vitro T-Cell Suppression Potency Determined in a 3D Biomaterial (PEG-4MAL)-Enabled Chip

T-cell suppression assay in 3D microfluidic biomaterial chips provides accurate single-cell pairing of BM γMSCs and donor-derived T-cells trapped within 3D gels that can provide quantitative models of unique inflammatory and anti-inflammatory secreted factors of BM γMSCs in x-y-z positions of the 3D chamber and corresponding distinct spatial multi-omics features of single BM γMSCs.

MSC potency chip for evaluating the immunomodulatory function of γMSCs. Conventional 2D T-cell suppression assays on plastic substrates are used to assess the MSCs' potency, a metric to evaluate the immunosuppressive role of MSCs quantitatively.^{45b} MSC potency has classically been assessed on 2D substrates, but the local microenvironment is different from native tissue compositions, especially in mechanical, physical, and chemical constituents. 3D PEG-4MAL hydrogels support cell growth in a physiologically relevant microenvironment.^{46b} In this platform, hydrogel encapsulation of MSCs or γMSCs enhances the secretory protein concentrations. The microfluidic chambers then allow stimulant delivery (e.g., IFN-γ) and supernatant removal from the 3D MSC assay in the cell-laden hydrogel that is embedded within a poly(dimethylsiloxane) (PDMS) chip. Perfusion capability across the 3D distributions of BM MSCs co-cultured with CD3+ T cells only, stimulated with IFN-γ only, and combined with CD3+ T cells and IFN-γ has the great potential for superior responses compared to static 2D controls and 3D static samples. The 3D microfluidic volume can also be rationally designed to mimic in-vivo response characteristics of individual BM MSCs, providing a strategy to predict MSC potency in vitro without the need for a tissue-mimicry system. High-throughput fabrication of these 3D MSC potency chips is possible for up to 40 devices that can be simultaneously operated at a low-cost (0.52 USD per chip). The spatial multi-omics features of MSC and T-cell neighbors can be probed optically in the same chip and the secretome in the supernatant can be collected by a collection syringe from the outlet port of the microfluidic chamber. Thus, the 3D potency chip will be optimized by microfluidic design parameters, spatial multi-omics features, secretome variability, rapid imaging compatibility, and clinical outcome metrics.

Single-cell and spatial transcriptional analysis of MSCs on 3D engineered substrates: MSC potency assays have been classically analyzed by an average data of the MSCs and the secretion profiles were a mean value of a large MSC population. For instance, secreted proteins are performed in the culture supernatant using multiplexed antibody arrays and Luminex assays, masking the heterogeneity across MSCs.^{47,48} Cytokine release mechanisms are coordinated by intricate Spatio-temporal mechanisms guided by cell-to-cell interactions and transient secretion dynamics.^{49b} Immunostaining of cytokine proteins yields a portion of cytokine molecules before releasing or during short-term storage in cells.^{50b} On the other hand, image-based functional characterization of MSC secretion potential is highly crucial for assessing gene expression for each MSC located at different spatial coordinates of the 3D-engineered hydrogels. We reasoned that gene expression measurements of cytokine genes would be a crucial metric to define 3D γMSC secretory potential, instead of targeting proteins. Spatially resolved efficient secretors will be identified using multiplexed gene expression analysis maps.

Single-cell and spatial protein analysis of MSCs on 3D engineered substrates: MSC currently is defined by the expression of CD105, CD73, and CD90 markers, and lacks expression of CD45, CD34, CD14 or CD11b, CD79a or CD19, and HLA-DR, and the potential to differentiate into osteoblasts, adipocytes, and chondrocytes in vitro^{8b,32b}. These markers have been evaluated by flow cytometry and immunostaining to target proteomic markers of MSCs. Thus, this proposal will integrate spatial transcriptional analysis with spatial proteomic mapping to accurately identify phenotypes of each γMSC located on 3D-engineered substrates.

### Preliminary Data

MSC potency chip in a 3D hydrogel: We demonstrated a 3D MSC potency chip using custom experimental workflows. A microfluidic chip was fabricated using PDMS patterning and the 3D PEG-4MAL gel was mixed with BM MSCs in separate experiments. The cell-laden 3D hydrogels were then embedded in the middle chamber of this microfluidic chamber connected to inlet and outlet ports and the top portion of the hydrogel was isolated with a thin film (FIG. 12). The stimulant IFN-γ in the supplemented media was then perfused across the cell-laden 3D hydrogel (microfluidic IFN-γ) and separately BM MSCs were treated with IFN-γ when cells are on 2D substrates (2D IFN-γ). After culturing the BM MSCs in stimulant IFN-γ containing media (co-cultures with CD3+ T cells also stimulated 2D IFN-γ assay) both for 2D and 3D conditions, the collected media was analyzed for secretome by a Luminex assay, and cells were extracted from hydrogel (after degradation) was profiled by flow cytometry. While the cell viability was reduced in the microfluidic IFN-γ culture compared to 2D IFN-γ and 2D Ctrl (microfluidic IFN-γ=70.9±4.2%, 2D IFN-γ=98.0±0.24%, 2D Ctrl=98.7±0.10%), the secretome profiles in microfluidic IFN-γ were distinct and superior for several cytokines/chemokines compared to 2D IFN-γ cultures, suggesting the dependency of MSC potency in 2D and 3D environments. Thus, the previous work evaluated a panel of 20-plex analytes using linear regression (R²) analysis on the donor-matched T cell suppression index against analyte secretion levels (normalized 2D Ctrl, μg pg-1) for microfluidic IFN-γ and 2D IFN-γ stimulated cultures. Interestingly, matrix metalloproteinase 13 (MMP-13) in the microfluidic IFN-γ culture system significantly performed the best using the CD3, CD4, and CD8 suppression index values among other 20 analytes. In both microfluidic IFN-γ and 2D IFN-γ platforms, lower MMP-13 secretion correlated to more potent MSC donor lines, while the microfluidic IFN-γ exhibited higher R² for all the T-cell subsets. These results demonstrated that the 3D potency chip robustly maintains BM MSC potency in a physiologically relevant 3D environment containing synthetic extracellular matrix and 3D MSC and T-cell interactions as in an in vivo tissue.

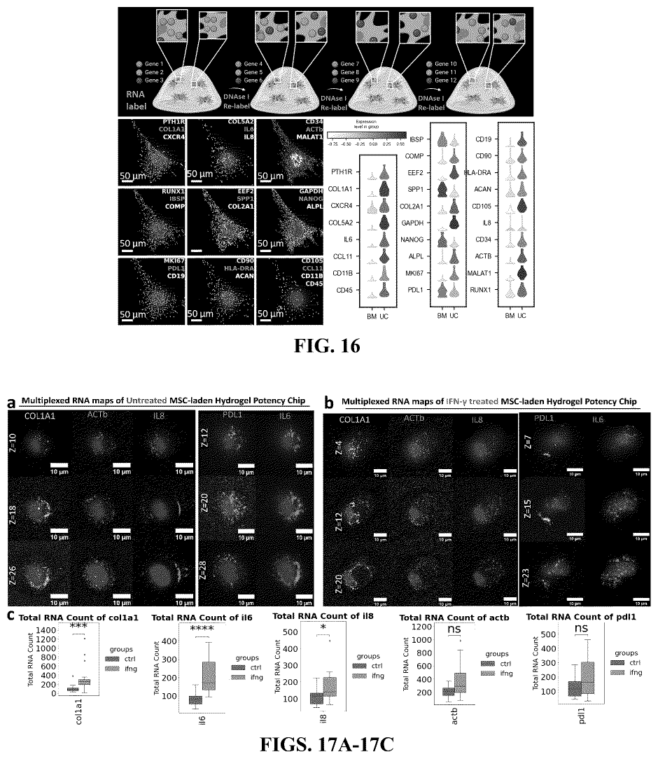

Spatial transcriptional profiles of single cells in 3D organoids. As proof of concept, we have developed a pipeline of spatial omics techniques that provide single-cell and subcellular molecular comparisons of BM-MSCs cultured in 3D physiological microenvironment conditions (FIG. 16). We conducted multiplexed FISH-HCR for 28 genes on BM- and UC-MSCs embedded in 3D hydrogels. The genes include β-actin, IL8, IL6, CCL11, PTHR1, CXCR4, SPP1, COL1A1, COL2A1, COL5A2, CD34, MALAT1, IBSP, COMP, EEF2, GAPDH, NANOG, ALPL, CD19, HLA-DRA, ACAN, RUNX1, CD90, CD105, CD11B, PDL1, and CD45, yielding higher gene expression in UC-MSCs than BM-MSCs. Organoid chemistry: The hydrogel containing 6% four arms PEG-MAL was synthesized using PEG-MAL macromer, adhesives peptides, and crosslinkers. PEG-MAL macromers were tethered with a 1:4 ratio of macromers and thiolated GFOGER peptide (SEQ ID NO: 5) at a pH of 7.8 HEPES by incubating them for 30 min 37° C. (Coskun, Advanced Materials^{53b}). All components were diluted using PBS++ solution with a pH of 7.4 and 1% HEPES. Covalent conjugation ingredients, a 1:1 mixture of MMP9-degradable VPM peptide to non-degradable DTT molar ratio, were suspended together at a pH of 6.4 HEPES buffer. Cells (BM-MSCs) were dissolved in the crosslinker solution. Hydrogels were crosslinked by a mixture of a 1:1 volume ratio of macromers and crosslinker-cell solutions on the nontreated plate surface. The mixture was mixed rapidly via pipet and cured at 37° C. for 15 min. Post-curing, the cell media, or buffers, were added to each organoid. The media was replenished every three days. We discovered that in MSCs cultured in the hydrogel in a 3D environment, gene expression was spatially heterogeneous. Thus, multiplexed RNA screening will be feasible for MSC potency assays.

### Design and Approach

T-cell suppression assay for MSCs on 3D PEG-4MAL treated substrates: T-cell suppression assay is a method for evaluating MSCs' immunomodulation of host immune cells (especially T cells) by MSCs secreted factors. Thus, MSC-mediated T-cell suppression in “MSC: CD3-isolated T-cell” co-cultures will be performed as a functional metric for MSC immunomodulatory potential, a recommended technique by the International Society for Cellular Therapy (ISCT).^{36b} Thus, we will evaluate the BM-derived MSCs from five healthy donors each that will be acquired from a third-party manufacturer (RoosterBio), and each cultured per the manufacturer's specifications [RoosterNourish-MSC (KT-001) and RPMI media]. Five donors from the same manufacturer were chosen to reveal the donor variability of BM MSC products. BM MSCs will be cultured on PEG-4MAL and will be used for 3D MSC potency analysis. Specifically, we will evaluate functional BM MSCs' potency by quantification of T cell suppression following ideally donor-matched hMSC: CD3+ T-cell co-culture, as per recommendations of the ISCT, but in this proposal, hMSCs will not match donor-derived T cells. T-cell activation will be performed with anti-CD3+/anti-CD28+ Dynabeads (ThermoFisher) at a 1:1 T cell to bead ratio and 12 U rIL-2 (PeproTech). In this assay, a lower suppression index value will correspond to greater T cell suppression and a more potent cell product. MSCs will suppress T cell proliferation in a dose-dependent manner with significant suppression for co-cultures at varying hMSC doses using three ratiometric mixes of 1:2 hMSC: T cell, 1:10 hMSC: T cell, and 1:50 hMSC: T cell only, and combined with 25 ng ml⁻¹ and 50 ng ml⁻¹ IFN-γ concentrations. Following 3 days of co-culture of MSCs with CD3+ T cells, image-based proliferation (Ki67) and survival (TUNEL) analysis will replace flow assays and be benchmarked using negative isotope controls using immunostaining.^{54b} In this proposal, BM MSCs' potency will be evaluated by this T-cell suppression assay both in 3D self-assemblies. Rigor: Demonstration that MSC cell suspension (1×10⁶, 5×10⁶, 10×10⁶ cells mL⁻¹) at passage 4 with >70% viability is mixed with Peripheral blood mononuclear cells (PBMC)-isolated CD3+ T-cells (2×10⁶, 5×10⁶, 10×10⁶, 50×10⁶, and 100×10⁶ T-cells mL⁻¹ per well after 3-days of MSC co-culture with CD3+ T-cells. We will test different MSC: T cell ratios to generate clinical insights into required cells per IV infusion.

Validating the stiffness of PEG-4MAL-based 3D substrates: Following these protocols, the hydrogel containing 6% polymer density will be synthesized using PEG-4MAL macromer, adhesive peptides, and crosslinkers. PEG-4MAL macromers will comprise a fixed amount (1 or 2 mM) of thiolated RGD (GRGDSPC—SEQ ID NO: 3) peptide at a pH of 7.8 HEPES by incubating them for 30 min 37° C. Covalent conjugation ingredients, a 1:1 mixture of MMP9-degradable VPM (GCRDVPMSMRGGDRCG—SEQ ID NO: 4) peptide to non-degradable DTT molar ratio, will be suspended together at a pH of 6.4 HEPES buffer. Cells (BM-MSCs or BM-γMSC) will be dissolved in the crosslinker solution. Hydrogels will be cross-linked by a mixture of a 1:1 volume ratio of macromers and crosslinker-cell solutions on the non-treated plate surface. The mixture will then be mixed rapidly via pipet and transferred to a 96-well substrate, followed by curing at 37° C. for 15 min. Post-curing, the cell media, or buffers, will be added to each hydrogel assembly. The media will be replenished every three days. To assist imaging, the hydrogel is prepared in 96 well plates. PEG-4MAL polymer density will be modified to tune the density of cross-links and identify a matrix stiffness that provides physical support and promotes the essential mechanosignals. Specifically, the hydrogel stiffness will be tuned to PEG-4MAL hydrogel mechanical properties (G′=60 Pa) and adhesive peptide density (1.0 mM) because this stiffness provided sufficient conditions for MSC survival in this protocol.^{41b} Rigor: The gels will be cross-linked at 37° C., removed from the isolators, and swollen in PBS overnight. The hydrogels will be rheologically assessed by dynamic oscillatory strain and frequency sweeps performed on an MCR 302 stress-controlled rheometer (Anton Paar).

Single-cell and spatial transcriptional analysis of γMSCs on bioengineered substrates: MSC potency assays have been classically analyzed by an average data of the MSCs and the secretion profiles were a mean value of a large MSC population. For instance, secreted proteins are performed in the culture supernatant using multiplexed antibody arrays and Luminex assays, masking the heterogeneity across MSCs.^{47b,48b} Cytokine release mechanisms are coordinated by intricate Spatio-temporal mechanisms.^{49b} Immunostaining of cytokine proteins yields a portion of cytokine molecules.^{50b} Thus, image-based functional characterization of γMSC secretion potential is highly crucial for assessing gene expression for each γMSCs in the 3D hydrogels. Highly sensitive transcriptional analysis of γMSCs from major donors will be performed using a seqFISH pipeline on a Nikon Ti2E single-molecule imaging microscope. Fifty targets will be identified from the previous RNA-seq and Real-Time Quantitative Reverse Transcription PCR (qRT-PCR) experiments. These targets include MSC-specific positive genes (Nanog, Sox2, Oct4, Sca-1, CD44, CD105, CD71, CD73, and CD90). To study auto/paracrine signaling^{56b} of BM γMSCs, RNA targets for TNF-α, TIMP-1, IL-17E/IL-25, IL-12 p70, IGFBP-rpl, FGF, IL-6, CCL2/MCP-1, IL-8, VEGF, VCAM-1/CD106, PD-L1, B7-H1, HGF, MMP-13, M-CSF, ICAM-1, TNF R1, FAP, CCL3/MIP-1α, and CXCL9/MIG will be included in the multiplexed panel. Single-cell boundaries will be isolated from phalloidin, WGA, Concanavalin A, and DAPI markers that will be labeled at the end of the experiments on the same sample. The number of barcodes and transcripts in seqFISH data will be quantified and each cell's gene expression map will be created. Rigor: We will use heatmaps with z-score normalization, hierarchical clustering, and network mapping to compare seqFISH data (correlations >0.3: significant) from triple MSC experiments from distinct sources. Specific gene expression maps will be integrated using Seurat v3 package^{57b} and visualized on a high-dimensional tSNE/UMAP/PCA map. For subcellular analysis, the cytosolic and nuclear regions will be segmented, followed by an analysis of multiplexed markers across the subcellular volumes to quantify the spatial variability of transcripts that are detected within individual γMSCs.

Spatial proteomic phenotyping in MSCs: In combination with seqFISH, a proteomic imaging method will be used to target proteins by DNA-conjugated antibodies. In this method, antibodies of interest are linked to an 18-mer oligonucleotide sequence and another complementary DNA sequence will be designed with a fluorophore attached to it. These DNA-tagged antibodies are designed for fifty proteins and initially incubated with the cells and tissues overnight at 4° C. The next day complementary DNA “barcodes” will be cycled using 3-colors that are uniquely assigned to a set of 3-target proteins. Using an automated fluidics system, the labeling and stripping reagents will be used to create a fifty-marker map of target proteins. This protocol is an alteration of seqFISH technology, as the DNA barcodes are the same as the seqFISH method. The only modification is to conjugate antibodies to DNA tags using maleimide chemistry. Thus, CODEX is a complementary technology to multiplex proteins in a single experiment, similar to the measurements done in SeqFISH. Thus, we will refer to seqFISH and CODEX side by side to achieve RNA and protein experiments for single γMSCs throughout the proposal. The phenotypes of single MSCs will be identified based on twenty-four major candidate protein targets^{58b} containing EEFA1, NNMT, ADAM9, CAV1, S100A11, MCFD2, ACTN1, ANXA5, PLOD2, SERPINE1, CTGF/CCN2, TPM4, SMURF2, CCND1, RHOC, LOXL2, TAGLN, COL4A1, CALU, LOX, COL5A1, KDELR3, SERPINF1, DDR2, IGFBP3, XK−, FRAS1−, and WAS−, & conventional stains^{8b} comprising CD105+, CD73+, and CD90+, CD45−, CD34−, CD14− or CD11b−, CD79a− or CD19−, and HLA−DR−. Rigor: Spatial features (20,000 cells with size, density, pixel distributions, and gene neighborhoods^{21b}) will classify BM γMSCs (5 donors) after 3 and 7 days of culture and spatial data integration from seqFISH and CODEX will be performed by the Seurat v4^{59b} single-cell multimodal single-cell data analysis package.

Spatial transcriptional and proteomic features of γMSCs and neighboring T cells: Spatial RNA maps will be measured directly in an γMSC potency chip using 20× spinning disk confocal imaging up to 0.8 mm depth. The microfluidic channel depth will be optimized to match the optical imaging depth capability. BM-γMSCs and T-cells will be acquired as they will be trapped among the 3D PEG-4MAL gels, providing the MSC phenotypes and suppressed T-cells at x-y-z positions of the 3D gel using 1:2, 1:10, and 1:50 γMSC: CD3+ T cell mixtures only. TUNEL and Ki67 will be targeted to determine the survival and proliferation of single γMSCs. Spatial transcriptional and proteomic features will be extracted at the pixel level and correlated to the CD8 and CD4 T-cell phenotypes. Rigor: Experimental demonstration of potency chip design with microfluidic channels (0.8-mm×2-cm×2-cm) for co-culture of γMSCs cell suspension (1×10⁶, 5×10⁶, 10×10⁶ cells mL-1) and PBMC-isolated CD3+ T-cells (2×10⁶, 5×10⁶, 10×10⁶, 50×10⁶, and 100×106 T-cells mL-1) per well.

### Results

Provided herein is a better understanding of how the 3D-treated substrate affects the growth and immunomodulatory potential of single γMSCs. We expect that spatial multi-omics features will provide the subset of γMSCs that exhibit similar or distinct gene regulatory programs to balance inflammatory and anti-inflammatory secretion profiles on 3D PEG-4MAL substrates. MSCs perform colony formation and the spatial proximity among different subsets of MSCs in colonies with similar or varying gene and protein expression levels will be revealed from the proposed experiments. Our study will advance our understanding of auto/paracrine signaling networks from spatial transcriptional features of individual BM γMSCs and T-cells using their molecular profiles and relative spatial positioning. We expect that a subset of γMSC phenotypes neighboring T cells exhibit a unique combination of inflammatory and anti-inflammatory markers. We also expect that BM γMSCs are more potent in pediatric donors than in adult ones using the spatial transcriptional features and secretome analysis in the chip.

Evaluate the Correlation of Spatial Transcriptional Features on BM γMSC Potency with In Vitro T-Cell Suppression and the Clinical Outcome of HCT Patients Receiving γMSCs

BM γMSCs regulate auto and paracrine signaling to secrete in locally responsive and personalized to the host in vivo environments and a correlative framework will provide distinct MSC potency maps in non-responder and responder GVHD patients treated with γMSCs.

Stem cell interventions fail to assess BM MSCs' potency in degraded patient tissues after therapies (we cannot isolate tissues) and the in-vivo models still suffer from the lack of accurate models of MSC potency,^{63b} making it challenging to predict clinical efficacy for MSCs' therapeutic outcome. Our key application of BM γMSCs to prevent GVHD in HCT patients will be an ideal platform to show clinical correlation to the γMSCs potency. In our clinical trial, we will have access to 21 adults and 12 pediatric donors. We will know which subset of patients exhibited prophylaxis of GVHD (defined as responder) and which others suffered from GVHD (defined as non-responder), after BM γMSC and donor-derived T cell delivery through IV infusion. Therefore, we will correlate spatial multi-omics and potency chip (size and potency) features of cell types and inflammatory and anti-inflammatory markers from BM γMSCs' potency analysis and their neighboring T-cell profiles to the HCT patient's prophylaxis of GVHD outcomes. Our clinical infusions include 2-20 million γMSCs per kilo per patient and 2-100 million T cells per kilo per patient. 3D potency assay will assist in determining the optimum cell density for our clinical trials. In addition, we will assess rapid light sheet imaging modalities to quickly screen 3D potency assays from 33 patients, allowing us to generate large data of γMSC and T-cell pairs in the chip.

### Preliminary Data

Predictive pipeline for optimizing MSC potency using in vitro and in vivo: In vivo experiments leveraged BM MSC-laden hydrogels that were polymerized in situ in the subcutaneous space of immunocompromised NSG mice. While this proposal will NOT use mice, this approach is only described to show the potential of adjustable potency assay parameters. Subcutaneous implantation of BM MSCs into the immunocompromised mice allows cytokine secretion in an in vivo environment. After 3 days of injection, the BM MSC-laden hydrogel and local tissue were collected and digested for human analyte analysis by multiplex Luminex. Interestingly, the local IFN-γ activity was observed in BM MSC-laden hydrogels in vivo compared to controls, suggesting that MSCs must have secreted IFN-γ. Thus, in vivo secretions were designed to adjust the in vitro microfluidic design parameters (flow rate, hydrogel size, stimulation treatment) to match the in vivo-in vitro secretory responses. This iterative modeling of the MSC Potency provided in-vivo-like 20-plex analyte distributions, not captured in conventional 2D IFN-γ cultures, opening doors to study clinical efficacy.

Multiplexed gene expression profiling in MSCs and γMSC in 3D Hydrogel chip: To validate the effect of IFN-γ treatments on gene expression of MSCs, we designed a proof-of-concept experiment. BM MSCs (Rooster Bio donor 00177) were treated with IFN-γ for 1 day in the 3D hydrogel chip, yielding γMSC phenotypes. As a control, another set of BM MSCs (same donor) was grown in the 3D hydrogel without IFN-γ. The 3D potency chip design is akin to the original platform, where the 3D MSC-laden hydrogel was prepared according to the protocol outlined in section E.4.1 on glass slides with a chamber in the middle. The multiplexed RNA imaging was also performed similarly to the procedures listed in section E.4.2., where γMSC/MSC-laden hydrogels were fixed with 4% formaldehyde and permeabilized with 70% cold ethanol. The γMSC/MSC-laden hydrogel was then re-mounted to a thin coverslip sample for high-resolution gene expression analysis (FIG. 17a). Multiplexed experiments profiled five gene targets, including COL1A1, ACTB, PDL1, IL8, AND IL6 in 15 BM MSCs (control) and 15 BM γMSCs using 2-cycles. Five genes were chosen to compare functional differences of MSCs and γMSC upon activation with IFN-γ for 24 hours at 50 ng/mL concentration using microfluidic flow at 1 μL/min for 24 hr over a supernatant collection of 1.44 mL. COL1A1 is a collagen depositome gene, IL8 and IL6 are the cytokines, PDL1 is the immunomodulatory gene, and ACTB is the housekeeping gene. γMSC exhibited higher COL1A1, IL6, and IL8 gene expression than unstimulated MSCs, while there was no significant difference in ACTB and PDL1 gene expression (FIG. 17b). Microfluidic IFN-γ treated BM MSC n=15 and Microfluidic untreated BM MSC n=15 across 3 donors performed in 3D hydrogel-based potency chip. Star significance for P<0.005; ns is non-significant, p>0.05. This experiment demonstrated the activation of γMSC and the feasibility of multiplexed gene analysis in a 3D potency chip.

Demonstration of MSCs and γMSC product-to-product consistency: Safety profiles and product quality of γMSC were characterized in preclinical and clinically-related in vitro procedures. MSCs obtained from three different healthy donors were isolated and IFN-γ primed according to our GMP protocol. RNA isolated from the MSCs and the corresponding γMSC from the same donor was assessed by RNA Seq. Principal component analysis (PCA) using the normalized expression values for the 500 genes with the highest variance yielded distinct clusters of the three γMSC samples from activated MSCs, with the first principal component explaining 86% of the variance. This analysis also distinguished the three patients' samples using the 8% of the variance. While the majority of variation was a result of IFN-γ priming, the cytokine caused a similar variance in each of the patients; thus, IFN-γ priming remains unaffected due to additional interpatient variation. In addition, Euclidean distance was calculated to assess the overall similarity between samples. This analysis separated hierarchical clusters of the three treated or untreated samples. To quantify the relative gene expression differences of the γMSC versus MSCs, the 20 most differentially expressed genes were shown graphically as a heat map. The most highly upregulated pathway was due to the antigen presentation when comparing IFN-γ primed MSCs with conventional MSCs. Note that immune-modulating cytokines, IL6, IL15, and IL32 were upregulated while IL16 and IL11 were downregulated; however, only IL6 was expressed at a considerable level. Upregulation or downregulation of proto-oncogenes or tumor suppressor genes is negligible, respectively.

### Design and Approach

GMP manufacturing of γMSC from distinct donors: We will use a 2-step manufacturing process^{1b} to prepare the clinical γMSC product. To validate the manufacturing protocols and assess the pharmacology/toxicology of the clinical product, we will prepare MSCs from 5 donors with and without IFN-γ priming of the cells. MSCs isolated from a donor and expanded ex vivo in parallel tissue culture flasks should exhibit unchanging gene expression profiles, indicating any observed differences between the cell populations in this study must be associated with IFN-γ. For MSC manufacturing intended for clinical trials, the bone marrow donor must satisfy the hematopoietic cell donor criteria described in the current standards of the Federation for Accreditation of Cellular Therapy (FACT). Viable MSCs will be recovered from cryopreserved stocks. After 7 days of expansion with and without IFN-γ (25 ng/ml-500 U/ml GMP-grade recombinant human IFNg, R&D systems) treating in the last 2 days of expansion, cells will be collected by trypsinization, washed with PBS 1×, and suspended in infusion media at a concentration of 1 to 10×10⁶ cells per ml. Next, MSCs (control) and γMSC (sample) mixed with T cells (2 to 100×10⁶ cells per ml) will be slowly passed through a syringe and tubing to simulate a clinical slow intravenous “push” infusion, followed by integration with 3D hydrogel assay and spatial-multiomics profiling of the MSCs with T cells.

Spatial transcriptional and proteomic features of 3D γMSC potency and pairing T cells in distinct donors: Spatial RNA and protein maps of previously mentioned targets (see sections 5.3-5.4) will be profiled in BM γMSCs (21 adults and 12 pediatric donors each), responding to the in vivo microenvironment after 3-days (chosen as the MSC responds within 24-72 hours64^{b}). IFN-γ primed (25 ng/ml-500 units/ml) BM-MSC sources from five donors each will be obtained from the Marcus Center for Pediatric Cell Therapy at Children's/Emory and T-cells will be isolated from 21 adult and 12 pediatric donors (i.e., IV infused at 2-10M γMSCs and 10-100M T cells per kg per patient). Thus, we will evaluate the co-cultures of MSC cell suspension (1×10⁶, 5×10⁶, 10×10⁶ cells mL-1) at passage 2 (see GMP guidelines)^{1b} with >70% viability mixed with PBMC-isolated CD3+ T-cells (2×10⁶, 5×10⁶, 10×10⁶, 50×10⁶, and 100×10⁶ T-cells mL⁻¹ per well after 3-days co-culture. We will test different γMSC: T cell ratios (1:2, 1:10, and 1:50) to generate clinical insights into required cells per IV infusion. The same rations will be compared with and without T-cell activation using anti-CD3+/anti-CD28+ Dynabeads (ThermoFisher) at a 1:1 T cell to bead ratio and 12 U rIL-2 (PeproTech). CD3+ T cells will be isolated from PBMCs using Dynabeads™ FlowComp™ Human CD3 Kit (ThermoFisher: 11365D). The spatial transcriptional and proteomic analysis will be performed after fixing the cells within the 3D hydrogels and directly optical imaging at 20×-60× without sectioning, an experimental RNA (ActB, IL6, and IL8, along with nucleus in DAPI) data of MSCs with PBMCs were presented in 3D gels for establishing the feasibility (FIG. 18). The supernatant will be collected and the secretome will be profiled by Luminex. Power: 1,000 spatial features can be detected at 80% power and a significance level of 0.05 for each sample and 5 replicate for each experiment.

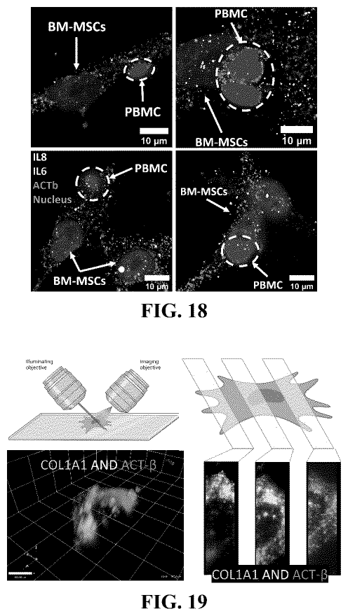

Rapid light sheet imaging of spatial multi-omics in γMSC potency chip: Confocal fluorescence imaging is the method of choice for 3D assays. Thus, in section F 4.2., we will be using Nikon spinning disk confocal to acquire spatial transcriptional and proteomic data from γMSC and T cells in 3D hydrogel specimens (1-mm×2-cm×2-cm) at 20× optical magnification. While the spatial resolution is sufficient to quantify the low to high signal distributions, we need fast imaging techniques to rapidly image the entire volume of each 3D potency chip. There are two commercially available solutions. The first one is a Lightsheet Z.1 system that can again use 20× optical magnification to acquire images at a 6-fold higher speed using comparable exposure times per channel. Z.1 Lightsheet reduces the photobleaching effects 5000 times compared to point-scan confocal imaging and performs image acquisition at 100 frames per second, in contrast to the confocal at 1-5 frames per second.^{65b} In addition, Z.1 light sheet system takes 10 seconds to image an area of 100 μm over a depth of 800 μm, while the spinning disk confocal takes 1 minute to cover a similar imaging area over a depth of 200 μm, which is 4-fold shallower compared to Z.1 light-sheet imaging.^{66b} Thus, we will use Z.1 light-sheet imaging to achieve 6-fold faster and 4-fold deeper imaging in the 3D γMSC potency chip. We will also modify the geometry of the hydrogel chip from a chamber on the slide to a capillary format to perform the necessary tasks of 3D hydrogel imaging. Next, to perform 60× imaging at the single molecule level, we will employ lattice light-sheet microscopy^{67b} (3i, Intelligent Imaging Innovations). As preliminary data, we validated imaging of single RNA molecules in fixed MSCs located on 5 mm round coverslips (Warner Instruments) at Emory University with Research Scientist Stoyan Ivanov. The resultant data from the 3i Lattice light-sheet microscope yielded highly sensitive detection of multi-color RNA-HCR labels for COL1A1 and ACTB genes at 300 ms per image acquisition (FIG. 19). Thus, we reason that rapid imaging of fixed 3D γMSC and T cell pairs in hydrogel can be image across 500 μm depth over 2 mm×2 mm area. This is akin to live single Drosophila embryos (˜500 μm×200 μm) using the Lattice light-sheet imaging.^{68b,69b}We will then prepare a high-throughput hydrogel array using laser-patterned PDMS substrates, allowing to profile of ˜50 3D potency chips at single-molecule resolution for evaluating the 3D γMSC and T cell interactions with different ratios.

Quantification of γMSC and T cell interactions using SpatialVizScore framework: To study immune infiltration in human tissues, we developed a data science approach, termed SpatialVizScore,^{37b} using graph-based spatially resolved cellular analysis (npj Precision Oncology 2022^{37b}). Using SpatialVizScore, we quantified the immune infiltration patterns within cancer H&E images with the drug response data to immune checkpoint inhibitors (ICIs) (FIG. 20). The feasibility of graph-based cell maps for predictive analysis of tumor-immune interactions was established using SpatialVizScore, providing a digital pipeline. Thus, we will establish a “SpatialPotencyScore” based on MSC and T cell interactions and suppression using spatial multi-omics. A neighborhood of d=3 cell diameter will be measured to create scores.

Correlative analysis of in vitro γMSC potency and in vivo GVHD outcome: A correlative framework will be performed to incorporate in vitro performance of γMSC potency chip with that of in vivo outcome using spatial transcriptional and proteomic features, and the secretome variability for BM γMSCs for 5 donors. Rigor: The multi-dimensional maps and the correlative pipeline predicts auto and para signaling factors for BM γMSCs associated with the poor or good outcome of GVHD therapies using γMSCs. Rigor: Spatial multi-omics will be analyzed by Seurat v4^{59b} single-cell multi-modal data integration. The multi-dimensional space will be classified by k-nearest neighbors (k=3, 7, & 10) to predict auto and para signaling factors in this pipeline for BM γMSCs and T-cells (FIG. 21).

### Results

A better understanding of BM γMSC potency with spatial molecular features and secretome profiles that can be used for predictive modeling of γMSCs' immunomodulatory potential. In vitro assay and spatial multi-omics profiling can be integrated into the rationally designed clinical use of BM γMSCs, particularly to shed light on superior GVHD treatments in pediatric patients compared to adults. On-chip assay clinical validation will enable us to correlate on-chip functional metrics with clinical outcomes.

### Example 4: Subcellular Spatially Resolved Gene Neighborhood Networks in Single Cells

### Summary

Image-based spatial omics methods such as fluorescence in situ hybridization (FISH) generate molecular profiles of single cells at single-molecule resolution. Current spatial transcriptomics methods focus on the distribution of single genes. However, the spatial proximity of RNA transcripts can play an important role in cellular function. We demonstrate a spatially resolved gene neighborhood network (spaGNN) pipeline for the analysis of subcellular gene proximity relationships. In spaGNN, machine-learning-based clustering of subcellular spatial transcriptomics data yields subcellular density classes of multiplexed transcript features. The nearest-neighbor analysis produces heterogeneous gene proximity maps in distinct subcellular regions. We illustrate the cell-type-distinguishing capability of spaGNN using multiplexed error-robust FISH data of fibroblast and U2-OS cells and sequential FISH data of mesenchymal stem cells (MSCs), revealing tissue-source-specific MSC transcriptomics and spatial distribution characteristics. Overall, the spaGNN approach expands the spatial features that can be used for cell-type classification tasks.

### Introduction

Discussed herein is the spaGNN pipeline to extract gene proximity relationships from subcellular spatial transcriptomics data. The algorithm detects the proximity of genes in subcellular compartments while constructing gene neighborhood networks. The gene spatial proximity relationships outperform gene expression in cell classification.

Spatially resolved transcriptomic technologies such as multiplexed error-robust fluorescence in situ hybridization (FISH) and sequential FISH generate high-dimensional datasets that reflect the organization of cells within tissues but also the organization of molecules within cells. We sought to develop an analysis method for inferring subcellular molecular interaction networks from image-based spatial omics data. This algorithm quantifies the physical proximity of RNA molecules when they are spatially located within the nearest neighborhood distances. Spatially resolved gene neighborhoods are used to generate networks that are distinct at different parts of a single cell. Decoding subcellular gene neighborhood networks enables downstream tasks such as cell classification better than overall gene expression per cell, thus expanding the use of subcellular spatial features for omics analysis.

Subcellular localization of RNA molecules and their associated physical mechanisms have previously been quantified and outlined using in situ hybridization methods (fluorescence or chromogenic) in model Organismsl® for the study of neuroscience,^{2c} development,^{3c} and mammalian subcellular organization.^{4c} Emerging spatially resolved and multiplexed gene-expression profiling technologies include two mainstream approaches. Spatial transcriptomics and Slide-seq leverage DNA-barcoded substrates and microbeads to capture multiple RNA targets, providing gene-expression maps of tissues at 10- to 100-mm resolution.^{5c,6c} The spatial resolution limitation and sample preparation complexity of these platforms are bottlenecks for transcriptomics analysis of single cells. Another approach utilizing image-based barcoding of gene-expression maps is the fluorescence in situ hybridization (FISH) method for the detection of RNA molecules in their native positions within cells and tissues. Sequential FISH (SeqFISH) and multiplexed error-robust FISH (MERFISH) methods have generated single-cell spatial maps of 10-10,000 RNA species in mouse tissues. These maps have since been utilized in neuroscience, developmental biology, and embryonic stem cell biology.^{7c-10c}

The subcellular organization of RNA molecules inside individual cells is crucial to position-dependent gene regulatory control of cellular decision-making processes, including stem cell differentiation.” ^{c} Thus, subcellular spatial transcriptomics provides regulatory and functional cell information otherwise masked by traditional molecular analyses. FISH-based multiplexing methods localize single RNA transcripts, enabling the study of the spatial organization of RNA-enriched compartments in subcellular volumes, a feature previously limited to live-cell imaging.^{12 c} For instance, b-actin molecules are observed to be localized at the cell edge and focal adhesion points, allowing cells to efficiently migrate and polarize.^{13 c} However, RNA regulation also occurs compartmentally, within neighborhoods defined by common features spanning 0.1-30 mm. Direct RNA and RNA interaction can also regulate the expression and localization of transcripts.^{14 c,15 c} This work presents a computational method to study RNA neighboring relationships, entitled spatially resolved gene neighborhood networks (spaGNN).

In cells, complex regulatory relationships between genes maintain gene-expression phenotypes.^{16 c} Gene transcription regulation is modulated by a cascade of biochemical processes, leading to the control of transcription levels.^{17 c} Gene regulatory networks (GRNs) have been widely used to describe the regulation of gene expression, and a wide range of tools have been developed to discover the regulatory relationship between genes (Table 3).^{18 c-21 c}

TABLE 3
Summary of current gene network analysis methods, related to FIGS. 22A-22I.

| Computation | | | |
|---|---|---|---|
| Approach | Summary | Technology and data | Examples |
| Co-expression | Discovery co- | scRNE seq | Stuart, et al. 2003^{1d} |
| | expression and | DNA microarray^{1d} | Tang, et al., 2018^{2d} |
| | correlation | | |
| | relationships. Does | | |
| | not infer a direct | | |
| | regulation | | |
| | relationship. | | |
| Transcription factor | Directly uses binidng | DNA sequencing, | Bailey, et al. |
| binding sites analysis | sire information and | sequence matching, | 2009^{4d} |
| | bio-chemical process | ChIP-seq | Kulkarni, et al. |
| | of regulation to study | | 2018^{5d} |
| | gene expression | | Verfaillie et al. |
| | regulation. ^{3d} The | | 2015^{6d} |
| | result an be | | |
| | confounded by | | |
| | multiple | | |
| | transcription | | |
| | regulation pathways. ^{3d} | | |
| Computational | Infers regulatory | scRNAseq | Liu, et al. 2022^{7d} |
| inference | network from gene | | Huynh-Thu et al. |
| | expression data using | | 2020^{8d} |
| | mathematical | | |
| | models. | | |
| Spatial regulation | Infers regulatory | FISH, spatial | Yang et al. 2019^{9d} |
| | relation from | transcriptomics | Puniyani and Xing |
| | spatially resolved | | 2013¹⁰ |
| | transcriptomics using | | |
| | spatial patterns. | | |
| SpaGNN | Examines cellular, | Image based spatial | |
| | subcellular, and | transcriptomics | |
| | localized co- | | |
| | expression | | |

GRNs provide intuitive visualization by representing genes as nodes in graphs, and regulatory relationships as edges. Coexpression networks reveal functional and regulatory commonalities between genes.^{22 c,23 c} However, they lack directional connection and miss the regulator or target information. Investigations of transcription factor binding sites in the genome dissect the biochemical process of regulation.^{19 c,24 c,25 c} However, regulatory relationships are frequently confounded by multiple regulatory pathways that lead to impre-cise models.^{18 c} Statistical inference models have also been proposed to infer regulatory networks from single-cell transcriptomic profiles.^{20 c,21 c} Spatial transcriptomics data has also been used to infer gene regulatory relationships.^{26 c,27 c} These computational approaches utilize existing transcriptomics data to predict regulatory relationships. Although these expedient methods utilize widely available data, they too lack subcellular spatial resolution (Table 4).^{28 c} The spaGNN pipeline seeks to examine and visualize the spatial proximity relationships between genes as networks.

TABLE 4
Selection consideration of current gene network analysis
methods and spaGNN, related to FIGS. 22A-22I

| Computation approach | Advantage | Disadvantage |
|---|---|---|
| Co-expression | Computationally identified | Lacks inference of regulator |
| | using a straightforward | and target genes. The |
| | metric | connection is not directional. |
| Transcription factor | Directly studies the regulator | The regulatory relationship |
| binding sites analysis | of target genes. Regulatory | can be weak due to multiple |
| | relationships are more likely | regulators controlling the |
| | to be realistic | same target.^{3d} |
| Computational inference | Data is widely available. Can | Computationally complex. |
| | analyze highly multiplexed | Prediction ability varies |
| | data | between different models |
| | | and datasets.^{11d} |
| Spatial regulation | Utilizes spatial distribution | Loss of spatial variation data |
| | of RNA molecules | of regulatory networks |
| Spa-GNN | Examines cellular, and | |
| | subcellular colocalization | |
| | and correlation. Examines | |
| | variation of subcellular RNA | |
| | spatial organization | |

### Results

The spaGNN pipeline reveals subcellular gene neighborhood relationships Transcripts have been shown to present subcellular spatial patterns. Therefore, we investigated the spatial segmentation of each cell into patches defined by high local RNA density utilizing the Leiden clustering algorithm.^{29 c} By applying the clustering algorithm to the positions of all detected transcripts, the algorithm separates transcripts into local communities based on RNA density. Pearson's correlation between markers among patches indicates the tendency for genes to colocalize in the same patch. The correlation value, denoted as R, is defined as the patch correlation of gene pairs. To demonstrate the pipeline, we analyzed the published MERFISH dataset on fibroblasts^{30 c} (FIG. 22A and FIGS. 37-42). Such a dataset contains the subcellular locations of 130 genes in human fibroblasts. The clustering algorithm divides the presented cell into spatially defined patches (FIG. 22B), and the pairwise correlations of all 130 genes were computed (FIG. 22C and FIGS. 37-42). The heatmap (FIG. 22D) highlights several gene pairs with a range of patch correlations. There are six pairs of genes with high and low patch correlations. To visually validate the patch correlation, the locations of several pairs of genes were plotted (FIG. 22E). FBN2-FLNA, PRPF8-SRRM2, and SRRM2-MKI67 all have positive patch correlation. The locations of transcripts show that they localize in similar subcellular spaces. MALAT1-FLNA, PRPF8-NUMA1, and THBS1-MALAT1 all have negative patch correlations. The locations of 130 unique transcripts show that they localize in different subcellular spaces of the MERFISH data. To further demonstrate the robustness of the method, we examined patch correlation if fewer genes were studied. The detected patches using 10, 50, and 100 genes are consistent with the patches detected using all 130 genes as shown by the Rand index comparison. The patch correlation also remains consistent, owing to the consistency in patch detection (FIGS. 30A-30E).

### Local Gene Neighborhood Network Reveals Gene Proximity Relationship

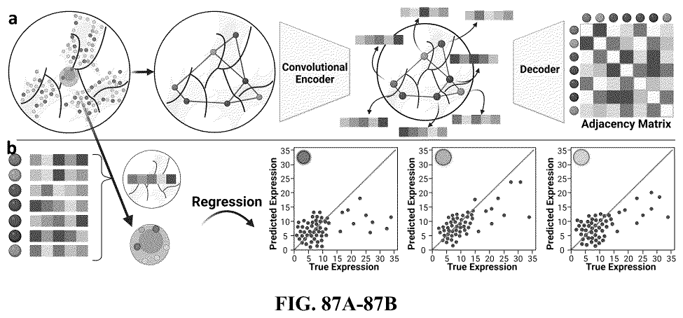

Localized spatial gene neighboring relationships were then analyzed through local neighborhood analysis (FIGS. 22F-22H). Within each patch, the center transcript and its surrounding nine neighbors form a ten-transcript neighborhood as shown by connected dots (FIG. 22F). Each dot is a detected transcript, and an edge indicates a neighboring event between two genes (FIG. 22F). Ten-gene neighborhoods were computed for all transcripts in the same patch, and the number of neighboring events was counted. We used a permutation analysis with 1,000 permutations to find the distribution of pairwise gene neighboring events given the gene count. We defined the Z score of the detected number of interaction events given the distribution as the proximity score (denoted Z) between the pair of genes. The proximity score was then visualized using the NetworkX package³¹° (FIGS. 22G and 22H). Scatterplots show the locations of several pairs of genes (FIG. 221). TEAD1-LRP1, IGF2R-FBN2, and FLN1-FBN2 pairs all have positive proximity scores, indicating that the gene pairs are neighbors of each other more frequently than expected. Meanwhile, TNC-FLNC, PRPF8-COL5A1, and THBS1-MALAT1 all have negative proximity scores. However, because of the nearest-neighbor approach of studying gene neighborhoods, the detected proximity relationship can be affected by the varying number of genes studied (FIGS. 31A-31E). Further analysis indicated that the mean and standard deviation of proximity score between the same pairs of genes were not significantly different when the number of genes studied was changed.

### Subcellular Spatial Gene Neighborhood Features are Distinct in Different Cells

In addition to the fibroblast MERFISH dataset, we analyzed the published U2-OS MERFISH dataset^{32 c} using the same methods. The common 84 genes in the two MERFISH datasets were compared. Cells of different types present distinct patch correlations between the same set of genes (FIGS. 23A and 23B; FIGS. 37-42). For example, the patch correlations between SRRM2 and PRPF8 are similar between fibroblast and U2-OS cells (FIG. 2C). However, the patch correlations between PRPF8 and IGF2R are different between fibroblast and U2-OS. The two genes have a negative correlation in fibroblast shown by R=−0.33, and a positive correlation in U2-OS shown by R=0:38 (FIG. 23D). When comparing the patch correlation of both cell populations, both SRRM2-PRPF8 and IGF2R-PRPF8 pairs show significant differences (FIG. 23E). Therefore, the two genes are more likely to locate in the same subcellular patch in U2-OS than in fibroblast.

We further investigated the variation of subcellular gene neighboring relationships. The proximity score, which quantifies the likelihood of two genes being neighbors of each other, varies across the same cell (FIGS. 24A and 24B). The networks between the same set of genes, THBS1, FBN2, and SRRM2, show variation across the same cell (FIGS. 24C and 24D). The neighboring relationships between transcripts were also highlighted (FIGS. 24E and 24F). The variation of proximity scores across patches allows the analysis of the mean and standard deviation of proximity scores across the same cell (FIG. 24G). The mean proximity scores between THBS1-FBN2, THBS1-SRRM2, and FBN2-SRRM2 of the fibroblast are higher than in the U2-OS cell.

Subcellular Neighborhood Networks Classify Cells Better than RNA Expression

With each cell presenting distinct patch correlations and unique distribution of local neighborhood proximity scores, we utilized t-distribution stochastic neighborhood embedding (t-SNE) to visualize the distribution of patch correlations and local proximity scores. First, the cells were clustered based on single-cell RNA count (FIG. 25A). All detected RNA dots were counted per cell to generate a single-cell expression profile of each cell. Next, the cells were clustered based on subcellular patch expression correlation (FIG. 25B). Finally, the cells were clustered by the variability of subcellular spatial neighborhood networks as measured by the mean and variance of proximity score between each pair of markers (FIG. 25C). The t-SNE plots highlight that all three sets of features can separate fibroblasts and U2-OS cells. We used the clustering result for cell-type classification. Cells in the same cluster are predicted to be the same type as the majority of cell types. Confusion matrices show that all three features predict fibroblast and U2-OS with high accuracy (FIGS. 25D-25F).

### Spatially Resolved Subcellular Transcriptomics Dissects Mesenchymal Stem Cell Heterogeneity

Mesenchymal stem cells (MSCs) show strong regenerative and immunoregulatory potential and are tested as cell-based therapies for a wide range of diseases.^{33 c,34 c} Owing to their differentiation potential and immunomodulatory roles, MSCs are widely studied and have been shown to display considerable spatial and temporal molecular heterogeneity.^{35 c} MSCs are isolated from multiple sources including human bone marrow (HBM), adipose tissue (HAT), and umbilical cord (HUC), making it challenging to clinically validate the efficacy and safety of cell-based therapies.^{34 c,36 c} HUCs, for example, show distinct differences in functional characteristics such as proliferation potential, differentiation efficiency, and immunomodulatory character when compared with HBMs.^{37 c,38 c} Donor age and general health status introduce further MSC heterogeneity.^{39 c} Allogeneic MSCs benefit biomanufacturing processes with wide availability.^{40 c} However, autologous MSCs eliminate the issue of immune rejection associated with allogeneic MSCs.^{41 c} The standardization of MSC-based therapies is further complicated by the intra-colony variability of MSCs due to clonal evolution during manufacturing.^{42 c} MSC phenotype is currently defined by the expression of CD105, CD73, and CD90 markers, lack of expression of CD45, CD34, CD14 or CD11b, CD79a or CD19, and HLA-DR, and the potential to differentiate into osteoblasts, adipocytes, and chondrocytes in vitro.^{43 c,44 c} However, deep molecular profiles beyond surface markers are required to understand MSC heterogeneity.

Bulk and single-cell molecular profiling has yielded functional subpopulation differences in differentiation potential, immunoregulatory function, and clinical efficacy at the cost of subcellular spatial details.^{45 c-47 c} Single-cell RNA sequencing analyses of human MSCs have yielded functional subpopulation differences in differentiation potential, immunoregulatory function, and clinical efficacy at the cost of subcellular spatial details.^{38 c,48 c-51 c} Label-free and morphological imaging methods have demonstrated the utility of image-based MSC classification for clinical cell manufacturing pipelines using only a limited number of biomarkers.^{52c-54 c} Spatially resolved molecular omics technologies can combine the advantages of imaging approaches and single-cell molecular analyses while providing more detailed spatial molecular profiling of MSCs. While these demonstrations were initially limited to murine models, emerging multiplexed FISH techniques combined with signal amplification assays open doors to the detailed study of human stem cell biology, deciphering subpopulation, donor, and source heterogeneity of MSCs.^{55 c} Utilizing the spaGNN workflow, we examined HUCs (n=121), HBMs (n=237), and human chondrocytes (HCHs; n=247).

MSC samples were obtained from RoosterBio based on clinical quality-control standards (see STAR Methods) to ensure that the studied cell populations exhibited bulk characteristics similar to those of the therapeutic product. To overcome the imaging limitations of traditional cell-culture methods on plastic substrates, we cultured cells at a density of 500 cells/cm² on an optically clear 2D glass substrate (100-150 mm thickness) coated with a sub-5-mm-thick layer of collagen. MSCs are structurally intricate stromal cells exhibiting a high cell autofluorescence background due to high subcellular protein density. Therefore, MSCs necessitate the use of the split-probe design and signal amplification of the hybridization chain reaction (HCR) assay,^{55 c} providing bright, single-molecule RNA images with high detection specificity (FIG. 26A and FIGS. 43-49). This multiplexing strategy simultaneously labels and images up to three RNA targets per cycle. Each cycle of HCR and imaging is followed by enzymatic digestion of HCR components using a DNase I enzyme. The removal protocol was validated by comparing post-enzyme treatment images with the image with the HCR structure still attached. The removal of diffraction-limited dots indicates the removal of the HCR structure (FIGS. 43-49). Subsequent rounds of labeling then target different sets of RNA species, providing a linearly scalable method of cycle-number-dependent multiplexing. For example, six sequential hybridizations with three RNA targets per hybridization would profile 18 unique RNA markers in a single MSC.

Spatially resolved gene-expression maps of single MSCs were provided in the form of individual images for multiple RNA targets (FIG. 26A). Observed MSC subcellular heterogeneity suggests region-dependent RNA densities within individual MSCs, a finding contrary to models of RNA distribution utilizing an assumption of the Poisson distribution of RNA within a cell. This observation is akin to chromatin density detected in stem cell differentiation.^{56 c,57 c} Such RNA densities have previously been observed in neuronal cells^{58 c} but not in MSCs.

Twelve RNA markers were measured in HBM and HUC, and four additional RNA markers were measured in HCHs (Table 5).

TABLE 5
Multiplexed RNA and protein detection panel in
single stem cells, related to FIGS. 26A-26E.

| Gene | Target | Conjugate | Label | Description |
|---|---|---|---|---|
| ACTb | mRNA | B1 | Alexa-647 | Cytosolic Actin |
| | | | | Filaments |
| GAPDH | mRNA | B3 | Alexa-555 | Glyceraldehyde |
| | | | | 3-phosphate |
| | | | | dehydrogenase |
| EEF2 | mRNA | B3 | Alexa-555 | Eukaryotic |
| | | | | Translation |
| | | | | Elongation |
| | | | | Factor |
| NANOG | mRNA | B1 | Alexa-647 | Transcription |
| | | | | Factor - |
| | | | | Pluripotency |
| SOX9 | mRNA | B5 | Alexa-488 | SRY-Box |
| | | | | Transcription |
| | | | | Factor 9 |
| RUNX1 | mRNA | B3 | Alexa-555 | RUNX Family |
| | | | | Transcription |
| | | | | Factor 1 |
| SPP1 | mRNA | B1 | Alexa-647 | Secreted |
| | | | | Phosphoprotein 1 |
| COL1A1 | mRNA | B3 | Alexa-555 | Collagen Type I |
| | | | | Alpha 1 Chain |
| COL5A2 | mRNA | B2 | Alexa-555 | Collagen Type V |
| | | | | Alpha 2 Chain |
| IL6 | mRNA | B3 | Alexa-555 | Interleukin 6 |
| | | | | Cytokine |
| IL8 | mRNA | B5 | Alexa-488 | Interleukin 8 |
| | | | | Cytokine |
| CCL11/Eotaxin-1 | mRNA | B1 | Alexa-647 | C-C Motif |
| | | | | Chemokine 11 |
| CXCR4 | mRNA | B1 | Alexa-647 | C-X-C Motif |
| | | | | Chemokine |
| | | | | Receptor 4 |
| CD274/PDL1 | mRNA | B1 | Alexa-647 | Programmed |
| | | | | death-ligand 1 |
| MK167 | mRNA | B3 | Alexa-555 | Marker of |
| | | | | Proliferation Ki- |
| | | | | 67 |
| MALATI | IncRNA | B2 | Alexa-555 | Metastasis |
| | | | | Associated Lung |
| | | | | Adenocarcinoma |
| | | | | Transcript 1 |
| GOLPH4 | Protein | Antibody | Alexa-555 | Golgi integral |
| | | | | membrane protein |
| | | | | 4 |
| CONCANAVALIN- | Protein | Antibody | Alexa-488 | Lectin binding to |
| A | | | | the glycoproteins |
| | | | | and stains |
| | | | | endoplasmic |
| | | | | reticulum |
| WGA | Protein | Antibody | Alexa-555 | Carbohydrate-free |
| | | | | protein binding to |
| | | | | glycoproteins and |
| | | | | stains Golgi |
| | | | | apparatus |
| PHALLOIDIN | Protein | Antibody | Alexa-647 | Bicyclic peptide |
| | | | | staining F-actin |
| | | | | filaments |

NANOG, a pluripotency marker, was detected in only a few RNA copies per cell and was predominantly clustered around the nuclear membrane. PDL1, IL6, IL8, and CCL11 are genes related to the immunomodulatory function of MSCs which exhibited differences in their locations relative to cytoplasmic membranes in this experiment. Subsequent cell painting stains demonstrated cell-source-specific RNA densities and Golgi apparatus locations, most likely due to ER enrichment of translational machinery and rapid turnover of collagen secretions through ER-Golgi transport.^{9 c, 59 c}. COL1A1 transcripts were observed to be enriched on ER structures as detected by Concanavalin A (ConA) staining, and were excluded from the Golgi region defined by the Wheat Germ Agglutinin (WGA) stains. Another important observation is that neighboring cells exhibited asymmetric RNA distributions, especially SPP1 and b-actin genes in pairs of HUC (FIGS. 43-49). This finding can likely be attributed to asymmetric cell division events leading to a unique differentiation potential for each MSC. The initial observations were further investigated using the spaGNN pipeline.

spaGNN Analysis Distinguishes Tissue-Specific Subcellular Spatial Transcriptomics of MSCs

To further examine the newly generated dataset, we applied the spaGNN pipeline to the spatially resolved transcriptomic profile of MSCs. The average patch correlation of HBM, HUC, and HCH is shown in FIGS. 26B, 26C, and 32A-32G. Notable differences include greater IL6 and IL8 colocalization in HUC when compared with HBM. Additionally, IL6 was more colocalized with GAPDH, EEF2, and b-actin in HCHs than HBM and HUC sources (FIGS. 32A-32G). Utilizing the known segmentation of each cell, we also examined the effect of changing cell morphology on patch detection. The same cell was segmented using several different masks. The resulting detected patches remain similar. On further analysis of the patch correlations utilizing principal component analysis, significant differences were detected between HBM, HUC, and HCH. Principal component 1 (PC1) captures separations between HCH and MSCs, while PC2 captures separations between HBMs and HUCs. All compared HCHs and MSCs in PC1 show statistically significant differences with α=0.05, except for IL8 and IL6 differences between HBM and HCHs. Noticeably, most differences between HCHs and MSCs were correlations involving IL6, such as IL6 and NANOG, IL6 and GAPDH, and IL6 and EEF2.

We further applied nearest neighborhood network analysis to the MSC spatial profile dataset. A different approach was used to find the proximity score. After ten-transcript local neighborhoods were detected, the copy number of each gene in each neighborhood was counted and the pairwise correlation computed. The resulting R value is defined as the correlation and network variability misclassify fewer HUCs and HCHs as HBMs (FIGS. 28D-28F). Such increased cell-type classification allows better proximity score (FIGS. 26D and 26E). A patch near the center of the cell displays COL1A1 enrichment (FIG. 26D). The presented neighborhood includes five copies of b-actin, one copy of GAPDH, three copies of COL1A1, and one copy of RUNX1. The second example demonstrates a patch in the cytosolic region and shows no COL1A1 expression (FIG. 26E). The neighborhood shown included six copies of b-actin, three copies of GAPDH, and one copy of RUNX1. The same neighborhood connectivity analysis was conducted for all patches among all cells. Across the same cell, patches exhibit varying neighborhood network features (FIG. 26E). The proximity scores from all patches in each HBM, HUC, and HCH were also visualized, with each patch showing distinct neighborhood networks (FIG. 27).

The cells were then clustered and presented in t-SNE plots (FIGS. 28A-28C and FIGS. 43-49). We then performed cell-type classification using the same method as for the MERFISH dataset. Confusion matrices indicate that classification based on patch The presented spaGNN workflow identifies cellular and subcellular heterogeneity in vitro. The methods can also be extended to further analyze subcellular molecule and structure localization. For example, RNA-FISH and immunofluorescence (IF) combined staining can help study the relative localization of RNA and structure. ConA staining enables visualization of the ER, and WGA works similarly for the Golgi apparatus. Using the average intensity of 3 3 3 pixels around each RNA in the IF image, we extended spaGNN analysis to include patch correlation and proximity score of RNA, as well as ConA and WGA. In a COL1A1-enriched patch, COL1A1 was highly colocalized with ConA, while b-actin was highly colocalized with WGA. In a COL1A1 sparse patch, COL1A1 was highly colocalized with WGA and ConA (FIGS. 50-55). The discovery was also validated by statistical comparison between COL1A1 enrichment in ConA-positive and WGA-positive regions.

The changes in the patch detection method also allow studies of unique subcellular spatial patterns. Secretion profiles are performed in the culture supernatant using multiplexed antibody arrays and Luminex assays, masking the heterogeneity across MSCs.^{60 c,61 c} Cytokine release mechanisms are coordinated by intricate spatiotemporal mechanisms guided by cell-to-cell interactions and transient secretion dynamics.^{62 c} We studied three key cytokines, IL6, IL8, and CCL11. We observed in some HUCs that cytokine RNAs, especially IL8 and CCL11, are located along the cell membrane (FIGS. 50-55). Therefore, a contour patch detection with 5-mm width from the edge of cells was applied. We then computed patch correlation and proximity score using the contour patches. In a cell with IL8 and CCL11 edge colocalization, the outermost patch of the cell presents IL6-negative proximity score between IL8 and CCL11 (FIGS. 50-55). The cells with edge-localized cytokine genes were also visualized on the uniform manifold approximation and projection (UMAP) of single-cell gene expression, patch correlation, and network variability (FIGS. 50-55).

Other methods were also used to support the gene neighborhood network analysis. Utilizing the raw images of RNA and protein markers, a pixel-wise clustering algorithm was used to visualize the spatial distribution of genes (FIGS. 56-59). Images were registered, and pixels were clustered based on the intensity of the image for each gene (FIGS. 56-59). Cell images were then recolored after upsampling based on the cluster assignment of each pixel previously determined in the downsampled dataset to visualize gene enrichment regions. The recolored cell images validate that COL1A1 mRNA is more abundant near the nucleus, while ACTb is more abundant in the cytosolic region. Other mRNA molecules are mostly distributed throughout the cell. Similarly, k-means clustering of pixels can also be applied to each image and obtains similar results (FIGS. 56-59).

A spatial statistical method was also utilized to show the subcellular spatial distribution of genes (FIGS. 56-59). The number of RNA signals as a function of distance from the cell center showed similar patterns between different RNA markers, as most RNA molecules analyzed in this study indicated cytosolic localization. However, different RNA signals exhibited maximum enrichment at different distances. The same analysis was expanded to all HBMs, HUCs, and HCHs; enrichment of each gene versus distance from the cell center was visualized as a heatmap. The subcellular distribution of RNA of different cells was then analyzed using a Kolmogorov-Smirnov hypothesis test, yielding cell-type comparisons. The p values are visualized in a heatmap.

While this MSC study was used to profile and quantify only HBM, HUC, and HCH from one donor per cell type, this image-based screening platform may be fully automated to enable a high-throughput screening assay for comparative analysis of many donors and tissue-specific MSCs. Such an MSC monitoring assay could prove essential in biomanufacturing processes such as endpoint measurements and donor screenings, providing a potential critical quality attribute to standardize functional MSC products. Integrating the aforementioned analytical methods in addition to the spaGNN pipeline would enable the discovery of biological mechanisms in MSCs not observable with bulk transcriptomics. Other stem cell sources such as induced pluripotent stem cells may also benefit from the spaGNN framework as a method to identify cell states, differentiation potential, and immunomodulatory functions. The spaGNN framework is an emerging single-cell spatial omics tool for characterizing stem cells and informing biomanufacturing pipelines.

While the spaGNN pipeline sheds light on the RNA neighborhoods, the biological mechanisms that govern such subcellular RNA organization require further investigation. We reason that several factors may lead to unique subcellular distributions of RNA. Protein secretion and distribution may be influenced by the relative position of the translation unit to one of many possible interacting features. For example, COL1A1 is secreted and deposited into the extracellular matrix; if COL1A1 RNA were colocalized with the ER, utilizing the attached translation enrichment units and subsequent Golgi apparatus delivery may increase secretion efficiency.^{9 c,59 c} Colocalization of RNA could also potentially be attributed to RNA-structural protein binding.^{9 c} These RNA-protein interactions can serve both structural purposes and post-transcriptional regulation purposes, among others.^{63 c} The localization of RNA may also assist the function of the translational product; for example, b-actin mRNA can localize near adhesion points to assist cell migration and adhesion.^{13 c,64 c} Similar effects of localized mRNA have been observed in neurons to maintain neural plasticity, a crucial factor in learning and memory.^{65 c} We also speculate that the specific localization of mRNA may increase energy efficiency. By transporting mRNA to various subcellular locations to translate proteins where they are needed, cells avoid the expensive transport of large, complicated molecules in favor of much smaller mRNA structures.

We computed Pearson's correlation to quantify the localization of gene pairs in similar subcellular patches. The statistical significance can be computed using the following equation:

$$t = r \sqrt{\frac{n - 2}{1 - {r}^{2}}} ,$$

where r is Pearson's correlation and n is the number of detected patches. |t|>1.96 indicates statistical significance with alpha=0.05.^{66 c} In the future implementations of spaGNN, such statistical testing can be incorporated to define the significance of correlation metrics.

In the spaGNN algorithm, the nearest neighborhood network is generated using the k-nearest-neighbor (k-NN) algorithm. Each local neighborhood is defined as the central transcript and the nine nearest neighbors of the central transcript. The k-NN method ensures that each local neighborhood has ten transcripts. However, the distance between transcripts in the same neighborhood can vary. An alternative approach to finding gene neighborhoods is through distance-based neighborhoods. Distance-based local neighborhoods ensure that transcripts within the same neighborhood are in spatial proximity to each other.^{67 c}

In the permutation analysis, a Z score was computed to quantify the tendency of a pair of genes to be neighbors of each other. A p value can be calculated by finding the proportion of cases that has a larger Z-score absolute value. However, the p value needs to be adjusted using Bonferroni correction, since the permutation analysis tries to find pairwise proximity relationships between multiple pairs of genes.^{68 c}

An alternative approach to compute the proximity score is by counting the copy number of each gene in each neighborhood and computing the correlation between genes among all neighborhoods in the same patch. However, the correlation method is not applicable if a large number of genes are studied. The low number of transcripts in each neighborhood causes the gene copy number per neighborhood in a patch to be sparse. Pearson's correlation would be high because of a large number of common zeros. In such cases, only the permutation analysis can be used.

Additionally, clonal heterogeneity has not been assessed in the MSC samples, making it difficult to decipher the genetic purity of MSC colonies. Multiplexed RNA imaging methods can be applied to MSCs after engineering each stem cell using a barcoded library of lentiviral vectors.^{69 c} Simple cell trackers can also be used to identify colony-forming units of individual MSCs, but these methods cannot be effectively utilized in the study of clonal mixing and evolution.^{70 c} Alternatively, single-cell time-lapse imaging can map the differentiation process by phase-contrast microscopy followed by multiplexed spatial gene-expression measurements.^{71 c} Synthetic lineage recording methods can also be applied to decipher clonal heterogeneity, but these molecular barcoding methods are difficult to execute in human stem cells.^{72 c} Other molecular analysis methods such as ATAC-seq can also be incorporated into the clonal mapping of single MSCs.^{73 c}

In the protein-RNA network analysis, the nearest-neighbor relationship was computed using Pearson's correlation. Pearson's correlation was used to compute the proximity score between RNA and protein.

Complementary RNA sequencing assays may also be used to predict the identity and function of individual stem cells. Data integration can be performed using developing multimodal data-integration techniques.^{74 c,75 c} In short, the spaGNN imagebased spatial omics framework assists in the identification of stem cell subpopulations utilized in cell-based regenerative treatments as well as the broader study of stem cell biology.

TABLE 6
Key Resources Table

| REAGENT or RESOURCE | SOURCE | IDENTIFIER |
|---|---|---|
| Antibodies | | |
| Phalloidin | Thermofisher | Cat# A34055 |
| | Scientific | |
| WGA | Thermofisher | Cat# W32466 |
| | Scientific | |
| Concanavalin A | Thermofisher | Cat# C11252 |
| | Scientific | |
| Chemicals, peptides, and | | |
| recombinant proteins | | |
| 1X DPBS | Thermofisher | Cat #D8537 |
| | Scientific | |
| 10X DPBS | Thermofisher | Cat #D8537 |
| | Scientific | |
| 16% paraformaldehyde | Thermofisher | Cat # 28908 |
| | Scientific | |
| DNase I | Thermofisher | Cat # 04716728001 |
| | Scientific | |
| UltraPure DNase, RNase free water | Thermofisher | Cat # 10977-015 |
| | Scientific | |
| MEM-a culture medium | Thermofisher | Cat # 12561-049 |
| | Scientific | |
| penicillin-streptomycin | Thermofisher | Cat #P4333 |
| | Scientific | |
| TrypLE | Thermofisher | Cat # 12605-010 |
| | Scientific | |
| Trolox | Sigma-Aldrich | Cat # 53188-07-1 |
| Pyranose oxidase | Sigma-Aldrich | Cat #P4234 |
| catalase | Sigma-Aldrich | Cat # 9001-05-2 |
| Critical commercial assays | | |
| Hybridization Chain Reaction RNA- | Molecular | hcr-rnafish |
| FISH | Instruments | |
| Deposited data | | |
| MSC seqFISH data | This study | |
| Experimental models: Cell lines | | |
| Human bone marrow-derived | RoosterBio, Inc. | MSC-031 |
| mesenchymal stem cells | | |
| Human umbilical cord-derived | RoosterBio, Inc. | C43001UC |
| mesenchymal stem cells | | |
| Human primary chondrocytes | PromoCell, GmbH | C-12710 |
| Oligonucleotides | | |
| ACTb probe | Molecular | |
| | Instruments | |
| GAPDH probe | Molecular | GenBank: NM_001289746 |
| | Instruments | NM_002046 |
| | | NM_1256799 |
| | | NM_1289745 |
| EEF2 probe | Molecular | GenBank: NM_001961.4 |
| | Instruments | |
| NANOG probe | Molecular | GenBank: NM_024865.4 |
| | Instruments | |
| SOX9 probe | Molecular | GenBank: NM_000346 |
| | Instruments | |
| RUNX1 probe | Molecular | GenBank: NM_001754 |
| | Instruments | |
| SPP1 probe | Molecular | GenBank: NM_001251830.1 |
| | Instruments | |
| COL1A1 probe | Molecular | GenBank: NM_000088.4 |
| | Instruments | |
| COL5A2 probe | Molecular | GenBank: NM_000393.3 |
| | Instruments | |
| IL6 probe | Molecular | GenBank: NM_000600.5 |
| | Instruments | |
| IL8 probe | Molecular | GenBank: NM_000584 |
| | Instruments | |
| CCL11/Eotaxin-1 probe | Molecular | GenBank: NM_002986.3 |
| | Instruments | |
| CXCR4 probe | Molecular | GenBank: NM_001348056.1 |
| | Instruments | |
| CD274/PDL1 probe | Molecular | GenBank: NM_014143.4 |
| | Instruments | |
| MK167 probe | Molecular | GenBank: NM_001145966.1 |
| | Instruments | |
| MALAT1 probe | Molecular | GenBank: NR_002819 |
| | Instruments | |
| B1-647 amplifiers | Molecular | N/A |
| | Instruments | |
| B3-555 amplifiers | Molecular | N/A |
| | Instruments | |
| B5-488 amplifiers | Molecular | N/A |
| | Instruments | |
| B2-488 amplifiers | Molecular | N/A software and algorithms |
| | Instruments | |

TABLE 7

| | | |
|---|---|---|
| Fiji image | Schindelin, et al., 2012^{76c} | |
| Statsannotations | Charlier, et al., 2022^{77c} | |
| Seaborn | Waskom, 2021^{78c} | |
| Matplotlib | Hunter, 1999^{79c} | |
| Networkx | Hagberg, et al., 2008^{80c} | Github: |
| | | Networkx |
| Pandas | The pandas development team, | Github: |
| | 2020^{81c} | pandas |
| Scanpy | Wolf, et al., 2018^{82c} | |
| Scikit-image | van der Walt, et al., 2014^{83c} | |
| Scikit-learn | Pedregosa, et al., 2011^{84c} | |
| SpaGNN | This study | |
| MERFISH fibroblasts | Chen, et al., 2015^{30c} | |
| dataset | | |
| U2-OS dataset | Mah, et al., 2022^{32c} | |

### Experimental Model and Subject Details

### Cell Line

Bone marrow-derived MSCs (BM-MSCs) and umbilical cord-derived MSCs (UC-MSCs) were purchased from RoosterBio, Inc. Cryopreserved human primary chondrocytes (HCH) (C-12710) and chondrocyte culture media (C-27101) were purchased from PromoCell, GmbH.

TABLE 8
Samples

| Cell | Source | Cat # | Age | Sex | Controls |
|---|---|---|---|---|---|
| MSC | Bone | #MSC-031 | 22 | M | CD90+ CD166+ |
| | Marrow | Lot#: 00238 | | | CD34− CD45−C/A/O |
| | | | | | differentiation |
| MSC | Umbilical | #C43001UC | N/A | F | CD90+ CD166+ |
| | Cord | Lot#: 210261 | | | CD34− CD45−C/A/O |
| | | | | | differentiation |
| Chondrocyte | Hip | C-12710 | 60 | M | Viability: 74% |
| | | Lot#: | | | PD Time: 66.3/PD |
| | | 445Z012.3 | | | |
| Fibroblast | Lung | N/A | N/A | N/A | N/A |
| IMR90³⁰ | | | | | |
| U2-OS | Bone | N/A | N/A | N/A | N/A |
| cells³² | | | | | |

### Cell Culture: Mesenchymal Stem Cells

The culture media consisted of 89% L-glutamine supplemented a-MEM media (Cat #12561-049), 10% heat-inactivated fetal bovine serum (HI-FBS), and 1% penicillin-streptomycin (Cat #P4333). The culture media was first mixed, then filtered before use. HBM and HUC were cultured in T-75 flasks with 15 mL of culture media. Cell passages were performed when cells reach 75% confluency using 5 mL of Trypsin LE cell detachment media (Cat #12605-010) per T-75 flask at 37° C. for 5 min. After confirming cell detachment, 5 mL of media was added to each flask to neutralize the trypsin. The cell suspension was then collected and centrifuged at 280 g for 6 min.

Cells were then resuspended in their respective culture media. Suspended cells were then seeded on collagen-coated glass coverslips. The cells were then cultured on coverslips for 24 h before fixation.

### Human Primary Chondrocytes

The media consisted of basal media and supplement mix. The basal media and supplement mix were mixed at a ratio specified by the instruction manual. Cell passages were performed when cells reach 75% confluency using Trypsin LE cell detachment media (Cat #12605-010) at room temperature and resuspension in respective culture media after centrifugation at 220 g for 3 min. Suspended cells were then seeded on collagen-coated coverslips. The seeded cells were cultured for 24 h before fixation.

### Cell Fixation

All cell lines were fixed and permeabilized using the same method. Cells were fixed with 4% paraformaldehyde fixation buffer for 10 min. (To prepare 10 mL of fixation buffer, mix 2.5 mL 16% paraformaldehyde (Cat #28908), 1 mL 10×DPBS (REF D1408), and 6.5 mL DNase, RNase free water (Cat #10977-015).) The samples were then washed with 1×DPBS (Cat #D8537) twice and stored in cold ethanol at −20° C. overnight for permeabilization.

### Multiplexed RNA Detection

The HCR staining follows the protocol provided by Molecular Instruments RNA-FISH on mammalian cells on slides protocol. After permeabilization, the samples were air-dried for 10 min and washed with 2×SSC buffer 3 times. The samples were then incubated in a 300 mL pre-warmed HCR probe hybridization buffer (LOT #BPH02023) at 37° C. for 30 min for prehybridization. 3 mL of HCR probes for desired targets were then added to 300 mL of pre-warmed probe hybridization buffer. The prehybridization buffer was aspirated, and a probe dilute was added to the samples. The samples were incubated at 37° C. for 12-16 h. The samples were then washed with 300 mL of pre-warmed probe wash buffer (LOT #BPW02922) for 5 min at 37° C. 4 times. Then, the samples were rinsed with 300 mL of 5×SSCT for 5 min at room temperature twice. The samples were then incubated with 300 mL of amplification buffer (LOT #BAM02422) at room temperature for 30 min for pre-amplification. 6 mL of each HCR amplifier hairpin was then snap-cooled to 95° C. for 90 s and cooled down to room temperature in a dark drawer for 30 min. The snap-cooled hairpins were then added to 300 mL of amplification buffer. The pre-amplification buffer was then aspirated, and diluted hairpins were added to the sample. The samples were then incubated for 1 h and 15 min. The amplification solution was then aspirated, and the samples were washed with 5×SSCT for 5 min at room temperature 5 times (see HCR protocol for cells on slide).

The samples were then mounted in an antifade mounting buffer containing Tris-HCL (20 mM), NaCl (50 mM), glucose (0.8%), saturated Trolox (Sigma, 53188-07-1), pyranose oxidase (Sigma: P4234), and catalase (Sigma, 9001 May 2, 1:1000 dilution).

To remove fluorescent mRNA signals, we used RNase-free DNase I (Sigma, 04716728001). After imaging, the samples were first washed with 2×SSC twice. We then diluted 50 mL of 10× concentration incubation buffer in 450 mL of RNase-free water to 1× concentration. The samples were then incubated with a 1× incubation buffer at room temperature for 5 min. Then 10 mL of DNase I, 50 mL of 10× incubation buffer, and 440 mL of RNase-free water were mixed. The samples were then incubated in the DNase I mixture for 4 h at room temperature. After incubation, the samples were then washed with 30% formamide in 2×SSC at room temperature for 5 min 3 times. Removal of signals was confirmed under a fluorescent imaging microscope. The samples were then ready to start the next cycle of RNA labeling.

### Cell Painting

After DNase I treatment after the last cycle of HCR-FISH, cell painting staining was conducted. Pre-conjugated antibodies were diluted in 1×PBS (Concanavalin A, 1:20; Phalloidin, 1:40; WGA, 1:400). Sufficient volume of the antibody solution was added to the cell. The sample was incubated for 30 min in the dark at room temperature. Then the sample is washed with 1×PBS for 5 min three times. The sample is then mounted in 1×PBS and imaged.

### Image Preprocessing

Images of cells in 2D culture all underwent the following preprocessing before further analysis. Each image contains multiple channels, and each channel is composed of images collected at various z-levels. The background of each gene-positive channel was first subtracted using a rolling ball method with a radius of 15 pixels. The maximum intensity projection of each channel was then generated. The projected image was the final image used for subcellular clustering and counting analysis. For the DAPI channel, the maximum intensity projection was generated from the raw image. Previous steps were conducted in a script implemented in FIJI, enabling automated high-throughput processing.

To apply clustering analysis, the images across multiple cycles were registered using the phase cross-correlation method in scikitimage.^{83 c} After detecting the translational and rotational shifts of the images, the reversed shifts were then applied to images, and overlapping regions were then cropped and saved.

RNA HCR-FISH signal was detected by finding the local maximum in each image above a manually set threshold. The detection algorithm was implemented using the local_peak_max( ) function in the scikit-image package. Parameters adjusted in local_peak_max( ) were the absolute threshold and minimum distance between local peaks. The parameters were adjusted by each gene. The custom-developed code detected all peaks in the image, then applied the segmentation mask to find RNA counts and locations per cell.

The image preprocessing was performed by running codes under the ‘image_processing’ folder. All image processing is performed under the skim environment provided by the skimEnv.yml file The 00_Registration.ipynb file performs multi-cycle image registration. The registered images were then saved. 01_dotDetectionThreCheck.ipynb allows the user to identify the appropriate threshold for detecting RNA. 02_2dDotDetection.ipynb performs dot detection. Thresholds identified by the user are provided to the code. 03_cytokineDetection.ipynb allows transcript detection of fewer genes with higher accuracy. All detected files are saved as.hdf5 files. To allow further processing and analysis of data, 00_dotFiles2pkl.ipynb under the ‘subcellular_analysis’ folder was used to convert the .hdf5 file to a.pkl file containing the position of the transcript of each gene.

Subcellular patch analysis Positions of all detected RNA transcripts were combined and clustered using the Leiden clustering algorithm with a resolution factor of 1. The Leiden clustering algorithm was implemented by the scanpy.tl.leiden( ) function in Scanpy package.^{82c} RNA transcripts of the same cluster were considered in the same patch. Copy numbers of genes in each patch were then counted. Pearson's correlation between genes was then computed among patches.

The patch-based analysis is conducted using 01_merFishPatchAnalyss.ipynb notebook running under scenv provided by scanpyEnv.yml file for MERFISH dataset, and 01_subcellularPatches.ipynb for seqFISH data. 02_neighborhoodCorrelationAnalyses.ipynb was used to perform statistical comparison and PCA on patch correlation for the seqFISH dataset.

Subcellular local neighborhood analysis Local neighborhoods were detected by applying the k-nearest-neighbor algorithm (k-NN) to the positions of all RNA in the same patch. We utilized sklearn.neighbors.NearestNeighbors package implemented in scikit-learn.^{84c} Each neighborhood consisted of the transcript and its 9 nearest neighbors. Copy numbers of genes in each local neighborhood were counted. Pearson's correlation between genes was computed among all local neighborhoods in the same patch. The gene neighborhood network is visualized using the networkx package.^{31c}

The analysis is performed using 02_neighborhoodNetworkAnalysis.ipynb notebook running under scenv provided by scanpy-Env.yml file. The notebook calls functions in utils.py script to perform the analysis. The network visualization is performed using 04_networkVisualization.ipynb notebook running under the network environment provided by networkEnv.yml. 03_subcellularNetworkInference.ipynb running under scenv provided by scanpyEnv.yml file was used to infer the gene neighborhood network in the seqFISH dataset. 05_connectivityToNetwork.ipynb running under the network environment provided by networkEnv.yml was used to visualize the network.

### Clustering and Visualization of Cell Spatial Transcriptomic Profile

In gene expression clustering analysis, the counts of genes were calculated by counting all detected RNA in the same cell mask. In patch correlation clustering, pairwise correlations among patches of each cell were collected. The correlations of different genes were used as features for clustering. In network variability clustering, the mean and standard deviation of proximity scores between each pair of genes of the same cell were used as features for clustering. After the data is compiled, the data were passed into AnnData objects. Visualization and clustering were then performed using the scanpy package.

The cluster assignment and true cell types were then compared using confusion matrices. Confusion matrices were generated using the confusion_matrixo function in sklearn.metrics module.

The clustering analysis is performed using 03_coclusteringAnalysis.ipynb notebook under the scenv environment provided by scanpyEnv.yml file for the MERFISH dataset, and 04_coclusteringAnalysis.ipynb for the seqFISH dataset. The clustering result comparison is performed using 05_clustering_eval.ipynb notebook under the scenv environment.

RNA-cellular structure colocalization The enrichment of cellular structure marker around each RNA was extracted by computing the total intensity of 3×3 pixels around the location of the RNA. Mean enrichment around all RNA in the same patch or local neighborhood was computed to indicate marker enrichment of the patch or local neighborhood. Correlations were computed similarly to all previous correlation analyses.

Enrichment of genes in measurement was calculated as follows. ConA and WGA images were manually thresholded and divided into marker-positive regions and marker-negative regions. The proportion of the marker-positive region in the cell is calculated by the following equation:

$${p}_{marker} = \frac{number of pixels in marker positive region}{number of pixels in cell mask}$$

Then, the proportion of COL1A1 transcript detected in the marker positive regions were calculated by the following equation:

$${p}_{RNA} = \frac{RNA count in marker positive region}{total RNA count}$$

The enrichment of RNA in the protein-positive region was calculated by the following equation:

$$enrichment = \frac{{p}_{RNA}}{{p}_{marker}}$$

The analysis is performed using 06_rnaProteinNetwork.ipynb notebook under scenv environment provided by scanpyEnv.yml file.

### Cytokine Gene Spatial Distribution

After a cell was segmented, a map of distances from all points inside the cell to the edge of the cell was generated. Distances to the edge of the cell were calculated for each RNA. Cells were then divided into contour patches of 5 mm width according to the distance to the edge of the cell. The same local neighborhood correlation analysis as before was conducted for each patch. The local neighborhood was reduced to the transcript and 4 nearest neighbors. Transcripts with a distance to the center transcript greater than 5 mm were excluded from the neighborhood. To visualize the distribution of cells with edge-localized cytokine RNA, Pearson's correlation between all local neighborhoods of the outermost patch was used as a feature to generate UMAP.

The analysis is performed using 07_circularNetworks.ipynb notebook under the scenv environment provided by the scanpyEn-v.yml file.

Subcellular Clustering from Multiplexed Images

After image registration, each pixel was clustered based on the intensity of the pixel of each image. Each image was first min-max normalized to a range of [0,1]. Pixels were then down-sampled to reduce data size. High-dimensional data for each pixel was clustered using the Leiden algorithm. All pixels were then assigned to a cluster based on the clustering result on the down-sampled data. Cells were then pseudo-colored based on cluster labels of each pixel to reveal the spatial distribution of each cluster.

The clustering is performed using codes provided under the HCR_subcellular_clustering folder. 01_extract_pixels.ipynb extracts all the pixels from cell masks into a matrix. 02_data_integration.ipynb combines the pixels and performs batch normalization using the scanaroma pipeline. 03_subsample_clustering.ipynb performs geometric sketching and clustering on the down-sampled data and generates the pseudo-colored cell images.

Spatial hypothesis testing among pairwise cells For each cell, the center of mass is defined from the cytosolic mask. The distance of each gene to the center of the mask is recorded as a distribution of spatial distances with respect to the center of mass. The Komologorov-Smirnov Hypothesis Test between pairwise cells of a single gene indicated if the pair of cells expressed the same gene in significantly different spatial patterns.

The spatial statistics were performed using code under the ‘spatial_statistics’ folder. All notebooks in the folder were run under the environment provided by the 1aug.2022.yml file. 01_combine_dataframes.ipynb combines single-cell data from HUC and HBM into a single data frame and exports it as a .pkl file. 02_compute_cell_centers.ipynb computes the center of mass of every cell according to the cell mask. 03_compute_dist_centers.ipynb computes the distance of each RNA to the center of the cell. 04_compute_ksTest_pairwiseCells.ipynb performs the cell-pairwise Komolgorov-Smirnov test. 05_spatial_histograms.ipynb visualizes the density of detected RNA at varying distances from the center of the cell.

### Quantification and Statistical Analysis

Pearson correlations method was used to correct pairwise marker correlation at the single-cell levels. Mann-Whitney-Wilcoxon test was used for the comparison of patch correlation between cell types. Paired t-test was used for the comparison of the enrichment of COL1A1 in subcellular regions. The Komologorov-Smirnov Hypothesis Test between pairwise cells of a single gene. The level of significance was set at not significant (ns): 0.05<p, *: 0.01<p % 0.05, **: 0.001<p % 0.01 ***: 0.0001<p % 0.001, ****: p<=0.0001. All data visualization and statistical analyses were carried out using in-house Python scripts.

### Example 5: Spatial Subcellular Organelle Networks in Single Cells

### Introduction

Organelles play important roles in human health and disease, such as maintaining homeostasis, regulating growth and aging, and generating energy. Organelle diversity in cells not only exists between cell types but also between individual cells. Therefore, studying the distribution of organelles at the single-cell level is important to understand cellular function. Mesenchymal stem cells are multipotent cells that have been explored as a therapeutic method for treating a variety of diseases. Studying how organelles are structured in these cells can answer questions about their function and potential. Herein, rapid multiplexed immunofluorescence was performed to understand the spatial organization of 10 organelle proteins and the interactions between them in the bone marrow (BM) and umbilical cord (UC) mesenchymal stem cells (MSCs). Spatial correlations, colocalization, clustering, statistical tests, texture, and morphological analyses were conducted at the single cell level, shedding light onto the interrelations between the organelles and comparisons of the two MSC subtypes. Such analytics toolsets indicated that UC MSC exhibited higher organelle expression and spatially spread distribution of mitochondria accompanied by several others organelles compared to BM MSCs. In the future, this data-driven single-cell approach provided by rapid subcellular proteomic imaging can enable personalized stem cell therapeutics.

Cells perform different functions like providing structure and support, facilitating growth, producing energy, etc. to support and sustain life. These activities are handled by various subcellular structures termed organelles such as the nucleus, mitochondria, endoplasmic reticulum, and Golgi apparatus^{1e}. Organelles cooperate to form a network of interactions that enable different cellular activities^{2e}. Cell-to-cell variability is observed not only between cell types but also between cells of the same type, resulting in molecularly and functionally distinct cells^{3e}. Such differences may contribute to the health and function of the entire organism. Multiple factors, such as microenvironment variability, differences in the cellular stages, genetics or epigenetics, or fluctuations in gene expression levels, can cause this heterogeneity. Single-cell analysis approaches are thus useful in investigating aspects of cellular mechanisms that are not revealed in bulk-level studies^{4 e,5 e}.

The use of mesenchymal stem cells (MSCs) is a therapeutic method for treating a variety of diseases, including myocardial infarction and diabetes, as they can repair damaged cells by differentiating into replacement cells and modulating immune responses^{6 e-11 e.} Therefore, analyzing the spatial organelle networks within MSCs can lead to a better understanding of cell functions to design appropriate treatment methods^{12 e-16 e}. Although there have been recent studies focusing on spatial organelle analysis, these studies have used spatial data of each organelle from different cells, which reduces the accuracy of the resulting spatial information.^{17 e}As such, a highly multiplexed protein imaging and analysis method obtains spatial information on organelles within the same cell, which can then be used to compare and understand differences in spatial organization between different cell types. In addition to intra-population variability, the most commonly used^{18 e}, readily available stem cells sourced from bone marrow (BM) and postnatal umbilical cord (UC) introduce additional variables for the study. These cells display different molecular profiles, differentiation potential, and therapeutic efficacy^{19 e}, and there is little consensus on how much, if any, the effect that MSC source has on outcome^{20 e}.

Highly multiplexed imaging technologies enable the detection of multiple proteins in a single cell. Imaging mass cytometry (IMC)^{21 e} and multiplexed ion beam imaging (MIBI)^{22 e} can image up to 36 proteins using isotope-labeled antibody libraries and specialized equipment. However, these measurements are limited by their low resolution of about 0.5 to 1-μm. High-dimensional fluorescence imaging methods can map up to 50 proteins and include DNA-barcoded imaging (CODEX)^{23 e}, multiplexed fluorescence microscopy (MxIF)^{24 e}, and cyclic and sequential IF techniques^{25e-28e}. While conventional IF is time-consuming, rapid multiplexed immunofluorescence provides multiplicity through quick multiple rounds of immunostaining and fluorophore inactivation. Rapid multiplexed immunofluorescence enables high-throughput in situ proteomic analysis using conventional microscopes.

Herein, we aim to establish a rapid protein analysis pipeline for deciphering spatial organelle networks within a single cell. To achieve this, proteins in key organelles in mesenchymal stem cells (MSCs) such as the nucleus, mitochondria, Golgi, and endoplasmic reticulum (ER) have been targeted using antibodies. Multiplexed protein imaging was performed to examine the spatial organization of organelles and the interactions between organelles in MSCs. The analysis of organelle interactions on a single-cell level can eventually aid the stem-cell field in better understanding cell functions and exploring treatment methods using MSCs. Differences in organelle localization, interactions, and associated energy highlight the MSC heterogeneity from donor sources that will better inform therapeutic cell designs.

### Results

### Multiplex Protein Labeling Reveals Spatially Resolved Subcellular Organelle Maps in Tissue-Specific MSCs

To measure single-cell organelle distributions, we profiled subcellular localization of organelle proteins in the bone marrow and umbilical cord MSCs using rapid multiplexed immunofluorescence (FIG. 60A, FIG. 67A). Multiple markers colocalized in different cellular regions, including mitochondria (TOM20 and HSP60), the Golgi (Sortilin, GOLPH4, and WGA), endoplasmic reticulum (ATF6 and Concanavalin A), nucleolus (Nucleolin), microtubules (β-Tubulin), and actin filaments (Phalloidin) (FIG. 60B, FIG. 67B, 68A-68B). These colocalizations were quantified using scatter plots, correlation coefficients, clustering, and texture analysis to understand the spatial organization of these markers and the interactions between them.

From the 10-plex data, up to 25 cells (BM and UC MSCs combined) were selected for each marker. The scatter plots of intensity were calculated by random sampling of 50,000 pixel intensity values from all the cells for each marker pair targeting the same organelle. The scatter plots showed similar distribution for BM and UC MSCs for mitochondria targeting markers, implying similar interactions between the mitochondria markers in the two cell types (FIG. 61A-61B). However, the point distribution difference in scatter plots for Golgi and ER markers between BM and UC MSCs can be attributed to differences in interactions and the subcellular targets of the markers in the two cell types.

### Uc MSC Organelles Exhibit Higher Protein Expression Over a Larger Spatial Area

To quantify organelle interrelation patterns, pairwise Pearson's correlation and pixel overlap colocalization values were obtained for each unique marker pair and plotted as boxplots with the Mann-Whitney test to observe significance (p<0.05) (FIG. 62A-62B, Tables 11 and 12).

TABLE 11
Minimum, mean, median, and maximum of Pearson's correlation for all marker pairs for BM and
UC MSCs, with the p-value obtained from the Mann-Whitney test (corresponding to FIG. 62A).

| | BM MSC | | | | UC MSC | | | | BM vs UC | |
|---|---|---|---|---|---|---|---|---|---|---|
| Markers | Min | Mean | Median | Max | Min | Mean | Median | Max | P_val | U_stat |
| DAPI_GOLPH4 | −0.39019 | 0.062123 | 0.074778 | 0.317464 | −0.09886 | 0.105488 | 0.0842 | 0.386037 | 0.576613 | 120 |
| DAPI_HSP60 | −0.42051 | 0.002756 | −0.01028 | 0.376206 | −0.23394 | 0.022768 | 0.008788 | 0.280216 | 0.806636 | 73 |
| DAPI_Nucleolin | 0.543317 | 0.875238 | 0.902931 | 0.975774 | 0.367504 | 0.673049 | 0.699571 | 0.883398 | 3.85E−05 | 322 |
| DAPI_Phalloidin | 0.216938 | 0.382776 | 0.348025 | 0.619993 | 0.488149 | 0.612673 | 0.625872 | 0.674625 | 0.000594 | 6 |
| DAPI_Sortilin | 0.325445 | 0.621453 | 0.654387 | 0.880571 | 0.17685 | 0.426505 | 0.394083 | 0.642554 | 0.003707 | 161 |
| DAPI_TOM20 | −0.35485 | 0.11469 | 0.074217 | 0.697917 | −0.16003 | 0.098451 | 0.056604 | 0.390662 | 0.797229 | 127 |
| DAPI_WGA | 0.347376 | 0.516248 | 0.484481 | 0.720834 | 0.529252 | 0.661049 | 0.687087 | 0.740772 | 0.029309 | 9 |
| Nucleolin_{—} | 0.194735 | 0.314293 | 0.29751 | 0.590012 | 0.343967 | 0.466714 | 0.449239 | 0.686738 | 0.006085 | 12 |
| Phalloidin | | | | | | | | | | |
| Nucleolin_{—} | 0.267203 | 0.629767 | 0.667747 | 0.890442 | 0.00239 | 0.312953 | 0.299362 | 0.608249 | 0.000317 | 188 |
| Sortilin | | | | | | | | | | |
| Nucleolin_TOM20 | −0.34576 | 0.088968 | 0.038012 | 0.680387 | −0.1461 | 0.121276 | 0.096396 | 0.448528 | 0.755208 | 104 |
| Nucleolin_WGA | 0.218807 | 0.436256 | 0.407739 | 0.633647 | 0.423519 | 0.497395 | 0.481296 | 0.603469 | 0.316792 | 22 |
| BetaTubulin_{—} | 0.496492 | 0.691746 | 0.7405 | 0.789489 | 0.413759 | 0.573477 | 0.498921 | 0.791367 | 0.412698 | 14 |
| ConcanavalinA | | | | | | | | | | |
| BetaTubulin_{—} | −0.31142 | 0.139395 | 0.108773 | 0.625378 | −0.06784 | 0.228074 | 0.261448 | 0.473662 | 0.203879 | 63 |
| DAPI | | | | | | | | | | |
| BetaTubulin_{—} | 0.283838 | 0.574685 | 0.568009 | 0.806661 | 0.137294 | 0.393023 | 0.328376 | 0.714282 | 0.021868 | 124 |
| GOLPH4 | | | | | | | | | | |
| BetaTubulin_{—} | 0.327023 | 0.67031 | 0.717532 | 0.865904 | 0.16363 | 0.409397 | 0.410274 | 0.60755 | 0.007184 | 73 |
| HSP60 | | | | | | | | | | |
| BetaTubulin_{—} | −0.3023 | 0.112284 | 0.097697 | 0.632351 | −0.0552 | 0.147151 | 0.156322 | 0.37754 | 0.633368 | 75 |
| Nucleolin | | | | | | | | | | |
| BetaTubulin_{—} | 0.244745 | 0.626865 | 0.672226 | 0.867982 | 0.494649 | 0.582824 | 0.528044 | 0.780561 | 0.720915 | 32 |
| Phalloidin | | | | | | | | | | |
| BetaTubulin_{—} | 0.307572 | 0.597394 | 0.597909 | 0.907544 | 0.124691 | 0.468492 | 0.458365 | 0.75202 | 0.069825 | 83 |
| Sortilin | | | | | | | | | | |
| BetaTubulin_{—} | 0.576419 | 0.772853 | 0.802629 | 0.92124 | 0.113598 | 0.409049 | 0.38787 | 0.678083 | 0.000138 | 124 |
| TOM20 | | | | | | | | | | |
| BetaTubulin_{—} | 0.428599 | 0.648152 | 0.696771 | 0.853219 | 0.365344 | 0.509655 | 0.521973 | 0.641648 | 0.197059 | 32 |
| WGA | | | | | | | | | | |
| Phalloidin_{—} | 0.338837 | 0.538326 | 0.546553 | 0.740659 | 0.547864 | 0.712553 | 0.722694 | 0.860582 | 0.031219 | 10 |
| Sortilin | | | | | | | | | | |
| Phalloidin_{—} | 0.100105 | 0.536913 | 0.592766 | 0.793341 | 0.173488 | 0.528836 | 0.564509 | 0.763669 | 0.963585 | 35 |
| TOM20 | | | | | | | | | | |
| Phalloidin_WGA | 0.511922 | 0.820922 | 0.855109 | 0.963523 | 0.812238 | 0.88441 | 0.919115 | 0.921877 | 0.357895 | 16 |
| ATF6_{—} | 0.230635 | 0.638931 | 0.651492 | 0.831835 | 0.111629 | 0.527598 | 0.532653 | 0.833342 | 0.074116 | 101 |
| BetaTubulin | | | | | | | | | | |
| ATF6_{—} | 0.537429 | 0.60885 | 0.606558 | 0.684853 | 0.400188 | 0.691459 | 0.721718 | 0.858331 | 0.460606 | 11 |
| ConcanavalinA | | | | | | | | | | |
| ATF6_DAPI | −0.37689 | 0.07511 | 0.079387 | 0.479719 | 0.23692 | 0.464934 | 0.469409 | 0.695617 | 9.47E−06 | 12 |
| ATF6_GOLPH4 | 0.438384 | 0.654637 | 0.655356 | 0.871873 | 0.207702 | 0.448072 | 0.41293 | 0.74775 | 0.000512 | 220 |
| ATF6_HSP60 | 0.288369 | 0.595477 | 0.621389 | 0.770715 | 0.343904 | 0.549817 | 0.575758 | 0.741718 | 0.402504 | 87 |
| ATF6_Nucleolin | −0.37893 | 0.053693 | 0.004319 | 0.46994 | 0.078385 | 0.35306 | 0.327777 | 0.641588 | 0.000504 | 37 |
| ATF6_Phalloidin | 0.125327 | 0.481911 | 0.549689 | 0.712088 | 0.684505 | 0.792287 | 0.79503 | 0.891105 | 0.000646 | 2 |
| ATF6_Sortilin | 0.11899 | 0.421913 | 0.408688 | 0.654826 | 0.633488 | 0.766087 | 0.757086 | 0.878789 | 2.62E−05 | 1 |
| ATF6_TOM20 | 0.559568 | 0.652566 | 0.634778 | 0.822792 | 0.311763 | 0.584473 | 0.565917 | 0.810053 | 0.117297 | 132 |
| ATF6_WGA | 0.252876 | 0.473902 | 0.383127 | 0.730323 | 0.699616 | 0.753623 | 0.725782 | 0.863312 | 0.010256 | 3 |
| ConcanavalinA_{—} | −0.05448 | 0.303461 | 0.362518 | 0.543288 | 0.000862 | 0.222493 | 0.181937 | 0.550333 | 0.604196 | 22 |
| DAPI | | | | | | | | | | |
| ConcanavalinA_{—} | 0.464922 | 0.57005 | 0.532831 | 0.749616 | 0.544043 | 0.689883 | 0.696733 | 0.816394 | 0.198601 | 9 |
| GOLPH4 | | | | | | | | | | |
| ConcanavalinA_{—} | 0.485728 | 0.610834 | 0.568922 | 0.819765 | 0.588811 | 0.688966 | 0.684291 | 0.785184 | 0.367677 | 10 |
| HSP60 | | | | | | | | | | |
| ConcanavalinA_{—} | −0.03387 | 0.299571 | 0.335504 | 0.561149 | 0.011404 | 0.216478 | 0.205355 | 0.577092 | 0.682828 | 19 |
| Nucleolin | | | | | | | | | | |
| ConcanavalinA_{—} | 0.309975 | 0.533999 | 0.533309 | 0.759403 | 0.47781 | 0.732062 | 0.744397 | 0.911059 | 0.171429 | 5 |
| Phalloidin | | | | | | | | | | |
| ConcanavalinA_{—} | 0.610423 | 0.641146 | 0.643976 | 0.666209 | 0.356439 | 0.604905 | 0.62096 | 0.864761 | 1 | 16 |
| Sortilin | | | | | | | | | | |
| ConcanavalinA_{—} | 0.569595 | 0.64355 | 0.611876 | 0.780851 | 0.560793 | 0.719975 | 0.720785 | 0.823378 | 0.282828 | 9 |
| TOM20 | | | | | | | | | | |
| ConcanavalinA_{—} | 0.433955 | 0.621926 | 0.607835 | 0.83808 | 0.546859 | 0.660384 | 0.609458 | 0.824837 | 1 | 6 |
| WGA | | | | | | | | | | |
| GOLPH4_HSP60 | 0.385765 | 0.601535 | 0.651802 | 0.790159 | 0.341387 | 0.558922 | 0.574245 | 0.73119 | 0.370844 | 88 |
| GOLPH4_{—} | −0.3879 | 0.044443 | 0.036952 | 0.267336 | −0.10264 | 0.097832 | 0.054791 | 0.413396 | 0.55227 | 119 |
| Nucleolin | | | | | | | | | | |
| GOLPH4_{—} | 0.08783 | 0.479072 | 0.525426 | 0.731147 | 0.224822 | 0.513823 | 0.533916 | 0.759271 | 0.820082 | 33 |
| Phalloidin | | | | | | | | | | |
| GOLPH4_{—} | 0.043945 | 0.366134 | 0.370333 | 0.68059 | 0.199859 | 0.388441 | 0.352215 | 0.682184 | 0.979483 | 83 |
| Sortilin | | | | | | | | | | |
| GOLPH4_TOM20 | 0.306794 | 0.599249 | 0.593468 | 0.803896 | 0.390917 | 0.570744 | 0.586689 | 0.722106 | 0.555735 | 119 |
| GOLPH4_WGA | 0.152016 | 0.4553 | 0.49672 | 0.727791 | 0.330933 | 0.434106 | 0.403628 | 0.598236 | 0.870588 | 28 |
| HSP60_Nucleolin | −0.41367 | −0.02945 | −0.07352 | 0.334086 | −0.18366 | 0.048875 | −0.00455 | 0.332282 | 0.417302 | 57 |
| HSP60_{—} | 0.265471 | 0.523268 | 0.450504 | 0.773128 | 0.286359 | 0.500554 | 0.541214 | 0.683799 | 0.949717 | 25 |
| Phalloidin | | | | | | | | | | |
| HSP60_Sortilin | 0.152477 | 0.52596 | 0.540497 | 0.753786 | 0.237453 | 0.494478 | 0.487629 | 0.694411 | 0.599361 | 69 |
| HSP60_TOM20 | 0.575209 | 0.705255 | 0.718745 | 0.827389 | 0.553234 | 0.681668 | 0.680737 | 0.797218 | 0.602068 | 81 |
| HSP60_WGA | 0.313627 | 0.527848 | 0.486146 | 0.795322 | 0.262485 | 0.404339 | 0.323262 | 0.627269 | 0.278788 | 18 |
| Sortilin_TOM20 | 0.223946 | 0.542002 | 0.525151 | 0.889835 | 0.223506 | 0.492665 | 0.467866 | 0.728453 | 0.55876 | 76 |
| Sortilin_WGA | 0.514945 | 0.656143 | 0.663811 | 0.80966 | 0.571951 | 0.666756 | 0.636943 | 0.791373 | 1 | 16 |
| TOM20_WGA | 0.30527 | 0.557022 | 0.545052 | 0.809731 | 0.298807 | 0.458673 | 0.405488 | 0.724908 | 0.379121 | 32 |

TABLE 12
Minimum, mean, median, and maximum colocalization for all marker pairs for BM and UC MSCs,
with the p-value obtained from the Mann-Whitney test (corresponding to FIG. 62B).

| | BM MSC | | | | UC MSC | | | | BM vs UC | |
|---|---|---|---|---|---|---|---|---|---|---|
| Markers | Min | Mean | Median | Max | Min | Mean | Median | Max | P_val | U_stat |
| DAPI_GOLPH4 | 0.029933 | 0.140341 | 0.110874 | 0.612356 | 0.038534 | 0.205634 | 0.129603 | 0.546821 | 0.33979 | 109 |
| DAPI_HSP60 | 0.032031 | 0.107235 | 0.104096 | 0.206923 | 0.025207 | 0.202978 | 0.128565 | 0.508919 | 0.567915 | 67 |
| DAPI_Nucleolin | 0.095349 | 0.217035 | 0.172187 | 0.701208 | 0.089205 | 0.238682 | 0.197883 | 0.53368 | 0.501915 | 157 |
| DAPI_Phalloidin | 0.068819 | 0.205241 | 0.150994 | 0.686085 | 0.195755 | 0.349284 | 0.328462 | 0.522633 | 0.012561 | 16 |
| DAPI_Sortilin | 0.080312 | 0.189254 | 0.15417 | 0.688467 | 0.07763 | 0.228077 | 0.179385 | 0.494266 | 0.461095 | 81 |
| DAPI_TOM20 | 0.031419 | 0.161131 | 0.12788 | 0.680571 | 0.047601 | 0.218104 | 0.148579 | 0.544187 | 0.593595 | 106 |
| DAPI_WGA | 0.082669 | 0.221316 | 0.170709 | 0.686617 | 0.201016 | 0.301321 | 0.277963 | 0.448344 | 0.049948 | 11 |
| Nucleolin_{—} | 0.08118 | 0.237942 | 0.184388 | 0.721285 | 0.183496 | 0.339912 | 0.324401 | 0.51136 | 0.177503 | 29 |
| Phalloidin | | | | | | | | | | |
| Nucleolin_{—} | 0.100414 | 0.22385 | 0.190346 | 0.720713 | 0.074958 | 0.224461 | 0.17072 | 0.480421 | 0.526843 | 120 |
| Sortilin | | | | | | | | | | |
| Nucleolin_{—} | 0.050427 | 0.186549 | 0.159006 | 0.701684 | 0.067648 | 0.220288 | 0.143254 | 0.538955 | 0.983417 | 111 |
| TOM20 | | | | | | | | | | |
| Nucleolin_WGA | 0.102003 | 0.255952 | 0.19887 | 0.721176 | 0.186831 | 0.291371 | 0.257728 | 0.463197 | 0.361571 | 23 |
| BetaTubulin_{—} | 0.16241 | 0.18445 | 0.171137 | 0.233117 | 0.095782 | 0.189548 | 0.108706 | 0.522169 | 0.190476 | 16 |
| ConcanavalinA | | | | | | | | | | |
| BetaTubulin_{—} | 0.038165 | 0.180007 | 0.122648 | 0.68698 | 0.041233 | 0.153385 | 0.108799 | 0.49191 | 0.548953 | 103 |
| DAPI | | | | | | | | | | |
| BetaTubulin_{—} | 0.085206 | 0.198218 | 0.176466 | 0.6367 | 0.053937 | 0.149621 | 0.09658 | 0.509404 | 0.042445 | 119 |
| GOLPH4 | | | | | | | | | | |
| BetaTubulin_{—} | 0.120676 | 0.181837 | 0.167848 | 0.294287 | 0.05642 | 0.148507 | 0.10222 | 0.492257 | 0.012979 | 71 |
| HSP60 | | | | | | | | | | |
| BetaTubulin_{—} | 0.063182 | 0.223111 | 0.169347 | 0.72395 | 0.038043 | 0.149407 | 0.103773 | 0.487062 | 0.125672 | 116 |
| Nucleolin | | | | | | | | | | |
| BetaTubulin_{—} | 0.052337 | 0.236681 | 0.156967 | 0.728057 | 0.107428 | 0.215013 | 0.123001 | 0.506623 | 0.277124 | 39 |
| Phalloidin | | | | | | | | | | |
| BetaTubulin_{—} | 0.118466 | 0.241298 | 0.204575 | 0.732019 | 0.050634 | 0.152346 | 0.114866 | 0.527398 | 0.003803 | 97 |
| Sortilin | | | | | | | | | | |
| BetaTubulin_{—} | 0.136339 | 0.232811 | 0.193219 | 0.695106 | 0.054592 | 0.154842 | 0.09975 | 0.473625 | 0.021489 | 100 |
| TOM20 | | | | | | | | | | |
| BetaTubulin_{—} | 0.094124 | 0.265419 | 0.191987 | 0.728109 | 0.101372 | 0.180874 | 0.119545 | 0.321705 | 0.432353 | 28 |
| WGA | | | | | | | | | | |
| Phalloidin_{—} | 0.077295 | 0.224957 | 0.171793 | 0.731656 | 0.173095 | 0.33182 | 0.325545 | 0.496152 | 0.093407 | 14 |
| Sortilin | | | | | | | | | | |
| Phalloidin_{—} | 0.059375 | 0.200287 | 0.155203 | 0.69615 | 0.101767 | 0.316583 | 0.325452 | 0.520102 | 0.493644 | 28 |
| TOM20 | | | | | | | | | | |
| Phalloidin_WGA | 0.127441 | 0.294336 | 0.231962 | 0.782358 | 0.231311 | 0.328896 | 0.235917 | 0.519462 | 0.545614 | 19 |
| ATF6_{—} | 0.07713 | 0.206393 | 0.177691 | 0.663276 | 0.049964 | 0.17242 | 0.117646 | 0.535966 | 0.095159 | 99 |
| BetaTubulin | | | | | | | | | | |
| ATF6_{—} | 0.140418 | 0.16471 | 0.15982 | 0.198783 | 0.136196 | 0.299043 | 0.213225 | 0.551848 | 0.460606 | 11 |
| ConcanavalinA | | | | | | | | | | |
| ATF6_DAPI | 0.039889 | 0.145055 | 0.111989 | 0.616466 | 0.078378 | 0.243452 | 0.195032 | 0.524616 | 0.008761 | 62 |
| ATF6_GOLPH4 | 0.108655 | 0.196016 | 0.170058 | 0.637351 | 0.072018 | 0.213853 | 0.135709 | 0.529135 | 0.406098 | 150 |
| ATF6_HSP60 | 0.104016 | 0.164404 | 0.157385 | 0.29108 | 0.06696 | 0.235376 | 0.151047 | 0.510431 | 0.885234 | 69 |
| ATF6_Nucleolin | 0.046195 | 0.170277 | 0.146255 | 0.637667 | 0.08512 | 0.235627 | 0.192502 | 0.515698 | 0.077286 | 84 |
| ATF6_Phalloidin | 0.050772 | 0.186974 | 0.142172 | 0.648206 | 0.191566 | 0.349565 | 0.341216 | 0.535523 | 0.027311 | 11 |
| ATF6_Sortilin | 0.084176 | 0.161247 | 0.151105 | 0.290217 | 0.103548 | 0.257465 | 0.202064 | 0.510202 | 0.069294 | 47 |
| ATF6_TOM20 | 0.110376 | 0.173772 | 0.169589 | 0.293165 | 0.091216 | 0.250017 | 0.181488 | 0.514897 | 0.890085 | 94 |
| ATF6_WGA | 0.084178 | 0.208003 | 0.157136 | 0.64311 | 0.197514 | 0.312 | 0.272604 | 0.50528 | 0.077656 | 8 |
| ConcanavalinA_{—} | 0.068039 | 0.134797 | 0.14854 | 0.174069 | 0.010144 | 0.250507 | 0.15323 | 0.52669 | 0.71049 | 15 |
| DAPI | | | | | | | | | | |
| ConcanavalinA_{—} | 0.116307 | 0.158268 | 0.166661 | 0.183443 | 0.098642 | 0.28318 | 0.190439 | 0.537021 | 0.26014 | 10 |
| GOLPH4 | | | | | | | | | | |
| ConcanavalinA_{—} | 0.128374 | 0.179046 | 0.182393 | 0.223023 | 0.112997 | 0.285997 | 0.195008 | 0.540085 | 0.682828 | 13 |
| HSP60 | | | | | | | | | | |
| ConcanavalinA_{—} | 0.086394 | 0.151197 | 0.166215 | 0.185966 | 0.126744 | 0.286909 | 0.195202 | 0.523105 | 0.460606 | 11 |
| Nucleolin | | | | | | | | | | |
| ConcanavalinA_{—} | 0.056748 | 0.138347 | 0.155869 | 0.184902 | 0.150698 | 0.347229 | 0.33882 | 0.55311 | 0.171429 | 5 |
| Phalloidin | | | | | | | | | | |
| ConcanavalinA_{—} | 0.138492 | 0.195578 | 0.199164 | 0.245491 | 0.130307 | 0.277163 | 0.178028 | 0.496645 | 1 | 16 |
| Sortilin | | | | | | | | | | |
| ConcanavalinA_{—} | 0.137467 | 0.175938 | 0.182917 | 0.200449 | 0.123607 | 0.300654 | 0.205623 | 0.533969 | 0.569697 | 12 |
| TOM20 | | | | | | | | | | |
| ConcanavalinA_{—} | 0.092071 | 0.174946 | 0.20021 | 0.207293 | 0.145763 | 0.286049 | 0.198647 | 0.513738 | 1 | 6 |
| WGA | | | | | | | | | | |
| GOLPH4_HSP60 | 0.099651 | 0.159818 | 0.163529 | 0.23327 | 0.068394 | 0.241964 | 0.129313 | 0.522892 | 0.750832 | 78 |
| GOLPH4_{—} | 0.038254 | 0.166481 | 0.140757 | 0.627265 | 0.057044 | 0.207773 | 0.137589 | 0.536126 | 0.842951 | 130 |
| Nucleolin | | | | | | | | | | |
| GOLPH4_{—} | 0.049873 | 0.183734 | 0.145713 | 0.638622 | 0.111599 | 0.31445 | 0.307422 | 0.520862 | 0.437083 | 27 |
| Phalloidin | | | | | | | | | | |
| GOLPH4_{—} | 0.073334 | 0.153295 | 0.142385 | 0.265553 | 0.070501 | 0.208047 | 0.11778 | 0.496356 | 0.625106 | 94 |
| Sortilin | | | | | | | | | | |
| GOLPH4_TOM20 | 0.099973 | 0.165855 | 0.170396 | 0.23814 | 0.073102 | 0.24121 | 0.171869 | 0.555621 | 0.878593 | 101 |
| GOLPH4_WGA | 0.068093 | 0.204101 | 0.163804 | 0.634983 | 0.107153 | 0.268693 | 0.245805 | 0.476008 | 0.548739 | 20 |
| HSP60_Nucleolin | 0.045298 | 0.130364 | 0.128896 | 0.273187 | 0.049015 | 0.198623 | 0.106824 | 0.506065 | 0.862015 | 75 |
| HSP60_{—} | 0.061138 | 0.152704 | 0.15024 | 0.259035 | 0.102167 | 0.3105 | 0.298625 | 0.524778 | 0.344988 | 16 |
| Phalloidin | | | | | | | | | | |
| HSP60_Sortilin | 0.104884 | 0.179364 | 0.159302 | 0.304518 | 0.063344 | 0.234236 | 0.140627 | 0.505232 | 0.511406 | 71 |
| HSP60_TOM20 | 0.116491 | 0.179458 | 0.174055 | 0.292918 | 0.081971 | 0.242916 | 0.165204 | 0.517226 | 0.862015 | 75 |
| HSP60_WGA | 0.083358 | 0.169871 | 0.165588 | 0.276312 | 0.098521 | 0.240743 | 0.170423 | 0.453287 | 0.921212 | 11 |
| Sortilin_TOM20 | 0.104367 | 0.224192 | 0.186117 | 0.694328 | 0.085092 | 0.235964 | 0.152351 | 0.491404 | 0.600878 | 75 |
| Sortilin_WGA | 0.116207 | 0.255394 | 0.212912 | 0.734139 | 0.175377 | 0.278567 | 0.188996 | 0.471329 | 0.884615 | 18 |
| TOM20_WGA | 0.093864 | 0.220697 | 0.176838 | 0.696823 | 0.09784 | 0.287557 | 0.27248 | 0.507427 | 0.446154 | 17 |

Some marker pairs consistently showed significance in both the plots (ATF6_DAPI, β-Tubulin_GOLPH4, and 03-Tubulin_TOM20) indicating a difference in colocalization between BM and UC MSCs. In general, marker pairs containing ATF6, β-Tubulin, DAPI, GOLPH4, and HSP60 expressed significant differences in distribution between the two cell types. Overall, UC MSCs showed more colocalization between ER (ATF6, Concanavalin A) and mitochondria (HSP60 TOM20) pairs, suggesting more crosstalk between their organelles. Other than the pairwise plots, the area and average intensity values of each marker were obtained per cell and compared between BM and UC MSCs (FIG. 62C-62D, Tables 13-14).

TABLE 13
Minimum, mean, median, and maximum area for all markers for BM and UC MSCs, with
the p-value obtained from the Mann-Whitney test (corresponding to FIG. 62C).

| | BM MSC | | | | UC MSC | | | | BM vs UC | |
|---|---|---|---|---|---|---|---|---|---|---|
| Marker | Min | Mean | Median | Max | Min | Mean | Median | Max | P_val | U_stat |
| ATF6 | 95613 | 251152.3 | 182058 | 540950 | 74862 | 354998.1 | 297711 | 919540 | 0.071296 | 90 |
| BetaTubulin | 95613 | 246384.8 | 187415 | 540950 | 192623 | 318666.6 | 272743 | 603029 | 0.08532 | 57 |
| ConcanavalinA | 125253 | 283039.8 | 258858.5 | 489189 | 62483 | 252557.1 | 248294 | 470513 | 0.825175 | 20 |
| DAPI | 95613 | 272233.7 | 248057 | 540950 | 62483 | 360352 | 297891 | 919540 | 0.114574 | 149 |
| GOLPH4 | 95613 | 243617.6 | 182058 | 540950 | 62483 | 321317.4 | 272743 | 919540 | 0.170779 | 111 |
| HSP60 | 95613 | 249348.2 | 184736.5 | 540950 | 62483 | 297553.1 | 293154.5 | 603029 | 0.425315 | 68 |
| Nucleolin | 95613 | 267434.1 | 236405 | 540950 | 74862 | 360970.9 | 297891 | 919540 | 0.096196 | 138 |
| Phalloidin | 95613 | 278803.2 | 274030 | 489189 | 117910 | 294907.7 | 293154.5 | 470513 | 0.922539 | 52 |
| Sortilin | 95613 | 258700.6 | 215057.5 | 540950 | 117910 | 362750.6 | 297711 | 919540 | 0.101707 | 67 |
| TOM20 | 95613 | 235855.4 | 187415 | 489189 | 62483 | 309172.9 | 288598 | 603029 | 0.174001 | 91 |
| WGA | 95613 | 268758.1 | 248110 | 489189 | 192623 | 319911.5 | 308255 | 470513 | 0.484484 | 27 |

TABLE 14
Minimum, mean, median, and maximum intensity for all markers for BM and UC MSCs,
with the p-value obtained from the Mann-Whitney test (corresponding to FIG. 62D).

| | BM MSC | | | | UC MSC | | | | BM vs UC | |
|---|---|---|---|---|---|---|---|---|---|---|
| Marker | Min | Mean | Median | Max | Min | Mean | Median | Max | P_val | U_stat |
| ATF6 | 33.17142 | 98.03184 | 100.1086 | 145.7242 | 40.8177 | 91.76635 | 81.2978 | 185.1369 | 0.071296 | 90 |
| BetaTubulin | 31.58946 | 91.9572 | 94.03891 | 145.7242 | 40.8177 | 104.4021 | 93.14664 | 185.1369 | 0.08532 | 57 |
| ConcanavalinA | 86.07068 | 98.85573 | 95.53423 | 118.2838 | 40.8177 | 78.65693 | 81.2978 | 127.5041 | 0.825175 | 20 |
| DAPI | 31.58946 | 92.09201 | 94.03891 | 145.7242 | 40.8177 | 94.06361 | 84.7404 | 185.1369 | 0.114574 | 149 |
| GOLPH4 | 33.17142 | 95.59522 | 94.61209 | 145.7242 | 40.8177 | 92.95852 | 84.7404 | 185.1369 | 0.170779 | 111 |
| HSP60 | 71.59402 | 103.3321 | 103.6725 | 145.7242 | 40.8177 | 97.97317 | 103.5249 | 177.7374 | 0.425315 | 68 |
| Nucleolin | 31.58946 | 89.83544 | 90.75636 | 145.7242 | 40.8177 | 93.85647 | 84.7404 | 185.1369 | 0.096196 | 138 |
| Phalloidin | 31.58946 | 82.7879 | 88.31179 | 118.2838 | 40.8177 | 65.02848 | 60.97885 | 98.11028 | 0.922539 | 52 |
| Sortilin | 31.58946 | 94.8242 | 95.05421 | 145.7242 | 40.8177 | 94.89408 | 88.183 | 185.1369 | 0.101707 | 67 |
| TOM20 | 31.58946 | 96.15309 | 94.61209 | 145.7242 | 40.8177 | 95.46001 | 88.183 | 185.1369 | 0.174001 | 91 |
| WGA | 31.58946 | 81.12397 | 85.34437 | 118.2838 | 42.43858 | 69.56205 | 68.84967 | 98.11028 | 0.484484 | 27 |

While the distribution of area and intensity exhibited weak significant p-values for any marker, UC MSC organelles, in general, exhibited higher protein expression for organelle markers and larger mean area per marker. The median area of UC MSCs was higher than the median area of BM MSCs except for Concanavalin A. The range of average intensity values for UC MSCs was higher than the range for BM MSCs. This could be due to higher cell-to-cell variability in intensity values for UC MSCs compared to BM MSCs.

Organelles share distinct pixel overlap colocalizations within single BM and UC MSCs. Because BM and UC MSCs expressed different spatial organelle patterns, we reasoned these differences could be recapitulated in their single-cell distributions. Single-cell analysis and comparisons were performed by selecting 4 organelle protein markers (ATF6, GOLPH4, Nucleolin, and TOM20) for 7 BM and UC MSCs. DAPI was used as an additional marker to locate the nucleus. These markers were selected based on their specificity to the target organelle (Table 9).

TABLE 9
Description of antibodies used in 10-plex organelle mapping in MSCs.

| Marker | Target | Function |
|---|---|---|
| HSP60 | Mitochondria | Heat Shock Protein - molecular chaperone; aids |
| | | protein folding; essential for the protein import |
| | | into the mitochondrial matrix ^{1f-3}f |
| TOM20 | Mitochondria | Translocase of the outer membrane of |
| | | mitochondria; assists the movement of nucleus- |
| | | encoded precursor proteins ^{4f, 5}f |
| ATF6 | Endoplasmic Reticulum | Activating transcription factor 6 - Acts during |
| | | ER stress by activating unfolding protein |
| | | response (UPR) target genes ^{6f, 7}f |
| GOLPH4/ | Golgi apparatus | Golgi integral membrane protein. Plays a role |
| GPP130 | | in endosome to Golgi protein trafficking ^{8, 9} |
| Sortilin | Localized to membranes | Sorting receptor in the Golgi compartment. |
| | of the Golgi, ER, | Clearance receptor on the cell surface. |
| | nucleus, endosomes, and | Regulates intracellular protein transport |
| | lysosomes. | through secretory or endocytic pathways ^{10f, 11}f |
| Concanavalin A | Endoplasmic reticulum | Selectively binds to a-mannopyranosyl and a- |
| | | glucopyranosyl residues found in the |
| | | mammalian cell membrane ^{12f, 13}f |
| Phalloidin | F-Actin | Binds to filamentous actin (F-actin) which |
| | | takes part in various cellular functions (cell |
| | | motility, maintenance of cell shape, and |
| | | regulation of transcription) ^{13f, 14}f |
| WGA | Golgi apparatus and | Carbohydrate-binding lectin binding to sialic |
| | plasma membrane | acid and N-acetylglucosamine residues of |
| | | glycoproteins. Labels the cell membrane of |
| | | mammalian cells and tissues. Also stains |
| | | multiple proteins and lipids in the Golgi that are |
| | | glycosylated ^{13f, 15f} |
| Nucleolin | Nucleolus | Plays a role in ribosome biogenesis and rRNA |
| | | synthesis ^{16f} |
| β-Tubulin | Cytosol | One of the forms of Tubulin is a cytoskeleton |
| | | component. Multiple β-Tubulin isoforms are |
| | | expressed in different regions of the cell ^{17f} |

Phalloidin, Concanavalin A, and WGA, while highlighting organelles and cellular components, also work as cell segmentation markers, and are therefore less specific^{29 e}. While TOM20 and HSP60 both colocalize in mitochondria, HSP60 can also be found in extramitochondrial regions such as the cytosol, vesicles, and the cell membrane^{30 e-32 e}. Similarly, Sortilin can be found in regions other than the Golgi, such as endosomes and lysosomes, whereas GOLPH4 is predominantly localized in the Golgi^{33 e,34 e}.

To explore how organelles colocalized in subcellular regions, we used two metrics, including Pearson's correlation and pixel overlap colocalization. Pearson's correlation coefficients between the selected markers for each cell were calculated, and the average values for BM and UC MSCs were plotted as a heatmap with dendrograms (FIG. 63A, FIG. 69A-70B, Table 15).

TABLE 15
Pearson's correlation coefficient values for selected marker pairs for 7 BM and UC MSCs (corresponding to FIG. 63A).

| | | | | | | | | BM | BM |
|---|---|---|---|---|---|---|---|---|---|
| Marker Pair | BM1 | BM2 | BM3 | BM4 | BM5 | BM6 | BM7 | Average | StdDev |
| ATF6_DAPI | 0.196423 | 0.044327 | 0.092174 | 0.101055 | 0.290203 | 0.084216 | 0.036656 | 0.120722 | 0.091189 |
| ATF6_GOLPH4 | 0.886905 | 0.743921 | 0.736341 | 0.746596 | 0.684851 | 0.625186 | 0.474311 | 0.69973 | 0.127224 |
| ATF6_Nucleolin | 0.16351 | 0.003114 | 0.063636 | 0.091991 | 0.249984 | 0.071487 | 0.035529 | 0.097036 | 0.083875 |
| ATF6_TOM20 | 0.830419 | 0.638131 | 0.704807 | 0.716485 | 0.709677 | 0.640397 | 0.625479 | 0.695056 | 0.070899 |
| DAPI_GOLPH4 | 0.258731 | −0.01459 | 0.032707 | 0.102173 | 0.326128 | 0.030444 | 0.002435 | 0.105432 | 0.134254 |
| DAPI_Nucleolin | 0.900525 | 0.552446 | 0.927836 | 0.949862 | 0.90688 | 0.888909 | 0.956599 | 0.869008 | 0.141837 |
| DAPI_TOM20 | 0.165633 | 0.047519 | 0.170235 | 0.126379 | 0.25699 | 0.070878 | 0.083174 | 0.131544 | 0.07246 |
| GOLPH4_Nucleolin | 0.218046 | −0.01295 | 0.012693 | 0.084748 | 0.244019 | 0.0105 | −0.00011 | 0.079564 | 0.108318 |
| GOLPH4_TOM20 | 0.785866 | 0.52624 | 0.64621 | 0.776565 | 0.675298 | 0.616704 | 0.358623 | 0.62650 | 0.148656 |
| Nucleolin_TOM20 | 0.138058 | 0.008803 | 0.1439 | 0.108819 | 0.191075 | 0.062622 | 0.084286 | 0.105366 | 0.059871 |
| | | | | | | | | UC | UC |
| Marker Pair | UC1 | UC2 | UC3 | UC4 | UC5 | UC6 | UC7 | Average | StdDev |
| ATF6_DAPI | 0.407033 | 0.335367 | 0.578863 | 0.517145 | 0.41034 | 0.624892 | 0.398562 | 0.467457 | 0.107062 |
| ATF6_GOLPH4 | 0.572879 | 0.297727 | 0.651611 | 0.430775 | 0.27216 | 0.494814 | 0.344256 | 0.437746 | 0.143218 |
| ATF6_Nucleolin | 0.36727 | 0.349333 | 0.583028 | 0.382364 | 0.110109 | 0.374694 | 0.123962 | 0.327251 | 0.16395 |
| ATF6_TOM20 | 0.808696 | 0.688347 | 0.801106 | 0.482706 | 0.392987 | 0.756106 | 0.621493 | 0.650206 | 0.16139 |
| DAPI_GOLPH4 | 0.114759 | 0.056249 | 0.205999 | 0.058438 | −0.00208 | 0.362382 | 0.058118 | 0.121981 | 0.124319 |
| DAPI_Nucleolin | 0.875816 | 0.884637 | 0.837625 | 0.726613 | 0.375265 | 0.575322 | 0.503603 | 0.682697 | 0.200883 |
| DAPI_TOM20 | 0.217138 | 0.026181 | 0.28817 | 0.067537 | 0.011137 | 0.446522 | 0.10885 | 0.166505 | 0.159544 |
| GOLPH4_Nucleolin | 0.109928 | 0.066897 | 0.29133 | 0.030311 | −0.008 | 0.210649 | −0.00062 | 0.100072 | 0.113055 |
| GOLPH4_TOM20 | 0.575517 | 0.299882 | 0.659152 | 0.678319 | 0.480347 | 0.511235 | 0.439772 | 0.520603 | 0.131685 |
| Nucleolin_TOM20 | 0.207655 | 0.077855 | 0.352141 | 0.004792 | −0.00877 | 0.258247 | 0.002558 | 0.127783 | 0.144732 |

The elements of the correlation heatmaps denote the location concordance between the nuclear and cytosolic markers^{17 e,35 e}. ATF6 has a higher correlation with nuclear markers in UC MSCs than in BM MSCs. On the other hand, GOLPH4 has a slightly lower correlation with ATF6 and TOM20 in UC cells compared to the BM cells. In general, UC MSCs exhibit higher spatial correlation than BM MSCs between nucleus and cytosol, suggesting that UC MSC organelles are spread over a larger area, agreeing well with FIG. 3c, and are thus considered as energetically more active cells in their function. To study the single-cell variation in correlation coefficients in the cells, the pairwise correlation coefficients were plotted as a heatmap across all cells (FIG. 63A, right). The heatmap shows the cell-to-cell variation in the correlation coefficients between marker pairs and also indicates the differences between BM and UC cells. Some markers exhibit higher cell-to-cell variability. The correlation coefficients between marker pairs Nucleolin_DAPI, GOLPH4_ATF6, TOM20_GOLPH4, and TOM20_ATF6 are higher than other marker pairs for most of the cells. These pairs also have a higher correlation in BM than UC MSCs, revealing that nuclear pairs and cytosol pairs are more separated in BM than in UC MSCs. Likewise, the pairwise correlation coefficients for Nucleolin_ATF6 and DAPI_ATF6 are higher in UC MSCs compared to BM MSCs, indicating a higher interaction between the nucleus and ER in UC MSCs. Interestingly, the same trend is less prominent for interaction between the nucleus and mitochondria (e.g. DAPI_TOM20, Nucleolin_TOM20). These differences in correlation values suggest that the UC MSCs possess more prevalent proteomic activity in their organelles than BM MSCs.

Pixel overlap colocalization, i.e., the metric that counts the number of overlapping pixels after thresholding normalized to the cell area, was another method to measure colocalization between the markers for each cell. These average values were then plotted as a heatmap (FIG. 63B, FIG. 71A-72B, Table 16).

TABLE 16
Colocalization values for selected marker pairs for 7 BM and UC MSCs (corresponding to FIG. 63B).

| | | | | | | | | BM | BM |
|---|---|---|---|---|---|---|---|---|---|
| Marker Pair | BM1 | BM2 | BM3 | BM4 | BM5 | BM6 | BM7 | Average | StdDev |
| DAPI_ATF6 | 0.198657 | 0.039487 | 0.086898 | 0.093114 | 0.171108 | 0.067664 | 0.067568 | 0.103499 | 0.058711 |
| DAPI_GOLPH4 | 0.213441 | 0.018068 | 0.049052 | 0.09539 | 0.189666 | 0.035477 | 0.0349 | 0.090856 | 0.07964 |
| DAPI_Nucleolin | 0.781629 | 0.432631 | 0.922132 | 0.944076 | 0.818159 | 0.871556 | 0.963145 | 0.819047 | 0.182802 |
| GOLPH4_ATF6 | 0.765817 | 0.618961 | 0.505556 | 0.597201 | 0.525747 | 0.468361 | 0.391373 | 0.553288 | 0.121021 |
| GOLPH4_DAPI | 0.213441 | 0.018068 | 0.049052 | 0.09539 | 0.189666 | 0.035477 | 0.0349 | 0.090856 | 0.07964 |
| GOLPH4_Nucleolin | 0.203745 | 0.008078 | 0.039074 | 0.091449 | 0.153867 | 0.027703 | 0.033591 | 0.079644 | 0.073752 |
| Nucleolin_ATF6 | 0.185392 | 0.013872 | 0.074979 | 0.090165 | 0.141186 | 0.05908 | 0.066248 | 0.090132 | 0.056658 |
| Nucleolin_DAPI | 0.781629 | 0.432631 | 0.922132 | 0.944076 | 0.818159 | 0.871556 | 0.963145 | 0.819047 | 0.182802 |
| Nucleolin_GOLPH4 | 0.203745 | 0.008078 | 0.039074 | 0.091449 | 0.153867 | 0.027703 | 0.033591 | 0.079644 | 0.073752 |
| TOM20_ATF6 | 0.713756 | 0.522471 | 0.444103 | 0.569148 | 0.48106 | 0.460904 | 0.446174 | 0.519659 | 0.096766 |
| TOM20_DAPI | 0.175406 | 0.038659 | 0.092029 | 0.109527 | 0.217628 | 0.059561 | 0.079293 | 0.1103 | 0.06424 |
| TOM20_GOLPH4 | 0.636476 | 0.491008 | 0.423903 | 0.651613 | 0.48217 | 0.437755 | 0.273494 | 0.485203 | 0.130031 |
| TOM20_Nucleolin | 0.165328 | 0.016058 | 0.08344 | 0.105156 | 0.172778 | 0.050871 | 0.078637 | 0.096039 | 0.057259 |
| | | | | | | | | UC | UC |
| Marker Pair | UC1 | UC2 | UC3 | UC4 | UC5 | UC6 | UC7 | Average | StdDev |
| DAPI_ATF6 | 0.196808 | 0.092944 | 0.285006 | 0.271964 | 0.152621 | 0.290183 | 0.171751 | 0.208754 | 0.075846 |
| DAPI_GOLPH4 | 0.07593 | 0.029461 | 0.183259 | 0.086865 | 0.014359 | 0.231524 | 0.044002 | 0.095057 | 0.081912 |
| DAPI_Nucleolin | 0.741429 | 0.761125 | 0.708505 | 0.665065 | 0.164178 | 0.370388 | 0.279771 | 0.527209 | 0.248358 |
| GOLPH4_ATF6 | 0.480053 | 0.303715 | 0.619116 | 0.419372 | 0.262292 | 0.418901 | 0.289242 | 0.398956 | 0.126258 |
| GOLPH4_DAPI | 0.07593 | 0.029461 | 0.183259 | 0.086865 | 0.014359 | 0.231524 | 0.044002 | 0.095057 | 0.081912 |
| GOLPH4_Nucleolin | 0.071209 | 0.040352 | 0.236832 | 0.066246 | 0.000957 | 0.093926 | 0.006353 | 0.073696 | 0.079605 |
| Nucleolin_ATF6 | 0.173156 | 0.110506 | 0.324521 | 0.210464 | 0.024332 | 0.109092 | 0.041439 | 0.14193 | 0.104087 |
| Nucleolin_DAPI | 0.741429 | 0.761125 | 0.708505 | 0.665065 | 0.164178 | 0.370388 | 0.279771 | 0.527209 | 0.248358 |
| Nucleolin_GOLPH4 | 0.071209 | 0.040352 | 0.236832 | 0.066246 | 0.000957 | 0.093926 | 0.006353 | 0.073696 | 0.079605 |
| TOM20_ATF6 | 0.670208 | 0.56118 | 0.709138 | 0.455341 | 0.35816 | 0.642097 | 0.492202 | 0.555475 | 0.127329 |
| TOM20_DAPI | 0.13406 | 0.025831 | 0.246479 | 0.077721 | 0.023136 | 0.27258 | 0.077346 | 0.12245 | 0.10107 |
| TOM20_GOLPH4 | 0.477261 | 0.30591 | 0.59053 | 0.585311 | 0.353276 | 0.419717 | 0.321136 | 0.436163 | 0.119095 |
| TOM20_Nucleolin | 0.127179 | 0.0478 | 0.291461 | 0.051048 | 0.001147 | 0.111043 | 0.007882 | 0.09108 | 0.100283 |

This parameter is similar to Mander's coefficients, except that it gives an absolute number of pixels that are overlapping between the two images^{36 c}. Similar to Pearson's correlation, UC MSCs show higher pixel overlaps than BM MSCs, especially between DAPI and ATF6, again suggesting more crosstalk between the nucleus and ER in UC MSCs. The colocalization for BM MSCs and UC MSCs was combined and plotted as another heatmap to compare the cell-to-cell differences (FIG. 63B, right). A few UC MSCs express marker pairs differently than the rest of the cells, suggesting more single-cell variability in that population. Among the organelle markers, there is higher pixel overlap colocalization in Nucleolin_DAPI, GOLPH4_ATF6, TOM20_GOLPH4, and TOM20_ATF6, implying fewer interactions between nucleus and cytosol except for a few UC MSCs: 10, 12, 13, and 14. The higher single variability in UC MSCs is consistent with the observation from Pearson's correlation coefficient. The pixel overlap colocalization between cytosolic markers and nuclear markers was expectedly low, which was also observed in Pearson's correlation coefficient.

Spatial spread over major and minor axes of organelles distinguishes UC and BM MSCs better than area only. In addition to colocalization differences, we hypothesized that there are considerable differences in morphology and size between UC and BM MSCs. The heatmaps of different markers' areas indicate that UC MSCs and BM MSCs show more differences along major and minor axes than area (FIG. 63C, Table 17), suggesting that organelles are expressed in different morphology and shapes rather than varying in area.

TABLE 17
Area of selected markers (in square pixels) for 7 BM and UC
MSCs obtained from CellProfiler (corresponding to FIG. 63C).

| Marker Pair | BM1 | BM2 | BM3 | BM4 | BM5 | BM6 | BM7 |
|---|---|---|---|---|---|---|---|
| ATF6 | 72077 | 53496 | 26147 | 92562 | 91513 | 137280 | 79126 |
| BetaTubulin | 110718 | 43207 | 29110 | 137453 | 118420 | 162026 | 177176 |
| ConcanavalinA | 74479 | 58762 | 30292 | 147074 | 103561 | 167414 | 118167 |
| DAPI | 18000 | 16198 | 10203 | 21401 | 26186 | 21884 | 24240 |
| GOLPH4 | 73537 | 44528 | 29841 | 86267 | 50304 | 127784 | 89119 |
| HSP60 | 106731 | 47291 | 43028 | 133622 | 92454 | 231791 | 156379 |
| Nucleolin | 2158 | 1765 | 2572 | 1027 | 2498 | 887 | 707 |
| Phalloidin | 116287 | 55108 | 36672 | 266663 | 174239 | 229626 | 289824 |
| Sortilin | 61584 | 35704 | 31165 | 88538 | 76510 | 180883 | 52895 |
| TOM20 | 88060 | 49251 | 28398 | 160884 | 173177 | 185333 | 137833 |
| WGA | 90379 | 50818 | 34451 | 138099 | 96925 | 231986 | 134474 |
| Marker Pair | UC1 | UC2 | UC3 | UC4 | UC5 | UC6 | UC7 |
| ATF6 | 95622 | 64911 | 64766 | 95730 | 40275 | 85629 | 254557 |
| BetaTubulin | 198615 | 148244 | 154339 | 327964 | 208441 | 390137 | 664577 |
| ConcanavalinA | 119233 | 88994 | 105803 | 191096 | 78745 | 140352 | 295241 |
| DAPI | 25724 | 23470 | 24846 | 23510 | 19394 | 26163 | 35329 |
| GOLPH4 | 105132 | 75821 | 69107 | 24267 | 31549 | 76274 | 77424 |
| HSP60 | 247370 | 125875 | 152705 | 348499 | 114527 | 298772 | 488934 |
| Nucleolin | 10989 | 9814 | 3180 | 3860 | 2257 | 338 | 13433 |
| Phalloidin | 145333 | 71630 | 76489 | 159617 | 53867 | 238990 | 301581 |
| Sortilin | 178156 | 89586 | 70557 | 231671 | 88344 | 112341 | 413930 |
| TOM20 | 122737 | 75906 | 101154 | 116887 | 65806 | 124685 | 311985 |
| WGA | 130533 | 83281 | 83649 | 110234 | 60433 | 110821 | 245917 |

This is especially crucial with TOM20, which suggests a stark difference in mitochondrial energetic activity. ATF6 and TOM20 have similar areas, while GOLPH4 has a smaller area by comparison. The nuclear markers are smaller in comparison to the cytosolic markers. The major and minor axis heatmaps show a better distinction between BM MSCs and UC MSCs (FIG. 63C, Tables 18-19).

TABLE 18
Minor axis length (in pixels) of selected markers for 7 BM and
UC MSCs obtained from CellProfiler (corresponding to FIG. 63C).

| Markers | BM1 | BM2 | BM3 | BM4 | BM5 | BM6 | BM7 |
|---|---|---|---|---|---|---|---|
| ATF6 | 176.3 | 174.7 | 170.49 | 346.84 | 365.07 | 364.79 | 204.44 |
| BetaTubulin | 242.35 | 152.8 | 182.2 | 408.64 | 429.67 | 383.02 | 278.02 |
| ConcanavalinA | 170.62 | 176.34 | 180.06 | 408.64 | 364.21 | 376.87 | 247.69 |
| DAPI | 112.56 | 106.95 | 108.17 | 136.3 | 162.94 | 141.53 | 134.99 |
| GOLPH4 | 176.07 | 165.82 | 177.4 | 350.53 | 206.8 | 368.23 | 213.95 |
| HSP60 | 262.32 | 164.47 | 231.04 | 358.46 | 413.57 | 415.93 | 267.94 |
| Nucleolin | 49.68 | 43.63 | 54.67 | 31.32 | 49.68 | 31.36 | 22.39 |
| Phalloidin | 294.52 | 189.62 | 205.06 | 483.99 | 529.59 | 450.02 | 392.95 |
| Sortilin | 168.3 | 139.98 | 194.53 | 253.56 | 286.93 | 416.2 | 183.64 |
| TOM20 | 194.56 | 167.36 | 187.51 | 449.22 | 571.29 | 418.18 | 249.73 |
| WGA | 205.62 | 190.17 | 203.32 | 477.82 | 343.91 | 427.62 | 338.99 |
| Marker Pair | UC1 | UC2 | UC3 | UC4 | UC5 | UC6 | UC7 |
| ATF6 | 185.82 | 212.52 | 214.96 | 245.46 | 200 | 269.98 | 408.79 |
| BetaTubulin | 265.05 | 266.07 | 290.35 | 459.55 | 407.43 | 725.51 | 784.57 |
| ConcanavalinA | 213.64 | 230.11 | 239.23 | 294.85 | 292.19 | 337.3 | 591.77 |
| DAPI | 137.03 | 137.16 | 141.49 | 138.21 | 122.38 | 141.31 | 162.41 |
| GOLPH4 | 232.78 | 221.8 | 172.72 | 141.47 | 159.68 | 281.32 | 344.52 |
| HSP60 | 276.84 | 254.91 | 293.33 | 544.46 | 315.08 | 687.99 | 668.44 |
| Nucleolin | 88.48 | 101.56 | 76.11 | 59.14 | 49.53 | 17.05 | 134.58 |
| Phalloidin | 222.84 | 215.17 | 211.21 | 284.31 | 235.53 | 660.83 | 651.93 |
| Sortilin | 262.86 | 245.3 | 250.9 | 428.94 | 288.3 | 325.11 | 596.86 |
| TOM20 | 196.72 | 226.27 | 224.92 | 296.1 | 248.79 | 319.57 | 520.81 |
| WGA | 212.29 | 232.73 | 240.43 | 249.75 | 247.39 | 289.91 | 473.24 |

TABLE 19
Major axis length (in pixels) of selected markers for 7 BM and
UC MSCs obtained from CellProfiler (corresponding to FIG. 63C).

| Markers | BM1 | BM2 | BM3 | BM4 | BM5 | BM6 | BM7 |
|---|---|---|---|---|---|---|---|
| ATF6 | 636.77 | 468.73 | 205 | 561.16 | 452.47 | 615.57 | 541.14 |
| BetaTubulin | 786.37 | 439.98 | 211.33 | 730.36 | 584.37 | 814.98 | 978.95 |
| ConcanavalinA | 640.94 | 506.47 | 223.28 | 761.49 | 528.2 | 820.95 | 720.3 |
| DAPI | 208.99 | 195.66 | 121.24 | 200.28 | 205 | 197.94 | 229.06 |
| GOLPH4 | 622.37 | 419.11 | 223.93 | 517.1 | 396.7 | 657.58 | 597.29 |
| HSP60 | 770.53 | 413.77 | 268.36 | 595.57 | 522.41 | 1021.36 | 905.55 |
| Nucleolin | 62.28 | 54.54 | 82.92 | 45.92 | 84.07 | 38.14 | 74.5 |
| Phalloidin | 778.68 | 466.74 | 246.71 | 930.7 | 648.23 | 1033.74 | 1100.64 |
| Sortilin | 597.71 | 365.05 | 210.77 | 557.17 | 423.54 | 888.56 | 445.49 |
| TOM20 | 684.36 | 426.75 | 201.95 | 699.62 | 720.06 | 777.22 | 882.11 |
| WGA | 636.04 | 372.98 | 223.66 | 592.63 | 494.12 | 987.44 | 626.13 |
| Marker Pair | UC1 | UC2 | UC3 | UC4 | UC5 | UC6 | UC7 |
| ATF6 | 706.87 | 411.92 | 421.99 | 599.38 | 277.71 | 434.69 | 1224.38 |
| BetaTubulin | 1250.36 | 838.92 | 809.9 | 1348.87 | 690.34 | 1107.8 | 2171.82 |
| ConcanavalinA | 789 | 526.44 | 588.51 | 1241.91 | 401.95 | 600 | 1221.41 |
| DAPI | 239.54 | 218.48 | 224.92 | 217.11 | 202.73 | 236.91 | 278.46 |
| GOLPH4 | 725.69 | 499.68 | 590.89 | 220.42 | 269.98 | 456.84 | 485.73 |
| HSP60 | 1671.15 | 854.78 | 775.68 | 1174.6 | 511.22 | 982.07 | 1895.63 |
| Nucleolin | 167.38 | 154.58 | 96.8 | 85.34 | 86.67 | 25.81 | 178.18 |
| Phalloidin | 949.26 | 451.44 | 498.97 | 1068.1 | 308.68 | 953.85 | 1273.32 |
| Sortilin | 990.55 | 505.19 | 397.59 | 1167.73 | 423.1 | 577.23 | 1374.41 |
| TOM20 | 858.92 | 482.38 | 649.79 | 879.06 | 393.58 | 583.23 | 1130.1 |
| WGA | 870.18 | 483.5 | 483.86 | 767.4 | 318.4 | 564.18 | 1079.46 |

The markers found in the nucleus, DAPI, and Nucleolin have smaller major and minor axis values across all cells. TOM20 has the largest major and minor axis values, followed by ATF6 and then GOLPH4. Differences in spatial spreading in TOM20 and ATF6 expression are attributable to more UC than BM MSCs. The mitochondria being larger than the ER also suggests a more energetically active state for these UC MSCs.

Uc-MSCs Display Consistently Higher Mitochondrial Expression than BM-MSCs

To statistically benchmark how differently single cells express the same organelle, we implemented the Kolmogorov-Smirnov (K-S) hypothesis test to highlight significant differences between the spatial distribution of organelle marker pairs, including GOLPH4/Sortilin and TOM20/HSP60 (FIG. 63D, Table 20).

TABLE 20
Raw p-values obtained from the KS Hypothesis test for TOM20
vs HSP60 and GOLPH4 vs Sortilin (corresponding to FIG. 63D).

| | |
|---|---|
| TOM20 vs HSP60 | GOLPH4 vs Sortilin |

| Cell | BM | UC | BM | UC |
|---|---|---|---|---|
| 1 | 4.9E−106 | 0 | 8.1E−193 | 0 |
| 2 | 0 | 0 | 2.5E−230 | 0 |
| 3 | 0 | 8.9E−288 | 0 | 0 |
| 4 | 3.61E−30 | 1.08E−21 | 0 | 0 |
| 5 | 1.05E−67 | 0 | 0 | 0 |
| 6 | 1.3E−190 | 0 | 0 | 0 |
| 7 | 4.41E−91 | 0 | 0 | 0 |

Concerning their spatial distributions around the cell's center of mass, GOLPH4 and Sortilin, which both target the Golgi apparatus, express similar patterns within most BM and UC MSCs. On the other hand, spatial distributions concerning the cell's center of mass for HSP60 and TOM20, which both target the mitochondria, exhibit different spatial patterns within more BM MSCs than UC MSCs (FIG. 63D). A few cells exhibit moderate or no levels of significance between HSP60 and TOM20 spatial expression. Between the two cell types, BM MSCs show a higher amount of significant differences between the spatial distributions of the markers compared to UC MSCs, indicating higher single-cell variability in the expression of markers. Since UC MSCs exhibit larger HSP60 and TOM20 areas (FIG. 62C) with more variable intensities (FIG. 62C), we conclude that UC MSCs are more uniformly expressing higher mitochondrial activity, and, thus, UC MSCs are more energetic than BM MSCs.

### Multiplexed Pixel Clustering Indicates Cell-Type Specific Organelle Interactions

We asked whether there are more organelle interactions beyond colocalized nucleus and cytosol or not. To uncover this puzzling question about organelle interactions, unsupervised, pixel-level clustering was performed on the dataset consisting of intensity values of all the markers^{26 e}. The goal of performing pixel-level clustering is to identify a subset of pixels that share a unique multiplexed intensity profile. These clusters could indicate an organelle pattern or regions of similar functionality, and their hierarchical relationship reveals communication among them. The K-Means clustering algorithm was used to group the pixels into 10 clusters. The two cell types were clustered independently, and the resultant clusters were colored back on the images of the single cells. The spatial mapping of a single cell's clustered regions was compared with the organelle colocalization calculated as the product of markers targeting that organelle (FIG. 64A-64B, left). The pixel-level clustering algorithm highlighted certain organelle patterns, notably showing a distinction between the nucleus and cytosol. The organelle marker distributions of each cluster were plotted as cluster maps (FIG. 64A-64B, right). The dendrograms show different hierarchical clustering patterns and cluster intensities across markers for BM and UC cells. TOM20 is directly associated with ATF6 or GOLPH4 in both UC and BM MSCs, suggesting crosstalk between mitochondria and ER (FIG. 64A-64B, right). DAPI and Nucleoli are also directly linked in both of these cell types, implying segregation of nuclear interaction before reaching the cytosol. The clustering results also indicate a stronger association of GOLPH4 and TOM20 in BM than in UC MSCs (FIG. 64A-64B, right bottom), which suggests more crosstalk between ER and mitochondria in BM MSCs. Given the potential higher energetic activity of UC MSCs observed in previous sections, the organelle energy is directed elsewhere than the ER in UC MSCs.

### Superpixel Segmentation Informs Content-Aware Spatially Distinct Features of Each Organelle in UC and BM MSCs

Finally, to evaluate the spatially distinct features of our area and colocalization results (FIG. 67c), we analyzed several organelles with a superpixel segmentation approach using K Means^{37 e}. In this technique, the content-aware texture features were calculated to observe the spatial patterns of each marker. The various texture features used pixel intensity, energy Laplacian, modified Laplacian, diagonal Laplacian, variance Laplacian, and gray level variance. The images were segmented into superpixels by grouping the pixels around dense organelle regions across several subcellular partitions (n=250), and the different texture features were calculated for each marker (FIG. 65A). The texture feature plots highlight the variations in the spatial organization of the marker. The superpixels containing the highest texture features indicate the region of maximum colocalization for each marker. Heatmaps of the texture features were obtained for each marker to compare the spatial patterns between BM and UC MSCs (FIG. 65B). The heatmaps indicate some similarities in texture features within BM and UC MSC superpixels. ATF6, DAPI, GOLPH4, and TOM20 in particular show higher similarity within each cell type, based on the number of superpixels clustered together. For nuclear markers, pixel intensity, gray level variance, and variance Laplacian illustrated clearer regions than the other methods (FIG. 65A). Modified and diagonal Laplacian features produced more spread results for all the markers, and are thus less informative. Interestingly, GOLPH4 and ATF6 were shown to localize in different hotspots around the cytosol as seen with energy Laplacian and variance Laplacian. This spread explains its colocalization with mitochondria seen in previous figures because a more discontinuous, spread organelle has a higher likelihood of colocalizing with other organelles in the cytosol. Quantification of texture feature demonstrated that modified and diagonal Laplacians yield higher superpixel values, especially in UC MSCs (FIG. 65B), yielding additional content-aware spatial features to complement the previously discussed higher energetic activity of UC MSCs compared to BM MSCs.

### Virtual Reality Enables Interactive Visualization and Quantification of Spatial Organelle Maps of MSCs

In addition to the open-source and user-based analysis of organelle data, the multiplexed proteomic images were interactively visualized in Virtual Reality (VR)^{38 e} platform using the software ConfocalVR and Genuage to visually explore our quantitative findings in an immersive experiential learning environment. In Confocal VR, organelle markers targeting the same organelle (Column 1: ATF6 & Concanavalin A, Column 2: Beta Tubulin & Nucleolin, Column 3: GOLPH4 & Sortilin, Column 4: Phalloidin & WGA, Column 5: TOM20 & HSP60) were visualized together to observe regions of colocalization (FIG. 66A, FIG. 73). Histograms of the count of pixels in bin widths of 12.5 pixels (108 nm/pixel, bin width 1.354 μm) were plotted for each marker in a single BM USC using VR headset using Genuage (FIG. 66B) to examine the difference in the spatial distribution of the markers within a cell. In VR-based explorations, TOM20, HSP60, ATF6, GOLPH4, and Beta Tubulin demonstrated non-overlapping regions of the cytosol and overlapping hotspots, yielding spatially variant and distinct histogram shape following organelle interactions and individual marker expressions (FIG. 70b). On the other hand, DAPI, Nucleolin, Sortilin, Concanavalin A, and Phalloidin exhibited more spatially continuous regional expression in the VR-based histograms. Thereby, as illustrated in the VR-based organelle visualizations and interactive quantifications, the incorporation of virtual reality into spatial omics datasets will enable the interactive discovery of spatially modulated patterns in multiplexed molecular datasets from health and disease settings.

### Discussion

Image analysis technology for investigating cell features in biological research has advanced significantly over the past several decades. Finding protein associations within cells through spatial image analysis has the potential to deepen our understanding of their microenvironment and how their structural organization changes as cells transition from normal to abnormal cell behaviors. This work focused on observing spatial organelle distribution in two types of MSCs—BM and UC—and studying their differences. Rapid multiplex immunofluorescence was performed using antibodies that target the organelles to understand colocalization across the proteins. Multiplex analysis of organelle markers indicated differences in the spatial organization of different organelle proteins in BM and UC MSCs. Interactions between the proteins were compared using Pearson's correlation coefficients and colocalization, which revealed differences between BM and UC cells. Pixel-level clustering, morphology, and texture analysis methods also showed a considerable organelle networking difference between the two cell types. Organelle phenotypes and their energy changes have been linked to stem cell fate, signaling, and therefore function^{39 e,40 e}. For example, the ER-mitochondria linkage has been implicated in lipid and energy metabolism as well as apoptosis signaling^{41 e}. Since organelle phenotypes indicate changes in cell energy that affect cell function, it is important to study these phenotypes to improve future therapeutics.

This study demonstrated a rapid multiplexed immunofluorescence protocol to perform spatial profiling of organelles in single cells using 10 protein markers. The targets of these proteins were limited to key organelles, namely mitochondria, the Golgi, ER, the nucleus, and the nucleolus. To obtain a comprehensive understanding of cell behavior, proteins targeting other organelles, such as the peroxisomes, need to be included in the study. An important point to consider while selecting these proteins is the target organelle. While some proteins colocalize in a single organelle, some are chaperones and therefore move between different cellular components. These proteins can be useful to understand communication between organelles, providing insights into the functionality of the cells. However, it is important to consider multiple roles in the spatial and temporal coordination of such proteins to answer questions about the structural organization of the organelles.

A limitation of this study is the number of cells considered for analysis. Imaging of the sample performed manually at high magnification (60×) is time-consuming, and therefore the number of regions captured was limited. Discarding cells that had image quality issues resulted in a small dataset of 14 cells (7 BM and 7 UC MSCs). Automated imaging is a potential way to overcome this limitation, as it can assist with capturing more regions, and therefore more cells. This study considered 2D data, but using 3D high-resolution data could improve organelle network mapping and provide more insights into interactions and colocalization. Additionally, organelle contacts and interdependency between the locations of different organelles could be better understood by using 3D data.

The differences between BM and UC MSCs have been well characterized in terms of regenerative, proliferative, differentiation, and clinical outcomes^{18 e,42 e-45 e}. Our study examines the organelle-specific activity differences among single cells from each tissue source. The detected activity differences can be attributed to energetic demand differences from their in situ origin. For example, since UC MSCs are typically derived from infants and BM MSCs originate from older, more mature adults, the difference in each population's energy needs can be observed in the MSC population. UC MSCs were observed to exhibit higher organelle colocalization patterns (FIG. 62A-62B), more variable intensity and expression areas (FIG. 62C-62D), and less single-cell variability (FIG. 4d) than BM MSCs, suggesting that UC MSCs may be more uniformly and energetically active to satisfy the high energy demands of rapidly changing infant developmental processes and growth. On the other hand, BM MSCs should be reflective of older, sedentary, less active adults. Thus, we observed more uniform, lower intensity, and lower area expression patterns than UC MSCs (FIG. 62A-62D). The similar patterns between BM and UC MSCs verified with superpixel segmentation suggest that these differences are less obvious to the eye and more minutely varying in multiplexed organelle maps (FIG. 64A-64B).

Previous studies have investigated energetic differences between BM and UC MSCs^{46 e,47 e}. For example, by measuring lactate from glycolysis, UC MSCs have been shown to produce less lactate than BM MSCs in hypoxic, normoxic, and hyperoxic conditions. Additionally, UC MSCs can adapt more than BM MSCs to a wider range of oxygen conditions as supported by oxygen consumption rates^{46 e}. UC MSCs were able to alter energetic and metabolic levels in response to oxygen-varying environments compared to other types of MSCs. Our study supports these findings at the single-cell level by illustrating more colocalized organelle patterns that possess more variable intensity over larger spatial areas (FIG. 62A-62D). UC MSCs also show less cell-to-cell variability in organelle expression (FIG. 63D). Overall, our findings support the energetically higher and adaptive nature of UC MSCs than BM MSCs that are attributable to energetic demands between the tissue sources of younger and older patients.

Energy levels between UC MSCs and BM MSCs can be compared by studying how organelles change when cultured in different oxygen conditions^{10 e}. Metabolic enzyme proteins can also be used to study energy differences. Combining live imaging with multiplexing will provide spatial and temporal resolution, thus enabling further understanding of organelle interactions^{48 e}. Comparing the difference in organelle enrichment, such as between mitochondria to cytoplasm ratio and ER abundance, provides another interesting point of research for future inquiry. These differences could also reveal details about the functioning of stem cells before their use in therapies.

Another experimental consideration is phase separation, which is a spatiotemporal process in cells^{49 e} and a mechanism used in biomolecular assembly. Various membrane-less organelles are formed through liquid-liquid phase separation; these organelles can also be added to the multiplex profiling panel^{50 e}. Combining spatial organelle network analysis with studying phase separation will shed light on the biophysics of the biomolecular condensates^{51 e}. Dynamic regulation of phase separation can also be used to understand how organelle interactions are affected during phase transitions in cellular molecules^{52 e}.

To evaluate the mechanisms of how different protein/organelle phenotypes contribute to cell-specific functions, the transcriptome and proteome levels of primary cells and cell lines could be compared. Cell line experiments are a well-established approach because they are readily available and cost-effective. However, culturing cells in the media makes them lack tissue architecture and heterogeneity. Cells in culture can have different molecular phenotypes from cells in vivo^{53 e}. Therefore, primary MSC cells and MSC cell lines can serve as models to compare cell-specific functions by quantifying the genetic and proteomic differences. Furthermore, the functions of primary cells and cell lines could be examined by designing a staining panel of organelle markers (ATF6, TOM20, β-Tubulin, GOLPH4, HSP60, Nucleolin, and Sortilin) and protein markers indicating proliferation or apoptosis (Ki67, BIM, and Cyclin E). Also, to relate gene expression with the change at the proteome level, RNA targets could be included in the staining panel. With the multiplexed experiments, the proteomic and transcriptomic phenotyping of cell lines and primary cells could provide a complete relationship between phenotypic differences and cellular function.

An accurate, reproducible, multiplexed protein imaging and analysis method could aid in better visualizing and understanding various disease states and microenvironments in single cells, which could then be used to find or design drugs and therapy methods that work best depending on patient needs^{54 e}. Subcellular organelle analysis using proteomics is a useful method for obtaining spatial cellular maps that can reveal details about the structure and functionality of organelles and the cell. The complex spatial organelle interaction is yet to be fully understood in the context of organelle-related diseases^{55 e,56 e} such as cancer^{57 e}, aging^{58 e}, and regenerative medicine^{59 e} to assess their potential in precision medicine and therapeutics.

### Methods

Cells: Bone marrow-derived MSCs (BM MSCs) and umbilical cord-derived MSCs (UC MSCs) were obtained from RoosterBio, Inc. The culture media was prepared using 89% α-MEM media (Cat #12561-049) with L-glutamine, 10% heat-inactivated fetal bovine serum (HI-FBS), and 1% penicillin-streptomycin (Cat #P4333). The culture media was mixed and filtered before use. BM and UC MSCs were cultured in T-75 flasks with 10 mL of culture media. Cell passages were performed when cells reached 75% confluency using Trypsin LE cell detachment media (Cat #12605-010) at 37° C. The cells were resuspended in respective culture media after centrifugation at 280 g for 6 minutes and then seeded on collagen-coated glass coverslips. The cells were then cultured on coverslips for 24 hours before fixation. Cells were then fixed in 1.6% paraformaldehyde in PBS for 10 minutes at room temperature, followed by another PBS washing and multiplexed staining of organelle protein markers. Duplicate experiments were used for each measurement. Cells were thawed at passage 2 and cultured until passage 20. BM and UC cells were from similar passage numbers.

Antibodies: The primary antibodies considered for this study were ATF6 (ab263955, Abcam), β-tubulin (sc-5274, Santa Cruz Biotechnology), GOLPH4/GPP130 (ab197595, Abcam), HSP60 (ab224528, Abcam), Nucleolin (ab226113, Abcam), Sortilin (ab263873, Abcam), Tom20 (sc-17764, Santa Cruz Biotechnology), Phalloidin (A34055, Invitrogen), WGA (W32466, Invitrogen), and Concanavalin (C11252, Invitrogen). They were used for cell segmentation and additional organelle markers (Table 9). The animal source and dilution of unconjugated antibodies used are as follows: ATF6 (rabbit, 1:200), β-tubulin (mouse, 1:200), GOLPH4 (rabbit, 1:500), HSP60 (rabbit, 1:200), Nucleolin (rabbit, 1:250), Sortilin (rabbit, 1:100) and Tom20 (mouse, 1:200). The dilutions of the antibodies were optimized by performing multiple rounds of IF assays to improve reproducibility.

Antibody Conjugation: The primary antibodies were conjugated with fluorescent dyes using a rapid conjugation kit (ab269823, Abcam). For each 10 μL of primary antibody, 1 μL of modifier reagent was added and mixed gently. The lyophilized powder was dissolved in 10 μL PBS (D8537, Sigma-Aldrich), and 1 μL of this solution was added to each antibody and mixed gently. The mixture was incubated at room temperature for 15 minutes in the dark. After incubation, 1 μL of quencher reagent was added for each 10 μL of antibody used and was then gently mixed. In this experiment, the antibodies were conjugated to Alexa Fluor 647, which is a bright dye with less background fluorescence^{25 e}. While Alexa Fluor 488 is the brightest among the dyes, the channel has higher background fluorescence. Alexa Fluor 555 is the weakest dye, suitable for staining proteins with high abundance and high affinity.

Rapid Multiplex Immunofluorescence: BM-MSCs and UC-MSCs were stained with 10 markers using a total of 8 cycles (Table 10).

TABLE 10
Order of antibodies used for rapid multiplexed immunofluorescence
of organelle markers in MSCs.

| Cycle | Channel | Antibody | Dilution |
|---|---|---|---|
| 1 | | DAPI | 1:1000 |
| | 488 | Sortilin | 1:100 |
| | 647 | β-Tubulin | 1:200 |
| 2 | | DAPI | 1:1000 |
| | 555 | Phalloidin | 1:40 |
| | 647 | WGA | 1:400 |
| 3 | | DAPI | 1:1000 |
| | 647 | ATF6 | 1:200 |
| 4 | | DAPI | 1:1000 |
| | 647 | TOM20 | 1:200 |
| 5 | | DAPI | 1:1000 |
| | 647 | GOLPH4 | 1:500 |
| 6 | | DAPI | 1:1000 |
| | 647 | HSP60 | 1:200 |
| 7 | | DAPI | 1:1000 |
| | 647 | Nucleolin | 1:250 |
| 8 | | DAPI | 1:1000 |
| | 488 | Concanavalin A | 1:20 |

The cells were permeabilized using 0.5% Triton X-100 for 10 minutes at room temperature and washed three times with PBS. At the start of each cycle, blocking was performed with Cell Staining Media (CSM containing 0.5% BSA, 0.02% sodium azide in PBS), 0.5% BSA, and 0.02% Sodium Azide (contains PBS 1×) for 1 hour at RT. After blocking, the coverslip with the cells was incubated at RT for 1 hour with diluted primary or conjugated antibody (250-500 μL per coverslip), followed by four washes with 1×PBS for 5 minutes. All primary antibodies (conjugated and unconjugated) were diluted in CSM. Since the experiment consisted of both conjugated and unconjugated antibodies, indirect immunofluorescence was performed in the first cycle using the unconjugated antibodies. For indirect immunofluorescence, after incubating with the primary antibodies, the coverslip was incubated in secondary antibodies diluted in PBS for 1 hour at RT, followed by four washes with 1×PBS for 5 minutes. The coverslip was then incubated with DAPI (D1306, Invitrogen) diluted in PBS for 10 minutes at RT, followed by four washes using 1×PBS for 5 minutes each. 1×PBS was used as the imaging buffer on the coverslip. After imaging, bleaching was performed using a freshly prepared bleaching buffer consisting of 4.5% (wt/vol) H₂O₂ and 20 mM NaOH in PBS for 1 hour at RT with white light. The sample was imaged to ensure that the fluorescence signal had fallen to background levels (FIG. 74A-74B). The coverslip was washed with 1×PBS three times before starting the next cycle.

Imaging: A wide-field microscope, Nikon Eclipse TE2000-U, was used for fluorescence imaging. For each cycle of imaging, 2-3 channels were captured: Channel 1 detects DAPI/Hoechst at 360 nm, while Channels 2-4 detect fluorophores at Alexa Fluor 488 nm (FITC), 555 nm (TRITC), and 647 nm (Cy5), respectively. The exposure time varied between the markers and the cells. The sample was imaged with a 60× oil lens, resulting in a high-resolution acquisition at 0.1083 μm/pixel. Each imaging region was imaged across 16 z-stacks with 0.5 μm/stack. The best focus z slices of each image were used for analysis. The images were stored as 16-bit multichannel images in ND2 format. They were converted to TIFF format using ImageJ and saved as single or multi-channel grayscale images. Each marker was thresholded with the Otsu method for all analyses^{60 e}.

Quality control: Registration was performed on sequential images from multiple cycles of immunofluorescence to adjust differences in alignment using the RNAscope HiPlex Image Registration Software^{61 e}. DAPI channel images of each cycle were aligned using the DAPI channel image of the first cycle as a reference to obtain the registration transforms, which were then applied to the remaining channels of each cycle. After registration, the best cells were manually selected through visual inspection based on image quality. For example, unreliable regions with intensity values too close to the local background (insufficient contrast) or saturated intensity values (making it hard to distinguish features due to high intensity) were filtered out. Cells that had overlapping cytosol and cells which were touching the image borders were also discarded. This quality control step resulted in 7 BM MSCs and 7 UC MSCs for analysis. Background subtraction was performed using a rolling ball algorithm in ImageJ to remove the background signal from the images. Finally, the pixel intensities were thresholded by calculating the lower 20 percentile (background pixels) and the upper 99.9 percentile (saturated pixels), rescaling the intensities using the two percentiles between 0 and 1 and clipping the values that were lower than 0 and greater than 1. The cells were segmented in ImageJ using cell masks obtained from the Phalloidin marker outline (FIG. 76A-76B).

Scatterplot: Scatter plots of intensity were obtained by random sampling of 50000 pixels for each marker pair colocalized in the same organelle in BM-MSCs and UC-MSCs. The intensity values were min-max normalized. The plots were colored using kernel density estimates obtained from Gaussian kernels.

Pearson's correlation: Pearson's correlation coefficient was calculated for each cell between the markers to evaluate the extent of colocalization between the organelle proteins in the cell. The results were averaged across the cells, and the variance of the values was calculated. The average and variance of correlation coefficient values were visualized as a heatmap with dendrograms for BM and UC cells. A clustered heatmap of Pearson's correlation was displayed with each pair of markers on the x-axis and each cell type on the y-axis. This result is useful to compare the organelle colocalization between BM and UC cells.

Pixel Overlap Colocalization: The pixel overlap colocalization between two given markers was calculated by counting the number of nonzero pixels that occupy the same coordinate. This value was normalized by dividing by the total area of both markers to obtain a final fraction of the common area covered by two markers.

Statistical analysis: The Kolmogorov-Smirnov (KS) hypothesis test was conducted between organelle marker pairs targeting the Golgi (GOLPH4 and Sortilin) and mitochondria (TOM20 and HSP60). For each cell, the center of mass was computed from the segmented mask. Then, each marker's pixel distance to the center of mass was calculated to convert 2D spatial image coordinates to 1D spatial distributions (FIG. 77A-77D). To determine if these spatial distributions differed from one another, the KS test is performed on the 1D spatial distribution (histogram). Since cell morphology is roughly symmetrical, the test examines differences in organelle spatial expression patterns between the nucleus and membrane. The null hypothesis considered was that similar proteins express similar spatial distribution within each cell.

Pairwise analysis: For each marker, multiple, Otsu-thresholded single-cell images were selected and the intensity values were converted into a column. The intensity values were min-max normalized. Pearson's correlation was calculated using intensity values of all marker pairs for each cell in the marker pair and the result was plotted as boxplots. Similarly, to verify Pearson values, pixel overlap colocalization between the markers was also calculated and the values were plotted as boxplots.

Cell area and intensity: For each marker, multiple single-cell images were selected and the average intensity values per cell were plotted as boxplots. Similarly, the area of each marker per cell was calculated by summing the number of non-zero pixels after morphological opening and closing. The area values were also plotted as boxplots.

Pixel Clustering: Pixel phenotypes were clustered in a two-step clustering pipeline. From each pixel location within the cell-segmented region, the intensity value of each marker expression was extracted. The resulting feature matrix consisted of n rows of a total number of pixels and p columns of marker expression. Each column of the feature matrix was min-max normalized. To determine the optimal number of clusters, the elbow method was implemented on the cluster scores for various numbers of clusters. The elbow point was determined to be 10. Thus, the K-Means clustering algorithm was used to cluster the pixels of the 5 markers into 10 clusters. The resultant clusters were then uniquely colored on the original cellular image. To look for any organelle pattern in the clusters, the K-Means clustering results were compared with images of markers grouped according to their organelle affinity and multiplied together pixel-wise.

Super-pixel segmentation and texture analysis: The images were segmented into superpixels using scikit-image^{62 e} (a Python library for scientific image processing) and K Means clustering into superpixels^{37 e}. Texture features were calculated on the superpixels: pixel intensity, energy Laplacian, modified Laplacian, diagonal Laplacian, variance Laplacian, and gray level variance. Pixel intensity computes the mean of the intensities within each superpixel. Energy Laplacian computes the square of the Laplacian of the image from skimage^{62 e}. Modified Laplacian also uses a Laplace function but with two kernels in which one is the transpose of the other; then, the resulting images are summed. Diagonal Laplacian uses convolution with 4 kernels to produce the resulting images that are then summed. Variance Laplacian consists of the square of the difference between the image Laplace and the mean of the image Laplace. Gray level variance involves the square of the difference between the image and the mean of the image. The Python code that contains all the texture functions is labeled “texture_analysis_functions.py” and is available on the Github. The superpixels and texture features were plotted as images and heatmaps were obtained for each marker to compare BM and UC MSCs.

Virtual Reality visualization: The images of organelle markers in BM and UC cells stored as TIF files were converted into Neuroimaging Informatics Technology Initiative file format (NIfTI or .nii) using ImageJ^{38 e}. These files were opened in ConfocalVR software to visualize them in an immersive 3D format^{63 e}. The pixel data was also visualized in another VR software, Genuage, and histograms of the pixel counts were obtained using histogram bins drawn using controllers^{64 e}.

CellProfiler: Morphological features, such as area, major axis, and minor axis, were obtained for each cell using a CellProfiler pipeline^{58 e}, and were then plotted as heatmaps with hierarchical clustering. To first identify and segment the nuclei of cells, “IdentifyPrimaryObject” was used on the DAPI marker with the global two-class Otsu thresholding method. Using the segmented nuclei, the “IdentifySecondaryObject” module was then used to segment the general shapes of the organelles marked by each marker using the same thresholding method. After images of every marker were segmented, all the size/shape features were measured using the “MeasureObjectSizeShape” module for each of the markers and exported into an excel file. The data stored in an excel spreadsheet was manually sorted to extract and analyze only the features of interest, which were the area, major axis, and minor axis of each marker. The measurements of interest were converted into CSV files for visualization. Heatmaps were generated using Python with dendrograms to compare the biophysical measurements and to determine any close relationships or associations between markers and between the two cell types (UC and BM). The z-score was calculated to normalize the output data.

### Software and Algorithms:

Cellprofiler
Numpy
Scipy
Dask
Statsannotations
Pandas
Seaborn

### Example 6: 3D Spatial Gene Neighborhood Networks in Gel-Embedded Mesenchymal Stromal Cells

### Introduction

Spatial-omics technique allows subcellular localization of important biomolecules. Such methods provide additional dimensions that are lost during bulk and single-cell omics analyses due to sample processing. RNA fluorescent in situ hybridization (RNA-FISH) methods allow identification of single RNA transcripts in human cells and tissue samples at single-transcript resolution^{1 g}. Sequential-FISH (seqFISH) and multiplexed-error-robust FISH (MERFISH) allows detection of 10 to 10000 genes in the same sample^{2 g-4 g}. Analysis methods approximate cells as a 2D projection on the cell growth surface or tissue sample adherent surface. However, ignoring 3D spatial resolution can alter the relative spatial relationship. Therefore, analysis using the 3D spatial information provides the highest spatial accuracy. 3D imaging becomes possible with advancements in experiment and imaging technique^{5 g,6 g}. Therefore, further analysis technique is required to extract information encompassed by the data.

One area that can benefit from 3D spatial transcriptomics study is cell-based therapy using mesenchymal stem cells (MSCs). Organoids, spheroids, and aggregates have utilized various synthetic ECM-like 3D networks to support stem cell functions^{7 g}. A recent formulation of PEG-4MAL hydrogel exhibited better physiological relevance compared to mouse-based Matrigel platforms for 3D cell cultures^{8 g-11 g}. Bioadhesive hydrogels incorporate unique peptides to mimic specific ECM proteins^{12 g-14 g}. For example GFOGER peptide (SEQ ID NO: 5) mimics collagen-I as a 3D network distributed across the cell cultures up to 0.5 mm above the glass, providing efficient stem cell growth and differentiation^{14 g}. Co-culture system studies can also benefit from 3D spatial transcriptomics. Co-culture studies allows systematic measurement of cell interactions^{15g,16g}. 3D spatial transcriptomics reveals further details about localized cell interactions beyond bulk measurements.

Graph can represent various forms of data in biological context, such as protein signaling networks and gene regulation networks.^{17 g} Multiple analysis methods have been developed that represent spatial omics data using graphs, then applying graph-based machine learning methods. For example, spaGCN represents spatial transcriptomics data in graph based on spatial distance. A graph convolutional networks integrates spatial transcriptomics and histology information to discover spatial domains^{18 g}. GraphST represents spatial transcriptomics data in nearest-neighbor graphs and applies self-supervised graph neural networks to embed the data into relevant latent features^{19 g}. However, these methods only utilize local gene expression level in spatial transcriptomic data. The subcellular distribution of RNA transcripts was ignored.

To address the above issue, we developed spatially resolved subcellular gene network embedding (3D-SpaGNNE spatially resolved gene neighborhood network embedding). We further advanced the spatially resolved subcellular gene neighborhood network (spaGNN). The RNA transcripts were first detected in 3D location. Each cell is separated into spatially resolved patches using clustering. Then we quantified the pairwise gene proximity relationships within each patch using permutation analysis. The gene proximity relationship can vary throughout the cell. To further extract features from the spatial distribution of gene proximity relationships, we constructed a nearest-neighbor graph between center positions of each spatially resolved patch and applied a graph autoencoder to embed the proximity relationships into a latent space. A graph-pooling layer combines embedded features of each patch in the same cell into a single vector encoding the subcellular gene proximity relationships.

### Results and Discussion

### 3D-SpaGNN-E Distinguishes Tissue Source of Mesenchymal Stem Cells

MSCs can be isolated from varying tissue sources such as bone marrow and umbilical cord. However, different tissue sources of MSCs can introduce heterogeneous immunomodulatory potential. Spatial transcriptomics methods capture dimensions in data that are masked by other analysis methods. 3D-SpaGNN-E analyzes single-transcript RNA-FISH data in MSC cultured in 3D hydrogel environment.

Multiplexed RNA detection experiments were performed in 3D Hydrogels with high stiffness (10 w/v %, PEGMAL) using the sequential labeling, imaging, and re-hybridization strategy. A library of 31 RNA targets 5-day cultured and fixed MSCs located across the 30-μm thick hydrogel layer using up to 100 optical slices on a wide-field microscope. The images provide 3D volumetric distributions of multiple RNA species in a single measurement. Single RNA transcripts were then detected in 3D. SpaGNN pipeline separates cell into spatially distinct patches by applying clustering on the positions of detected RNA transcripts. Within each patch, we established nearest-neighbor networks by connecting transcripts and their surrounding 9 nearest neighbors. A permutation analysis generates the significance of pairwise gene proximity relationships for each patch. By visualizing gene enrichment and pairwise gene proximity relationship in a network, we show subcellularly heterogeneous transcript spatial distribution.

To further extract feature describing the gene proximity relationships in a cell, we applied a graph autoencoder consisting of two layers of convolutional encoder and an inner-product decoder^{20g}. We use the center positions of patches as nodes positions, and the pairwise gene proximity relationships as node features. The nodes related to their three nearest neighbors to form a patch graph. The graph autoencoder was then applied to the patch graph. Each patch was assigned a new set of features. The embedded features of all patches in the same cell were pooled using an average pooling, and the pooled features were used to classify cell types. The embedded features outperform single-cell RNA count using both support vector machine (SVM) and Logistic regression classifiers.

3d-spaGNN-E of MSC-Immune Cell Co-Culture System Correlates Microenvironment with PBMC Expression

MSCs were cultured on poly-1-lysine coated glass coverslips. MSCs were cultured for 24 hours, allowing the MSCs to adhere. The regular culture medium was then changed to culture medium containing 50 ng/mL interferon-γ (IFN-γ). At the same time, human isolated PBMC were thawed from liquid nitrogen, and allowed to rest in culture medium. After 24 hours of PBMC culture and MSC priming with IFN-γ, PBMCs were diluted. The PBMC dilution were mixed with Cultrex gel at 4:1 ratio, and seeded on top of MSCs on the coverslips. The embedded system was then co-cultured in Ionomycin/PMA t-cell activation mix for three days. A multiplexed RNA-HCR was performed on the co-culture gel. To examine the interaction between MSCs and PBMCs, we defined all RNA within 50 μm to a center PBMC as a local RNA environment.

We then analyzed each RNA microenvironment using the 3d-spaGNN-E pipeline. All MSC transcripts were grouped into patches based on spatial location. The permutation analysis computed the pairwise proximity scores. The resulting nearest neighbor graph between patches were then embedded using the same graph autoencoder. An average pooling generates features that encode the gene proximity relationships within the microenvironment.

We then performed regression analysis between the microenvironment features and RNA expression of the center PBMCs. The embedded microenvironment features reduce regression error when predicting expression of MKI67 and IL8 expression of the center PBMC compared to the RNA count of the microenvironment. The embedding features did not outperform RNA count for regression to IL6 or PDL1.

### Diagonal Integration Reduces Experiment Complexity and Time

Image-based spatial transcriptomics poses unique image-processing challenges. Cyclic experiments increase multiplexing capability of RNA-FISH^{2g,3g}. However, experiment and data processing can be complex. Diagonal integration of features and cells can alleviate such challenges^{21g}.

Priming of MSCs can increase the immunosuppressive potential of MSCs^{22g,23g} MSCs were embedded in PEG-MAL hydrogel. The hydrogel was then encapsulated in a PDMS microfluidic chip. The cell culture medium with 50 ng/mL of INF-y were perfused through the microfluidic chip for 24 hours. The gel was then extracted from the microfluidic chip and fixed. The samples were sectioned and immobilized on coverslips for imaging. We performed RNA-HCR on the immobilized samples.

The primed samples show elevated expression of key markers indicating effect of INF-γ, including IL6, IL8, and PDL1. The soft samples limit multiplexing potential.

### Methods

### MSC 3D Hydrogel Assembly

The hydrogel containing 6% 4 arms PEG-MAL was synthesized using PEG-MAL macromer, adhesive peptides, and crosslinkers. PEG-MAL macromers were tethered with a 1:4 ratio of macromers and thiolated GFOGER peptide (SEQ ID NO: 5) at a pH of 7.8 HEPES by incubating them for 30 min 37° C. All components were diluted using PBS++ solution with a pH of 7.4 and 1% HEPES. Covalent conjugation ingredients, a 1:1 mixture of MMP9-degradable VPM peptide to non-degradable DTT molar ratio, were suspended together at a pH of 6.4 HEPES buffer. Cells (UC and BM-MSCs) were dissolved in the crosslinker solution. Hydrogels were crosslinked by a mixture of a 1:1 volume ratio of macromers and crosslinker-cell solutions on the non-treated plate surface. The mixture was mixed rapidly via pipet and cured at 37° C. for 15 min. Post-curing, the cell media, or buffers, were added to each hydrogel assembly. The media was replenished every three days. To assist imaging, the hydrogel is prepared in 96 well plates (Cat #265300).

### 3D Hydrogel Compounds

4 arms (20 kDa, ≥90% purity) and dithiothreitol (DTT) were purchased from Laysan Bio and Sigma Aldrich, respectively. MMP-9 degradable VPM peptide (GCRDVPMSMRGGDRCG, >90% purity—SEQ ID NO: 4) and collagen-mimetic GFOGER peptide (GYGGGP(GPP)5GFOGER(GPP)5GPC, >90% purity—SEQ ID NO: 6) were purchased from AAPPTec (Louisville, KY). All ingredients were reconstituted in 0.01 M 4-(2-hydroxyethyl)-1-piperazineethanesulfonic acid (HEPES) buffer with a pH of 7.4.

### Msc-PBMC Co-Culture System

MSCs were passaged and seeded on ploy-1-lysine coated coverslips. Human INF-γ were diluted to 50 ng/mL in MSC culture medium and used as MSC priming medium. The MSCs were cultured in the priming medium for 24 hours. At the same time, immune cells were thawed and cultured for 24 hours. After 24 hours, immune cells were centrifuged and resuspended in serum-free RPMI 1640 medium. The cell dilution was then mixed with Cultrex gel at 4:1 ratio.

### Statistics

MSC culture: MSC donor 0177 (RoosterBio) was expanded and cryopreserved prior to use in MSC culture media of alpha-MEM (ThermoFisher) with 16.5% MSC-qualified fetal bovine serum (FBS) (ThermoFisher), 2-4 mM L-glutamine (ThermoFisher), 100 U/mL Penicillin (ThermoFisher), and 100 μg/mL Steptomycin (ThermoFisher). Cells were maintained at PDL<20.

Synthesis and set up of on-chip microfluidic devices: The synthesis of the microfluide chips is described in Schneider et al. 2022. In brief, microfluidic chips were cast of polydimethylsiloxane (PDMS, Sylgard 184 Dow Chemical) in an aluminum macro-mold. A steel wire was used to for a microfluidic channel and the material was bonded to a glass slide by oxygen plasma. For experimentation, cross-linked cell-laden hydrogels were placed in the chip device, and the system was sealed by plasma treatment.

The microfluidic chips were connected to syringes secured in PHD Ultra Pumps with attached 6/10 multi-racks (Harvard Apparatus) with PTFE tubing (#30 AWG, Cole Parmer), PE/PVC tubing adaptors (0.024×0.064 in, Instech Labs) and 20-gauge blunt tip needles (industrial Dispensing Tips) and perfused at 1.0 μL min⁻¹ for 24 hr with MCS culture medium with 50 ng/mL IFN-γ (Biolegend). At that time, the hydrogels were removed and prepared for imaging.

Hydrogel synthesis: As previously reported in Schneider et al. 2022, MSC cells were encapsulated within on-chip hydrogels as outlined here. 20 kDa 4-arm maleimide-functionalized poly(ethylene-glycol) (PEG-4MAL, Laysan Bio) was dissolved in PBS and mixed with 5 mM adhesive peptide RGD (GRGDSPC, Genscript—SEQ ID NO: 7) at a 2:1 ratio. Crosslinkers VPM (GCRDVPMSMRGGDRCG, Genscript—SEQ ID NO: 4) and dithiothreitol (DTT, Sigma-Aldrich) were dissolved at 24.8 nM and mixed 80:20 ratio. PEG-4MAL and RGD components were allowed to functionalize at room temperature (RT) for 30 min. Cells were lifted, counted, and added to the PEG-4MAL/RGD solution for a final concentration of 40,000 cells/gel (2,000 cells/uL) in a 3:1 ratio. Hydrogel gelation occurred by mixing PEG-4MAL+RGD+cell solution and crosslinking solution at 4:1 vol ratio for a 20 μL hydrogel onto a sterilized hydrophobic surface.

Other advantages which are obvious, and which are inherent to the invention, will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

### REFERENCES

1a. Frantz, C. et al. J Cell Sci 123, 4195-4200 (2010).
2a. Daley, W. P. et al. J Cell Sci 121, 255-264 (2008).
3a. Naba, A. et al. Matrix Biol 49, 10-24 (2016).
4a. Sonbol, H. S. J Microsc Ultrastruct 6, 123-128 (2018).
5a. Mao, A. S. et al. PNAS 112, 14452-14459 (2015).
6a. Prestwich, G. D. et al. Expert Opin Biol Ther 15, 3-7 (2015).
7a. Han, Y. et al. Sig Transduct Target Ther 7, 1-19 (2022).
8a. Sorrell, J. M. et al. Tissue Eng. Part A 15, 1751-1761 (2009).
9a. Rusch, R. M. et al. Int J Mol Sci 22, 4895 (2021).
10a. Lee, M.-H. et al. J Cell Physiol 235, 3874-3885 (2020).
11a. Cai, L. et al. Expert Rev Gastroenterol Hepatol 3, 1-3 (2009).
12a. Patel, D. et al. Bio. Biophys Res Commun 499, 642-647 (2018).
13a. Bardsley, K. et al. Tissue Eng Part C Meth. 23, 228-236 (2017).
14a. Anderson, L. E. et al. Ann Biomed Eng 49, 3698-3710 (2021).
15a. Salem, E. S. B. et al. Frontiers in Cardio. Medicine 6, (2019).
16a. McHugh, C. A. et al. Genome Biology 15, 203 (2014).
17a. Corley, M. et al. Molecular Cell 78, 9-29 (2020).
18a. Ramanathan, M. et al. Nat Methods 16, 225-234 (2019).
19a. Marchese, D. et al. WIREs RNA 7, 793-810 (2016).
20a. Kaewsapsak, P. et al. eLife 6, e29224 (2017).
21a. Eliscovich, C. et al. PNAS 114, E1875-E1884 (2017).
22a. Duan, N. et al. Nucleic Acids Research 49, e107 (2021).
23a. Huppertz, I. et al. Methods 65, 274-287 (2014).
24a. Porter, D. F. et al. Nat Commun 12, 1569 (2021).
25a. Hafner, M. et al. Nat Rev Methods Primers 1, 1-23 (2021).
26a. Van Nostrand, E. L. et al. Nat Methods 13, 508-514 (2016).
27a. Lang, B. et al. Nucleic Acids Res 49, 6702-6721 (2021).
28a. Castello, A. et al. Mol Cell 63, 696-710 (2016).
29a. Brannan, K. W. et al. Nat Methods 18, 507-519 (2021).
30a. Qin, W. et al. Nat Commun 12, 4980 (2021).
31a. Huranovi, M. et al. RNA 15, 2063-2071 (2009).
32a. Patel, V. L. et al. Genes Dev. 26, 43-53 (2012).
33a. Stefanovic, B. WIREs RNA 4, 535-545 (2013).
34a. Stefanovic, B. et al. Mol Cell Biol 17, 5201-5209 (1997).
35a. Zuo, S. et al. The EMBO Journal 40, e107403 (2021).
36a. Stefanovic, L. et al. J Mol Biol 434, 167394 (2022).
37a. Chen, H.-S. et al. Cell Transplant 22, 677-684 (2013).
38a. Awad, H. A. et al. Tissue Engineering 5, 267-277 (1999).
39a. Choi, H. M. T. et al. Development 145, dev165753 (2018).
40a. Lubeck, E. et al. Nat Methods 11, 360-361 (2014).
41a. Fang, Z. et al. BioRxiv DOI: 10.1101/2022.08.03.502409 (2022).
42a. Xia, C. et al. PNAS 116, 19490-19499 (2019).
43a. McCaughey, J. et al. Journal of Cell Biology 218, 929-948 (2019).
44a. Zhang, W. et al. RNA 22, 1785-1792 (2016).
45a. Söderberg, O. et al. Nat Methods 3, 995-1000 (2006).
46a. Doroski, D. M. et al. Tissue Eng Part A 16, 3457-3466 (2010).
47a. Ogle, M. E et al. Tissue Eng Part A 26, 1259-1271 (2020).
48a. Qiu, Y. et al. J Tissue Eng Regen Med 10, 989-999 (2016).
49a. Deng, X. et al. Angewandte Chemie Int Ed, 23289-23298 (2021).
50a. Reddish, F. N. et al. iScience 24, 102129 (2021).
51a. Coskun, A. F et al. Nature Methods 13, 657-660 (2016).
52a. Mosquera, M. J. et al. Advanced Materials 34, 2100096 (2022).
53a. Tanaka, T. et al. Life Science Alliance 5, (2022).
54a. Chen, B.-C. et al. Science 346, 1257998 (2014).
55a. Cho, W. S. et al. Ann Rehabil Med 45, 274-283 (2021).
56a. Trevino, E. A. et al. J Orthop Res 37, 490-502 (2019).
57a. Baker, E. A. G. et al. BioRxiv DOI: 10.1101/2022.01.26.477748 (2022).
58a. Parks, A. N. et al. J Orthop Res 35, 1910-1918 (2017).
59a. Stefanovic, B. et al. Sci Rep 9, 326 (2019).
60a. Roy, S. et al. J Biol Chem 295, 15013-15028 (2020).
61a. Carter, E. M. et. al. Cur. Op. in Ped. 21, 46-54 (2009).
62a. Fujii, K. K. et al. Sci Rep 9, 17433 (2019).
63a. Ganesh, S. et al. Science Advances 7, eabd0957 (2021).
1b. Guess, A. J. et al. Safety Profile of Good Manufacturing Practice Manufactured Interferon γ-Primed Mesenchymal Stem/Stromal Cells for Clinical Trials. Stem Cells Translational Medicine 6, 1868-1879 (2017).
2b. Schneider, R. S. et al. High-Throughput On-Chip Human Mesenchymal Stromal Cell 50 Potency Prediction. Advanced Healthcare Materials 11, 2101995 (2022).
3b. Lubeck, E., Coskun, A. F., Zhiyentayev, T., Ahmad, M. & Cai, L. Single cell in situ RNA profiling by sequential hybridization. Nat Methods 11, 360-361 (2014).
4b. Coskun, A. F. & Cai, L. Dense transcript profiling in single cells by image correlation decoding I. Nature Methods 13, 657-660 (2016).
5b. Cai, S. et al. Multiplexed protein profiling reveals spatial subcellular signaling networks. iScience 25, 104980 (2022).
6b. Venkatesan, M. et al. Virtual and augmented reality for biomedical applications. CR Med 2, (2021).
7b. Burnham, A. J., Daley-Bauer, L. P. & Horwitz, E. M. Mesenchymal stromal cells in hematopoietic cell transplantation. Blood Advances 4, 5877-5887 (2020).
8b. Dominici, M. et al. Minimal criteria for defining multipotent mesenchymal stromal cells. The International Society for Cellular Therapy position statement. Cytotherapy 8, 315-317 (2006).
9b. Horwitz, E. Interferon γ-Primed Mesenchymal Stromal Cells as Prophylaxis for Acute Graft v Host Disease After Allogeneic Hematopoietic Cell Transplantation for Patients With Hematologic Malignancies and Myelodysplasia. https://clinicaltrials.gov/ct2/show/NCT04328714 (2022).
10b. Noronha, N. de C. et al. Priming approaches to improve the efficacy of mesenchymal stromal cell-based therapies. Stem Cell Research & Therapy 10, 131 (2019).
11b. Wobma, H. M. et al. The influence of hypoxia and IFN-γ on the proteome and metabolome of therapeutic mesenchymal stem cells. Biomaterials 167, 226-234 (2018).
12b. Kehl, D. et al. Proteomic analysis of human mesenchymal stromal cell secretomes: a systematic comparison of the angiogenic potential. npj Regenerative Medicine 4, 1-13 (2019).
13b. Lamas, J. R. et al. RNA sequencing of mesenchymal stem cells reveals a blocking of differentiation and immunomodulatory activities under inflammatory conditions in rheumatoid arthritis patients. Arthritis Research & Therapy 21, 112 (2019).
14b. Carrow, J. K. et al. Widespread changes in transcriptome profile of human mesenchymal stem cells induced by two-dimensional nanosilicates. PNAS 115, E3905-E3913 (2018).
15b. Riis, S. et al. Mass spectrometry analysis of adipose-derived stem cells reveals a significant effect of hypoxia on pathways regulating extracellular matrix. Stem Cell Research & Therapy 7, 52 (2016).
16b. Lu, S. & Qiao, X. Single-cell profiles of human bone marrow-derived mesenchymal stromal cells after IFN-γ and TNF-α licensing. Gene 771, 145347 (2021).
17b. Huang, Y. et al. Single cell transcriptomic analysis of human mesenchymal stem cells reveals limited heterogeneity. Cell Death & Disease 10, 1-12 (2019).
18b. Sun, C. et al. Single-cell RNA-seq highlights heterogeneity in human primary Wharton's jelly mesenchymal stem/stromal cells cultured in vitro. Stem Cell Research & Therapy 11, 149 (2020).
19b. Wang, Z. et al. Single-cell RNA sequencing deconvolutes the in vivo heterogeneity of human bone marrow-derived mesenchymal stem cells. bioRxiv 2020.04.06.027904 (2020) doi:10.1101/2020.04.06.027904.
20b. Medrano-Trochez, C. et al. Single-Cell RNAseq of Out-of-Thaw Mesenchymal Stromal Cells Shows Striking Tissue-of-Origin Differences and Inter-donor Cell-Cycle Variations. bioRxiv 2020.09.10.290155 (2020) doi:10.1101/2020.09.10.290155.
21b. Fang, Z. et al. Subcellular spatially resolved gene neighborhood networks in single cells. 2022.08.03.502409 Preprint at https://doi.org/10.1101/2022.08.03.502409 (2022).
22b. Laurent, A. et al. ChIP-Seq and RNA-Seq Analyses Identify Components of the Wnt and Fgf Signaling Pathways as Prep1 Target Genes in Mouse Embryonic Stem Cells. PLOS ONE 10, e0122518 (2015).
23b. Eng, C.-H. L. et al. Transcriptome-scale super-resolved imaging in tissues by RNA seqFISH. Nature 568, 235-239 (2019).
24b. Xia, C., Fan, J., Emanuel, G., Hao, J. & Zhuang, X. Spatial transcriptome profiling by MERFISH reveals subcellular RNA compartmentalization and cell cycle-dependent gene expression. PNAS 116, 19490-19499 (2019).
25b. Lin, J.-R. et al. Highly multiplexed immunofluorescence imaging of human tissues and tumors using t-CyCIF and conventional optical microscopes. eLife 7, e31657 (2018).
26b. Goltsev, Y. et al. Deep Profiling of Mouse Splenic Architecture with CODEX Multiplexed Imaging. Cell 174, 968-981.e15 (2018).
27b. Giesen, C. et al. Highly multiplexed imaging of tumor tissues with subcellular resolution by mass cytometry I. Nature Methods 11, 417-422 (2014).
28b. Keren, L. et al. MIBI-TOF: A multiplexed imaging platform relates cellular phenotypes and tissue structure. Science Advances 5, eaax5851 (2019).
29b. Hartmann, F. J. et al. Single-cell metabolic profiling of human cytotoxic T cells. Nat Biotechnol (2020) doi:10.1038/s41587-020-0651-8.
30b. Pittenger, M. F. et al. Mesenchymal stem cell perspective: cell biology to clinical progress. npj Regenerative Medicine 4, 1-15 (2019).
31b. Marks, P. & Gottlieb, S. Balancing Safety and Innovation for Cell-Based Regenerative Medicine. New England Journal of Medicine 378, 954-959 (2018).
32b. Lv, F.-J., Tuan, R. S., Cheung, K. M. C. & Leung, V. Y. L. Concise Review: The Surface Markers and Identity of Human Mesenchymal Stem Cells. STEM CELLS 32, 1408-1419 (2014).
33b. Stihl, P. L. et al. Visualization and analysis of gene expression in tissue sections by spatial transcriptomics. Science 353, 78-82 (2016).
34b. Zhu, Q., Shah, S., Dries, R., Cai, L. & Yuan, G.-C. Identification of spatially associated subpopulations by combining scRNA-seq and sequential fluorescence in situ hybridization data. Nat Biotechnol (2018) doi:10.1038/nbt.4260.
35b. Berglund, E. et al. Spatial maps of prostate cancer transcriptomes reveal an unexplored landscape of heterogeneity. Nat Commun 9, 2419 (2018).
36b. Galipeau, J. et al. International Society for Cellular Therapy perspective on immune functional assays for mesenchymal stromal cells as potency release criterion for advanced phase clinical trials. Cytotherapy 18, 151-159 (2016).
37b. Allam, M. et al. Spatially variant immune infiltration scoring in human cancer tissues. npj Precis. Onc. 6, 1-21 (2022).
38b. Ganesh, S. et al. Spatially resolved 3D metabolomic profiling in tissues. Science Advances 7, eabd0957 (2021).
39b. Coskun, A. F. et al. Nanoscopic subcellular imaging enabled by ion beam tomography. Nature Communications 12, 789 (2021).
40b. Schneider, R. S. et al. High-Throughput On-Chip Human Mesenchymal Stromal Cell Potency Prediction. Advanced Healthcare Materials n/a, 2101995.
41b. Clark, A. Y. et al. Integrin-specific hydrogels modulate transplanted human bone marrow-derived mesenchymal stem cell survival, engraftment, and reparative activities. Nat Commun 11, 114 (2020).
42b. Burnham, A. J., Foppiani, E. M. & Horwitz, E. M. Key Metabolic Pathways in MSC-Mediated Immunomodulation: Implications for the Prophylaxis and Treatment of Graft Versus Host Disease. Front Immunol 11, 609277 (2020).
43b. Zhang, X., Xu, C. & Yosef, N. Simulating multiple faceted variability in single cell RNA sequencing. Nat Commun 10, 2611 (2019).
44b. Zhang, Z. & Zhang, X. Inference of high-resolution trajectories in single-cell RNA-seq data by using RNA velocity. Cell Reports Methods 1, 100095 (2021).
45b. Bloom, D. D. et al. A reproducible immunopotency assay to measure mesenchymal stromal cell-mediated T-cell suppression. Cytotherapy 17, 140-151 (2015).
46b. Cruz-Acuña, R. et al. PEG-4MAL hydrogels for human organoid generation, culture, and in vivo delivery. Nat Protoc 13, 2102-2119 (2018).
47b. Park, C. W. et al. Cytokine Secretion Profiling of Human Mesenchymal Stem Cells by Antibody Array. Int J Stem Cells 2, 59-68 (2009).
48b. Cruz-Barrera, M. et al. Integrated Analysis of Transcriptome and Secretome From Umbilical Cord Mesenchymal Stromal Cells Reveal New Mechanisms for the Modulation of Inflammation and Immune Activation. Front Immunol 11, (2020).
49b. Cuerquis, J. et al. Human mesenchymal stromal cells transiently increase cytokine production by activated T cells before suppressing T-cell proliferation: effect of interferon-y and tumor necrosis factor-α stimulation. Cytotherapy 16, 191-202 (2014).
50b. Zhang, T. et al. Bone marrow-derived mesenchymal stem cells promote growth and angiogenesis of breast and prostate tumors. Stem Cell Res Ther 4, 70 (2013).
51b. Choi, H. M. T. et al. Third-generation in situ hybridization chain reaction: multiplexed, quantitative, sensitive, versatile, robust. Development 145, (2018).
52b. SchUrch, C. M. et al. Coordinated Cellular Neighborhoods Orchestrate Antitumoral Immunity at the Colorectal Cancer Invasive Front. Cell 182, 1341-1359.e19 (2020).
53b. Mosquera, M. J. et al. Extracellular Matrix in Synthetic Hydrogel-Based Prostate Cancer Organoids Regulate Therapeutic Response to EZH2 and DRD2 Inhibitors. Advanced Materials n/a, 2100096.
54b. Murali, V. S. et al. An image-based assay to quantify changes in proliferation and viability upon drug treatment in 3D microenvironments. BMC Cancer 19, 502 (2019).
55b. Cruz-Acuña, R. et al. Synthetic hydrogels for human intestinal organoid generation and colonic wound repair. Nat Cell Biol 19, 1326-1335 (2017).
56b. Xue, Q. et al. Analysis of single-cell cytokine secretion reveals a role for paracrine signaling in coordinating macrophage responses to TLR4 stimulation. Sci Signal 8, ra59 (2015).
57b. Stuart, T. et al. Comprehensive Integration of Single-Cell Data. Cell 177, 1888-1902.e21 (2019).
58b. Wiese, D. M. & Braid, L. R. Transcriptome profiles acquired during cell expansion and licensing validate mesenchymal stromal cell lineage genes. Stem Cell Research & Therapy 11, 357 (2020).
59b. Hao, Y. et al. Integrated analysis of multimodal single-cell data. Cell 184, 3573-3587.e29 (2021).
60b. Bodein, A., Scott-Boyer, M.-P., Perin, O., Lê Cao, K.-A. & Droit, A. Interpretation of network-based integration from multi-omics longitudinal data. Nucleic Acids Research gkab1200 (2021) doi:10.1093/nar/gkab1200.
61b. Picard, M., Scott-Boyer, M.-P., Bodein, A., Périn, O. & Droit, A. Integration strategies of multi-omics data for machine learning analysis. Computational and Structural Biotechnology Journal 19, 3735-3746 (2021).
62b. Subramanian, I., Verma, S., Kumar, S., Jere, A. & Anamika, K. Multi-omics Data Integration, Interpretation, and Its Application. Bioinform Biol Insights 14, 1177932219899051(2020).
63b. Galipeau, J. & Sensébé, L. Mesenchymal Stromal Cells: Clinical Challenges and 50 Therapeutic Opportunities. Cell Stem Cell 22, 824-833 (2018).
64b. de Witte, S. F. H. et al. Immunomodulation By Therapeutic Mesenchymal Stromal Cells (MSC) Is Triggered Through Phagocytosis of MSC By Monocytic Cells. Stem Cells 36, 602-615 (2018).
65b. de Luis Balaguer, M. A. et al. Multi-sample Arabidopsis Growth and Imaging Chamber (MAGIC) for long term imaging in the ZEISS Lightsheet Z.1. Developmental Biology 419, 19-25 (2016).
66b. Lallemant, L., Lebreton, C. & Garfa-Traord, M. Comparison of different clearing and acquisition methods for 3D imaging of murine intestinal organoids. J Biol Methods 7, e141 (2020).
67b. Chen, B.-C. et al. Lattice light-sheet microscopy: Imaging molecules to embryos at high spatiotemporal resolution. Science 346, 1257998 (2014).
68b. Mir, M. et al. Single Molecule Imaging in Live Embryos Using Lattice Light-Sheet Microscopy. Methods Mol Biol 1814, 541-559 (2018).
69b. Mir, M. et al. Dense Bicoid hubs accentuate binding along the morphogen gradient. Genes Dev 31, 1784-1794 (2017).
1c. Hughes, S. C., and Simmonds, A. J. (2019). Drosophila mRNA localization during later development: past, present, and future. Front. Genet. 10, 135. https://doi.org/10.3389/fgene.2019.00135.
2c. Hobson, B. D., Kong, L., Angelo, M. F., Lieberman, O. J., Mosharov, E. V., Herzog, E., Sulzer, D., and Sims, P. A. (2022). Subcellular and regional localization of mRNA translation in midbrain dopamine neurons. Cell Rep. 38, 110208. https://doi.org/10.1016/j.celrep.2021.110208.
3c. Bashirullah, A., Cooperstock, R. L., and Lipshitz, H. D. (1998). Rna localization in development. Annu. Rev. Biochem. 67, 335-394. https://doi.org/10. 1146/annurev.biochem.67.1.335.
4c. Samacoits, A., Chouaib, R., Safieddine, A., Traboulsi, A.-M., Ouyang, W., Zimmer, C., Peter, M., Bertrand, E., Walter, T., and Mueller, F. (2018). A computational framework to study sub-cellular RNA localization. Nat. Commun. 9, 4584. https://doi.org/10.1038/s41467-018-06868-w.
5c. Stahl, P. L., Salmén, F., Vickovic, S., Lundmark, A., Navarro, J. F., Magnusson, J., Giacomello, S., Asp, M., Westholm, J. O., Huss, M., et al. (2016). Visualization and analysis of gene expression in tissue sections by spatial transcriptomics. Science 353, 78-82. https://doi.org/10.1126/science. aaf2403.
6c. Rodriques, S. G., Stickels, R. R., Goeva, A., Martin, C. A., Murray, E., Van-derburg, C. R., Welch, J., Chen, L. M., Chen, F., and Macosko, E. Z. (2019). Slide-seq: a scalable technology for measuring genome-wide expression at high spatial resolution. Science 363, 1463-1467. https://doi.org/10.1126/science.aawl219.
7c. Eng, C.-H. L., Lawson, M., Zhu, Q., Dries, R., Koulena, N., Takei, Y., Yun, J., Cronin, C., Karp, C., Yuan, G.-C., and Cai, L. (2019). Transcriptome-scale super-resolved imaging in tissues by RNA seqFISH+. Nature 568, 235-239. https://doi.org/10.1038/s41586-019-1049-y.
8c. Takei, Y., Yun, J., Zheng, S., Ollikainen, N., Pierson, N., White, J., Shah, S., Thomassie, J., Suo, S., Eng, C.-H. L., et al. (2021). Integrated spatial genomics reveals global architecture of single nuclei. Nature 590, 344-350. https://doi.org/10.1038/s41586-020-03126-2.
9c. Xia, C., Fan, J., Emanuel, G., Hao, J., and Zhuang, X. (2019). Spatial transcriptome profiling by MERFISH reveals subcellular RNA compartmentalization and cell cycle-dependent gene expression. Proc. Natl. Acad. Sci. USA 116, 19490-19499. https://doi.org/10.1073/pnas.1912459116.
10c. Moffitt, J. R., Bambah-Mukku, D., Eichhorn, S. W., Vaughn, E., Shekhar, K., Perez, J. D., Rubinstein, N. D., Hao, J., Regev, A., Dulac, C., and Zhuang, X. (2018). Molecular, spatial, and functional single-cell profiling of the hypothalamic preoptic region. Science 362, eaau5324. https://doi.org/10. 1126/science.aau5324.
11c. Buxbaum, A. R., Haimovich, G., and Singer, R. H. (2015). In the right place at the right time: visualizing and understanding mRNA localization. Nat. Rev. Mol. Cell Biol. 16, 95-109. https://doi.org/10.1038/nrm3918.
12c. Imboden, S., Liu, X., Lee, B. S., Payne, M. C., Hsieh, C.-J., and Lin, N. Y. C. (2021). Investigating heterogeneities of live mesenchymal stromal cells using AI-based label-free imaging. Sci. Rep. 11, 6728. https://doi.org/10. 1038/s41598-021-85905-z.
13c. Katz, Z. B., Wells, A. L., Park, H. Y., Wu, B., Shenoy, S. M., and Singer, R. H. (2012). b-Actin mRNA compartmentalization enhances focal adhesion stability and directs cell migration. Genes Dev. 26, 1885-1890. https://doi. org/10.1101/gad.190413.112.
14c. Guil, S., and Esteller, M. (2015). RNA-RNA interactions in gene regulation: the coding and noncoding players. Trends Biochem. Sci. 40, 248-256. https://doi.org/10.1016/j.tibs.2015.03.001.
15c. Hartswood, E., Brodie, J., Vendra, G., Davis, I., and Finnegan, D. J. (2012). RNA:RNA interaction can enhance RNA localization in Drosophila oocytes. RNA 18, 729-737. https://doi.org/10.1261/rna.026674.111.
16c. Kitano, H. (2002). Systems biology: a brief overview. Science 295, 1662-1664. https://doi.org/10.1126/science.1069492.
17c. Yáñez-Cuna, J. O., Kvon, E. Z., and Stark, A. (2013). Deciphering the transcriptional cis-regulatory code. Trends Genet. 29, 11-22. https://doi.org/10.1016/j.tig.2012.09.007.
18c. Mercatelli, D., Scalambra, L., Triboli, L., Ray, F., and Giorgi, F. M. (2020). Gene regulatory network inference resources: a practical overview. Biochim. Biophys. Acta. Gene Regul. Mech. 1863, 194430. https://doi.org/10.1016/j.bbagrm.2019.194430.
19c. Kulkarni, S. R., Vaneechoutte, D., Van de Velde, J., and Vandepoele, K. (2018). TF2Network: predicting transcription factor regulators and gene regulatory networks in Arabidopsis using publicly available binding site information. Nucleic Acids Res. 46, e31. https://doi.org/10.1093/nar/gkx1279.
20c. Liu, Z., Gao, J., Li, T., Jing, Y., Xu, C., Zhu, Z., Zuo, D., and Chen, J. (2022). A novel approach GRNTSTE to reconstruct gene regulatory interactions applied to a case study for rat pineal rhythm gene. Sci. Rep. 12, 10227. https://doi.org/10.1038/s41598-022-14903-6.
21c. Huynh-Thu, V. A., Irrthum, A., Wehenkel, L., and Geurts, P. (2010). Inferring regulatory networks from expression data using tree-based methods. PLoS One 5, e12776. https://doi.org/10.1371/journal.pone.0012776.
22c. Stuart, J. M., Segal, E., Koller, D., and Kim, S. K. (2003). A gene-coexpression network for global discovery of conserved genetic modules. Science 302, 249-255. https://doi.org/10.1126/science.1087447.
23c. Tang, J., Kong, D., Cui, Q., Wang, K., Zhang, D., Gong, Y., and Wu, G. (2018). Prognostic genes of breast cancer identified by gene Co-expression network analysis. Front. Oncol. 8, 374. https://doi.org/10.3389/fonc.2018.00374.
24c. Verfaillie, A., Imrichova, H., Janky, R., and Aerts, S. (2015). iRegulon and icisTarget: reconstructing regulatory networks using motif and track enrichment. Curr. Protoc. Bioinformatics 52, 2.16.1-2.16.39. https://doi. org/10.1002/0471250953.bi0216s52.
25c. Bailey, T. L., Boden, M., Buske, F. A., Frith, M., Grant, C. E., Clementi, L., Ren, J., Li, W. W., and Noble, W. S. (2009). MEME SUITE: tools for motif discovery and searching. Nucleic Acids Res. 37, W202-W208. https://doi. org/10.1093/nar/gkp335.
26c. Yang, Y., Fang, Q., and Shen, H.-B. (2019). Predicting gene regulatory interactions based on spatial gene expression data and deep learning. PLoS Comput. Biol. 15, e1007324. https://doi.org/10.1371/journal.pcbi. 1007324.
27c. Puniyani, K., and Xing, E. P. (2013). GINI: from ISH images to gene interaction networks. PLoS Comput. Biol. 9, e1003227. https://doi.org/10.1371/journal.pcbi.1003227.
28c. Banf, M., and Rhee, S. Y. (2017). Computational inference of gene regulatory networks: approaches, limitations and opportunities. Biochim. Biophys. Acta. Gene Regul. Mech. 1860, 41-52. https://doi.org/10.1016/j. bbagrm.2016.09.003.
29c. Traag, V. A., Waltman, L., and van Eck, N. J. (2019). From Louvain to Leiden: guaranteeing well-connected communities. Sci. Rep. 9, 5233. https://doi.org/10.1038/s41598-019-41695-z.
30c. Chen, K. H., Boettiger, A. N., Moffitt, J. R., Wang, S., and Zhuang, X. (2015). Spatially resolved, highly multiplexed RNA profiling in single cells. Science 348, aaa6090. https://doi.org/10.1126/science.aaa6090.
31c. Hagberg, A., Swart, P., and S Chult, D. (2008). Exploring Network Structure, Dynamics, and Function Using NetworkX (Los Alamos National Lab.(LANL)).
32c. Mah, C. K., Ahmed, N., Lam, D., Monell, A., Kern, C., Han, Y., Cesnik, A. J., Lundberg, E., Zhu, Q., Carter, H., et al. (2022). Bento: a toolkit for subcellular analysis of spatial transcriptomics data. Bioinformatics. https://doi. org/10.1101/2022.06.10.495510.
33c. Marks, P., and Gottlieb, S. (2018). Balancing safety and innovation for cell-based regenerative medicine. N. Engl. J. Med. 378, 954-959.
34c. Ankrum, J., and Karp, J. M. (2010). Mesenchymal stem cell therapy: two steps forward, one step back. Trends Mol. Med. 16, 203-209.
35c. Pittenger, M. F., Discher, D. E., Péault, B. M., Phinney, D. G., Hare, J. M., and Caplan, A. I. (2019). Mesenchymal stem cell perspective: cell biology to clinical progress. NPJ Regen. Med. 4, 22. https://doi.org/10.1038/s41536-019-0083-6.
36c. Kern, S., Eichler, H., Stoeve, J., Klu€ter, H., and Bieback, K. (2006). Comparative analysis of mesenchymal stem cells from bone marrow, umbilical cord blood, or adipose tissue. Stem cells 24, 1294-1301.
37c. Mattar, P., and Bieback, K. (2015). Comparing the immunomodulatory properties of bone marrow, adipose tissue, and birth-associated tissue mesenchymal stromal cells. Front. Immunol. 6, 560. https://doi.org/10. 3389/fimmu.2015.00560.
38c. Zhong, L., Yao, L., Tower, R. J., Wei, Y., Miao, Z., Park, J., Shrestha, R., Wang, L., Yu, W., Holdreith, N., et al. (2020). Single cell transcriptomics identifies a unique adipose lineage cell population that regulates bone marrow environment. Elife 9, e54695.
39c. Zha, K., Li, X., Yang, Z., Tian, G., Sun, Z., Sui, X., Dai, Y., Liu, S., and Guo, Q. (2021). Heterogeneity of mesenchymal stem cells in cartilage regeneration: from characterization to application. NPJ Regen. Med. 6, 14-15.
40c. Aijaz, A., Li, M., Smith, D., Khong, D., LeBlon, C., Fenton, O. S., Olabisi, R. M., Libutti, S., Tischfield, J., Maus, M. V., et al. (2018). Biomanufacturing for clinically advanced cell therapies. Nat. Biomed. Eng. 2, 362-376.
41c. Hoogduijn, M. J., Roemeling-van Rhijn, M., Korevaar, S. S., Engela, A. U., Weimar, W., and Baan, C. C. (2011). Immunological aspects of allogeneic and autologous mesenchymal stem cell therapies. Hum. Gene Ther. 22, 1587-1591.
42c. Rennerfeldt, D. A., and Van Vliet, K. J. (2016). Concise review: when colonies are not clones: evidence and implications of intracolony heterogeneity in mesenchymal stem cells. Stem cells 34, 1135-1141.
43c. Lv, F.-J., Tuan, R. S., Cheung, K. M. C., and Leung, V. Y. L. (2014). Concise review: the surface markers and identity of human mesenchymal stem cells. Stem cells 32, 1408-1419.
44c. Dominici, M., Le Blanc, K., Mueller, I., Slaper-Cortenbach, I., Marini, F., Krause, D., Deans, R., Keating, A., Prockop, D., and Horwitz, E. (2006). Minimal criteria for defining multipotent mesenchymal stromal cells. The International Society for Cellular Therapy position statement. Cytotherapy 8, 315-317.
45c. Cho, K.-A., Park, M., Kim, Y.-H., Woo, S.-Y., and Ryu, K.-H. (2017). RNA sequencing reveals a transcriptomic portrait of human mesenchymal stem cells from bone marrow, adipose tissue, and palatine tonsils. Sci. Rep. 7, 17114-17119.
46c. Lamas, J. R., Fernandez-Gutierrez, B., Mucientes, A., Marco, F., Lopiz, Y., Jover, J. A., Abasolo, L., and Rodríguez-Rodríguez, L. (2019). RNA sequencing of mesenchymal stem cells reveals a blocking of differentiation and immunomodulatory activities under inflammatory conditions in rheumatoid arthritis patients. Arthritis Res. Ther. 21, 112.
47c. Kehl, D., Generali, M., Mallone, A., Heller, M., Uldry, A.-C., Cheng, P., Gantenbein, B., Hoerstrup, S. P., and Weber, B. (2019). Proteomic analysis of human mesenchymal stromal cell secretomes: a systematic comparison of the angiogenic potential. NPJ Regen. Med. 4, 8-13.
48c. Sun, C., Wang, L., Wang, H., Huang, T., Yao, W., Li, J., and Zhang, X. (2020). Single-cell RNA-seq highlights heterogeneity in human primary Wharton's jelly mesenchymal stem/stromal cells cultured in vitro. Stem Cell Res. Ther. 11, 149. https://doi.org/10.1186/s13287-020-01660-4.
49c. Zheng, G., Xie, Z.-Y., Wang, P., Wu, Y.-F., and Shen, H.-Y. (2020). Recent advances of single-cell RNA sequencing technology in mesenchymal stem cell research. WJSC 12, 438-447. https://doi.org/10.4252/wjsc. v12.i6.438.
50c. Zhou, W., Lin, J., Zhao, K., Jin, K., He, Q., Hu, Y., Feng, G., Cai, Y., Xia, C., Liu, H., et al. (2019). Single-cell profiles and clinically useful properties of human mesenchymal stem cells of adipose and bone marrow origin. Am. J. Sports Med. 47, 1722-1733. https://doi.org/10.1177/0363546519848678.
51c. Zhang, S., Wang, J. Y., Li, B., Yin, F., and Liu, H. (2021). Single-cell transcriptome analysis of uncultured human umbilical cord mesenchymal stem cells. Stem Cell Res. Ther. 12, 25. https://doi.org/10.1186/s13287-020-02055-1.
52c. Oja, S., Komulainen, P., Penttilä, A., Nystedt, J., and Korhonen, M. (2018). Automated image analysis detects aging in clinical-grade mesenchymal stromal cell cultures. Stem Cell Res. Ther. 9, 6. https://doi.org/10.1186/s13287-017-0740-x.
53c. Sasaki, H., Takeuchi, I., Okada, M., Sawada, R., Kanie, K., Kiyota, Y., Honda, H., and Kato, R. (2014). Label-free morphology-based prediction of multiple differentiation potentials of human mesenchymal stem cells for early evaluation of intact cells. PLoS One 9, e93952. https://doi.org/10.1371/journal.pone.0093952.
54c. Lee, Y. J., Vega, S. L., Patel, P. J., Aamer, K. A., Moghe, P. V., and Cicerone, M. T. (2014). Quantitative, label-free characterization of stem cell differentiation at the single-cell level by broadband coherent anti-Stokes Raman scattering microscopy. Tissue Eng. C Methods 20, 562-569. https://doi. org/10.1089/ten.tec.2013.0472.
55c. Choi, H. M. T., Schwarzkopf, M., Fornace, M. E., Acharya, A., Artavanis, G., Stegmaier, J., Cunha, A., and Pierce, N. A. (2018). Third-generation in situ hybridization chain reaction: multiplexed, quantitative, sensitive, versatile, robust. Development 145, dev165753. https://doi.org/10.1242/dev. 165753.
56c. Golkaram, M., Jang, J., Hellander, S., Kosik, K. S., and Petzold, L. R. (2017). The role of chromatin density in cell population heterogeneity during stem cell differentiation. Sci. Rep. 7, 13307. https://doi.org/10.1038/s41598-017-13731-3.
57c. Ricci, M. A., Cosma, M. P., and Lakadamyali, M. (2017). Super resolution imaging of chromatin in pluripotency, differentiation, and reprogramming. Curr. Opin. Genet. Dev. 46, 186-193. https://doi.org/10.1016/j.gde.2017. 07.010.
58c. Ainsley, J. A., Drane, L., Jacobs, J., Kittelberger, K. A., and Reijmers, L. G. (2014). Functionally diverse dendritic mRNAs rapidly associate with ribosomes following a novel experience. Nat. Commun. 5, 4510. https://doi. org/10.1038/ncomms5510.
59c. McCaughey, J., Stevenson, N. L., Cross, S., and Stephens, D. J. (2019). ER-to-Golgi trafficking of procollagen in the absence of large carriers. J. Cell Biol. 218, 929-948. https://doi.org/10.1083/jcb.201806035.
60c. Park, C. W., Kim, K.-S., Bae, S., Son, H. K., Myung, P.-K., Hong, H. J., and Kim, H. (2009). Cytokine secretion profiling of human mesenchymal stem cells by antibody array. Int. J. Stem Cells 2, 59-68.
61c. Cruz-Barrera, M., Flórez-Zapata, N., Lemus-Diaz, N., Medina, C., Galindo, C.-C., González-Acero, L. X., Correa, L., Camacho, B., Gruber, J., and Salguero, G. (2020). Integrated analysis of transcriptome and secretome from umbilical cord mesenchymal stromal cells reveal new mechanisms for the modulation of inflammation and immune activation. Front. Immunol. 11, 575488. https://doi.org/10.3389/fimmu.2020.575488.
62c. Cuerquis, J., Romieu-Mourez, R., Franc, ois, M., Routy, J.-P., Young, Y. K., Zhao, J., and Eliopoulos, N. (2014). Human mesenchymal stromal cells transiently increase cytokine production by activated T cells before sup-pressing T-cell proliferation: effect of interferon-g and tumor necrosis factor-α stimulation. Cytotherapy 16, 191-202. https://doi.org/10.1016/j.jcyt. 2013.11.008.
63c. Pan, X., and Shen, H.-B. (2018). Predicting RNA-protein binding sites and motifs through combining local and global deep convolutional neural networks. Bioinformatics 34, 3427-3436. https://doi.org/10.1093/bioinformatics/bty364.
64c. Ryder, P. V., and Lerit, D. A. (2018). RNA localization regulates diverse and dynamic cellular processes. Traffic 19, 496-502. https://doi.org/10.1111/tra.12571.
65c. Martin, K. C., Barad, M., and Kandel, E. R. (2000). Local protein synthesis and its role in synapse-specific plasticity. Curr. Opin. Neurobiol. 10, 587-592. https://doi.org/10.1016/50959-4388(00)00128-8.
66c. Obilor, E. I., and Amadi, E. C. (2018). Test for significance of Pearson's correlation coefficient. International Journal of Innovative Mathematics, Statistics & Energy Policies 6, 11-23.
67c. Chen, X., and Gu€ttel, S. (2022). Fast exact fixed-radius nearest neighbor search based on sorting. Preprint at arXiv. https://doi.org/10.48550/ARXIV.2212.07679.
68c. Sedgwick, P. (2012). Multiple significance tests: the Bonferroni correction. BMJ 344, e509. https://doi.org/10.1136/bmj.e509.
69c. Selich, A., Zimmermann, K., Tenspolde, M., Dittrich-Breiholz, O., von Kaisenberg, C., Schambach, A., and Rothe, M. (2019). Umbilical cord as a long-term source of activatable mesenchymal stromal cells for immunomodulation. Stem Cell Res. Ther. 10, 285. https://doi.org/10.1186/s13287-019-1376-9.
70c. Russell, K. C., Phinney, D. G., Lacey, M. R., Barrilleaux, B. L., Meyertholen, K. E., and O'Connor, K. C. (2010). In vitro high-capacity assay to quantify the clonal heterogeneity in trilineage potential of mesenchymal stem cells reveals a complex hierarchy of lineage commitment. Stem Cell. 28, 788-798. https://doi.org/10.1002/stem.312.
71c. Rennerfeldt, D. A., Raminhos, J. S., Leff, S. M., Manning, P., and Van Vliet, K. J. (2019). Emergent heterogeneity in putative mesenchymal stem cell colonies: single-cell time lapsed analysis. PLoS One 14, e0213452. https://doi.org/10.1371/journal.pone.0213452.
72c. Chow, K.-H. K., Budde, M. W., Granados, A. A., Cabrera, M., Yoon, S., Cho, S., Huang, T. H., Koulena, N., Frieda, K. L., Cai, L., et al. (2021). Imaging cell lineage with a synthetic digital recording system. Science 372, eabb3099. https://doi.org/10.1126/science.abb3099.
73c. Ho, Y.-T., Shimbo, T., Wijaya, E., Ouchi, Y., Takaki, E., Yamamoto, R., Kikuchi, Y., Kaneda, Y., and Tamai, K. (2018). Chromatin accessibility identifies diversity in mesenchymal stem cells from different tissue origins. Sci. Rep. 8, 17765. https://doi.org/10.1038/s41598-018-36057-0.
74c. Boehm, K. M., Khosravi, P., Vanguri, R., Gao, J., and Shah, S. P. (2022). Harnessing multimodal data integration to advance precision oncology. Nat. Rev. Cancer 22, 114-126. https://doi.org/10.1038/s41568-021-00408-3.
75c. Garali, I., Adanyeguh, I. M., Ichou, F., Perlbarg, V., Seyer, A., Colsch, B., Moszer, I., Guillemot, V., Durr, A., Mochel, F., and Tenenhaus, A. (2018). A strategy for multimodal data integration: application to biomarkers identification in spinocerebellar ataxia. Briefings Bioinf. 19, 1356-1369. https://doi.org/10.1093/bib/bbx060.
76c. Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nat. Methods 9, 676-682. https://doi.org/10.1038/nmeth.2019.
77c. Charlier, F., Weber, M., Izak, D., Harkin, E., Magnus, M., Lalli, J., Fresnais, L., Chan, M., Markov, N., Amsalem, O., et al. (2022). trevismd/statannotations: v0.5. Preprint at Zenodo. https://doi.org/10.5281/ZENODO.7213391.
78c. Waskom, M. (2021). seaborn: statistical data visualization. JOSS 6, 3021. https://doi.org/10.21105/joss.03021.
79c. Hunter, J. D. (2007). Matplotlib: a 2D graphics environment. Comput. Sci. Eng. 9, 90-95. https://doi.org/10.1109/MCSE.2007.55.
80c. Hagberg exploring network structure, dynamics, and function using NetworkX. In
81c. Team, T. P. D. (2023). pandas-dev/pandas: pandas. Preprint at Zenodo. https://doi.org/10.5281/ZENODO.3509134.
82c. Wolf, F. A., Angerer, P., and Theis, F. J. (2018). SCANPY: large-scale single-cell gene expression data analysis. Genome Biol. 19, 15. https://doi.org/10.1186/s13059-017-1382-0.
83c. van der Walt, S., Scho nberger, J. L., Nunez-Iglesias, J., Boulogne, F., Warner, J. D., Yager, N., Gouillart, E., and Yu, T.; scikit-image contributors (2014). scikit-image: image processing in Python. PeerJ 2, e453. https://doi.org/10.7717/peerj.453.
84c. Pedregosa, F., Varoquaux, G., Gramfort, A., Michel, V., Thirion, B., Grisel, O., Blondel, M., Prettenhofer, P., Weiss, R., and Dubourg, V. (2011). Scikitlearn: machine learning in Python. the Journal of machine Learning research 12, 2825-2830.
1d. Stuart, J. M., Segal, E., Koller, D., and Kim, S. K. (2003). A Gene-Coexpression Network for Global Discovery of Conserved Genetic Modules. Science 302, 249-255.10.1126/science.1087447.
2d. Tang, J., Kong, D., Cui, Q., Wang, K., Zhang, D., Gong, Y., and Wu, G. (2018). Prognostic Genes of Breast Cancer Identified by Gene Co-expression Network Analysis. Front. Oncol. 8, 374. 10.3389/fonc.2018.00374.
3d. Mercatelli, D., Scalambra, L., Triboli, L., Ray, F., and Giorgi, F. M. (2020). Gene regulatory network inference resources: A practical overview. Biochimica et Biophysica Acta (BBA)—Gene Regulatory Mechanisms 1863, 194430. 10.1016/j.bbagrm.2019.194430.
4d. Bailey, T. L., Boden, M., Buske, F. A., Frith, M., Grant, C. E., Clementi, L., Ren, J., Li, W. W., and Noble, W. S. (2009). MEME SUITE: tools for motif discovery and searching. Nucleic Acids Research 37, W202-W208. 10.1093/nar/gkp335.
5d. Kulkarni, S. R., Vaneechoutte, D., Van de Velde, J., and Vandepoele, K. (2018). TF2Network: predicting transcription factor regulators and gene regulatory networks in Arabidopsis using publicly available binding site information. Nucleic Acids Research 46, e31-e31. 10.1093/nar/gkx1279.
6d. Verfaillie, A., Imrichova, H., Janky, R., and Aerts, S. (2015). iRegulon and icisTarget: Reconstructing Regulatory Networks Using Motif and Track Enrichment. Current Protocols in Bioinformatics 52. 10.1002/0471250953.bi0216s52.
7d. Liu, Z., Gao, J., Li, T., Jing, Y., Xu, C., Zhu, Z., Zuo, D., and Chen, J. (2022). A novel approach GRNTSTE to reconstruct gene regulatory interactions applied to a case study for rat pineal rhythm gene. Scientific Reports 12. 10.1038/s41598-022-14903-6.
8d. Huynh-Thu, V. A., Irrthum, A., Wehenkel, L., and Geurts, P. (2010). Inferring Regulatory Networks from Expression Data Using Tree-Based Methods. PLoS ONE 5, e12776. 10.1371/journal.pone.0012776.
9d. Yang, Y., Fang, Q., and Shen, H.-B. (2019). Predicting gene regulatory interactions based on spatial gene expression data and deep learning. PLoS Comput Biol 15, e1007324. 10.1371/journal.pcbi.1007324.
10d. Puniyani, K., and Xing, E. P. (2013). GINI: From ISH Images to Gene Interaction Networks. PLoS Comput Biol 9, e1003227. 10.1371/journal.pcbi.1003227.
11d. Banf, M., and Rhee, S. Y. (2017). Computational inference of gene regulatory networks: Approaches, limitations and opportunities. Biochimica et Biophysica Acta (BBA) Gene Regulatory Mechanisms 1860, 41-52. 10.1016/j.bbagrm.2016.09.003.
1e. Cohen, S., Valm, A. M. & Lippincott-Schwartz, J. Interacting organelles. Current Opinion in Cell Biology 53, 84-91 (2018).
2e. Kurz, F. T. et al. Network dynamics: quantitative analysis of complex behavior in metabolism, organelles, and cells, from experiments to models and back. WIREs Systems Biology and Medicine 9, e1352 (2017).
3e. Mondal, M., Liao, R. & Guo, J. Highly Multiplexed Single-Cell Protein Analysis. Chemistry— A European Journal 24, 7083-7091 (2018).
4e. Gatto, L., Vizcaino, J. A., Hermjakob, H., Huber, W. & Lilley, K. S. Organelle proteomics experimental designs and analysis. PROTEOMICS 10, 3957-3969 (2010).
5e. Allam, M. et al. Spatially visualized single-cell pathology of highly multiplexed protein profiles in health and disease. Commun Biol 4, 1-16 (2021).
6e. Fan, X.-L., Zhang, Y., Li, X. & Fu, Q.-L. Mechanisms underlying the protective effects of mesenchymal stem cell-based therapy. Cell. Mol. Life Sci. 77, 2771-2794 (2020).
7e. Miao, C., Lei, M., Hu, W., Han, S. & Wang, Q. A brief review: the therapeutic potential of bone marrow mesenchymal stem cells in myocardial infarction. Stem Cell Research & Therapy 8, 242 (2017).
8e. Uccelli, A., Moretta, L. & Pistoia, V. Mesenchymal stem cells in health and disease. Nat Rev Immunol 8, 726-736 (2008).
9e. Wei, X. et al. Mesenchymal stem cells: a new trend for cell therapy. Acta Pharmacol Sin 34, 747-754 (2013).
10e. Salazar-Noratto, G. E. et al. Understanding and leveraging cell metabolism to enhance mesenchymal stem cell transplantation survival in tissue engineering and regenerative medicine applications. STEM CELLS 38, 22-33 (2020).
11e. Saeedi, P., Halabian, R. & Imani Fooladi, A. A. A revealing review of mesenchymal stem cells therapy, clinical perspectives and Modification strategies. Stem Cell Investig 6, 34 (2019).
12e. Wang, S. et al. ATF6 safeguards organelle homeostasis and cellular aging in human mesenchymal stem cells. Cell Discov 4, 1-19 (2018).
13e. Rodriguez, A.-M., Nakhle, J., Griessinger, E. & Vignais, M.-L. Intercellular mitochondria trafficking highlighting the dual role of mesenchymal stem cells as both sensors and rescuers of tissue injury. Cell Cycle 17, 712-721 (2018).
14e. Mahrouf-Yorgov, M. et al. Mesenchymal stem cells sense mitochondria released from damaged cells as danger signals to activate their rescue properties. Cell Death Differ 24, 1224-1238 (2017).
15e. Mokarizadeh, A. et al. Microvesicles derived from mesenchymal stem cells: Potent organelles for induction of tolerogenic signaling. Immunology Letters 147, 47-54 (2012).
16e. Paliwal, S., Chaudhuri, R., Agrawal, A. & Mohanty, S. Regenerative abilities of mesenchymal stem cells through mitochondrial transfer. J Biomed Sci 25, 31 (2018).
17e. Viana, M. P. et al. Robust integrated intracellular organization of the human iPS cell: where, how much, and how variable? bioRxiv (2020) doi:10.1101/2020.12.08.415562.
18e. Kabat, M., Bobkov, I., Kumar, S. & Grumet, M. Trends in mesenchymal stem cell clinical trials 2004-2018: Is efficacy optimal in a narrow dose range?Stem Cells Transl Med 9, 17-27 (2019).
19e. Du, W. J. et al. Heterogeneity of proangiogenic features in mesenchymal stem cells derived from bone marrow, adipose tissue, umbilical cord, and placenta. Stem Cell Research & Therapy 7, 163 (2016).
20e. Yousefifard, M. et al. Human bone marrow-derived and umbilical cord-derived mesenchymal stem cells for alleviating neuropathic pain in a spinal cord injury model. Stem Cell Research & Therapy 7, 36 (2016).
21e. Giesen, C. et al. Highly multiplexed imaging of tumor tissues with subcellular resolution by mass cytometry. Nat Methods 11, 417-422 (2014).
22e. Keren, L. et al. MIBI-TOF: A multiplexed imaging platform relates cellular phenotypes and tissue structure. Science Advances 5, eaax5851 (2019).
23e. Goltsev, Y. et al. Deep Profiling of Mouse Splenic Architecture with CODEX Multiplexed Imaging. Cell 174, 968-981.e15 (2018).
24e. Gerdes, M. J. et al. Highly multiplexed single-cell analysis of formalin-fixed, paraffin-embedded cancer tissue. PNAS 110, 11982-11987 (2013).
25e. Lin, J.-R., Fallahi-Sichani, M. & Sorger, P. K. Highly multiplexed imaging of single cells using a high-throughput cyclic immunofluorescence method. Nat Commun 6, 8390 (2015).
26e. Gut, G., Herrmann, M. D. & Pelkmans, L. Multiplexed protein maps link subcellular organization to cellular states. Science 361, (2018).
27e. Cappi, G., Dupouy, D. G., Comino, M. A. & Ciftlik, A. T. Ultra-fast and automated immunohistofluorescent multistaining using a microfluidic tissue processor. Sci Rep 9, 4489 (2019).
28e. Ko, J., Oh, J., Ahmed, M. S., Carlson, J. C. T. & Weissleder, R. Ultra-fast Cycling for Multiplexed Cellular Fluorescence Imaging. Angewandte Chemie 132, 6906-6913 (2020).
29e. Bray, M.-A. et al. Cell Painting, a high-content image-based assay for morphological profiling using multiplexed fluorescent dyes. Nat Protoc 11, 1757-1774 (2016).
30e. Merendino, A. M. et al. Hsp60 Is Actively Secreted by Human Tumor Cells. PLOS ONE 5, e9247 (2010).
31e. Cappello, F., Conway de Macario, E., Marasa, L., Zummo, G. & Macario, A. J. L. Hsp60 expression, new locations, functions, and perspectives for cancer diagnosis and therapy. Cancer Biology & Therapy 7, 801-809 (2008).
32e. Bhagawati, M. et al. The receptor subunit Tom20 is dynamically associated with the TOM complex in mitochondria of human cells. MBoC 32, br1 (2021).
33e. Lujan, P. & Campelo, F. Should I stay or should I go?Golgi membrane spatial organization for protein sorting and retention. Archives of Biochemistry and Biophysics 707, 108921 (2021).
34e. Kjolby, M., Nielsen, M. S. & Petersen, C. M. Sortilin, Encoded by the Cardiovascular Risk Gene SORT1, and Its Suggested Functions in Cardiovascular Disease. Curr Atheroscler Rep 17, 18 (2015).
35e. Adler, J. & Parmryd, I. Quantifying colocalization by correlation: the Pearson correlation coefficient is superior to the Mander's overlap coefficient. Cytometry A 77, 733-742 (2010).
36e. Dunn, K. W., Kamocka, M. M. & McDonald, J. H. A practical guide to evaluating colocalization in biological microscopy. American Journal of Physiology-Cell Physiology 300, C723-C742 (2011).
37e. Zhou, B. Image Segmentation using SLIC Superpixels and Affinity Propagation Clustering. in (2015).
38e. Venkatesan, M. et al. Virtual and augmented reality for biomedical applications. CR Med 2, (2021).
39e. Schwarz, N. & Leube, R. E. Intermediate Filaments as Organizers of Cellular Space: How They Affect Mitochondrial Structure and Function. Cells 5, 30 (2016).
40e. Kaasik, A. et al. Energetic Crosstalk Between Organelles. Circulation Research 89, 153-159 (2001).
41e. de Brito, O. M. & Scorrano, L. An intimate liaison: spatial organization of the endoplasmic reticulum-mitochondria relationship. The EMBO Journal 29, 2715-2723 (2010).
42e. Del Fattore, A. et al. Differential effects of extracellular vesicles secreted by mesenchymal stem cells from different sources on glioblastoma cells. Expert Opinion on Biological Therapy 15, 495-504 (2015).
43e. Mazini, L., Rochette, L., Amine, M. & Malka, G. Regenerative Capacity of Adipose Derived Stem Cells (ADSCs), Comparison with Mesenchymal Stem Cells (MSCs). International Journal of Molecular Sciences 20, 2523 (2019).
44e. Contentin, R. et al. Comparison of the Chondrogenic Potential of Mesenchymal Stem Cells Derived from Bone Marrow and Umbilical Cord Blood Intended for Cartilage Tissue Engineering. Stem Cell Rev Rep 16, 126-143 (2020).
45e. Heo, J. S., Choi, Y., Kim, H.-S. & Kim, H. O. Comparison of molecular profiles of human mesenchymal stem cells derived from bone marrow, umbilical cord blood, placenta and adipose tissue. International Journal of Molecular Medicine 37, 115-125 (2016).
46e. Lavrentieva, A., Majore, I., Kasper, C. & Hass, R. Effects of hypoxic culture conditions on umbilical cord-derived human mesenchymal stem cells. Cell Communication and Signaling 8, 18 (2010).
47e. Schop, D. et al. Growth, Metabolism, and Growth Inhibitors of Mesenchymal Stem Cells. Tissue Engineering Part A 15, 1877-1886 (2009).
48e. Wang, W. et al. Live-cell imaging and analysis reveal cell phenotypic transition dynamics inherently missing in snapshot data. Science Advances 6, eaba9319 (2020).
49e. Boeynaems, S. et al. Protein Phase Separation: A New Phase in Cell Biology. Trends in Cell Biology 28, 420-435 (2018).
50e. Berchtold, D., Battich, N. & Pelkmans, L. A Systems-Level Study Reveals Regulators of Membrane-less Organelles in Human Cells. Molecular Cell 72, 1035-1049.e5 (2018).
51e. Banani, S. F., Lee, H. O., Hyman, A. A. & Rosen, M. K. Biomolecular condensates: organizers of cellular biochemistry. Nat Rev Mol Cell Biol 18, 285-298 (2017).
52e. Shin, Y. & Brangwynne, C. P. Liquid phase condensation in cell physiology and disease. Science 357, (2017).
53e. Pan, C., Kumar, C., Bohl, S., Klingmueller, U. & Mann, M. Comparative Proteomic Phenotyping of Cell Lines and Primary Cells to Assess Preservation of Cell Type-specific Functions. Mol Cell Proteomics 8, 443-450 (2009).
54e. Cai, S., Allam, M. & Coskun, A. F. Multiplex Spatial Bioimaging for Combination Therapy Design. Trends in Cancer 6, 813-818 (2020).
55e. Shamekhi Amiri, F. Intracellular organelles in health and kidney disease. Néphrologie & Thérapeutique 15, 9-21 (2019).
56e. Redhai, S. & Boutros, M. The Role of Organelles in Intestinal Function, Physiology, and Disease. Trends in Cell Biology 31, 485-499 (2021).
57e. C. Ubah, O. & M. Wallace, H. Cancer Therapy: Targeting Mitochondria and other Sub-cellular Organelles. Current Pharmaceutical Design 20, 201-222 (2014).
58e. Terman, A., Gustafsson, B. & Brunk, U. T. Autophagy, organelles and ageing. The Journal of Pathology 211, 134-143 (2007).
59e. Levin, M. The wisdom of the body: future techniques and approaches to morphogenetic fields in regenerative medicine, developmental biology and cancer. Regenerative Medicine 6, 667-673 (2011).
60e. Yousefi, J. Image Binarization using Otsu Thresholding Algorithm. (2015). doi:10.13140/RG.2.1.4758.9284.
61e. Software: RNAscope HiPlex Image Registration|In Situ Hybridization, RNA-ISH I ACDBio. https://acdbio.com/product-type2/software-rnascope-hiplex-image-registration.
62e. van der Walt, S. et al. scikit-image: image processing in Python. PeerJ2, e453 (2014).
63e. Stefani, C., Lacy-Hulbert, A. & Skillman, T. ConfocalVR: Immersive Visualization for Confocal Microscopy. Journal of Molecular Biology 430, 4028-4035 (2018).
64e. Blanc, T., El Beheiry, M., Caporal, C., Masson, J.-B. & Hajj, B. Genuage: visualize and analyze multidimensional single-molecule point cloud data in virtual reality. Nat Methods 17, 1100-1102 (2020).
1f. Cappello, F., Conway de Macario, E., Marasa, L., Zummo, G. & Macario, A. J. L. Hsp60 expression, new locations, functions, and perspectives for cancer diagnosis and therapy. Cancer Biology & Therapy 7, 801-809 (2008).
2f. Merendino, A. M. et al. Hsp60 Is Actively Secreted by Human Tumor Cells. PLOS ONE 5, e9247 (2010).
3f. Recombinant Anti-Hsp60 antibody [EPR18245-93]— BSA and Azide free (ab224528). https://www.abcam.com/Hsp60-antibody-EPR18245-93-BSA-and-Azide-free-ab224528.html.
4f. Bhagawati, M. et al. The receptor subunit Tom20 is dynamically associated with the TOM complex in mitochondria of human cells. MBoC 32, br1 (2021).
5f. Anti-Tom20 Antibody (F-10). https://www.scbt.com/p/tom20-antibody-f-10.
6f. Hillary, R. F. & FitzGerald, U. A lifetime of stress: ATF6 in development and homeostasis. Journal of Biomedical Science 25, 48 (2018).
7f. Recombinant Anti-ATF6 antibody [EPR22690-84]— BSA and Azide free KO Tested (ab263955). https://www.abcam.com/atf6-antibody-epr22690-84-bsa-and-azide-free-50 ab263955.html.
8f. Lujan, P. & Campelo, F. Should I stay or should I go?Golgi membrane spatial organization for protein sorting and retention. Archives of Biochemistry and Biophysics 707, 108921 (2021).
9f. Recombinant Anti-GOLPH4/GPP130 antibody [EPR13439] KO Tested (ab197595) 1 Abcam. https://www.abcam.com/golph4gpp130-antibody-epr13439-ab197595.html.
10f. Kjolby, M., Nielsen, M. S. & Petersen, C. M. Sortilin, Encoded by the Cardiovascular Risk Gene SORT1, and Its Suggested Functions in Cardiovascular Disease. Curr Atheroscler Rep 17, 18 (2015).
11f. Recombinant Anti-Sortilin/NT3 antibody [EPR23093-58]— BSA and Azide free (ab263873). https://www.abcam.com/sortilinnt3-antibody-epr23093-58-bsa-and-azide-free-ab263873.html.
12f. Concanavalin A, Alexa Fluor 488 Conjugate. https://www.thermofisher.com/order/catalog/product/C11252.
13f. Bray, M.-A. et al. Cell Painting, a high-content image-based assay for morphological profiling using multiplexed fluorescent dyes. Nat Protoc 11, 1757-1774 (2016).
14f. Alexa Fluor 555 Phalloidin. https://www.thermofisher.com/order/catalog/product/A34055.
15f. Wheat Germ Agglutinin, Alexa Fluor 647 Conjugate. https://www.thermofisher.com/order/catalog/product/W32466.
16f. Recombinant Anti-Nucleolin antibody [EPR7952]— BSA and Azide free (ab226113). https://www.abcam.com/nucleolin-antibody-epr7952-bsa-and-azide-free-ab226113.html.
17f. Anti-β Tubulin Antibody (D-10). https://www.scbt.com/p/beta-tubulin-antibody-d-10;jsessionid=g0cw-4HJEYyJsQi4LQp75hxE-9PtyQdjUrVTyytURt6f_laA6hUh!1267813408.
1g. Battich, N., Stoeger, T. & Pelkmans, L. Image-based transcriptomics in thousands of single human cells at single-molecule resolution. Nature methods 10, 1127-1133 (2013).
2g. Eng, C.-H. L. et al. Transcriptome-scale super-resolved imaging in tissues by RNA seqFISH+. Nature 568, 235-239 (2019).
3g. Xia, C., Fan, J., Emanuel, G., Hao, J. & Zhuang, X. Spatial transcriptome profiling by MERFISH reveals subcellular RNA compartmentalization and cell cycle-dependent gene expression. Proc. Natl. Acad. Sci. U.S.A. 116, 19490-19499 (2019).
4g. Moffitt, J. R. et al. Molecular, spatial, and functional single-cell profiling of the hypothalamic preoptic region. Science 362, eaau5324 (2018).
5g. Fang, R. et al. Three-dimensional single-cell transcriptome imaging of thick tissues. bioRxiv 2023-07 (2023).
6g. Wang, Y. et al. EASI-FISH for thick tissue defines lateral hypothalamus spatio-molecular organization. Cell 184, 6361-6377.e24 (2021).
7g. Nicolas, J. et al. 3D Extracellular Matrix Mimics: Fundamental Concepts and Role of Materials Chemistry to Influence Stem Cell Fate. Biomacromolecules 21, 1968-1994 (2020).
8g. Cruz-Acuña, R. et al. Synthetic hydrogels for human intestinal organoid generation and colonic wound repair. Nat Cell Biol 19, 1326-1335 (2017).
9g. Phelps, E. A. et al. Maleimide Cross-Linked Bioactive PEG Hydrogel Exhibits Improved Reaction Kinetics and Cross-Linking for Cell Encapsulation and In Situ Delivery. Adv. Mater. 24, 64-70 (2012).
10g. Lee, T. T. et al. Light-triggered in vivo activation of adhesive peptides regulates cell adhesion, inflammation and vascularization of biomaterials. Nature Mater 14, 352-360 (2015).
11g. Graney, P. L. et al. Organoid Polymer Functionality and Mode of Klebsiella pneumoniae Membrane Antigen Presentation Regulates Ex Vivo Germinal Center 50 Epigenetics in Young and Aged B Cells. Adv. Funct. Mater. 30, 2001232 (2020).
12g. Patel, R. G. et al. Microscale Bioadhesive Hydrogel Arrays for Cell Engineering Applications. Cel. Mol. Bioeng. 7, 394-408 (2014).
13g. Purwada, A. et al. Ex vivo synthetic immune tissues with T cell signals for differentiating antigen-specific, high affinity germinal center B cells. Biomaterials 198, 27-36 (2019).
14g. Clark, A. Y. et al. Integrin-specific hydrogels modulate transplanted human bone marrow-derived mesenchymal stem cell survival, engraftment, and reparative activities. Nat Commun 11, 114 (2020).
15g. Schneider, R. S. et al. High-Throughput On-Chip Human Mesenchymal Stromal Cell Potency Prediction. Adv Healthcare Materials 11, 2101995 (2022).
16g. Aggarwal, S. & Pittenger, M. F. Human mesenchymal stem cells modulate allogeneic immune cell responses. Blood 105, 1815-1822 (2005).
17g. Li, M. M., Huang, K. & Zitnik, M. Graph representation learning in biomedicine and healthcare. Nat. Biomed. Eng 6, 1353-1369 (2022).
18g. Hu, J. et al. SpaGCN: Integrating gene expression, spatial location and histology to identify spatial domains and spatially variable genes by graph convolutional network. Nat Methods 18, 1342-1351 (2021).
19g. Long, Y. et al. Spatially informed clustering, integration, and deconvolution of spatial transcriptomics with GraphST. Nat Commun 14, 1155 (2023).
20g. Fey, M. & Lenssen, J. E. Fast graph representation learning with PyTorch Geometric. arXiv preprint arXiv:1903.02428 (2019).
21g. Argelaguet, R., Cuomo, A. S. E., Stegle, O. & Marioni, J. C. Computational principles and challenges in single-cell data integration. Nat Biotechnol 39, 1202-1215 (2021).
22g. Dunn, C. M. et al. Interferon-Gamma Primed Human Clonal Mesenchymal Stromal Cell Sheets Exhibit Enhanced Immunosuppressive Function. Cells 11, 3738 (2022).
23g. Guess, A. J. et al. Safety Profile of Good Manufacturing Practice Manufactured Interferon γ-Primed Mesenchymal Stem/Stromal Cells for Clinical Trials. Stem Cells Translational Medicine 6, 1868-1879 (2017).

What is claimed is:

1. A method of characterizing a nucleic acid network comprising:
(i) providing a biological sample on a substrate, wherein the biological sample comprises the nucleic acid network;
(ii) applying spatially-aware graph neural networks (spaGNN) to the biological sample to obtain characterization data; and
(iii) analyzing the characterization data.

2. The method of claim 1, wherein the substrate comprises a coverslip, a co-culture, or any combination thereof.

3. The method of claim 1, wherein the substrate comprises glass.

4. The method of claim 1, wherein the substrate is from 100 to 150 μm thick.

5. The method of claim 1, wherein the substrate is coated with a layer of a hydrogel, Matrigel, collagen, cultrex, or any combination thereof.

6. The method of claim 1, wherein the biological sample comprises a tissue sample.

7. The method of claim 6, wherein the tissue sample comprises an umbilical cord, adipose tissue, or bone marrow.

8. The method of any claim 1, wherein the biological sample comprises a stem cell.

9. The method of claim 8, wherein the stem cell comprises a mesenchymal stem cell (MSC).

10. The method of claim 9, wherein the MSC comprises an organelle.

11. The method of claim 10, wherein the organelle is a nucleus, mitochondria, a Golgi, an endoplasmic reticulum, or any combination thereof.

12. The method of claim 1, wherein the biological sample comprises an immune cell.

13. The method of claim 1, wherein the nucleic acid network comprises RNA, DNA, or any combination thereof.

14. The method of claim 1, wherein the characterization data comprises information relating to the nucleic acid network potency, identity, proximity, communication, therapeutic efficacy, interactions, associated energy, or a combination thereof.

15. The method of claim 1, wherein analyzing the characterization data comprises obtaining a scatter plot of intensity, calculating a Pearson's correlation coefficient, calculating a pixel overlap colocalization, conducting the Kolmogorov-Smirnov hypothesis test, plotting average intensity values per cell as a boxplot, clustering pixel phenotypes, calculating pixel intensity, energy Laplacian, modified Laplacian, diagonal Laplacian, variance Laplacian, or gray level variance, or obtaining morphological features for each cell.

16. The method of claim 1, wherein providing the biological sample on the substrate comprises culturing the cells on the substrate.

17. The method of claim 16, wherein the biological sample comprises one or more mesenchymal stem cells (MSCs) and one or more immune cells, wherein the MSCs and immune cells are co-cultured on a 3D substrate.

18. The method of claim 1, wherein providing the biological sample on the substrate comprises performing immunofluorescence on the biological sample, thereby staining the stem cell with a marker.

19. The method of claim 1, wherein artificial intelligence and/or machine learning is used to analyze the characterization data.

20. A method of providing stem cell therapy to a subject in need thereof comprising:
(i) performing the method of claim 1 to a stem cell; and
(ii) administering the stem cell to the subject.

21. The method of claim 20, wherein performing the method on the stem cell comprises identifying RNA expression on the stem cell.

22. The method of claim 20, wherein the subject has anemia, a blood disorder, an inherited red cell abnormality, a bone marrow cancer, leukemia, lymphoma, an inherited immune disorder, an inherited metabolic disorder, an inherited platelet abnormality, a phagocyte disorder, a solid tumor, or an immune or other system disorder.

23. A kit comprising a stem cell targeting reagent and an organelle map, wherein the organelle map comprises data indicating identification and proximity of RNA inside of an organelle and outside of an organelle.

24. The kit of claim 23, wherein the map is software-based.


SEQUENCE LISTING (7 sequences)

SEQ ID NO: 1 - DNA, 22 bp, synthetic construct
  misc_feature 1..22: note = Description of sequence: Oligonucleotide, primer
  source 1..22: mol_type = other DNA
tatgacagaactagacactctt

SEQ ID NO: 2 - DNA, 32 bp, synthetic construct
  misc_feature 1..32: note = Description of sequence: Oligonucleotide, PLA-based sequence
  source 1..32: mol_type = other DNA
  modified_base 1: mod_base = OTHER; note = nucleotide bound to thiol group
aaaaaaaaaagacgctaatagttaagacgctt

SEQ ID NO: 3 - protein, 7 aa, synthetic construct
  REGION 1..7: note = Description of sequence: Peptide, thiolated RGD peptide
  MOD_RES 1: note = Amino bound to thiol group
GRGDSPC

SEQ ID NO: 4 - protein, 16 aa, synthetic construct
  REGION 1..16: note = Description of sequence: Peptide, MMP9-degradable VPM
GCRDVPMSMRGGDRCG

SEQ ID NO: 5 - protein, 6 aa, synthetic construct
  REGION 1..6: note = Description of sequence: Peptide, GFOGER peptide mimic
  SITE 3: note = 3Hyp
GFXGER

SEQ ID NO: 6 - protein, 45 aa, synthetic construct
  REGION 1..45: note = Description of sequence: Peptide, collagen-mimetic GFOGER peptide
  SITE 24: note = 3Hyp
GYGGGPGPPGPPGPPGPPGPPGFXGERGPPGPPGPPGPPGPPGPC

SEQ ID NO: 7 - protein, 7 aa, synthetic construct
  REGION 1..7: note = Description of sequence: Peptide, adhesive RGD peptide
GRGDSPC